SEQUENCE LISTING (77 sequences)

SEQ ID NO: 1 - protein, 464 aa, Influenza virus
MNPNQKIITIGSVSLTISTICFFMQIAILITAVTLHFKQYEFNSPMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNNIVHDRTPYRTLLMNELGVPFHLGTKQVCIAWSSSSCHDGKAWLHVCVTGDDENATASFIYNGRLADSIVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKDDSSSSSHCLDPNNEEGGQGVKGWAFDDGNDVWMGRTISEKLRSGYETFKVIEGWSNPNSKLQINRQVIVDRGNRSGYSGIFSVEGKSCINRCFYVELIRGRKQETEVLWTSNSIVVFCGTSGTYGTGSWPDGADINLMPI

SEQ ID NO: 2 - protein, 469 aa, Influenza virus
MNPNQKIITIGSVSLTISTICFFMQIAILITTVTLHFKQYEFNSPPNNQVMLCEPTIIERNVTEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSKLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 3 - protein, 469 aa, Influenza virus
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDIVHDRTPYRTLLMNELGVPFHLGTKQVCIAWSSSSCHDGKAWLHVCVTGDDENATASFIYNGRLADSIVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKQETEVLWTSNSIVVFCGTSGTYGTGSWPDGADINLMPI

SEQ ID NO: 4 - DNA, 2341 bp, Influenza virus
  source 1..2341: mol_type = other DNA
agcaaaagcaggtcaattatattcagtatggaaagaataaaagaactacggaacctgatgtcgcagtctcgcactcgcgagatactgacaaaaaccacagtggaccatatggccataattaagaagtacacatcggggagacaggaaaagaacccgtcacttaggatgaaatggatgatggcaatgaaatacccaatcactgctgacaaaaggataacagaaatggttccggagagaaatgaacaaggacaaactctatggagtaaaatgagtgatgctggatcagatcgagtgatggtatcacctttggctgtgacatggtggaatagaaatggacccgtgacaagtacggtccattacccaaaagtatacaagacttattttgacaaagtcgaaaggttaaaacatggaacctttggccctgttcattttagaaatcaagtcaagatacgccgaagagtagacacaaaccctggtcatgcggacctcagtgccaaggaggcacaagatgtaattatggaagttgtttttcccaatgaagtgggagccaggatactaacatcagaatcgcaattaacaataactaaagagaaaaaagaagaactccgagattgcaaaatttctcccttgatggttgcatacatgttagagagagaacttgtccgaaaaacaagatttctcccagttgctggcggaacaagcagtatatacattgaagttttacatttgactcaagggacgtgttgggaacaaatgtacactccaggtggagaagtgaggaatgacgatgttgaccaaagcctaattattgcagccaggaacatagtaagaagagccgcagtatcagcagatccactagcatctttattggagatgtgccacagcacacaaattggcgggacaaggatggtggacattcttagacagaacccgactgaagaacaagctgtggatatatgcaaggctgcaatgggattgagaatcagctcatccttcagctttggtgggtttacatttaaaagaacaagcgggtcatcagtcaaaaaagaggaagaagtgcttacaggcaatctccaaacattgaagataagagtacatgaggggtatgaggagttcacaatggtggggaaaagagcaacagctatactcagaaaagcaaccagaagattggttcagctcatagtgagtggaagagacgaacagtcaatagccgaagcaataattgtggccatggtgttttcacaagaggattgcatgataaaagcagttagaggtgacctgaatttcgtcaacagagcaaatcagcggttgaaccccatgcatcagcttttaaggcattttcagaaagatgcgaaagtgctttttcagaattggggaattgaacacatcgacagtgtaatgggaatggttggagtattaccagatatgactccaagcacagagatgtcaatgagaggaataagagtcagcaaaatgggtgtggatgaatactccagtacagagagggtggtggttagcattgatcggtttttgagagttcgagaccaacgcgggaatgtattattatctcctgaagaggttagtgaaacacagggaactgagagactgacaataacttattcatcgtcgatgatgtgggagattaacggtcctgagtcggttttggtcaatacttatcaatggatcatcagaaattgggaagctgtcaaaattcaatggtctcagaatcctgcaatgttgtacaacaaaatggaatttgaaccatttcaatctttagtccccaaggccattagaagccaatacagtgggtttgtcagaactctattccaacaaatgagagacgtacttgggacatttgacaccacccagataataaagcttctcccttttgcagccgctccaccaaagcaaagcagaatgcagttctcttcactgactgtaaatgtgaggggatcagggatgagaatacttgtaaggggcaattctcctgtattcaactacaacaagaccactaaaagactaacaattctcggaaaagatgccggcactttaattgaagacccagatgaaagcacatccggagtggagtccgctgtattgagagggtttctcattataggtaaggaagacagaagatacgggccagcattaagcatcaatgaactgagtaaccttgcaaaaggggaaaaggctaatgtgctaatcgggcaaggagacgtggtgttggtaatgaaacgaaaacgggactctagcatacttactgacagccagacagcgaccaaaagaattcggatggccatcaattaatgttgaatagtttaaaaacgaccttgtttctact

SEQ ID NO: 5 - DNA, 2341 bp, Influenza virus
  source 1..2341: mol_type = other DNA
agcaaaagcaggcaaaccatttgaatggatgtcaatccgactctactgttcctaaaggttccagcgcaaaatgccataagcaccacattcccttatactggagatcctccatacagccatggaacaggaacagggtacaccatggacacagtcaacagaacacaccaatattcagataaggggaagtggacgacaaatacagaaactggggcaccccaactcaacccaattgatggaccactacctgaggataatgagccaagtggatatgcacaaacagactgtgtcctggaggctatggccttccttgaagaatcccacccaggtatctttgagaactcatgccttgaaacaatggaagtcgttcaacaaacaagggtggacaaactaacccaaggtcgccagacttatgattggacattaaacagaaatcaaccggcagcaactgcattagccaacaccatagaagtttttagatcgaatggactaacagctaatgaatcaggaaggctaatagatttcctcaaggatgtgatggaatcaatggataaagaggaaatggagataacaacacactttcaaagaaaaaggagagtaagagacaacatgaccaagaaaatggtcacacaaagaacaatagggaagaaaaaacaaagagtgaataagagaggctatctaataagagctttgacattgaacacgatgaccaaagatgcagagagaggtaaattaaaaagaagggctattgcaacacccgggatgcaaattagagggttcgtgtacttcgttgaaactttagctagaagcatttgcgaaaagcttgaacagtctggacttccggttgggggtaatgaaaagaaggccaaactggcaaatgttgtgagaaaaatgatgactaattcacaagacacagagctttctttcacaatcactggggacaacactaagtggaatgaaaatcaaaaccctcgaatgtttttggcgatgattacatatatcacaaaaaatcaacctgagtggttcagaaacatcctgagcatcgcaccaataatgttctcaaacaaaatggcaagactgggaaaaggatacatgttcgagagtaagagaatgaaactccgaacacaaatacccgcagaaatgctagcaaacattgacctgaagtatttcaatgaatcaacaaggaagaaaattgagaaaataaggcctcttctaatagatggcacagcatcattgagccctgggatgatgatgggcatgttcaacatgctaagtacggttttaggagtctcgatactgaatcttgggcaaaagaaatacaccaagacaacatactggtgggatgggctccaatcctccgacgattttgccctcatagtgaatgcaccaaatcatgagggaatacaagcaggagtggatagattttacaggacctgcaagttagtgggaatcaacatgagcaaaaagaagtcctatataaataaaacagggacatttgaattcacaagctttttttatcgatatggatttgtggctaattttagcatggagctgcccagttttggagtgtctggaataaacgagtcagctgatatgagcattggagtaacagtgataaagaacaacatgataaacaatgaccttggaccagcaacagcccagatggctctccaattgttcatcaaagactacagatatacatataggtgccatagaggagacacacaaattcagacgagaagatcattcgagctaaagaagctgtgggatcaaacccaatcaagggcaggactattggtatcagatgggggaccaaacttatacaatatccggaatcttcacatccctgaagtctgcttaaagtgggagctaatggatgagaattatcggggaagactttgtaatcccctgaatccctttgtcagccataaagaaattgagtctgtaaacaatgctgtagtgatgccagcccatggtccggccaaaagtatggaatatgatgccgttgcaactacacactcctggattcccaagaggaaccgctctattctcaacacaagccaaaggggaattcttgaggatgaacagatgtaccagaagtgctgcaacttgttcgagaaatttttccctagtagttcatataggagaccgattggaatttctagcatggtggaggccatggtgtctagggcccggattgatgccagaattgacttcgagtctggacggattaagaaggaagagttctctgagatcatgaagatctgttccaccattgaagaactcagacggcaaaaataatgaatttagcttgtccttcatgaaaaaatgccttgtttctact

SEQ ID NO: 6 - DNA, 2233 bp, Influenza virus
  source 1..2233: mol_type = other DNA
agcaaaagcaggtactgattcgaaatggaagattttgtgcgacaatgcttcaacccgatgattgtcgaacttgcagaaaaagcaatgaaagagtatggggaggatctgaaaattgaaacaaacaaatttgcagcaatatgcactcacttggaggtatgtttcatgtattcagattttcatttcatcaatgaacaaggcgaatcaatagtggtagaacttgatgatccaaatgcactgttaaagcacagatttgaaataatcgaggggagagacagaacaatggcctggacagtagtaaacagtatctgcaacactactggagctgaaaaaccgaagtttctaccagatttgtatgattacaaggagaacagattcatcgaaattggagtgacaaggagagaagtccacatatattaccttgaaaaggccaataagattaaatctgagaacacacacattcacattttctcattcactggggaggaaatggccacaaaggcagactacactctcgacgaggaaagcagggctaggattaagaccaggctatttaccataagacaagaaatggccaacagaggcctctgggattcctttcgtcagtccgaaagaggcgaagaaacaattgaagaaaaatttgaaatctcaggaactatgcgtaggcttgccgaccaaagtctcccaccgaacttctcctgccttgagaattttagagcctatgtggatggattcgaaccgaacggctgcattgagggcaagctttctcaaatgtccaaagaagtgaatgcccaaattgaaccttttctgaagacaacaccaagaccaatcaaacttccgaatggacctccttgttatcagcggtccaagttcctcctgatggatgctttaaaattgagcattgaagacccaagtcacgaaggagaagggatcccattatatgatgcgatcaagtgcataaaaacattctttggatggaaagaaccttatatagtcaaaccacacgaaaagggaataaattcaaattacctgctgtcatggaagcaagtattgtcagaattgcaggacattgaaaatgaggagaagattccaaggactaaaaacatgaagaaaacgagtcaactaaagtgggctcttggtgagaacatggcaccagagaaagtagactttgaaaactgcagagacataagcgatttgaagcaatatgatagtgacgaacctgaattaaggtcactttcaagctggatacagaatgagttcaacaaggcctgcgagctaactgattcaatctggatagagctcgatgaaattggagaggacgtagccccaattgaatacattgcaagcatgaggaggaattatttcacagcagaggtgtcccattgtagagccactgagtacataatgaagggggtatacattaatactgccctgctcaatgcatcctgtgcagcaatggacgattttcaactaattcccatgataagcaagtgcagaactaaagagggaaggcgaaaaaccaatttatatggattcatcataaagggaagatctcatttaaggaatgacacagatgtggtaaactttgtgagcatggagttttctctcactgacccgagacttgagccacataaatgggagaaatactgtgtccttgagataggagatatgttactaagaagtgccataggccaaatttcaaggcctatgttcttgtatgtgaggacaaacggaacatcaaaggtcaaaatgaaatggggaatggagatgagacgttgcctccttcagtcactccagcagatcgagagcatgattgaagccgagtcctcggttaaagagaaagacatgaccaaagagttttttgagaataaatcagaagcatggcccattggggagtcccccaagggagtggaagaaggttccattgggaaagtctgtaggactctattggctaagtcagtgttcaatagcctgtatgcatcaccacaattggaaggattttcagcggagtcaagaaaactgctccttgttgttcaggctcttagggacaacctcgaacctgggacctttgatcttggggggctatatgaagcaattgaggagtgcctgattaatgatccctgggttttgctcaatgcgtcttggttcaactccttcctgacacatgcattaaaatagttatggcagtgctactatttgttatccgtactgtccaaaaaagtaccttgtttctact

SEQ ID NO: 7 - DNA, 1762 bp, Influenza virus
  source 1..1762: mol_type = other DNA
agcaaaagcaggggataattctattaaccatgaagactatcattgctttgagctacattctatgtctggttttcgctcaaaagcttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtggaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagcttcaattggactggagtcactcagaatggaacaagctctgcttgcaaaaggagatctaataaaagtttctttagtagattgaattggttgacccacttaaaatacaaatacccagcattgaacgtgactatgccaaacaatgaaaaatttgacaaattgtacatttggggggttcaccacccgggtacggacagtgatcaaatcagcctatatgctcaagcatcaggaagaatcacagtctctaccaaaagaagccaacaaactgtaatcccgaatatcggatctagacccagggtaagggatgtctccagcagaataagcatctattggacaatagtaaaaccgggagacatacttttgattaacagcacagggaatctaattgctcctcggggttacttcaaaatacgaagtgggaaaagctcaataatgagatcagatgcacccattggcaaatgcaattctgaatgcatcactccaaatggaagcattcccaatgacaaaccatttcaaaatgtaaacaggatcacatatggggcctgtcccagatatgttaagcaaaacactctgaaattggcaacagggatgcgaaatgtaccagagaaacaaactagaggcatatttggcgcaatcgcgggtttcatagaaaatggttgggagggaatggtggacggttggtacggtttcaggcatcaaaattctgagggcacaggacaagcagcagatctcaaaagcactcaagcagcaatcaaccaaatcaatgggaaactgaataggttaatcgggaaaacaaacgagaaattccatcagattgaaaaagaattctcagaagtagaagggagaattcaggacctcgagaaatatgttgaggacactaaaatagatctctggtcatacaacgcggagcttcttgttgccctggagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcatagagtcaatcagaaatggaacttatgaccatgatgtatacagagatgaagcattaaacaaccggttccagatcaaaggtgttgagctgaagtcaggatacaaagattggatcctatggatttcctttgccatatcatgttttttgctctgtgttgctttgttggggttcatcatgtgggcctgccaaaaaggcaacattaggtgcaacatttgcatttgagtgcattaattaaaaacacccttgtttctact

SEQ ID NO: 8 - DNA, 1565 bp, Influenza virus
  source 1..1565: mol_type = other DNA
agcaaaagcagggttaataatcactcactgagtgacatcaaaatcatggcgtcccaaggcaccaaacggtcttatgaacagatggaaactgatggggatcgccagaatgcaactgagattagggcatccgtcgggaagatgattgatggaattgggagattctacatccaaatgtgcactgaacttaaactcagtgattatgaagggcggttgatccagaacagcttgacaatagagaaaatggtgctctctgcttttgatgaaagaaggaataaatatctggaagaacaccccagcgcggggaaagatcctaagaaaactggggggcccatatacaggagagtagatggaaaatggatgagggaactcgtcctttatgacaaagaagaaataaggcgaatctggcgccaagccaacaatggtgaggatgcgacagctggtctaactcacataatgatctggcattccaatttgaatgatgcaacataccagaggacaagagctcttgttcgaaccggaatggatcccagaatgtgctctctgatgcagggctcgactctccctagaaggtccggagctgcaggtgctgcagtcaaaggaatcgggacaatggtgatggagctgatcagaatggtcaaacgggggatcaacgatcgaaatttctggagaggtgagaatgggcggaaaacaagaagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattgatattgagaggatcagttgctcacaaatcttgcctacctgcctgtgtgtatggacctgcagtatccagtgggtacgacttcgaaaaagagggatattccttggtgggaatagaccctttcaaactacttcaaaatagccaagtatacagcctaatcagacctaacgagaatccagcacacaagagtcagctggtatggatggcatgccattctgctgcatttgaagatttaagattgttaagcttcatcagagggacaaaagtatctccacgagggaaactttcaactagaggagtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaagaacttcagacatgagggcagaaatcataagaatgatggaaggtgcaaaaccagaagaagtgtcgttccgggggaggggagttttcgagctctcagacgagaaggcaacgaacccgatcgtgccctcttttgatatgagtaatgaaggatcttatttcttcggagacaatgcagaagagtacgacaattaaggaaaaatacccttgtttctact

SEQ ID NO: 9 - DNA, 1467 bp, Influenza virus
  source 1..1467: mol_type = other DNA
agcaaaagcaggagtaaagatgaatccaaatcaaaagataataacgattggctctgtttccctcaccatttccacaatatgcttcttcatgcaaattgccatcctgataactactgtaacattgcatttcaagcaatatgaattcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgtaacattacaggatttgcacctttttctaaggacaattcgattcggctttccgctggtggggacatctgggtgacaagagaaccttatgtgtcatgcgatcctgacaagtgttatcaatttgcccttggacagggaacaacactaaacaacgtgcattcaaatgacatagtacatgataggaccccttatcggaccctattgatgaatgagttgggtgttccatttcatctggggaccaagcaagtgtgcatagcatggtccagctcaagttgtcacgatggaaaagcatggctgcatgtttgtgtaacgggggatgatgaaaatgcaactgctagcttcatttacaatgggaggcttgcagatagtattgtttcatggtccaaaaaaatcctcaggacccaggagtcagaatgcgtttgtatcaatggaacttgtacagtagtaatgactgatgggagtgcttcaggaaaagctgatactaaaatactattcattgaggaggggaaaattgttcatactagcacattatcaggaagtgctcagcatgtcgaggagtgctcctgttatcctcgatatcctggtgtcagatgtgtctgcagagacaactggaaaggctccaataggcccatcgtagatataaacataaaggattatagcattgtttccagttatgtgtgctcaggacttgttggagacacacccagaaaaaacgacagctccagcagtagccattgcttggatccaaacaatgaggaaggtggtcatggagtgaaaggctgggcctttgatgatggaaatgacgtgtggatgggaagaacgatcagcgagaagttacgctcaggatatgaaaccttcaaagtcattgaaggctggtccaaccctaactccaaattgcagataaataggcaagtcatagttgacagaggtaacaggtccggttattctggtattttctctgttgaaggcaaaagctgcatcaatcggtgcttttatgtggagttgataaggggaagaaaacaggaaactgaagtcttgtggacctcaaacagtattgttgtgttttgtggcacctcaggtacatatggaacaggctcatggcctgatggggcggacatcaatctcatgcctatataagctttcgcaattttagaaaaaaactccttgtttctact

SEQ ID NO: 10 - DNA, 1027 bp, Influenza virus
  source 1..1027: mol_type = other DNA
agcaaaagcaggtagatattgaaagatgagccttctaaccgaggtcgaaacgtatgttctctctatcgttccatcaggccccctcaaagccgagatcgcgcagagacttgaagatgtctttgctgggaaaaacacagatcttgaggctctcatggaatggctaaagacaagaccaattctgtcacctctgactaaggggattctggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaataggatgggggctgtaaccactgaagtggcatttggcctggtatgtgcaacatgtgagcagattgctgactcccagcacaggtctcataggcaaatggtggcaacaaccaatccattaataaggcatgagaacagaatggttttggccagcactacagctaaggctatggagcaaatggctggatcaagtgagcaggcagcggaggccatggagattgctagtcaggccaggcaaatggtgcaggcaatgagagccattgggactcatcctagctccagtactggtctaagagatgatcttcttgaaaatttgcagacctatcagaaacgaatgggggtgcagatgcaacgattcaagtgacccacttgttgttgccgcgagtatcattgggatcttgcacttgatattgtggattcttgatcgtctttttttcaaatgcgtctatcgactcttcaaacacggccttaaaagaggcccttctacggaaggagtacctgagtctatgagggaagagtatcgaaaggaacagcagaatgctgtggatgctgacgacagtcattttgtcagcatagagttggagtaaaaaactaccttgtttctact

SEQ ID NO: 11 - DNA, 890 bp, Influenza virus
  source 1..890: mol_type = other DNA
agcaaaagcagggtgacaaagacataatggattccaacactgtgtcaagtttccaggtagattgctttctttggcatatccggaaacaagttgtagaccaagaactgagtgatgccccattccttgatcggcttcgccgagatcagaggtccctaaggggaagaggcaatactctcggtctagacatcaaagcagccacccatgttggaaagcaaattgtagaaaagattctgaaagaagaatctgatgaggcacttaaaatgaccatggtctccacacctgcttcgcgatacataactgacatgactattgaggaattgtcaagaaactggttcatgctaatgcccaagcagaaagtggaaggacctctttgcatcagaatggaccaggcaatcatggagaaaaacatcatgttgaaagcgaatttcagtgtgatttttgaccgactagagaccatagtattactaagggctttcaccgaagagggagcaattgttggcgaaatctcaccattgccttcttttccaggacatactattgaggatgtcaaaaatgcaattggggtcctcatcggaggacttgaatggaatgataacacagttcgagtctctaaaaatctacagagattcgcttggagaagcagtaatgagaatgggggacctccacttactccaaaacagaaacggaaaatggcgagaacagctaggtcaaaagtttgaagagataagatggctgattgaagaagtgagacacagactaaaaacaactgaaaatagctttgaacaaataacattcatgcaagcattacaactgctgtttgaagtggaacaggagataagaactttctcatttcagcttatttaatgataaaaaacacccttgtttctact

SEQ ID NO: 12 - DNA, 1433 bp, Influenza virus
  source 1..1433: mol_type = other DNA
atgaatccaaatcaaaagataataacgattggctctgtttccctcaccatttccacaatatgcttcttcatgcaaattgccatcctgataactgctgtaacattgcatttcaagcaatatgaattcaactcccccatgctgtgtgaaccaacaataatagaaagaaacataacagagatagtgtatctgaccaacaccaccatagagaaggaaatatgccccaaactagcagaatacagaaattggtcaaagccgcaatgtaacattacaggatttgcacctttttctaaggacaattcgattcggctttccgctggtggggacatctgggtgacaagagaaccttatgtgtcatgcgatcctgacaagtgttatcaatttgcccttggacagggaacaacactaaacaacgtgcattcaaataacatagtacatgataggaccccttatcggaccctattgatgaatgagttgggtgttccatttcatctggggaccaagcaagtgtgcatagcatggtccagctcaagttgtcacgatggaaaagcatggctgcatgtttgtgtaacgggggatgatgaaaatgcaactgctagcttcatttacaatgggaggcttgcagatagtattgtttcatggtccaaaaaaatcctcaggacccaggagtcagaatgcgtttgtatcaatggaacttgtacagtagtaatgactgatgggagtgcttcaggaaaagctgatactaaaatactattcattgaggaggggaaaattgttcatactagcacattatcaggaagtgctcagcatgtcgaggagtgctcctgttatcctcgatatcctggtgtcagatgtgtctgcagagacaactggaaaggctccaataggcccatcgtagatataaacataaaggattatagcattgtttccagttatgtgtgctcaggacttgttggagacacacccagaaaagacgacagctccagcagtagccattgcttggatccaaacaatgaggaaggtggtcaaggagtgaaaggctgggcctttgatgatggaaatgacgtgtggatgggaagaacgatcagcgagaagttacgctcaggatatgaaaccttcaaagtcattgaaggctggtccaaccctaactccaaattgcagataaataggcaagtcatagttgacagaggtaacaggtccggttattctggtattttctctgttgaaggcaaaagctgcatcaatcggtgcttttatgtggagttgataaggggaagaaaacaggaaactgaagtcttgtggacctcaaacagtattgttgtgttttgtggcacctcaggtacatatggaacaggctcatggcctgatggggcggacatcaatctcatgcctatataagctttcgcaattttagaaaaaaactccttgtttctact

SEQ ID NO: 13 - DNA, 1733 bp, Influenza virus
  source 1..1733: mol_type = other DNA
atgaagactatcattgctttgagctacattctatgtctggttttcgctcaaaagcttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtggaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagcttcaattggactggagtcactcagaatggaacaagctctgcttgcaaaaggagatctaataaaagtttctttagtagattgaattggttgacccacttaaaatacaaatacccagcattgaacgtgactatgccaaacaatgaaaaatttgacaaattgtacatttggggggttcaccacccgggtacggacagtgatcaaatcagcctatatgctcaagcatcaggaagaatcacagtctctaccaaaagaagccaacaaactgtaatcccgaatatcggatctagacccagggtaagggatgtctccagcagaataagcatctattggacaatagtaaaaccgggagacatacttttgattaacagcacagggaatctaattgctcctcggggttacttcaaaatacgaagtgggaaaagctcaataatgagatcagatgcacccattggcaaatgcaattctgaatgcatcactccaaatggaagcattcccaatgacaaaccatttcaaaatgtaaacaggatcacatatggggcctgtcccagatatgttaagcaaaacactctgaaattggcaacagggatgcgaaatgtaccagagaaacaaactagaggcatatttggcgcaatcgcgggtttcatagaaaatggttgggagggaatggtggacggttggtacggtttcaggcatcaaaattctgagggcacaggacaagcagcagatctcaaaagcactcaagcagcaatcaaccaaatcaatgggaaactgaataggttaatcgggaaaacaaacgagaaattccatcagattgaaaaagaattctcagaagtagaagggagaattcaggacctcgagaaatatgttgaggacactaaaatagatctctggtcatacaacgcggagcttcttgttgccctggagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcatagagtcaatcagaaatggaacttatgaccatgatgtatacagagatgaagcattaaacaaccggttccagatcaaaggtgttgagctgaagtcaggatacaaagattggatcctatggatttcctttgccatatcatgttttttgctctgtgttgctttgttggggttcatcatgtgggcctgccaaaaaggcaacattaggtgcaacatttgcatttgagtgcattaattaaaaacacccttgtttctact

SEQ ID NO: 14 - DNA, 1002 bp, Influenza virus
  source 1..1002: mol_type = other DNA
atgagccttctaaccgaggtcgaaacgtatgttctctctatcgttccatcaggccccctcaaagcccagatcgcgcagagacttgaagatgtctttgctgggaaaaacacagatcttgaggctctcatggaatggctaaagacaagaccaattctgtcacctctgactaaggggattctggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaataggatgggggctgtaaccactgaagtggcatttggcctggtatgtgcaacatgtgagcagattgctgactcccagcacaggtctcataggcaaatggtggcaacaaccaatccattaataaggcatgagaacagaatggttttggccagcactacagctaaggctatggagcaaatggctggatcaagtgagcaggcagcggaggccatggagattgctagtcaggccaggcaaatggtgcaggcaatgagagccattgggactcatcctagctccagtactggtctaagagatgatcttcttgaaaatttgcagacctatcagaaacgaatgggggtgcagatgcaacgattcaagtgacccacttgttgttgccgcgagtatcattgggatcttgcacttgatattgtggattcttgatcgtctttttttcaaatgcgtctatcgactcttcaaacacggccttaaaagaggcccttctacggaaggagtacctgagtctatgagggaagagtatcgaaaggaacagcagaatgctgtggatgctgacgacagtcattttgtcagcatagagttggagtaaaaaactaccttgtttctact

SEQ ID NO: 15 - DNA, 1520 bp, Influenza virus
  source 1..1520: mol_type = other DNA
atggcgtcccaaggcaccaaacggtcttatgaacagatggaaactgatggggatcgccagaatgcaactgagattagggcatccgtcgggaagatgattgatggaattgggagattctacatccaaatgtgcactgaacttaaactcagtgattatgaagggcggttgatccagaacagcttgacaatagagaaaatggtgctctctgcttttgatgaaagaaggaataaatatctggaagaacaccccagcgcggggaaagatcctaagaaaactggggggcccatatacaggagagtaaatggaaaatggatgagggaactcgtcctttatgacaaagaagaaataaggcgaatctggcgccaagccaacaatggtgaggatgcgacagctggtctaactcacataatgatctggcattccaatttgaatgatgcaacataccagaggacaagagctcttgttcgaaccggaatggatcccagaatgtgctctctgatgcagggctcgactctccctagaaggtccggagctgcaggtgctgcagtcaaaggaatcgggacaatggtgatggagctgatcagaatggtcaaacgggggatcaacgatcgaaatttctggagaggtgagaatgggcggaaaacaagaagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattgatattgagaggatcagttgctcacaaatcttgcctacctgcctgtgtgtatggacctgcagtatccagtgggtacgacttcgaaaaagagggatattccttggtgggaatagaccctttcaaactacttcaaaatagccaagtatacagcctaatcagacctaacgagaatccagcacacaagagtcagctggtatggatggcatgccattctgctgcatttgaagatttaagattgttaagcttcatcagagggacaaaagtatctccacgagggaaactttcaactagaggagtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaagaacttcagacatgagggcagaaatcataagaatgatggaaggtgcaaaaccagaagaagtgtcgttccgggggaggggagttttcgagctctcagacgagaaggcaacgaacccgatcgtgccctcttttgatatgagtaatgaaggatcttatttcttcggagacaatgcagaagagtacgacaattaaggaaaaatacccttgtttctact

SEQ ID NO: 16 - DNA, 864 bp, Influenza virus
  source 1..864: mol_type = other DNA
atggattccaacactgtgtcaagtttccaggtagattgctttctttggcatatccggaaacaagttgtagaccaagaactgagtgatgccccattccttgatcggcttcgccgagatcagaggtccctaaggggaagaggcaatactctcggtctagacatcaaagcagccacccatgttggaaagcaaattgtagaaaagattctgaaagaagaatctgatgaggcacttaaaatgaccatggtctccacacctgcttcgcgatacataactgacatgactattgaggaattgtcaagaaactggttcatgctaatgcccaagcagaaagtggaaggacctctttgcatcagaatggaccaggcaatcatggagaaaaacatcatgttgaaagcgaatttcagtgtgatttttgaccgactagagaccatagtattactaagggctttcaccgaagagggagcaattgttggcgaaatctcaccattgccttcttttccaggacatactattgaggatgtcaaaaatgcaattggggtcctcatcggaggacttgaatggaatgataacacagttcgagtctctaaaaatctacagagattcgcttggagaagcagtaatgagaatgggggacctccacttactccaaaacagaaacggaaaatggcgagaacagctaggtcaaaagtttgaagagataagatggctgattgaagaagtgagacacagactaaaaacaactgaaaatagctttgaacaaataacattcatgcaagcattacaactgctgtttgaagtggaacaggagataagaactttctcatttcagcttatttaatgataaaaaacacccttgtttctact

SEQ ID NO: 17 - DNA, 2317 bp, Influenza virus
  source 1..2317: mol_type = other DNA
atggatgtcaatccgactctactgttcctaaaggttccagcgcaaaatgccataagcaccacattcccttatactggagatcctccatacagccatggaacaggaacagggtacaccatggacacagtcaacagaacacaccaatattcagataaggggaagtggacgacaaatacagaaactggggcaccccaactcaacccaattgatggaccactacctgaggataatgagccaagtggatatgcacaaacagactgtgtcctggaggctatggccttccttgaagaatcccacccaggtatctttgagaactcatgccttgaaacaatggaagtcgttcaacaaacaagggtggacaaactaacccaaggtcgccagacttatgattggacattaaacagaaatcaaccggcagcaactgcattagccaacaccatagaagtttttagatcgaatggactaacagctaatgaatcaggaaggctaatagatttcctcaaggatgtgatggaatcaatggataaagaggaaatggagataacaacacactttcaaagaaaaaggagagtaagagacaacatgaccaagaaaatggtcacacaaagaacaatagggaagaaaaaacaaagagtaaataagagaggctatctaataagagctttgacattgaacacgatgaccaaagatgcagagagaggtaaattaaaaagaagggctattgcaacacccgggatgcaaattagagggttcgtgtacttcgttgaaactttagctagaagcatttgcgaaaagcttgaacagtctggacttccggttgggggtaatgaaaagaaggccaaactggcaaatgttgtgagaaaaatgatgactaattcacaagacacagagctttctttcacaatcactggggacaacactaagtggaatgaaaatcaaaaccctcgaatgtttttggcgatgattacatatatcacaaaaaatcaacctgagtggttcagaaacatcctgagcatcgcaccaataatgttctcaaacaaaatggcaagactgggaaaaggatacatgttcgagagtaagagaatgaaactccgaacacaaatacccgcagaaatgctagcaaacattgacctgaagtatttcaatgaatcaacaaggaagaaaattgagaaaataaggcctcttctaatagatggcacagcatcattgagccctgggatgatgatgggcatgttcaacatgctaagtacggttttaggagtctcgatactgaatcttgggcaaaagaaatacaccaagacaacatactggtgggatgggctccaatcctccgacgattttgccctcatagtgaatgcaccaaatcatgagggaatacaagcaggagtggatagattttacaggacctgcaagttagtgggaatcaacatgagcaaaaagaagtcctatataaataaaacagggacatttgaattcacaagctttttttatcgatatggatttgtggctaattttagcatggagctgcccagttttggagtgtctggaataaacgagtcagctgatatgagcattggagtaacagtgataaagaacaacatgataaacaatgaccttggaccagcaacagcccagatggctctccaattgttcatcaaagactacagatatacatataggtgccatagaggagacacacaaattcagacgagaagatcattcgagctaaagaagctgtgggatcaaacccaatcaagggcaggactattggtatcagatgggggaccaaacttatacaatatccggaatcttcacatccctgaagtctgcttaaagtgggagctaatggatgagaattatcggggaagactttgtaatcccctgaatccctttgtcagccataaagaaattgagtctgtaaacaatgctgtagtgatgccagcccatggtccggccaaaagtatggaatatgatgccgttgcaactacacactcctggattcccaagaggaaccgctctattctcaacacaagccaaaggggaattcttgaggatgaacagatgtaccagaagtgctgcaacttgttcgagaaatttttccctagtagttcatataggagaccgattggaatttctagcatggtggaggccatggtgtctagggcccggattgatgccagaattgacttcgagtctggacggattaagaaggaagagttctctgagatcatgaagatctgttccaccattgaagaactcagacggcaaaaataatgaatttagcttgtccttcatgaaaaaatgccttgtttctact

SEQ ID NO: 18 - DNA, 2209 bp, Influenza virus
  source 1..2209: mol_type = other DNA
atggaagattttgtgcgacaatgcttcaacccgatgattgtcgaacttgcagaaaaagcaatgaaagagtatggggaggatctgaaaattgaaacaaacaaatttgcagcaatatgcactcacttggaggtatgtttcatgtattcagattttcatttcatcaatgaacaaggcgaatcaatagtggtagaacttgatgatccaaatgcactgttaaagcacagatttgaaataatcgaggggagagacagaacaatggcctggacagtagtaaacagtatctgcaacactactggagctgaaaaaccgaagtttctaccagatttgtatgattacaaggagaacagattcatcgaaattggagtgacaaggagagaagtccacatatattaccttgaaaaggccaataagattaaatctgagaacacacacattcacattttctcattcactggggaggaaatggccacaaaggcagactacactctcgacgaggaaagcagggctaggattaagaccaggctatttaccataagacaagaaatggccaacagaggcctctgggattcctttcgtcagtccgaaagaggcgaagaaacaattgaagaaaaatttgaaatctcaggaactatgcgtaggcttgccgaccaaagtctcccaccgaacttctcctgccttgagaattttagagcctatgtggatggattcgaaccgaacggctgcattgagggcaagctttctcaaatgtccaaagaagtgaatgcccaaattgaaccttttctgaagacaacaccaagaccaatcaaacttccgaatggacctccttgttatcagcggtccaagttcctcctgatggatgctttaaaattgagcattgaagacccaagtcacgaaggagaagggatcccattatatgatgcgatcaagtgcataaaaacattctttggatggaaagaaccttatatagtcaaaccacacgaaaagggaataaattcaaattacctgctgtcatggaagcaagtattgtcagaattgcaggacattgaaaatgaggagaagattccaaggactaaaaacatgaagaaaacgagtcaactaaagtgggctcttggtgagaacatggcaccagagaaagtagactttgaaaactgcagagacataagcgatttgaagcaatatgatagtgacgaacctgaattaaggtcactttcaagctggatacagaatgagttcaacaaggcctgcgagctaactgattcaatctggatagagctcgatgaaattggagaggacgtagccccaattgaatacattgcaagcatgaggaggaattatttcacagcagaggtgtcccattgtagagccactgagtacataatgaagggggtatacattaatactgccctgctcaatgcatcctgtgcagcaatggacgattttcaactaattcccatgataagcaagtgcagaactaaagagggaaggcgaaaaaccaatttatatggattcatcataaagggaagatctcatttaaggaatgacacagatgtggtaaactttgtgagcatggagttttctctcactgacccgagacttgagccacataaatgggagaaatactgtgtccttgagataggagatatgttactaagaagtgccataggccaaatttcaaggcctatgttcttgtatgtgaggacaaacggaacatcaaaggtcaaaatgaaatggggaatggagatgagacgttgcctccttcagtcactccagcagatcgagagcatgattgaagccgagtcctcggttaaagagaaagacatgaccaaagagttttttgagaataaatcagaagcatggcccattggggagtcccccaagggagtggaagaaggttccattgggaaagtctgtaggactctattggctaagtcagtgttcaatagcctgtatgcatcaccacaattggaaggattttcagcggagtcaagaaaactgctccttgttgttcaggctcttagggacaacctcgaacctgggacctttgatcttggggggctatatgaagcaattgaggagtgcctgattaatgatccctgggttttgctcaatgcgtcttggttcaactccttcctgacacatgcattaaaatagttatggcagtgctactatttgttatccgtactgtccaaaaaagtaccttgtttctact

SEQ ID NO: 19 - DNA, 2314 bp, Influenza virus
  source 1..2314: mol_type = other DNA
atggaaagaataaaagaactacggaacctgatgtcgcagtctcgcactcgcgagatactgacaaaaaccacagtggaccatatggccataattaagaagtacacatcggggagacaggaaaagaacccgtcacttaggatgaaatggatgatggcaatgaaatacccaatcactgctgacaaaaggataacagaaatggttccggagagaaatgaacaaggacaaactctatggagtaaaatgagtgatgctggatcagatcgagtgatggtatcacctttggctgtgacatggtggaatagaaatggacccgtgacaagtacggtccattacccaaaagtatacaagacttattttgacaaagtcgaaaggttaaaacatggaacctttggccctgttcattttagaaatcaagtcaagatacgccgaagagtagacataaaccctggtcatgcggacctcagtgccaaggaggcacaagatgtaattatggaagttgtttttcccaatgaagtgggagccaggatactaacatcagaatcgcaattaacaataactaaagagaaaaaagaagaactccgagattgcaaaatttctcccttgatggttgcatacatgttagagagagaacttgtccgaaaaacaagatttctcccagttgctggcggaacaagcagtatatacattgaagttttacatttgactcaagggacgtgttgggaacaaatgtacactccaggtggagaagtgaggaatgacgatgttgaccaaagcctaattattgcagccaggaacatagtaagaagagccgcagtatcagcagatccactagcatctttattggagatgtgccacagcacacaaattggcgggacaaggatggtggacattcttagacagaacccgactgaagaacaagctgtggatatatgcaaggctgcaatgggattgagaatcagctcatccttcagctttggtgggtttacatttaaaagaacaagcgggtcatcagtcaaaaaagaggaagaagtgcttacaggcaatctccaaacattgaagataagagtacatgaggggtatgaggagttcacaatggtggggaaaagagcaacagctatactcagaaaagcaaccagaagattggttcagctcatagtgagtggaagagacgaacagtcaatagccgaagcaataattgtggccatggtgttttcacaagaggattgcatgataaaagcagttagaggtgacctgaatttcgtcaacagagcaaatcagcggttgaaccccatgcatcagcttttaaggcattttcagaaagatgcgaaagtgctttttcagaattggggaattgagcacatcgacagtgtaatgggaatggttggagtattaccagatatgactccaagcacagagatgtcaatgagaggaataagagtcagcaaaatgggtgtggatgaatactccagtacagagagggtggtggttagcattgatcggtttttgagagttcgagaccaacgcgggaatgtattattatctcctgaagaggttagtgaaacacagggaactgagagactgacaataacttattcatcgtcgatgatgtgggagattaacggtcctgagtcggttttggtcaatacttatcaatggatcatcagaaattgggaagctgtcaaaattcaatggtctcagaatcctgcaatgttgtacaacaaaatggaatttgaaccatttcaatctttagtccccaaggccattagaagccaatacagtgggtttgtcagaactctattccaacaaatgagagacgtacttgggacatttgacaccacccagataataaagcttctcccttttgcagccgctccaccaaagcaaagcagaatgcagttctcttcactgactgtaaatgtgaggggatcagggatgagaatacttgtaaggggcaattctcctgtattcaactacaacaagaccactaaaagactaacaattctcggaaaagatgccggcactttaattgaagacccagatgaaagcacatccggagtggagtccgctgtattgagagggtttctcattataggtaaggaagacagaagatacgggccagcattaagcatcaatgaactgagtaaccttgcaaaaggggaaaaggctaatgtgctaatcgggcaaggagacgtggtgttggtaatgaaacgaaaacgggactctagcatacttactgacagccagacagcgaccaaaagaattcggatggccatcaattaatgttgaatagtttaaaaacgaccttgtttctact

SEQ ID NO: 20 - DNA, 2314 bp, Influenza virus
  source 1..2314: mol_type = other DNA
atggaaagaataaaagaactacggaacctgatgtcgcagtctcgcactcgcgagatactgacaaaaaccacagtggaccatatggccataattaagaagtacacatcggggagacaggaaaagaacccgtcacttaggatgaaatggatgatggcaatgaaatacccaatcactgctgacaaaaggataacagaaatggttccggagagaaatgaacaaggacaaactctatggagtaaaatgagtgatgctggatcagatcgagtgatggtatcacctttggctgtgacatggtggaatagaaatggacccgtgacaagtacggtccattacccaaaagtatacaagacttattttgacaaagtcgaaaggttaaaacatggaacctttggccctgttcattttagaaatcaagtcaagatacgccgaagagtagacataaaccctggtcatgcggacctcagtgccaaggaggcacaagatgtaattatggaagttgtttttcccaatgaagtgggagccaggatactaacatcagaatcgcaattaacaataactaaagagaaaaaagaagaactccgagattgcaaaatttctcccttgatggttgcatacatgttagagagagaacttgtccgaaaaacaagattcctcccagttgctggcggaacaagcagtatatacattgaagttttacatttgactcaagggacgtgttgggaacaaatgtacactccaggtggagaagtgaggaatgacgatgttgaccaaagcctaattattgcagccaggaacatagtaagaagagccgcagtatcagcagatccactagcatttttattggagatgtgccacagcacacaaattggcgggacaaggatggtggacattcttagacagaacccgactgaagaacaagctgtggatatatgcaaggctgcaatgggattgagaatcagctcatccttcagctttggtgggtttacatttaaaagaacaagcgggtcatcagtcaaaaaagaggaagaactgcttacaggcaatctccaaacattgaagataagagtacatgaggggtatgaggagttcacaatggtggggaaaagagcaacagctatactcagaaaagcaaccagaagattggttcagctcatagtgagtggaagagacgaacagtcaatagccgaagcaataattgtggccatggtgttttcacaagaggattgcatgataaaagcagttagaggtgacctgaatttcgtcaacagagcaaatcagcggttgaaccccatgcatcagcttttaaggcattttcagaaagatgcgaaagtgctttttcagaattggggaattgagcacatcgacagtgtaatgggaatggttggagtattaccagatatgactccaagcacagagatgtcaatgagaggaataagagtcagcaaaatgggtgtggatgaatactccagtacagagagggtggtggttagcattgatcggtttttgagagttcgagaccaacgcgggaatgtattattatctcctgaagaggttagtgaaacacagggaactgagagactgacaataacttattcatcgtcgatgatgtgggagattaacggtcctgagtcggttttggtcaatacttatcaatggatcatcagaaattgggaagctgtcaaaattcaatggtctcagaatcctgcaatgttgtacaacaaaatggaatttgaaccatttcaatctttagtccccaaggccattagaagccaatacagtgggtttgtcagaactctattccaacaaatgagagacgtacttgggacatttgacaccacccagataataaagcttctcccttttgcagccgctccaccaaagcaaagcagaatgcagttctcttcactgactgtaaatgtgaggggatcagggatgagaatacttgtaaggggcaattctcctgtattcaactacaacaagaccactaaaagactaacaattctcggaaaagatgccggcactttaattgaagacccagatgaaagcacatccggagtggagtccgctgtattgagagggtttctcattataggtaaggaagacagaagatacgggccagcattaagcatcaatgaactgagtaaccttgcaaaaggggaaaaggctaatgtgctaatcgggcaaggagacgtggtgttggtaatgaaacgaaaacgggactctagcatacttactgacagccagacagcgaccaaaagaattcggatggccatcaattaatgttgaatagtttaaaaacgaccttgtttctact

SEQ ID NO: 21 - DNA, 2314 bp, Influenza virus
  source 1..2314: mol_type = other DNA
atggaaagaataaaagaactacggaacctgatgtcgcagtctcgcactcgcgagatactgacaaaaaccacagtggaccatatggccataattaagaagtacacatcggggagacaggaaaagaacccgtcacttaggatgaaatggatgatggcaatgaaatacccaatcactgctgacaaaaggataacagaaatggttccggagagaaatgaacaaggacaaactctatggagtaaaatgagtgatgctggatcagatcgagtgatggtatcacctttggctgtgacatggtggaatagaaatggacccgtgacaagtacggtccattacccaaaagtatacaagacttattttgacaaagtcgaaaggttaaaacatggaacctttggccctgttcattttagaaatcaagtcaagatacgccgaagagtagacataaaccctggtcatgcggacctcagtgccaaggaggcacaagatgtaattatggaagttgtttttcccaatgaagtgggagccaggatactaacatcagaatcgcaattaacaataactaaagagaaaaaagaagaactccgagattgcaaaatttctcccttgatggttgcatacatgttagagagagaacttgtccgaaaaacaagattcctcccagttgctggcggaacaagcagtatatacattgaagttttacatttgactcaagggacgtgttgggaacaaatgtacactccaggtggagaagtgaggaatgacgatgttgaccaaagcctaattattgcagccaggaacatagtaagaagagccgcagtatcagcagatccactagcatctttattggagatgtgccacagcacacaaattggcgggacaaggatggtggacattcttagacagaacccgactgaagaacaagctgtggatatatgcaaggctgcaatgggattgagaatcagctcatccttcagctttggtgggtttacatttaaaagaacaagcgggtcatcagtcaaaaaagaggaagaactgcttacaggcaatctccaaacattgaagataagagtacataaggggtatgaggagttcacaatggtggggaaaagagcaacagctatactcagaaaagcaaccagaagattggttcagctcatagtgagtggaagagacgaacagtcaatagccgaagcaataattgtggccatggtgttttcacaagaggattgcatgataaaagcagttagaggtgacctgaatttcgtcaacagagcaaatcagcggttgaaccccatgcatcagcttttaaggcattttcagaaagatgcgaaagtgctttttcagaattggggaattgagcacatcgacagtgtaatgggaatggttggagtattaccagatatgactccaagcacagagatgtcaatgagaggaataagagtcagcaaaatgggtgtggatgaatactccagtacagagagggtggtggttagcattgatcggtttttgagagttcgagaccaacgcgggaatgtattattatctcctgaagaggttagtgaaacacagggaactgagagactgacaataacttattcatcgtcgatgatgtgggagattaacggtcctgagtcggttttggtcaatacttatcaatggatcatcagaaattgggaagctgtcaaaattcaatggtctcagaatcctgcaatgttgtacaacaaaatggaatttgaaccatttcaatctttagtccccaaggccattagaagccaatacagtgggtttgtcagaactctattccaacaaatgagagacgtacttgggacatttgacaccacccagataataaagcttctcccttttgcagccgctccaccaaagcaaagcagaatgcagttctcttcactgactgtaaatgtgaggggatcagggatgagaatacttgtaaggggcaattctcctgtattcaactacaacaagaccactaaaagactaacaattctcggaaaagatgccggcactttaattgaagacccagatgaaagcacatccggagtggagtccgctgtattgagagggtttctcattataggtaaggaagacagaagatacgggccagcattaagcatcaatgaactgagtaaccttgcaaaaggggaaaaggctaatgtgctaatcgggcaaggagacgtggtgttggtaatgaaacgaaaacgggactctagcatacttactgacagccagacagcgaccaaaagaattcggatggccatcaattaatgttgaatagtttaaaaacgaccttgtttctact

SEQ ID NO: 22 - DNA, 1775 bp, Influenza virus
  source 1..1775: mol_type = other DNA
agcaaaagcaggggaaaataaaaacaaccaaaatgaaggcaaacctactggtcctgttatgtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacattccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagagatatgaggaaaaacacccttgtttctact

SEQ ID NO: 23 - DNA, 1413 bp, Influenza virus
  source 1..1413: mol_type = other DNA
agcaaaagcaggggtttaaaatgaatccaaatcagaaaataataaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaagtgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaattggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgtgaggcaagatgttgtggcaatgactgattggtcagggtatagcggaagtttcgttcaacatcctgagctgacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 24 - DNA, 2233 bp, Influenza virus
  source 1..2233: mol_type = other DNA
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggagtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 25 - DNA, 2341 bp, Influenza virus
  source 1..2341: mol_type = other DNA
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaacaaagaagaaatggggatcacaactcattttcagagaaagagacgggtgagagacaatatgactaagaaaatgataacacagagaacaatgggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatataggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaggatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 26 - DNA, 2341 bp, Influenza virus
  source 1..2341: mol_type = other DNA
agcgaaagcaggtcaattatattcaatatggaaagaataaaagaactacgaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcaacgattgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 27 - DNA, 1565 bp, Influenza virus
  source 1..1565: mol_type = other DNA
agcaaaagcagggtagataatcactcactgagtgacatcaaaatcatggcgtctcaaggcaccaaacgatcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaataccttgaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcagaatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagggagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 28 - DNA, 1027 bp, Influenza virus
  source 1..1027: mol_type = other DNA
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 29 - DNA, 890 bp, Influenza virus
  source 1..890: mol_type = other DNA
agcaaaagcagggtgacaaaaacataatggatccaaacactgtgtcaagctttcaggtagattgctttctttggcatgtccgcaaacgagttgcagaccaagaactaggcgatgccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 30 - protein, 468 aa, Influenza virus
MNPNQKIITIGVVNTTLSTIALLIGVGNLIFNTVIHEKIGDHQTVIHPTTTTPAIPNCSDTIITYNNTVINNITTIITEAERLFKPPLPLCPFRGFFPFHKDNAIRLGENKDVIVTREPYVSCDNDNCWSFALAQGALLGTKHSNGTIKDRTPYRSLIQFPIGTAPVLGNYKEICIAWSSSSCFDGKEWMHVCMTGNDNDASAQIIYAGRMTDSIKSWKRDILRTQESECQCIDGTCVVAVTDGPAANSADHRVYWIREGRIVKYENVPKTKIQHLEECSCYVDIDVYCICRDNWKGSNRPWMRINNETILETGYVCSKFHSDTPRPADPSTVSCDSPSNVNGGPGVKGFGFKVGNDVWLGRTMSTSGRSGFEIIKVAEGWINSPNHAKSVTQTLVSNNDWSGYSGSFIVKTKACFQPCFYVELIRGRPNKNDDVSWTSNSIVTFCGLDNEPGSGNWPDGSNIGFMPK

SEQ ID NO: 31 - protein, 470 aa, Influenza virus
MNPNQKIITIGSVSIILTTIGLLLQITSLCSIWFSHYNQVTQTHEQPCSNNTTNYYNETFVNVTNVQNNYTTVIEPSAPDVVHYSSGRDLCPIRGWAPLSKDNGIRIGSRGEVFVIREPFISCSISECRTFFLTQGALLNDKHSNGTVKDRSPFRTLMSCPIGVAPSPSNSRFESVAWSATACSDGPGWLTLGITGPDATAVAVLKYNGIITDTLKSWKGNIMRTQESECVCQDEFCYTLITDGPSDAQAFYKILKIRKGKIVSMKDVDATGFHFEECSCYPSGTDIECVCRDNWRGSNRPWIRFNSDLDYQIGYVCSGIFGDNPRPVDGTGSCNSPVNNGKGRYGVKGFSFRYGDGVWIGRTKSLESRSGFEMVWDANGWVSTDKDSNGVQDIIDNDNWSGYSGSFSIRGETTGRNCTVPCFWVEMIRGQPKEKTIWTSGSSIAFCGVNSDTTGWSWPDGALLPFDIDK

SEQ ID NO: 32 - protein, 470 aa, Influenza virus
MNPNQKIICISATGMTLSVVSLLIGIANLGLNIGLHYKMGDTPDVNIPNMNETNSTTTIINNHTQNNFTNITNIIVNKNEEGTFLNLTKPLCEVNSWHILSKDNAIRIGEDAHILVTREPYLSCDPQGCRMFALSQGTTLRGRHANGTIHDRSPFRALISWEMGQAPSPYNVRVECIGWSSTSCHDGISRMSICMSGANNNASAVVWYGGRPVTEIPSWAGNILRTQESECVCHKGICPVVMTDGPANNRAATKIIYFKEGKIQKIEELAGNTQHIEECSCYGAVGVIKCICRDNWKGANRPVITIDPEMMTHTSKYLCSKILTDTSRPNDPTNGNCDAPITGGSPDPGVKGFAFLDRENSWLGRTISKDSRSGYEMLKVPNAETDTQSGPISHQVIVNNQNWSGYSGAFIDYWANKECFNPCFYVELIRGRPKESSVLWTSNSIVALCGSKERLGSWSWHDGAEIIYFK

SEQ ID NO: 33 - protein, 470 aa, Influenza virus
MNPNQKLFALSGVAIALSILNLLIGISNVGLNVSLHLKGSSDQDKNWTCTSVTQNNTTLIENTYVNNTTVIDKETGTAKPNYLMLNKSLCKVEGWVVVAKDNAIRFGESEQIIVTREPYVSCDPLGCKMYALHQGTTIRNKHSNGTIHDRTAFRGLISTPLGSPPVVSNSDFLCVGWSSTSCHDGIGRMTICVQGNNDNATATVYYDRRLTTTIKTWAGNILRTQESECVCHNGTCVVIMTDGSASSQAYTKVLYFHKGLVIKEEALKGSARHIEECSCYGHNSKVTCVCRDNWQGANRPVIEIDMNAMEHTSQYLCTGVLTDTSRPSDKSMGDCNNPITGSPGAPGVKGFGFLDSSNTWLGRTISPRSRSGFEMLKIPNAETDPNSKITERQEIVDNNNWSGYSGSFIDYWDESSECYNPCFYVELIRGRPEEAKYVGWTSNSLIALCGSPISVGSGSFPDGAQIQYFS

SEQ ID NO: 34 - protein, 470 aa, Influenza virus
MNPNQKIITVGSVSLGLVVLNILLHIVSITVTVLVLPGNGNNKNCNETVIREYNETVRIEKVTQWHNTNVIEYIEKPESGHFMNNTEALCDAKGFAPFSKDNGIRIGSRGHVFVIREPFVSCSPTECRTFFLTQGSLLNDKHSNGTVKDRSPYRTLMSVEIGQSPNVYQARFEAVAWSATACHDGKKWMTIGVTGPDAKAVAVVHYGGIPTDVINSWAGDILRTQESSCTCIQGECYWVMTDGPANRQAQYRAFKAKQGKIVGQTEISFNGSHIEECSCYPNEGKVECVCRDNWTGTNRPVLVISPDLSYRAGYLCAGLPSDTPRGEDSQFTGSCTSPVGNQGYGVKGFGFRQGNDVWMGRTISRTSRSGFEILKVRNGWVQNSKEQIKRQVVVDNLKWSGYSGSFTLPVELTKRNCLVPCFWVEMIRGKPEEKTIWTSSSSIVMCGVDHEIADWSWHDGAILPFDIDKM

SEQ ID NO: 35 - protein, 465 aa, Influenza virus
MNPNQKILCTSATAIIIGAIAVLIGIANLGLNIGLHLKPGCNCSHSQPETTNTSQTIINNYYNETNITNIQMEERTSRNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVTEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

SEQ ID NO: 36-38 - skipped (3 entries)

SEQ ID NO: 39 - DNA, 2341 bp, Influenza virus
  source 1..2341: mol_type = other DNA
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaatgacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaagaatgatgatgttgatcaaagcttgattattgctgctaggaacatagtgagaagagctgcagtatcagcagacccactagcatctttattggagatgtgccacagcacacagattggtggaattaggatggtagacatccttaagcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatctgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagttagaggtgatctgaatttcgtcaatagggcgaatcagcgactgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccggttcttgagagtcagggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactacaacaaggccacgaagagactcacagttctcggaaaggatgctggcactttaaccgaagacccagatgaaggcacagctggagtggagtccgctgttctgaggggattcctcattctgggcaaagaagacaggagatatgggccagcattaagcatcaatgaactgagcaaccttgcgaaaggagagaaggctaatgtgctaattgggcaaggagacgtggtgttggtaatgaaacgaaaacgggactctagcatacttactgacagccagacagcgaccaaaagaattcggatggccatcaattagtgtcgaatagtttaaaaacgaccttgtttctact

SEQ ID NO: 40 - DNA, 2341 bp, Influenza virus
  source 1..2341: mol_type = other DNA
agcgaaagcaggcaaaccatttgaatggatgtcaatccgaccttacttttcttaaaagtgccagcacaaaatgctataagcacaactttcccttataccggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactttaaatagaaaccagcctgctgcaacagcattggccaacacaatagaagtgttcagatcaaatggcctcacggccaatgagtcaggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaggaaacagagattgaacaaaaggggttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcattgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 41 - DNA, 2233 bp, Influenza virus
  source 1..2233: mol_type = other DNA
agcgaaagcaggtactgattcaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaagggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 42 - DNA, 1565 bp, Influenza virus
  source 1..1565: mol_type = other DNA
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 43 - DNA, 1027 bp, Influenza virus
  source 1..1027: mol_type = other DNA
agcaaaagcaggtagatattgaaagatgagtcttctaaccgaggtcgaaacgtacgttctctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaacccactaatcagacatgagaacagaatggttttagccagcactacagctaaggctatggagcaaatggctggatcgagtgagcaagcagcagaggccatggaggttgctagtcaggctaggcaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgatattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 44 - DNA, 890 bp, Influenza virus
  source 1..890: mol_type = other DNA
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggcctctgtacctgcgtcgcgttacctaaccgacatgactcttgaggaaatgtcaagggaatggtccatgctcatacccaagcagaaagtggcaggccctctttgtatcagaatggaccaggcgatcatggataaaaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaaggtaacagagaatagttttgagcaaataacatttatgcaagccttacatctattgcttgaagtggagcaagagataagaactttctcatttcagcttatttaataataaaaaacacccttgtttctact

SEQ ID NO: 45 - skipped

SEQ ID NO: 46 - DNA, 1701 bp, Influenza virus
  source 1..1701: mol_type = other DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaaaaatgaaagcttcaattggactggagtcactcaaaacggaacaagttctgcttgcataaggggatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaataggttgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcatagaatcaataagaaatgaaacttatgaccacaatgtgtacagggatgaagcattgaacaaccggttccagatcaagggagttgagctgaagtcaggatacaaagattggatcctatggatttcctttgccatatcatgttttttgctttgtgttgctttgttggggttcatcatgtgggcctgccaaaagggcaacattagatgcaacatttgcatttga

SEQ ID NO: 47 - DNA, 1410 bp, Influenza virus
  source 1..1410: mol_type = other DNA
atgaatccaaatcaaaagataataacgattggctctgtttctctcaccatttccacaatatgcttcttcatgcaaattgccatcctgataactactgtaacattgcatttcaagcaatatgaattcaactcccccccaaacaaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttatagatagtgttgtttcatggtccaaagatattctcaggacccaggagtcagaatgcgtttgtatcaatggaacttgtacagtagtaatgactgatggaaatgctacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcgtagatataaacataaaggatcatagcattgtttccagttatgtgtgttcaggacttgttggagacacacccagaaaaaacgacagctccagcagtagccattgtttgaatcctaacaatgaagaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 48 - protein, 469 aa, Influenza virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLIDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 49 - protein, 469 aa, Influenza virus
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 50 - skipped

SEQ ID NO: 51 - protein, 469 aa, Influenza virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 52 - protein, 470 aa, Influenza virus; identical to SEQ ID NO: 33

SEQ ID NO: 53 - protein, 465 aa, Influenza virus
MNPNQKILCTSATAIIIGAIAVLIGIANLGLNIGLHLKPGCNCSHSQPETTNTSQTIINNYYNETNITNIQMEERTSRNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

SEQ ID NO: 54 - protein, 469 aa, Influenza virus; identical to SEQ ID NO: 3

SEQ ID NO: 55 - DNA, 1434 bp, Influenza virus
  source 1..1434: mol_type = other DNA
atgaatccaaatcaaaagataataacgattggctctgtttctctcacaatttccacaatatgcttcttcatgcaaattgccatcctgataactactgtaacattgcatttcaagcaatatgaattcaactcccccccaaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcttgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtctcatggtccaatgatattctcaggacccaggaatcagaatgcgtttgtatcaatggaacttgtacagtagtaatgactgatggaaatgctacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcaggacttgttggagacacacccagaaaaagcgacagctccagcagtagccattgtttgaatcctaacaatgaagaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcttagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataagctttcgcaattttagaaaaaact

SEQ ID NO: 56 - DNA, 1448 bp, Influenza virus
  source 1..1448: mol_type = other DNA
atgaatccaaatcaaaagataataacgattggctctgtttctctcacaatttccacaatatgcttcttcatgcaaattgccatcctgataactactgtaacattgcatttcaagcaatatgaattcaactcccccccaaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcttgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaacgatattctcaggacccaggagtcagaatgcgtttgtatcaatggaacttgtacagtagtaatgactgatggaaatgctacaggaaaggctgacactaaaatactattcattgaggaggggaaaatcgtacatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccaggtatgtgtgttcaggacttgttggagacacacccagaaaaagcgacagctccagcagtagccattgtttgaaccctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtrgagttgattaggggaagaaaagaggaaactgaagtcttgtggacctcaaacagtattgttgtgttttgtggcacctcaggtacatatggaacaggctcatggcctgatggggcggacctcaatctcatgcatatataagctttcgcaattttagaaaaaactccttgtttctactg

SEQ ID NO: 57 - DNA, 1448 bp, Influenza virus
  source 1..1448: mol_type = other DNA
atgaatccaaatcaaaagataataacgattggctctgtttctctcacaatttccacaatatgcttcttcatgcaaattgccatcctgataactactgtaacattgcatttcaagcaatatgaattcaactcccccccaaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcttgacaagtgttatcaatttgcccttggacaggggacaacactaaacaacgtgcattcaaataacacagtacgtgataggaccccttaccggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaacgatattctcaggacccaggaatcagaatgcgtttgtatcaatggaacttgtacagtagtaatgactgatggaaatgctacaggaaaagctgatactaaaatactattcatcgaggaggggaaaatcattcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcaggacttgttggagacacacccagaaaaagcgacagctccagcagtagccattgtttgaatcctaacaatgaagaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataagctttcgcaattttagaaaaaaactccttgtttctact

SEQ ID NO: 58 - DNA, 1410 bp, Influenza virus
  source 1..1410: mol_type = other DNA
atgaatccaaatcaaaagataataacgattggctctgtttctctcacaatttccacaatatgcttcttcatgcaaattgccatcctgataactactgtaacattgcatttcaagcaatatgaattcaactcccccccaaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcttgacaagtgttatcaatttgcccttggacagggaacaacactaaacaacgtgcattcaaataacacagtacgtgatagaaccccttatcggactctattgatgaatgagttgggtgttcctttccatctggggaccaagcaagtgtgcatagcatggtccagctcaagctgtcacgatggaaaagcatggctgcatgtttgtataacgggggatgataaaaatgcaactgctagcttcatttacaatgggaggcttgtagatagtgttgtttcatggtccaacgatattctcaggacccaggagtcagaatgcgtttgtatcaatggaacttgtacagtagtaatgactgatggaaatgctacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccaggtatgtgtgttcaggacttgttggagacacacccagaaaaagcgacagctccagcagtagccattgtttgaaccctaacaatgaaaaaggtgatcatggagtgaaaggctgggcctttgatgatggaaatgacgtgtggatggggagaacaatcaacgagacgtcgcgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 59 - DNA, 1434 bp, Influenza virus
  source 1..1434: mol_type = other DNA
atgaatccaaatcaaaagataataacgattggctctgtttctctcaccatttccacaatatgcttcttcatgcaaattgccatcctgataactactgtaacattgcatttcaagcaatatgaattcaactcccccccaaacaaccaagtgatgctgtgtgaaccaacaataatagaaagaaacataacagagatagtgtatttgaccaacaccaccatagagagggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacaataaacaacgtgcattcaaataacacagcacgtgataggacccctcatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagtttcatttacaatgggaggcttgtagatagtgttgtttcatggtccaaagatattctcaggacccaggagtcagaatgcgtttgtatcaatggaacttgtacagtagtaatgactgatggaaatgctacaggaaaagctgatactaaaatattattcattgaggaggggaaaatcgttcatactagcaaattgtcaggaagtgctcagcatgtcgaagagtgctcttgctatcctcgataccctggtgtcagatgtgtctgcagagacaactggaaaggatccaaccggcccatcgtagatataaacataaaggatcatagcattgtttccagttatgtgtgttcaggacttgttggagacacacccagaaaaaccgacagctccagcagcagccattgcttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataagctttcgcaattttagaaaaaact

SEQ ID NO: 60 - DNA, 1720 bp, Influenza virus
  source 1..1720: mol_type = other DNA
atgaagactatcattgctttgggctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacctagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgccttgttggggttcatcatgtgggcctgccaaaagggcaacattaggtgcaacatttgcatttgagtgcattaattaaaaacac

SEQ ID NO: 61 - DNA, 1701 bp, Influenza virus
  source 1..1701: mol_type = other DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattggactggagtcactcaaaacggaacaagttctgcttgcataaggagatctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcggttgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatttgcatttga

SEQ ID NO: 62 - DNA, 1733 bp, Influenza virus
  source 1..1733: mol_type = other DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagctttaattggactggagtcactcaaaacggaaaaagttctgcttgcataaggggatctagtagtagtttctttagtagattaaattggttgacccacttaaactacacatatccagcactgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatattggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaaaaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatttgcatttgagtgcattaattaaaaacacccttgtttctact

SEQ ID NO: 63 - DNA, 1730 bp, Influenza virus
  source 1..1730: mol_type = other DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagaaaactgcacactaatagatgctctattgggagaccctcagtgtgatggctttcaaaataagaaatgggacctttttgttgaaagaagcaaagcctacagcaactgttacccttacgatgtgccggattatgcctcccttaggtcactagttgcctcatccggcacactggagtttaacaatgaaagcttcaattggactggagtcaaacaaaacggaacaagttctgcttgtataaggaaatctagtagtagtttctttagtagattaaattggttgacccacttaaactacacatatccagcattgaacgtgactatgccaaacaatgaacaatttgacaaattgtacatttggggggttcaccacccgggtacggacaaggaccaaatcttcctgtatgctcaatcatcaggaaggatcacagtatctaccaaaagaagccaacaaactgtaatcccaaatatcggatccaggcccagaataagggatatccctagcagaataagcatctattggacaatagtaaaaccgggagacatacttttgattaacagcacagggaatctaattgctcctaggggttacttcaaaatacaaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactaggggcatatttggcgcaatagcgggtttcatagaaaatggttgggagggaatggtggatggttggtacggtttcaggcatcaaaattctgaaggaagaggacaagcagcagatctcaaaagcactcaagcagcaatcgatcaaatcaatgggaagctgaatcgattgatcgggaaaaccaacgagaaattccatcagattgaaaaagaattctcagaagtagaaggaagaattcaggaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggttcaataagaaatggaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaatatttgcatttgagtgcattaattaaaaacacccttgtttct

SEQ ID NO: 64 - DNA, 1720 bp, Influenza virus
  source 1..1720: mol_type = other DNA
atgaaggctatcattgctttgagctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagggaactgcacactaatagatgctctattgggggaccctcaatgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattgggctggagtcactcaaaacggaaaaagttctgcttgcataaggggatctagtagtagtttctttagtagattaaattggttgacccacttaaactacacatatccagcactgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatataggatctagacccagaataagggatatccctagcagaataagcatctattggacaatagtaaaaccgggagacatacttttgattaacagcacagggaatctaattgctcctaggggttacttcaaaatacgragtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatttgcatttgagtgcattaattaaaaacac

SEQ ID NO: 65 - DNA, 1729 bp, Influenza virus
  source 1..1729: mol_type = other DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtaccggattatgcctcccttaggtcactagttgcctcatccggcacactggagtttaaaaatgaaagcttcaattggactggagtcaaacaaaacggaacaagttctgcttgcataaggggatctagtagtagtttctttagtagattaaattggttgacccacttaaactacacatatccagcactgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatttagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcagagcactctgaaattggcaacaggaatgcgaaatgtaccagagaaacaaactagaggcatatttggcgcaatagcgggtttcatagaaaatggttgggagggaatgatggatggttggtacggtttcaggcatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgacctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtattgctttgttggggttcatcatgtgggcctgccaaaagggcaacattagatgcaacatttgcatttgagtgcattaattaaaaacacccttgtttc

SEQ ID NO: 66 - DNA, 1733 bp, Influenza virus
  source 1..1733: mol_type = other DNA
atgaagactatcattgctttgagctacattctatgtctggttttcgctcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcaacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattgggctggagtcactcaaaacggaaaaagttctgcttgcataaggggttctagtagtagtttctttagtagattaaattggttgacccacttaaactacacatatccagcactgaacgtgactatgccaaacaaggaacaatttgacaaattgtacatttggggggttcaccacccgggtacggacaaggaccaaatcttcctgtatgctcaaccatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacagaatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagggttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatggggaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatttgcatttgagtgcattaattaaaaacacccttgtttctact

SEQ ID NO: 67 - DNA, 1701 bp, Influenza virus
  source 1..1701: mol_type = other DNA
atgaagactatcattgctttgagctacattctatgtcttgttttcgctcaagaaatccctggaaatgacaatagcacggcaacgctgtgtcttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattggactggagtcaaacaaaacggaacaagttctgcgtgcataaggggatctagtagtagtttcttcagtagattaaattggttgacccacttaaactacacatatccagcactgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatattggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccgttccaaaatgtaaacaggatcacatacggggcctgtcccagatatgttaagcaaagcactctgaaattggcaacaggaatgcgaaatgtaccagagaaacaaaccagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgacctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtattgctttgttggggttcatcatgtgggcctgccaaaagggcaacattagatgcaacatttgcatttga

SEQ ID NO: 68 - DNA, 1720 bp, Influenza virus
  source 1..1720: mol_type = other DNA
atgaagactatcattgctttgagctgcattctatgtctggttttcgctcaaaaaattcctggaaatgacaatagcacggcaacgctgtgccttgggcaccatgcagtaccaaacggaacgatagtgaaaacaatcacgaatgaccgaattgaagttactaatgctactgagctggttcagaactcctcaataggtgaaatatgcgacagtcctcatcagatccttgatggagaaaactgcacactaatagatgctctattgggagaccctcagtgtgatggctttcaaaataagaaatgggaccttttcgttgaacgaaacaaagcctacagcaactgttacccttatgatgtgccggattatgcatcccttagatcactagttgcctcatccggcacactggagtttaacaatgaaagcttcaattgggctggagtcactcaaaacggaacaagttcttcttgcataaggggatctaaaagtagtttctttagtagattaaattggttgacccacttaaactccaaatacccagcattaaacgtgactatgccaaacaatgaacaatttgacaaattgtacatttggggtgttcaccacccgggtacggacaaggaccaaatctccctgtatgcacaatcatcaggaagaatcacagtatctaccaaaagaagccaacaagctgtaatcccgaatatcggatctagacccagaataagggatatccctagcagaataagcatctattggacaatagtaaaaccaggagacatacttttgattaacagcacagggaatctaattgctcctaggggttacttcaaaatacgaagtgggaaaagctcaataatgagatcagatgcacccattggcaagtgcaagtctgaatgcatcactccaaatggaagcattccaaatgacaaaccattccaaaatgtaaacaggatcacatacggggcatgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatcttaaaagcactcaagcagcaatcgatcaaatcaatgggaagctgaatcgattgatcgggaaaaccaacgagaaattccatcagattgaaaaagagttctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatggggtcaatcagaaatggaacttatgaccacaatgtatacagggatgaagcattaaacaaccggttccagatcaagggagttgagctgaagtcagggtacaaagattggatcctatggatttcctttgccatatcatgttttttgctttgtgttgctctgttggggttcatcatgtgggcctgccaaaagggcaacattaggtgcaacatttgcatttgagtgcattaattaaaaacac

SEQ ID NO: 69 - DNA, 1434 bp, Influenza virus
  source 1..1434: mol_type = other DNA
atgaatccaaatcaaaagataataacgattggctctgtttctctcaccatttccacaatatgctttttcatgcaaattgccattttgataactactgtaacattgcatttcaagcaatatgaattcaactcccccccaaacaaccaagtgatgctgtgtgaaccaacaataatagaaagaaacataacagagatagtgtatttaaccaacaccaccatagagaaggaaatatgccccaaaccagcagaatacagaaattggtcaaaaccgcaatgtggcattacaggatttgcacctttctctaaggacaattcgatcaggctttccgctggtggggacatctgggtgacaagagaaccttatgtgtcatgcgatcctgacaagtgttatcaatttgcccttggacagggaacaacactaaacaacgtgcattcaaataacaaagtacgtgaaaggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcatttgtatcaatggaacttgtacagtagtaatgactgatggaagtgcttcaggaaaagctgatactaaaatactattcattgaggaggggaaaatcgttcatactagcacattgtcaggaagtgctcagcatgtcgaagagtgctcttgctatcctcgatatcctggtgtcagatgtgtctgcagagacaactggaagggctccaatcggcccatcgtagatataaacataaaggatcatagcattgtttccagttatgtgtgttcaggacttgttggagacacacccagaaaaaacgacagctccagcagtagccattgtttggatcctaacaatgaagaaggtggtggcggagtgaaaggctgggcctttgatgatggaaatgacgtgtggatgggaagaacaatcaacgagaagtcacgcttagggtatgaaaccttcaaagtcattgaaggctggtccaaccctaagtccaaattgcagacaaataggcaagtcatagttgacagaggtgataggtccggttattctggtattttctctgttgaaggcaaaagctgcataaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcctatataagctttcgcaattttagaaaaaact

SEQ ID NO: 70 - DNA, 1410 bp, Influenza virus
  source 1..1410: mol_type = other DNA
atgaatccaaatcaaaagataataacgattggctctgtttctctcaccatttccacaatatgcttcttcatgcaaattgccatcctgataactactgtaacattgcatttcaagcaatatgaattcaactcccccccaaacaaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacaaagtacgtgagaggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcgtttgtatcaatggaacttgtacagtagtaatgactgatggaagtgctacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcgtagatataaacataaaggatcatagcattgtttccagttatgtgtgttcaggacttgttggagacacacccagaaaaaacgacagctccagcagtagccattgtttgaatcctaacaatgaagaaggtgttcatggagtgaaaggctgggcctttgatgatggaaatgacgtgtggatggggagaacaatcaacgagaagtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 71 - DNA, 1434 bp, Influenza virus
  source 1..1434: mol_type = other DNA
atgaatccaaatcaaaagataataacgattggctctgtttctctcaccatttccacaatatgctttttcatgcaaattgccattttgataactactgtaacattgcatttcaagcaatatgaattcaactcccccccaaacaaccaagtgatgctgtgtgaaccaacaataatagaaagaaacataacagagatagtgtatttaaccaacaccaccatagagaaggaaatatgccccaaaccagcagaatacagaaattggtcaaaaccgcaatgtggcattacaggatttgcacctttctctaaggacaattcgatcaggctttccgctggtggggacatctgggtgacaagagaaccttatgtgtcatgcgatcctgacaagtgttatcaatttgcccttggacagggaacaacactaaacaacgtgcattcaaataacaaagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcatttgtatcaatggaacttgtacagtagtaatgactgatggaagtgcttcaggaaaagctgatactaaaatactattcattgaggaggggaaaatcgttcatactagcacattgtcaggaagtgctcagcatgtcgaagagtgctcttgctatcctcgatatcctggtgtcagatgtgtctgcagagacaactggaagggctccaatcggcccatcgtagatataaacataaaggatcatagcattgtttccagttatgtgtgttcaggacttgttggagacacacccagaaaaaacgacagctccagcagtagccattgtttggatcctaacaatgaagaaggtggtcatggagtgaaaggctgggcctttgatgatggaaatgacgtgtggatgggaagaacaatcaacgagacgtcacgcttagggtatgaaaccttcaaagtcattgaaggctggtccaaccctaagtccaaattgcagacaaataggcaagtcatagttgacagaggtgataggtccggttattctggtattttctctgttgaaggcaaaagctgcataaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcctatataagctttcgcaattttagaaaaaact

SEQ ID NO: 72 - DNA, 1410 bp, Influenza virus
  source 1..1410: mol_type = other DNA
atgaatccaaatcaaaagataataacgattggctctgtttctctcaccatttccacaatatgcttcttcatgcaaattgccatcctgataactactgtaacattgcatttcaagcaatatgaattcaactcccccccaaacaaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaaagatattctcaggacccaggagtcagaatgcgtttgtatcaatggaacttgtacagtagtaatgactgatggaaatgctacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcgtagatataaacataaaggatcatagcattgtttccagttatgtgtgttcaggacttgttggagacacacccagaaaaaacgacagctccagcagtagccattgtttgaatcctaacaatgaagaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 73 - DNA, 1411 bp, Influenza virus
  source 1..1411: mol_type = other DNA
atgaatccaaatcaaaagataataacgattggctctgtttctctcacaatttccacaatatgcttcttcatgcaaattgccatcctgataactactgtaacattgcatttcaagcaatatgaattcaactcccccccaaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaagacaattcgattaggctttccgctggtggggacatctgggtgacaagagaaccttatgtgtcatgcgatcttgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacawatgggaggcttgtagatagtgttgtttcatggtccaacgatattctcaggacccaggagtcagaatgcgtttgtatcaatggaacttgtacagtagtaatgactgatggaaatgctacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcaggacttgttggagacacacccagaaaaagcgacagctccagcagtagccattgtttgaatcctaacaatgaagaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgatcaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 74 - DNA, 1410 bp, Influenza virus
  source 1..1410: mol_type = other DNA
atgaatccaaatcaaaagataataacgattggctctgtttctctcaccatttccacaatatgcttcttcatgcaaattgccatcctgataactactgtaacattgcatttcaagcaatatgaattcaactcccccccaaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacactaaacaacgtgcattcaaataacacagtacgtgataggaccccttatcggactctattgatgaatgagttgggtgttcctttccatctggggaccaagcaagtgtgcatggcatggtccagctcaagttgtcacgatggaaaagcatggctgcatgtttgtataactggggatgataaaaatgcaactgctagcttcatttacaatgggaggcttgtagatagtgttgtttcatggtccaaagatattctcaggacccaggagtcagaatgcgtttgcatcaatggaacttgtacagtagtaatgactgatggaaatgctacaggaaaagctgatactaaaatactattcattgaggaggggaaaatcgttcatactagcaaattgtcaggaagtgctcagcatgtcgaagagtgctcctgctatcctcgatatcctggtgtcagatgtgtctgcagagacaactggaaaggatccaaccggcccattgtagatataaacataaaggatcatagcattgtttccagttatgtgtgttcaggacttgttggagacacacccagaaaaagcgacagctccagcagtagccattgtttgaatcctaacaatgaagaaggtggtcatggagtgaaaggctgggcctttgatgatggaaatgacgtgtggatggggagaacaatcaacgagacgtcacgcttagggtatgaaaccttcaaagtcgttgaaggctggtccaactctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 75 - protein, 469 aa, Influenza virus; identical to SEQ ID NO: 3

SEQ ID NO: 76 - DNA, 1467 bp, Influenza virus; identical to SEQ ID NO: 9
  source 1..1467: mol_type = other DNA

SEQ ID NO: 77 - protein, 469 aa, Influenza virus
MNPNQKIITIGSVSLTIATICFLMQIAILVTTVTLHFKQYECNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDGNATASFIYNGRLVDSIGSWSKKILRTQESECVCINGTCTVVMTDGSASGKADTKILFIEEGKIVHTSLLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRNQETEVLWTSNSIVVFCGTSGTYGTGSWPDGADINLMPI

(19) United States
(12) Patent Application Publication
(10) Pub. No.: US 2024/0076632 A1
(43) Pub. Date: Mar. 7, 2024

(54) RECOMBINANT INFLUENZA VIRUSES WITH STABILIZED HA FOR REPLICATION IN EGGS

(71) Applicants: The University of Tokyo, Tokyo (JP); Wisconsin Alumni Research Foundation (WARF), Madison, WI (US)

(72) Inventors: Yoshihiro Kawaoka, Middleton, WI (US); Shinya Yamada, Bunkyo-ku (JP); Shiho Chiba, Madison, WI (US)

(21) Appl. No.: 18/461,321

(22) Filed: Sep. 5, 2023

Related U.S. Application Data
(62) Division of application No. 17/004,583, filed on Aug. 27, 2020, now Pat. No. 11,807,872.
(60) Provisional application No. 62/892,241, filed on Aug. 27, 2019.

(51) Int. Cl. C12N 7/00; A61K 39/00

(52) U.S. Cl. CPC C12N 7/00; A61K 39/00; C12N 2760/16021; C12N 2760/16022; C12N 2760/16034; C12N 2760/16051

(57) ABSTRACT

Modified influenza virus neuraminidases are described herein that improve viral replication, thus improving the yield of vaccine viruses. Expression of such modified neuraminidases by influenza virus may also stabilize co-expressed hemagglutinins so that the hemagglutinins do not undergo mutation or decrease the need for HA binding to cells.

### CROSS-REFERENCE TO RELATED APPLICATIONS

This application is a divisional of U.S. patent application Ser. No. 17/004,583, filed on Aug. 27, 2020, which claims the benefit of priority from U.S. Provisional Application Ser. No. 62/892,241, filed on Aug. 27, 2019, both of which are incorporated by reference as if fully set forth herein.

### Statement of Government Funding

This invention was made with government support under HHSN272201400008C awarded by the National Institutes of Health. The government has certain rights in the invention.

### SEQUENCE LISTING

This application contains a Sequence Listing which has been submitted electronically in ST26 format and is hereby incorporated by reference in its entirety. Said ST26 file, created on Sep. 5, 2023, is named “800109US2.xml” and is 163,264 bytes in size.

### BACKGROUND

Influenza is a major respiratory disease in some mammals including horses and is responsible for substantial morbidity and economic losses each year. In addition, influenza virus infections can cause severe systemic disease in some avian species, leading to death. The segmented nature of the influenza virus genome allows for reassortment of segments during virus replication in cells infected with two or more influenza viruses. The reassortment of segments, combined with genetic mutation and drift, can give rise to a myriad of divergent strains of influenza virus over time. The new strains exhibit antigenic variation in their hemagglutinin (HA) and/or neuraminidase (NA) proteins, and in particular the gene coding for the HA protein has a high rate of variability. The predominant current practice for the prevention of flu is vaccination. Most commonly, inactivated virus vaccines are used. As the influenza HA protein is the major target antigen for the protective immune responses of a host to the virus and is highly variable, the isolation of influenza virus and the identification and characterization of the HA antigen in viruses associated with recent outbreaks is important for vaccine production. Based on prevalence and prediction, a vaccine is designed to stimulate a protective immune response against the predominant and expected influenza virus strains.

There are four general types of influenza viruses, Type A, Type B, Type C, and Type D, which are defined by the absence of serological cross reactivity between their internal proteins. Influenza Type A viruses are further classified into subtypes based on antigenic and genetic differences of their glycoproteins, the HA and NA proteins. All the known HA and NA subtypes (H1 to H18 and N1 to N11) have been isolated from aquatic birds, which are thought to act as a natural reservoir for influenza.

Most influenza vaccines are produced in embryonated chicken eggs. However, the WHO-recommended influenza vaccine strains often do not replicate efficiently in embryonated chicken eggs, requiring serial passages in eggs in order to allow for adaptation of the virus. During adaptation and amplification in eggs, the hemagglutinin (HA) protein of influenza viruses often acquires egg-adapting mutations. These egg-adapting mutations in HA often alter the antigenicity of the viruses, resulting in vaccine viruses that are no longer optimally matched to the circulating virus strains.

### SUMMARY

As described herein, an influenza virus was passaged 7 times in eggs (in triplicate) to study the mutations that occurred in the 6 non-immunogenic viral segments during adaptation. Surprisingly, the virus acquired no HA mutations and instead had mutations in the NA, PB2, NP, and M1 proteins. The NA mutations were identical in all three experiments, and they included a deletion and 4 amino acid mutations. The NA mutations were tested alone and it was found that they, e.g., alone or in various combinations, were responsible for the effect, which permitted efficient growth in eggs without HA mutations.

The present disclosure thus relates to influenza mutations that prevent the acquisition of antigenicity-compromising mutations in the hemagglutinin (HA) segment of influenza virus during growth in eggs. The mutations in the neuraminidase (NA) protein of human influenza viruses were found to ‘stabilize’ the HA during egg-passages, e.g., in the presence of the mutations in NA, the HA protein did not acquire egg-adapting mutations. Those NA mutations may also increase the vaccine virus yield.

The disclosure provides isolated recombinant, e.g., reassortant, influenza viruses with selected amino acid residues or deletions at specified positions in NA.

In one embodiment, the NA is selected to not encode a threonine at residue 32. In one embodiment, the NA is selected to not encode an aspartic acid (D) at position 147. In one embodiment, the NA is selected to not encode an asparagine (N) at residue 329. In one embodiment, the NA is selected to not encode a threonine (T) at residue 148 or residue 329. In one embodiment, the NA is selected to not encode a lysine (K) at residue 148 or residue 344. In one embodiment, the NA is selected to not encode a glycine (G) at residue 346. In one embodiment, the NA is selected to not encode a histidine (H) at residue 347. In one embodiment, the NA is selected to not encode an arginine (R) or an asparagine at residue 347. In one embodiment, the NA is selected to not encode a threonine at residue 369. In one embodiment, the NA is selected to not encode a NA having a threonine or lysine at position 148. In one embodiment, the NA is selected to not encode a NA having an aspartic acid at position 151. In one embodiment, the NA is selected to not encode a NA having an asparagine at position 245. In one embodiment, the NA is selected to not encode a NA having a glycine at position 346. In one embodiment, the NA is selected to have a deletion of one or more of residues 46 to 50. The numbering for NA is based on N2. In one embodiment, the disclosure provides an isolated recombinant reassortant influenza virus having six “internal” viral segments from a vaccine influenza virus, e.g., PR8UW, a NA viral segment with one or more of the specified residues at particular positions or a deletion of specified residues, or any combination thereof, and a HA viral segment, e.g., any of H1-H18, e.g., from a circulating influenza virus. Also provided are compositions comprising the recombinant influenza virus, pharmaceutical compositions such as vaccines.

Thus, for vaccine viruses that are to be grown or passaged in cells, e.g., in eggs, replacement of the residue at position 32, 147, 329, 347, or a deletion of one or more of residues 46 to 50, or any combination thereof, in NA, e.g., by mutation, or selection of a NA viral segment for a NA to not encode a threonine at residue 32, to not encode an aspartic acid at position 147, to not encode an asparagine at residue 329, to not encode a histidine at residue 347, to not encode a threonine at residue 369, or to have a deletion of one or more of residues 46 to 50, or any combination thereof, wherein the numbering is based on N2, may result in stabilization of HA and/or higher viral titers. In one embodiment, for vaccine viruses that are to be grown or passaged in cells, e.g., in eggs, replacement of the residue at position 148, 151, 245, 346, or any combination thereof, in NA, e.g., by mutation, or selection of a NA viral segment for a NA to not encode a threonine or lysine at residue 148, to not encode an aspartic acid at position 151, to not encode an asparagine at residue 245, to not encode a glycine at residue 346, or any combination thereof, wherein the numbering is based on N2, may result in stabilization of HA and/or higher viral titers.

In one embodiment, the disclosure provides an isolated recombinant influenza virus comprising PA, PB1, PB2, NP, NS, M, and HA viral segments and a NA viral segment that encodes an NA selected to not encode a threonine at residue 32, to not encode an aspartic acid at position 147, to not encode an asparagine at residue 329, to not encode a histidine at residue 347, to not encode a threonine at residue 369, or to have a deletion of one or more of residues 46 to 50, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine at residue 32, does not have a deletion of residues 46 or 50, encodes an aspartic acid at position 147, encodes an asparagine at residue 329, encodes a histidine at residue 347, or any combination thereof. In one embodiment, the disclosure provides an isolated recombinant influenza virus comprising PA, PB1, PB2, NP, NS, M, and HA viral segments and a NA viral segment that encodes an NA selected to not encode a threonine or lysine at residue 148, to not encode an aspartic acid at position 151, to not encode an asparagine at residue 245, to not encode a glycine at residue 346, to not encode a threonine at residue 369, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine, or lysine at residue 148, encodes an aspartic acid at position 151, encodes an asparagine at residue 245, encodes a glycine at residue 346, any combination thereof. In one embodiment, the isolated recombinant influenza virus is a reassortant. In one embodiment, the NA viral segment encodes a NA that has at least 80%, 85%, 90%, 95%, or 99% amino acid sequence identity to any one of SEQ ID Nos. 1-3, 30-38, 48-50, or 54. In one embodiment, the NA viral segment encodes a NA that has less than 100% amino acid sequence identity to SEQ ID NO:2 or SEQ ID NO:3. In one embodiment, the NA viral segment encodes a N2, N3, N7, or N9 and the positions in N3, N7, or N9 with the specified residue(s) correspond to the specified positions in N2. In one embodiment, the NA viral segment encodes a N1, N4, N5, N6, N8, N10 or N11 and the positions in N1, N4, N5, N6, N8, N10 or N11 with the specified residue(s) correspond to the specified positions in N2. In one embodiment, the residue at position 32 is A, I, G, or L. In one embodiment, the deletion is a deletion of residues 46 to 50. In one embodiment, the residue at position 147 is N or Q. In one embodiment, the residue at position 148 is I or K. In one embodiment, the residue at position 151 is E, Q, H or K. In one embodiment, the residue at position 245 is S, T, A, I, G, or L. In one embodiment, the residue at position 329 is S, V, I, L, A, G, D or E. In one embodiment, the residue at position 344 is E, Q, N, H or D. In one embodiment, the residue at position 346 is V, I, A, S, T, L, or L. In one embodiment, the residue at position 347 is G, Q, N, S, T, Y, C or W. In one embodiment, the residue at position 369 is K, H, R, E, P, or D. In one embodiment, the HA is H1, H3, H7, or H9. In one embodiment, the virus is an influenza A virus. In one embodiment, the PA, PB1, PB2, NP, M, and NS viral segments have at least 85% nucleic acid sequence identity to SEQ ID Nos. 24 to 29 or encode a polypeptide having at least 80%, 85%, 90%, 95%, or 99 amino acid sequence identity to a polypeptide encoded by SEQ ID Nos. 24 to 29 or 39-44. In one embodiment, the PB2 has I, A, L, or G at residue 147. In one embodiment, the virus is an influenza B virus. In one embodiment, the selected NA viral segment does not have an aspartic acid at position 147, does not have an asparagine at residue 329, and does not have an arginine or a histidine at residue 347. In one embodiment, the selected NA viral segment does not a threonine or lysine at position 148, does not have an aspartic acid at position 151, and does not have an asparagine at position 245. In one embodiment, the selected NA viral segment has at least two of: N or Q at position 147, D or E at residue 329, or Q or G at residue 347. In one embodiment, the selected NA viral segment has at least two of: I, L, G or A at position 148, E or Q at position 151, or S, I, T, V or G at position 245. In one embodiment, the selected NA viral segment has at least two of: I or L at position 148, E or Q at position 151, or S, I, T, V or G at position 245. In one embodiment, the selected NA viral segment has N or Q at position 147, S, D or E at residue 329, and Q or G at residue 347. In one embodiment, the selected NA viral segment has N or Q at position 147, S, D or E at residue 329, and V, S, I or L at residue 346. In one embodiment, the residue at position 369 is K, H, R, E, P, or D. In one embodiment, the selected NA viral segment has I, L, G or A at position 148, E or Q at position 151, S, I, T, V or G at position 245 and K, H, R, E, P, or D at position 369. In one embodiment, the selected NA viral segment has I or L at position 148, E or Q at position 151, S, I, T, V or G at position 245 and K, H, R, E, P, or D at position 369. In one embodiment, the residue at position 369 is K, H, R, E, or D.

Further provided is an isolated recombinant nucleic acid, e.g., a vector such as a viral vector, comprising a nucleic acid sequence that encodes an influenza virus NA selected to not encode a threonine at residue 32, to have a deletion of one or more of residues 46-50, to not encode an aspartic acid at position 147, to not encode an asparagine at residue 329, or to not encode a histidine at residue 347, or any combination thereof, wherein the numbering is based on N2. In one embodiment, the isolated recombinant nucleic acid does not encode a threonine or lysine at residue 148, to not encode an aspartic acid at position 151, to not encode an asparagine at residue 245, to not encode a glycine at residue 346, to not encode a threonine at residue 369, or any combination thereof. In one embodiment, the NA has at least 95% amino acid sequence identity to SEQ ID NO:1, SEQ ID NO:3, SEQ ID NO:48, or SEQ ID NO:49. In one embodiment, the NA has less than 100% amino acid sequence identity to SEQ ID NO:2 or SEQ ID NO:3. In one embodiment, the NA is a N2, N3, N7, or N9. In one embodiment, the NA is a N1, N4, N5, N6, N8, N10 or N11. In one embodiment, the residue at position 32 is A, I, G, or L. In one embodiment, the deletion is a deletion of residues 46 to 50. In one embodiment, the residue at position 147 is N or Q. In one embodiment, the residue at position 329 is D or E. In one embodiment, the residue at position 347 is Q, N, S, T, Y, C or W. In one embodiment, the residue at position 148 is I, L, G or A. In one embodiment, the residue at position 148 is I or L. In one embodiment, the residue at position 151 is E, N or Q. In one embodiment, the residue at position 245 is S, T, I, L, A, N, or V. In one embodiment, the residue at position 369 is K, H, R, E, P, or D.

Also provided is a method to prepare influenza virus. The method includes contacting a cell with: a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS DNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production encodes an NA selected to not encode a threonine at residue 32, to not encode an aspartic acid at position 147, to not encode an asparagine at residue 329, to not encode a histidine at residue 347, to not encode a threonine or lysine at residue 148, to not encode an aspartic acid at position 151, to not encode an asparagine at residue 245, to not encode a glycine at residue 346, to not encode a threonine at residue 369, or to have a deletion of one or more of residues 46 to 50, or any combination thereof, wherein the numbering for NA residues is that for N2; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally comprising one or more of: a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, a vector for mRNA production comprising a promoter operably linked to a DNA segment encoding influenza virus NS1, or a vector for mRNA production comprising a promoter operably linked to a DNA segment encoding influenza virus NS2; in an amount effective to yield infectious influenza virus. In one embodiment, the NA has at least 80%, 85%, 90%, 95%, or 99% amino acid sequence identity to, for example, SEQ ID NO:1 SEQ ID NO:2, SEQ ID NO:3, SEQ ID NO:48 or SEQ ID NO:49. In one embodiment, the NA has at least 80%, 85%, 90%, 95%, or 99% amino acid sequence identity to, for example, SEQ ID Nos. 51-59 or 69-70. In one embodiment, the NA has less than 100% amino acid sequence identity to SEQ ID NO:2 or SEQ ID NO:3. In one embodiment, the NA is N2, N3, N7, or N9. In one embodiment, the NA is N1, N4, N5, N6, N8, N10 or N11. In one embodiment, the residue at position 32 is A, I, G, or L. In one embodiment, the deletion is a deletion of residues 46 to 50. In one embodiment, the residue at position 147 is N or Q. In one embodiment, the residue at position 329 is S, D or E. In one embodiment, the residue at position 347 is Q, N, S, T, Y, C or W. In one embodiment, the residue at position 148 is I, L, G or A. In one embodiment, the residue at position 148 is I or L. In one embodiment, the residue at position 151 is E, N or Q. In one embodiment, the residue at position 245 is S, T, I, L, A, N, or V. In one embodiment, the residue at position 329 is S, I, L, A, N, or V. In one embodiment, the residue at position 344 is E, Q, N, H or D. In one embodiment, the residue at position 346 is V, S, T, I, L, A, or V. In one embodiment, the residue at position 347 is G, S, T, I, L, A, or V. In one embodiment, the residue at position 369 is K, H, R, E, P, or D.

In one embodiment, the HA is H1, H3, H5, H7, or H9. In one embodiment, the virus is an influenza A virus. In one embodiment, PA, PB1, PB2, NP, M, and NS viral segments have at least 85%, 85%, 90%, 95%, or 99% nucleic acid sequence identity to SEQ ID Nos. 24 to 29 or 39 to 44 or encode a polypeptide having at least 80% , 85%, 90%, 95%, or 99% amino acid sequence identity to a polypeptide encoded by SEQ ID Nos. 24 to 29 or 39 to 44. In one embodiment, PB2 has I, A, L, or G at residue 147. In one embodiment, HA is H2, H4, H5, H6, H8, or any of H10-H18. In one embodiment, the virus is an influenza B virus.

Further provided is a method of immunizing an avian or a mammal with a composition having an effective amount of the virus described herein. In one embodiment, the composition comprises at least one other different influenza virus. In one embodiment, the mammal is a human. In one embodiment, the composition is administered intranasally or via injection.

Thus, the invention provides a method to select for influenza viruses with enhanced replication in cell culture, e.g., enhanced replication in embryonated eggs. The method includes providing cells suitable for influenza vaccine production; serially culturing one or more influenza virus isolates in eggs; and isolating serially cultured virus with enhanced growth relative to the one or more isolates prior to serial culture. Also provided is a method to identify a NA that stabilizes HA and/or that confers altered growth of a recombinant influenza virus, e.g., in eggs. The method includes introducing one or more substitutions or deletions as described herein into a NA viral segment to yield a mutant NA viral segment; and optionally identifying whether the mutant NA viral segment, when present in a replication competent recombinant influenza virus, results in enhanced replication of the recombinant influenza virus in eggs and optionally inhibits HA mutations, relative to a corresponding replication competent influenza virus without the one or more substitutions and/or deletions in NA.

In one embodiment, the disclosure provides isolated influenza type A virus with a characteristic residue(s) and/or deletion, or a combination thereof, in NA described herein. In one embodiment, the isolated influenza type A virus with a characteristic residue(s) and/or deletion, or a combination thereof, has an NA amino acid sequence with at least 80%, e.g., 90%, 92%, 95%, 97% or 99%, including any integer between 80 and 99, contiguous amino acid sequence identity to a polypeptide encoded by one of SEQ ID NOs:1, 2, 3, or 30-38. In one embodiment, the isolated influenza type A virus of the invention with a characteristic residue(s) and/or deletion, or a combination thereof, has an HA from any one of subtypes 1-18 of HA. In one embodiment the characteristic residue is a conservative substitution, e.g., relative to SEQ ID NO:2 or SEQ ID NO:3. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In one embodiment, conservative amino acid substitution groups are: threonine-valine-leucine-isoleucine-alanine; phenylalanine-tyrosine; lysine-arginine; alanine-valine; glutamic-aspartic; and asparagine-glutamine.

In one embodiment, a mutation is introduced into a NA viral segment of an influenza virus isolate, e.g., via recombinant DNA techniques including site-specific mutagenesis, or replacing a portion of the NA coding sequence with a portion that includes the characteristic residue(s) or deletion. In one embodiment, a NA viral segment with a characteristic residue and/or deletion described herein is combined with a HA segment, and internal viral segments of an influenza vaccine virus.

The disclosure provides a plurality of influenza virus vectors of the invention, e.g., those useful to prepare reassortant viruses including 6:1:1 reassortants, 6:2 reassortants and 7:1 reassortants. A 6:1:1 reassortant is an influenza virus with 6 internal viral segments from a vaccine virus, a HA viral segment that is from a different (second) viral isolate than the vaccine virus, and a NA viral segment with a characteristic residue(s) and/or deletion, or a combination thereof, as described herein, which is from a different viral source than the HA segment and the vaccine virus; a 6:2 reassortant is an influenza virus with 6 internal viral segments from a vaccine virus, and a NA viral segment having a characteristic residue(s) and/or deletion, or a combination thereof, which segment is from the same source as the HA segment, and a HA viral segment from a different viral isolate than the vaccine virus; and a 7:1 reassortant, in one embodiment, is an influenza virus with 6 internal viral segments and a HA segment from a vaccine virus, and a NA segment that is modified to include the characteristic residue(s) and/or deletion, or a combination thereof, which NA segment is from a different viral source than the vaccine virus.

In one embodiment of the invention, the plurality includes vectors for vRNA production selected from a vector comprising a promoter operably linked to an influenza virus PA DNA linked to a transcription termination sequence, a vector comprising a promoter operably linked to an influenza virus PB1 DNA linked to a transcription termination sequence, a vector comprising a promoter operably linked to an influenza virus PB2 DNA linked to a transcription termination sequence, a vector comprising a promoter operably linked to an influenza virus HA DNA linked to a transcription termination sequence, a vector comprising a promoter operably linked to an influenza virus NP DNA linked to a transcription termination sequence, a vector comprising a promoter operably linked to an influenza virus NA DNA linked to a transcription termination sequence, a vector comprising a promoter operably linked to an influenza virus M DNA linked to a transcription termination sequence, and a vector comprising a operably linked to an influenza virus NS DNA linked to a transcription termination sequence. In one embodiment, the DNAs for vRNA production of PB1, PB2, PA, NP, M, and NS, have sequences from an influenza virus that replicates to high titers in cultured mammalian cells such as MDCK cells, Vero cells or PER.C6® cells or embryonated eggs, and/or from a vaccine virus, e.g., one that does not cause significant disease in humans. The DNA for vRNA production of NA may be from any NA, e.g., any of N1-N11, and the DNA for vRNA production of HA may be from any HA, e.g., H1-H18. In one embodiment, the DNAs for vRNA production may be for an influenza B or C virus. For example, the DNAs for vRNA production include influenza B virus PA, PB1, PB2, NP, NS, and M or influenza B virus PA, PB1, PB2, NP, NS, M, and NA, wherein the vRNA for NA has a NA with a characteristic residue and/or deletion as described herein. The DNAs for vRNA production of NA and HA may be from different strains or isolates (6:1:1 reassortants) or from the same strain or isolate (6:2 reassortants), or the NA or HA may be from the same strain or isolate as that for the internal genes (7:1 reassortant). The plurality also includes vectors for mRNA production selected from a vector encoding influenza virus PA, a vector encoding influenza virus PB1, a vector encoding influenza virus PB2, and a vector encoding influenza virus NP, and optionally one or more vectors encoding NP, NS, M, e.g., M1 and M2, HA or NA. The vectors encoding viral proteins may further include a transcription termination sequence.

Viruses that may provide the internal genes for reassortants within the scope of the invention include viruses that have high titers, e.g., titers of at least about 10⁵ PFU/mL, e.g., at least 10⁶ PFU/mL, 10⁷ PFU/mL or 10⁸ PFU/mL; high titers in embryonated eggs, e.g., titers of at least about 10⁷ EID₅₀/mL, e.g., at least 10⁸ EID₅₀/mL, 10⁹ EID₅₀/mL or 10¹⁰ EID₅₀/mL; high titers in MDCK cells, e.g., titers of at least about 10⁷ PFU/mL, e.g., at least 10⁸ PFU/mL, or high titers in two of more of those host cells.

Other reassortants with internal genes from other PR8 isolates or vaccine viruses may be employed in recombinant reassortant viruses.

In one embodiment, the DNAs for the internal genes for PB1, PB2, PA, NP, M, and NS encode proteins with substantially the same activity as a corresponding polypeptide encoded by one of SEQ ID NOs:24-29 or 39 to 44. As used herein, “substantially the same activity” includes an activity that is about 0.1%, 1%, 10%, 30%, 50%, 90%, e.g., up to 100% or more, or detectable protein level that is about 80%, 90% or more, the activity or protein level, respectively, of the corresponding full-length polypeptide. In one embodiment, the nucleic acid a sequence encoding a polypeptide which is substantially the same as, e.g., having at least 80%, e.g., 90%, 92%, 95%, 97% or 99%, including any integer between 80 and 99, contiguous amino acid sequence identity to, a polypeptide encoded by one of SEQ ID NOs:24-29 or 39 to 44. In one embodiment, the isolated and/or purified nucleic acid molecule comprises a nucleotide sequence which is substantially the same as, e.g., having at least 50%, e.g., 60%, 70%, 80% or 90%, including any integer between 50 and 100, or more contiguous nucleic acid sequence identity to one of SEQ ID NOs:24-29 and, in one embodiment, also encodes a polypeptide having at least 80%, e.g., 90%, 92%, 95%, 97% or 99%, including any integer between 80 and 99, contiguous amino acid sequence identity to a polypeptide encoded by one of SEQ ID NOs:24-29 or 39 to 44. In one embodiment, the influenza virus polypeptide has one or more, for instance, 2, 5, 10, 15, 20 or more, conservative amino acids substitutions, e.g., conservative substitutions of up to 10% or 20% of the residues, relative to a polypeptide encoded by one of SEQ ID NOs:24-29 or 39 to 44. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In one embodiment, conservative amino acid substitution groups are: valine-leucine-isoleucine; phenylalanine-tyrosine; lysine-arginine; alanine-valine; glutamic-aspartic; and asparagine-glutamine. In one embodiment, the influenza virus polypeptide has one or more, for instance, 2, 3 or 4, nonconservative amino acid substitutions, relative to a polypeptide encoded by one of SEQ ID NOs:24-29.

In one embodiment, the nucleic acid a sequence encoding a NA polypeptide which is substantially the same as, e.g., having at least 80%, e.g., 90%, 92%, 95%, 97% or 99%, including any integer between 80 and 99, contiguous amino acid sequence identity to, one of SEQ ID Nos. 1-3 or 48-49, or a polypeptide encoded by one of SEQ ID NOs:51-59, or one of Accession Nos. ACP41107.1 (N1) (SEQ ID NO:36) AIK26357.1 (N7) (SEQ ID NO:37), ALH21372.1 (N9) (SEQ ID NO:45), or BAK86313.1 (N2) (SEQ ID NO:50), the sequences of which are incorporated by reference herein. In one embodiment, the isolated and/or purified nucleic acid molecule encodes a polypeptide having at least 80%, e.g., 90%, 92%, 95%, 97% or 99%, including any integer between 80 and 99, contiguous amino acid sequence identity to an one of SEQ ID NOs:1, 3, 30-35, or 48-49, one of Accession Nos. ACP41107.1 (N1) AIK26357.1 (N7), ALH21372.1 (N9), or BAK86313.1 (N2), or to a NA encoded by one of SEQ ID Nos. 51-59, the sequences of which are incorporated by reference herein. In one embodiment, the influenza virus polypeptide has one or more, for instance, 2, 5, 10, 15, 20 or more, conservative amino acids substitutions, e.g., conservative substitutions of up to 10% or 20% of the residues, relative to SEQ ID NOs:1, 3, 30-35, 48-49, or one of Accession Nos. ACP41107.1 (N1) AIK26357.1 (N7), ALH21372.1 (N9), or BAK86313.1 (N2), or a NA encoded by one of SEQ ID Nos. 51-59, the sequences of which are incorporated by reference herein. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In one embodiment, conservative amino acid substitution groups are: valine-leucine-isoleucine; phenylalanine-tyrosine; lysine-arginine; alanine-valine; glutamic-aspartic; and asparagine-glutamine. In one embodiment, the influenza virus polypeptide has one or more, for instance, 2, 3 or 4, nonconservative amino acid substitutions, relative to a polypeptide having one of SEQ ID NOs:1, 3, 30-35, 48-49, or one of Accession Nos. ACP41107.1 (N1) AIK26357.1 (N7), ALH21372.1 (N9), or BAK86313.1 (N2), or a NA encoded by one of SEQ ID Nos. 51-59, the sequences of which are incorporated by reference herein.

The invention thus includes the use of isolated and purified vectors or plasmids, which express or encode influenza virus proteins, or express or encode influenza vRNA, both native and recombinant vRNA. The vectors comprise influenza cDNA, e.g., influenza A (e.g., any influenza A gene including any of the 18 HA or 11 NA subtypes), B or C DNA (see Fields Virology (Fields et al. (eds.), Lippincott, Williams and Wickens (2013), which is specifically incorporated by reference herein). Any suitable promoter or transcription termination sequence may be employed to express a protein or peptide, e.g., a viral protein or peptide, a protein or peptide of a nonviral pathogen, or a therapeutic protein or peptide.

A composition or plurality of vectors of the invention may also comprise a heterologous gene or open reading frame of interest, e.g., a foreign gene encoding an immunogenic peptide or protein useful as a vaccine or in gene replacement, for instance may encode an epitope useful in a cancer therapy or vaccine, or a peptide or polypeptide useful in gene therapy. When preparing virus, the vector or plasmid comprising the gene or cDNA of interest may substitute for a vector or plasmid for an influenza viral gene or may be in addition to vectors or plasmids for all influenza viral genes. Thus, another embodiment of the invention comprises a composition or plurality of vectors as described above in which one of the vectors is replaced with, or further comprises, 5′ influenza virus sequences optionally including 5′ influenza virus coding sequences or a portion thereof, linked to a desired nucleic acid sequence, e.g., a desired cDNA, linked to 3′ influenza virus sequences optionally including 3′ influenza virus coding sequences or a portion thereof. In one embodiment, the desired nucleic acid sequence such as a cDNA is in an antisense (antigenomic) orientation. The introduction of such a vector in conjunction with the other vectors described above to a host cell permissive for influenza virus replication results in recombinant virus comprising vRNA corresponding to the heterologous sequences of the vector.

The promoter in a vector for vRNA production may be a RNA polymerase I promoter, a RNA polymerase II promoter, a RNA polymerase III promoter, a T7 promoter, or a T3 promoter, and optionally the vector comprises a transcription termination sequence such as a RNA polymerase I transcription termination sequence, a RNA polymerase II transcription termination sequence, a RNA polymerase III transcription termination sequence, or a ribozyme. Ribozymes within the scope of the invention include, but are not limited to, tetrahymena ribozymes, RNase P, hammerhead ribozymes, hairpin ribozymes, hepatitis ribozyme, as well as synthetic ribozymes. In one embodiment, the RNA polymerase I promoter is a human RNA polymerase I promoter.

The promoter or transcription termination sequence in a vRNA or virus protein expression vector may be the same or different relative to the promoter or any other vector. In one embodiment, the vector or plasmid which expresses influenza vRNA comprises a promoter suitable for expression in at least one particular host cell, e.g., avian or mammalian host cells such as canine, feline, equine, bovine, ovine, or primate cells including human cells, or for expression in more than one host.

In one embodiment, at least one vector for vRNA comprises a RNA polymerase II promoter linked to a ribozyme sequence linked to viral coding sequences linked to another ribozyme sequences, optionally linked to a RNA polymerase II transcription termination sequence. In one embodiment, at least 2, e.g., 3, 4, 5, 6, 7 or 8, vectors for vRNA production comprise a RNA polymerase II promoter, a first ribozyme sequence, which is 5′ to a sequence corresponding to viral sequences including viral coding sequences, which is 5′ to a second ribozyme sequence, which is 5′ to a transcription termination sequence. Each RNA polymerase II promoter in each vRNA vector may be the same or different as the RNA polymerase II promoter in any other vRNA vector. Similarly, each ribozyme sequence in each vRNA vector may be the same or different as the ribozyme sequences in any other vRNA vector. In one embodiment, the ribozyme sequences in a single vector are not the same.

In one embodiment, at least one vector comprises sequences corresponding to those encoding PB1, PB2, PA, NP, M, or NS, or a portion thereof, having substantially the same activity as a corresponding polypeptide encoded by one of SEQ ID NOs:24-29 or 39 to 44, e.g., a sequence encoding a polypeptide with at least 80%, e.g., 85%, 90%, 92%, 95%, 98%, 99% or 100%, including any integer between 80 and 100, amino acid identity to a polypeptide encoded by one of SEQ ID NOs:24-29. Optionally, two vectors may be employed in place of the vector comprising a promoter operably linked to an influenza virus M cDNA linked to a transcription termination sequence, e.g., a vector comprising a promoter operably linked to an influenza virus M1 cDNA linked to a transcription termination sequence and a vector comprising a promoter operably linked to an influenza virus M2 cDNA linked to a transcription termination sequence.

A plurality of the vectors of the invention may be physically linked or each vector may be present on an individual plasmid or other, e.g., linear, nucleic acid delivery vehicle. In one embodiment, each vRNA production vector is on a separate plasmid. In one embodiment, each mRNA production vector is on a separate plasmid.

The invention also provides a method to prepare influenza virus. The method comprises contacting a cell with a plurality of the vectors of the invention, e.g., sequentially or simultaneously, in an amount effective to yield infectious influenza virus. The invention also includes isolating virus from a cell contacted with the plurality of vectors. Thus, the invention further provides isolated virus, as well as a host cell contacted with the plurality of vectors or virus of the invention. In another embodiment, the invention includes contacting the cell with one or more vectors, either vRNA or protein production vectors, prior to other vectors, either vRNA or protein production vectors. In one embodiment, the promoter for vRNA vectors employed in the method is a RNA polymerase I promoter, a RNA polymerase II promoter, a RNA polymerase III promoter, a T3 promoter or a T7 promoter. In one embodiment, the RNA polymerase I promoter is a human RNA polymerase I promoter. In one embodiment, each vRNA vector employed in the method is on a separate plasmid. In one embodiment, the vRNA vectors employed in the method are on one plasmid or on two or three different plasmids. In one embodiment, each mRNA vector employed in the method is on a separate plasmid. In one embodiment, the mRNA vectors for PA, PB1, PB2 and NP employed in the method are on one plasmid or on two or three different plasmids.

The methods of producing virus described herein, which do not require helper virus infection, are useful in viral mutagenesis studies, and in the production of vaccines (e.g., for AIDS, influenza, hepatitis B, hepatitis C, rhinovirus, filoviruses, malaria, herpes, and foot and mouth disease) and gene therapy vectors (e.g., for cancer, AIDS, adenosine deaminase, muscular dystrophy, ornithine transcarbamylase deficiency and central nervous system tumors). Thus, a virus for use in medical therapy (e.g., for a vaccine or gene therapy) is provided.

The invention also provides isolated viral polypeptides, and methods of preparing and using recombinant virus of the invention. The methods include administering to a host organism, e.g., a mammal, an effective amount of the influenza virus of the invention, e.g., an inactivated virus preparation, optionally in combination with an adjuvant and/or a carrier, e.g., in an amount effective to prevent or ameliorate infection of an animal such as a mammal by that virus or an antigenically closely related virus. In one embodiment, the virus is administered intramuscularly while in another embodiment, the virus is administered intranasally. In some dosing protocols, all doses may be administered intramuscularly or intranasally, while in others a combination of intramuscular and intranasal administration is employed. The vaccine may further contain other isolates of influenza virus including recombinant influenza virus, other pathogen(s), additional biological agents or microbial components, e.g., to form a multivalent vaccine. In one embodiment, intranasal vaccination, for instance containing with inactivated influenza virus, and a mucosal adjuvant may induce virus-specific IgA and neutralizing antibody in the nasopharynx as well as serum IgG.

The influenza virus of the invention may employed with other anti-virals, e.g., amantadine, rimantadine, and/or neuraminidase inhibitors, e.g., may be administered separately in conjunction with those anti-virals, for instance, administered before, during and/or after.

Thus, the modified neuraminidase comprises at least one, or at least two, or at least three modifications, wherein the modification comprise one or more amino acids within positions 29-35, one or more amino acids within positions 44-52, one or more amino acids within positions 144-154, one or more amino acid positions within 240-250, one or more amino acids within positions 326-333, one or more amino acid positions within 344-350, one or more amino acid positions within 365-375, or combinations thereof, wherein the numbering is that for N2. In one embodiment, the NA comprises a deletion of at least one proline, asparagine, glutamine, valine, or a combination of a proline, one or more asparagine(s), a glutamine, and a valine within positions 44-52; a substitution (replacement) of a threonine within positions 29-35; a substitution (replacement) of an threonine or an aspartic acid within positions 145-155; a substitution (replacement) of an asparagine within positions 240 to 250 or 326-333; a substitution (replacement) of a histidine within positions 345-350; or a combination thereof.

### BRIEF DESCRIPTION OF FIGURES

FIGS. 1A-1L. Nucleotide sequences for the viral segments of A/Yokohama/2017/2003 (SEQ ID Nos. 4-11), and amino acid sequence of the NA of A/Yokohama/2017/2003 (SEQ ID NO:3).

FIG. 2. Amino acid sequence for the NA of A/Saitama/103/2014 (SEQ ID NO:2)

FIGS. 3A-3G. Nucleotide sequence of NA viral segment (SEQ ID NO:12) and amino acid sequences for NA of mutant of A/Yokohama/2017/2003 (SEQ ID NO:1), and nucleotide sequence of other viral segments of the mutant (SEQ ID Nos.12-21)

FIG. 4. Graph showing titers in eggs of various reassortants with the PB2, M, NA and NP segments of mutant and wild-type A/Yokohama/2017/2003. Virus inoculation: 2×10³ pfu/egg into allantoic fluid, 72 h incubation at 37° C.

FIGS. 5A-5B. Locations of the NA mutations on the 3D structure of N2 NA.

FIG. 6. Graph showing titers in eggs for recombinant viruses with specific mutations found in the mutant of A/Yokohama/2017/2003 (“Y2017-M3L4”). Virus inoculation: 2×10³ pfu/egg into allantoic fluid, 72 h incubation at 37° C.

FIG. 7. Graph of virus titer in eggs for reassortants with two different backbones (PA, PB1, PB2, NP, NS and M) and two different HA and NA combinations (e.g., PB2-I504V, PB1-M40L/G180W, PA-R401K, NP-I116L, NS1-A30P/R118K; and NA of Y2017-M3L4 contains mutations; NA-T32A, D147N, N329D, H347Q and deletion of 46-50aa). Virus inoculation: 2×10³ pfu/egg into allantoic fluid, 72 h incubation at 37° C.

FIG. 8. Amino acid sequence comparison of Yokohama/2017/2003 NA wild-type (SEQ ID NO:3) and Y2017-M3L4 (SEQ ID NO:1).

FIGS. 9A-9B. Exemplary NA sequences for N3, N4, N6, N7, N8, and N9 (SEQ ID Nos. 30-35).

FIGS. 10A-10F. Exemplary sequences for the internal viral segments for a master vaccine strain (SEQ ID Nos. 39-44).

FIGS. 11A-11B. Exemplary NA sequences (SEQ ID Nos. 71-74).

FIG. 12. Titers in eggs for various NA mutants.

FIG. 13. Titers of HK4801HA, Y2017-M3L4NA and HY-PR8 (PB2 C4U, I504V; PB1 C4U, M40L/G180W; PA C4U, R401K; NP I116L; NS A30P/R118K) and analyses for HA mutations in infected eggs over time.

FIG. 14 shows data for viruses passaged in eggs that had certain NA mutants but did not result in substitutions in HA.

FIG. 15 is a schematic of the positions of certain NA residues.

FIG. 16 is a schematic of the positions of certain NA residues.

FIG. 17 shows virus titers for egg passaged isolates (HK4801NA (T148K, D151E, H347G, and T369K)) conferred efficient replication in the allantoic cavity to viruses possessing either HK4801HA or Singapore0019 HA (HY-PR8 backbone).

FIG. 18 shows egg titers for different combinations of selected residues at positions 153, 329, 347, and 369 in NA.

FIG. 19 summarizes virus titers (HK4801HA, Y2017-M3L4NA and HY-PR8 (PB2 C4U, I504V; PB1 C4U, M40L/G180W; PA C4U, R401K; NP I116L; NS A30P/R118K) and HA status over time.

FIG. 20 summarizes virus titers and HA status for viruses with different NAs.

FIG. 21 provides inoculation and harvested virus titers in allantoic passages (HA-K189E/N158K/A212T mutant virus).

FIG. 22 shows detection of HA status after multiple passages.

FIGS. 23A-23B show egg titers for viruses with different NAs.

FIG. 24 is an enlarged view of the NA activity center. Most egg-adapted mutations are located in/around the NA active site.

FIG. 25. Locations of amino acid substitutions in the neuraminidase proteins of egg-adapted influenza A/Hong Kong/4801/2014(H3N2) and A/Alaska/232/2015(H3N2) (SEQ ID Nos. 51-52).

FIG. 26. Introduction of NA mutations (see FIG. 25) into the NA of H3N2 viruses from the 2017/18 season (SEQ ID NO:53) enhanced HY-PR8 backbone virus growth without HA mutations.

FIG. 27. Mutations observed in NA mutant viruses (HY-PR8 backbone) in FIG. 26 during egg passages.

FIG. 28. Introduction of NA mutations into the NA of H3N2 viruses from the 2017/18 season enhanced HY-PR8 backbone virus growth without HA mutations.

FIG. 29. Mutations observed in NA mutant viruses (HY-PR8 backbone) in FIG. 28 during egg passages.

FIG. 30. The HY-PR8 backbone virus possessing A/Yokohama/48/2018HA and A/Yokohama/48/2018NA (T148K, D151E, N245S, H347G, and T369K) acquired the same NA-K148I mutation, and no HA mutations were detected (SEQ ID NO:54).

FIG. 31. A HY-PR8 backbone virus possessing A/Yokohama/48/2018HA and A/Yokohama/48/2018NA(T148I, D151 E, N245S, H347G, and T369K) only acquired the HA-435L mutation in the stem region.

FIG. 32. HA-H435L locates to the stem region of the HA trimer. previous study reported that HA-H435L did not affect antigenicity (Kuwahara et al., Jpn. J. Infect. Dis., 2018).

FIG. 33. Effect of introducing NA-T148I, D151 E, N245S, H347G, and T369K into the NA of H3N2 viruses from the 2017/18 season.

FIG. 34. Effect of introducing NA-T148I, D151E, N245S, H347G, and T369K into the NA of H3N2 viruses from the 2018/19 season (SEQ ID Nos. 55-56).

FIG. 35. Yokohama48NA (T148I, D151 E, N245S, H347G, and T369K) enhanced the growth of viruses possessing the HA of H3N2 viruses of the 2017-18 and 2018-19 seasons (SEQ ID Nos. 57-58).

FIG. 36. Yokohama48NA (T148I, D151 E, N245S, H347G, and T369K) has reduced sialidase activity.

FIG. 37. The growth of Kansas/14/2017 (SEQ ID NO:59) was enhanced by introducing the NA mutations T148I, D151 E, N245S, H347G, and T369K or by possessing Yokohama48NA (T148I, D151E, N245S, H347G, and T369K).

FIG. 38. Neutralization by human monoclonal IgG clone F045-092 against viruses possessing Aichi/2/68HA and wild-type or mutant NA from 2017-18 season H3N2 viruses.

FIG. 39A. Position of sialic acid relative to residues in NA.

FIG. 39B. Enlarged view of FIG. 38A.

FIGS. 40A-40N. Exemplary NA sequences (SEQ ID Nos. 55-59 and 71-74) for modification and modified NA sequences (SEQ ID Nos 69-70).

FIGS. 41A-41N. Exemplary HA sequences (SEQ ID Nos. 60-68) from strains that were stabilized.

FIG. 42. Exemplary NA residues in 6M virus which were found in egg-grown A/Hong Kong/4801/2014 and A/Alaska/232/2015.

FIG. 43. Viruses in which 6M residues were introduced into the NA of A/Delaware/33/2018 and A/Saint-Petersburg/RII-324S/2019 and viruses possessing Yokohama/147/2017NA(6M) NA enhanced the virus growth of HY-PR8-backbone virus possessing wild type HA of A/Delaware/33/2018 or A/Saint-Petersburg/RII-324S/2019. Harvested viruses possessing each strain's NA(6M) or Yokohama/147/2017NA(6M) were sequenced however none had additional mutations in HA and NA.

FIG. 44. Viruses in which 6M was introduced into the NA of A/Tokyo/UT-GR85/2019 and A/Kanagawa/IC1820/2019 did not enhance HY-PR8-backbone virus growth. However, viruses possessing Yokohama/147/2017NA(6M) showed enhanced the virus growth of HY-PR8-backbone virus possessing wild type HA of A/Tokyo/UT-GR85/2019 or A/Kanagawa/IC1820/2019 without HA mutations. Harvested viruses possessing Yokohama/147/2017NA(6M) were sequenced however none had additional mutations in HA and NA.

FIG. 45. Mutations observed in the HA and NA proteins of HY-PR8 backbone viruses possessing Yokohama147NA(6M) during 10 passages in eggs.

FIG. 46. Location of HA mutations occurred during egg passages (shown in FIG. 43) on the 3D structure of HA protein.

FIG. 47. Location of NA mutations occurred during egg passages (shown in FIG. 43) on the 3D structure of NA protein.

FIG. 48. Reduced molecular weight of Yokohama147NA(6M). VP40-induced VLPs bearing FLAG-tagged Yokohama147NA or Yokohama147NA(6M) were analyzed by immunoblotting with anti-FLAG and anti-VP40 antibodies.

FIG. 49. Loss of glycosylation sites of NA protein due to mutations.

FIG. 50. Receptor-binding specificities of Yokohama147HA, Yokohama147NA, and Yokohama147NA(6M).

FIG. 51. Introduction of 6M into Yokohama147NA decreased sialidase activity.

FIG. 52. HY-PR8 backbone virus possessing wild type HA and mutant NA(T148I, D151 E, N245S, T329S, K344E, H347G and T369K) (=6M+T148I+T329S+K344E) from A/Kansas/14/2017NA acquired none of HA and NA mutations during egg passages.

FIG. 53. Location of NA mutations; T148I, D151 E, N245S, T329S, K344E, G346V, H347G and T369K on the 3D structure of NA protein. 6M shown in purple and T148I, T329S, K344E shown in green.

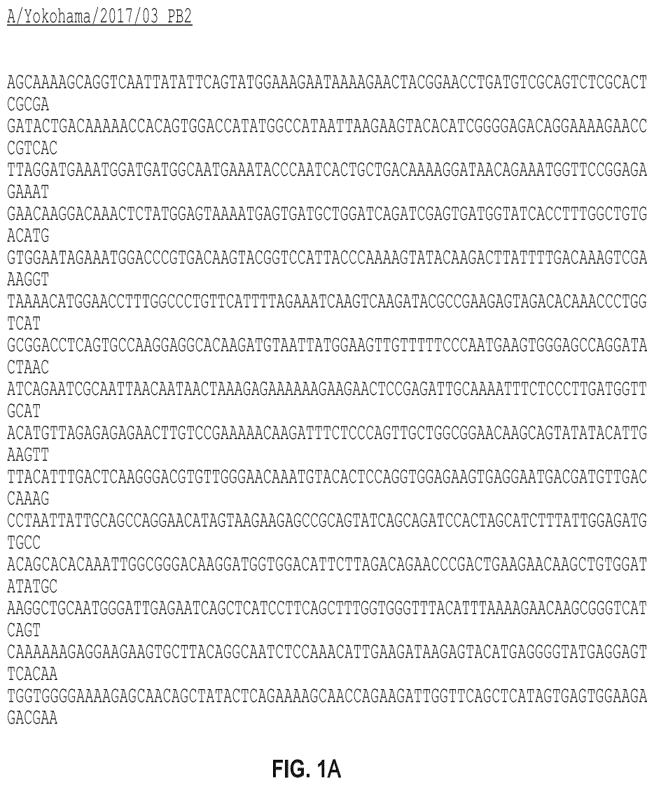

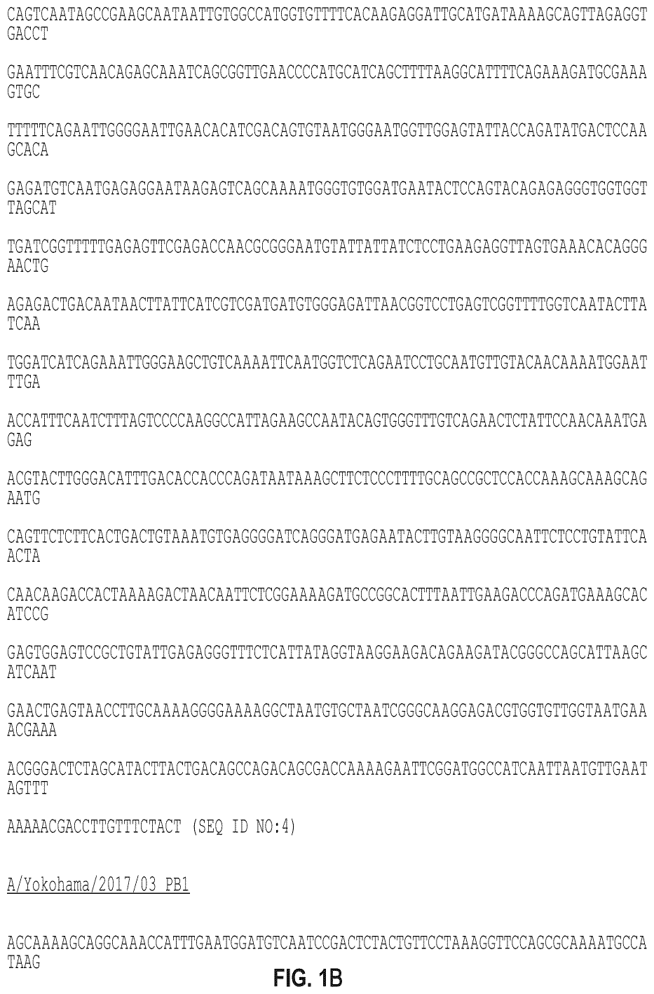

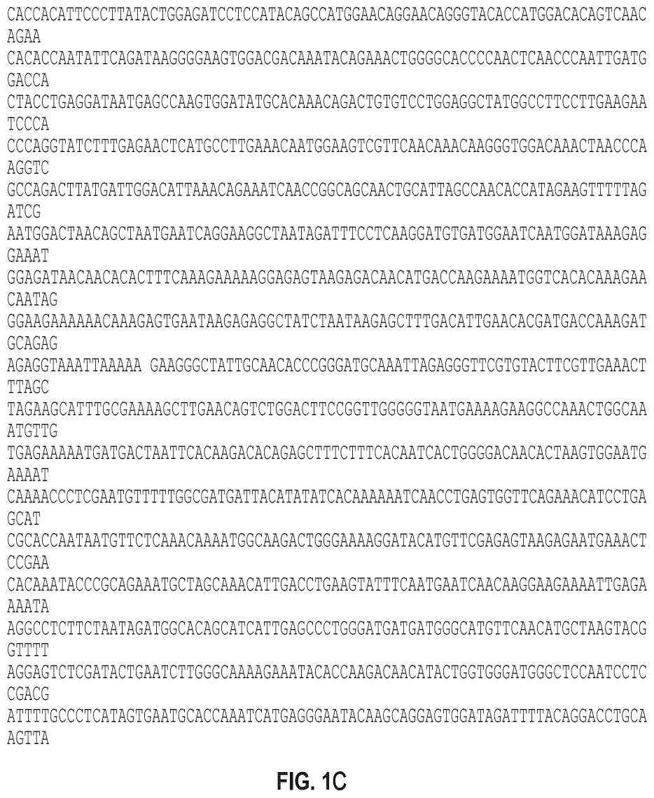

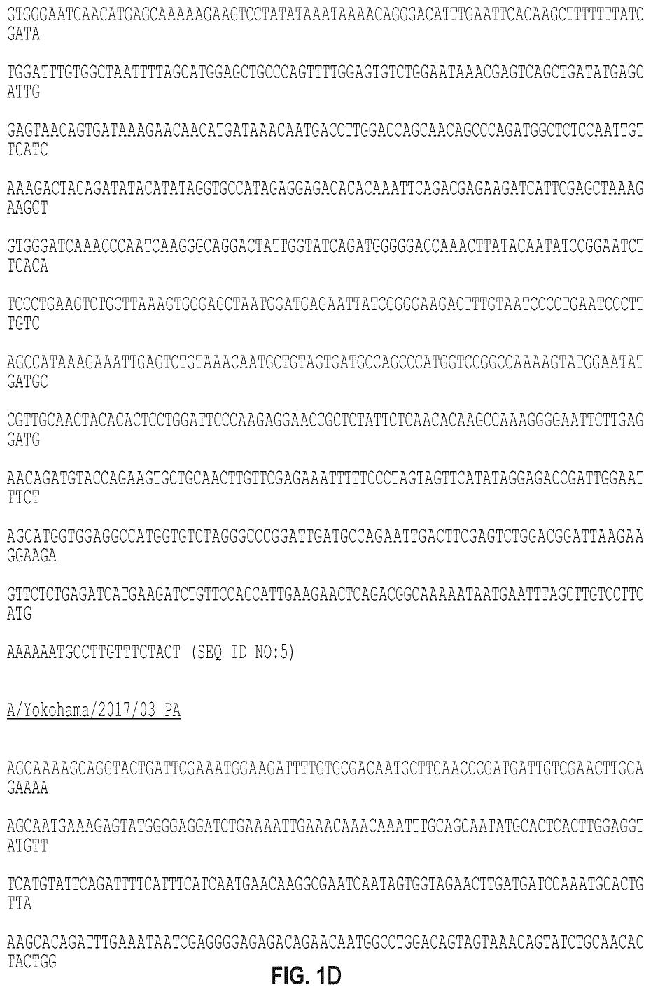

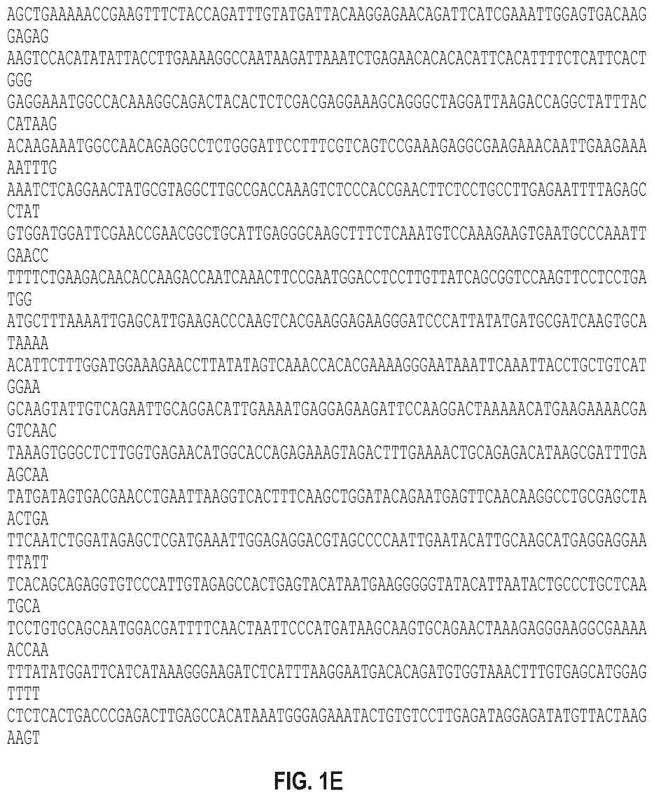

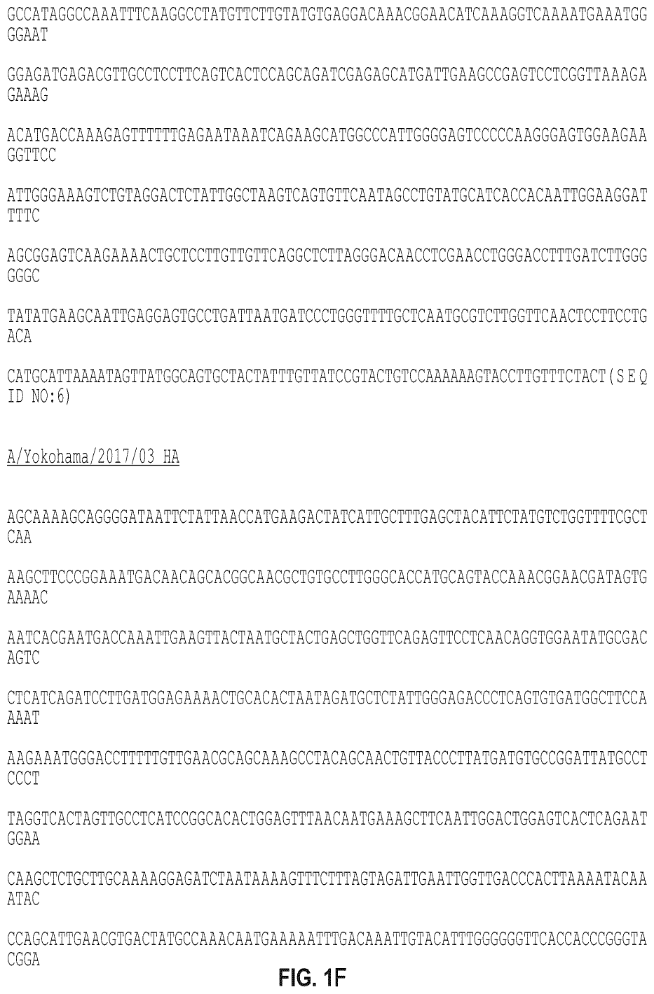

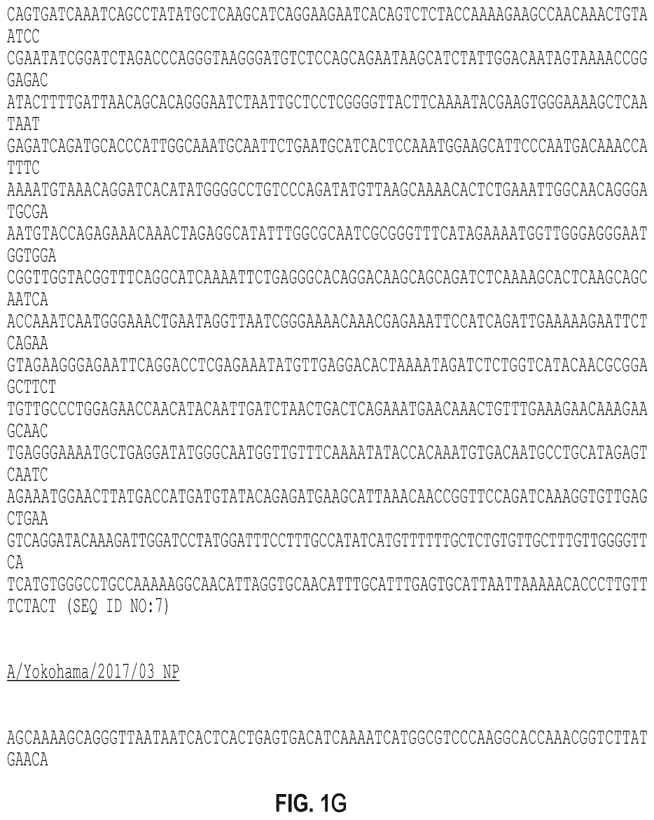

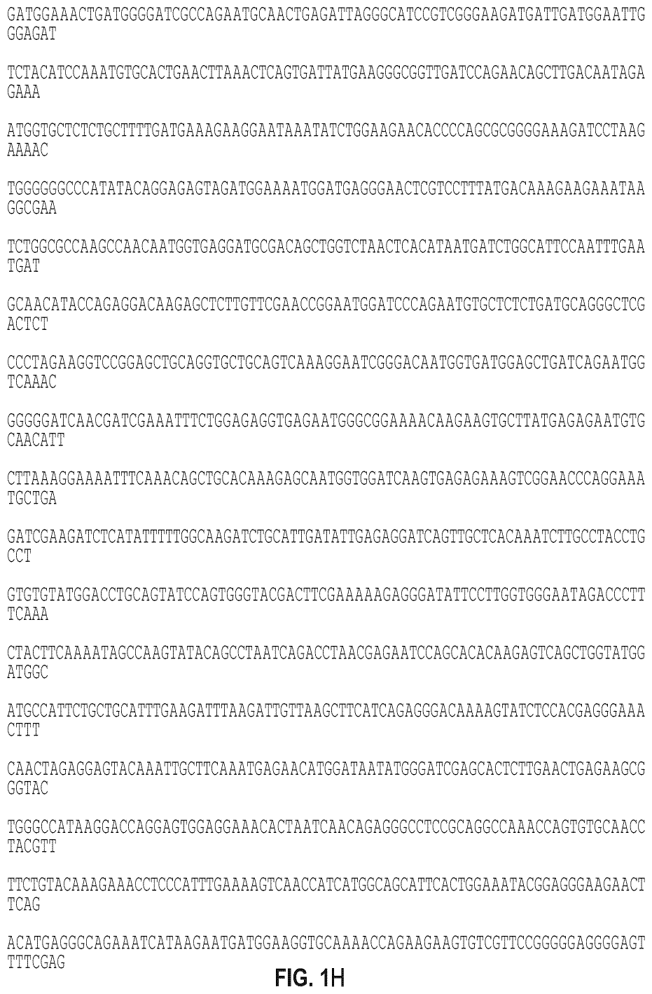

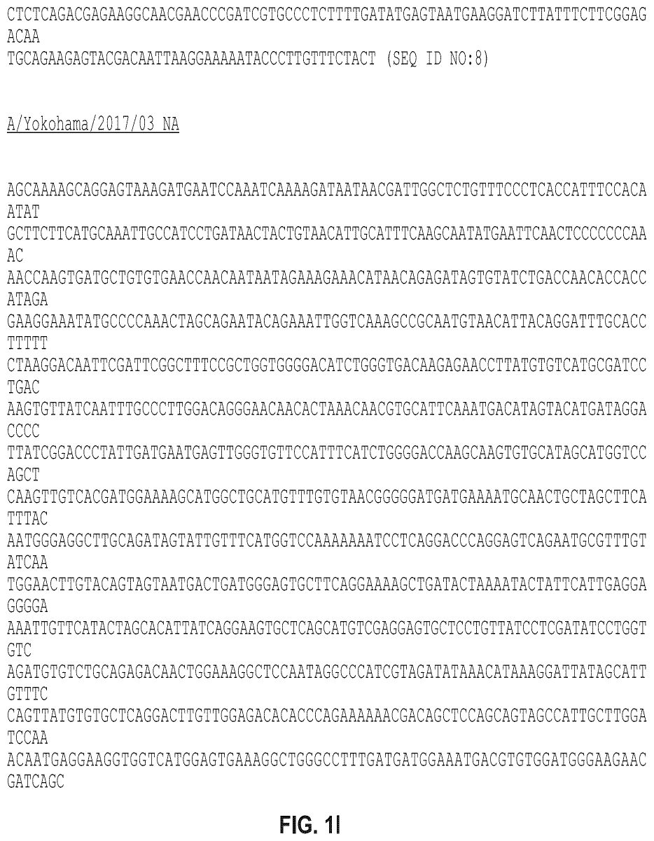

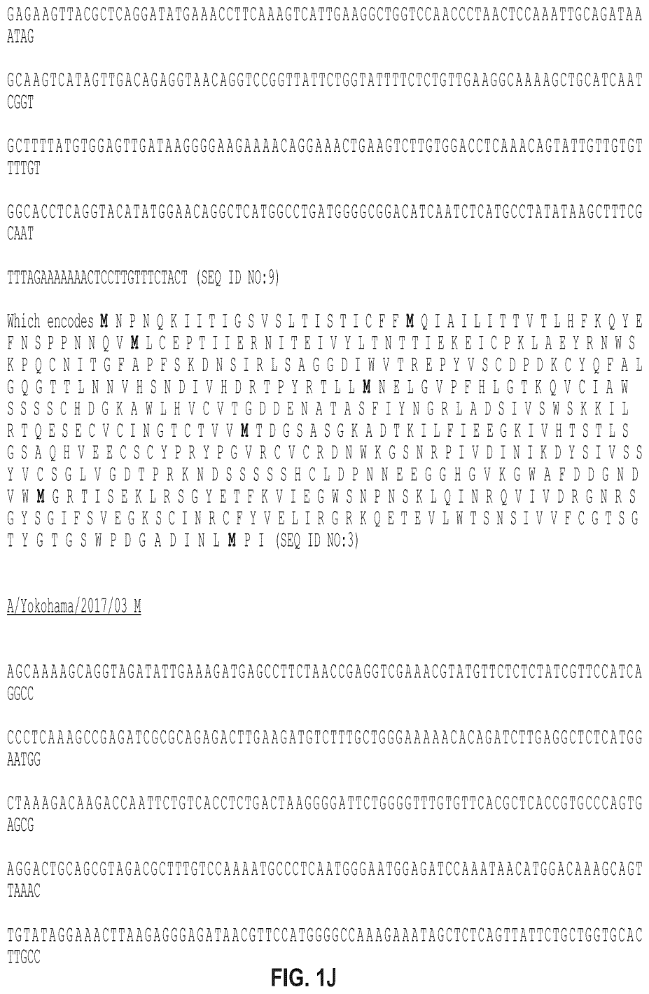

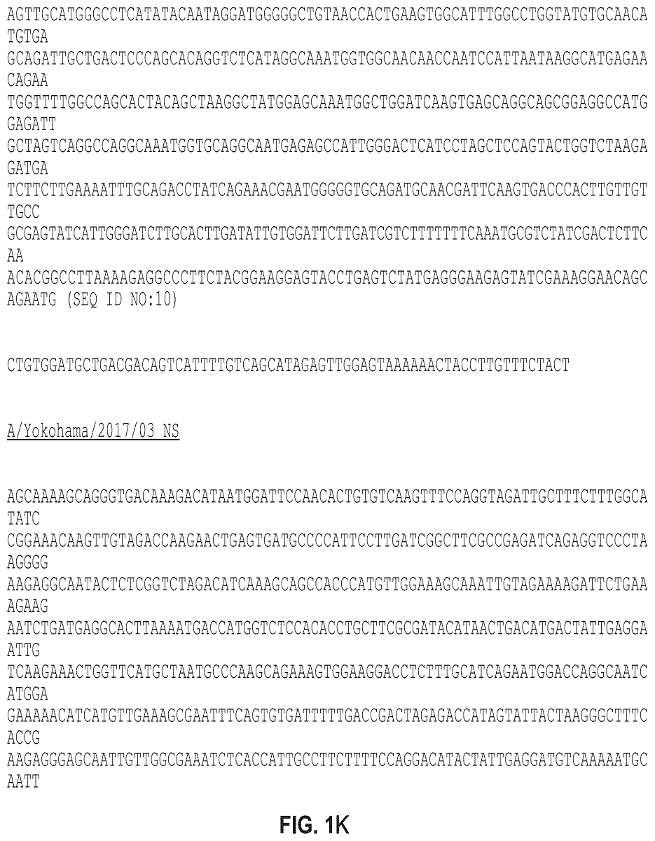

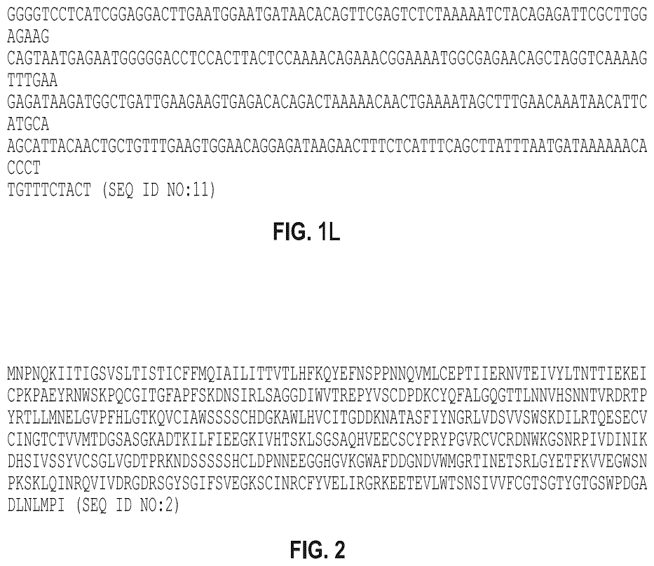

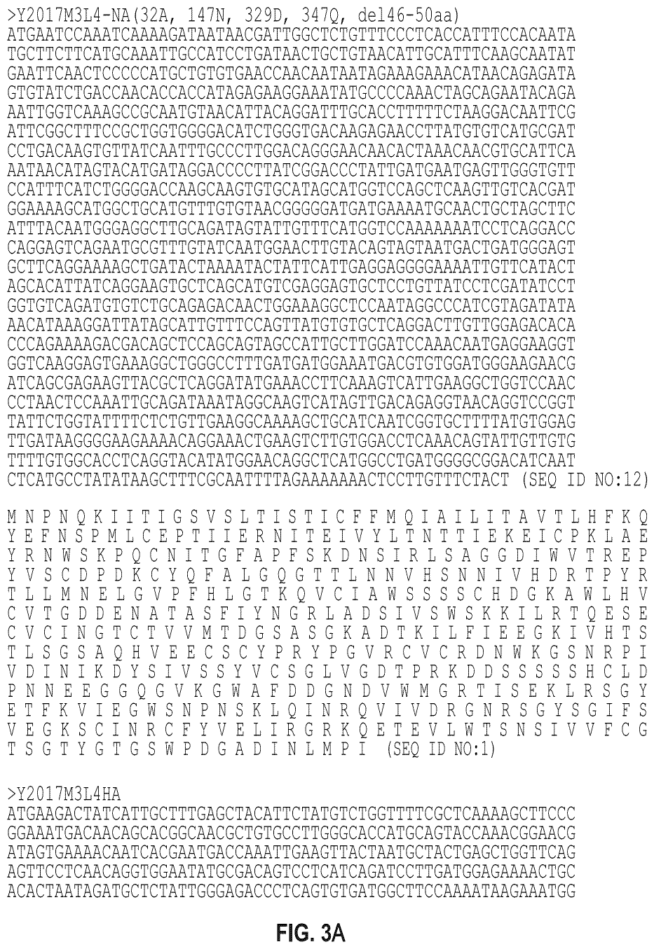

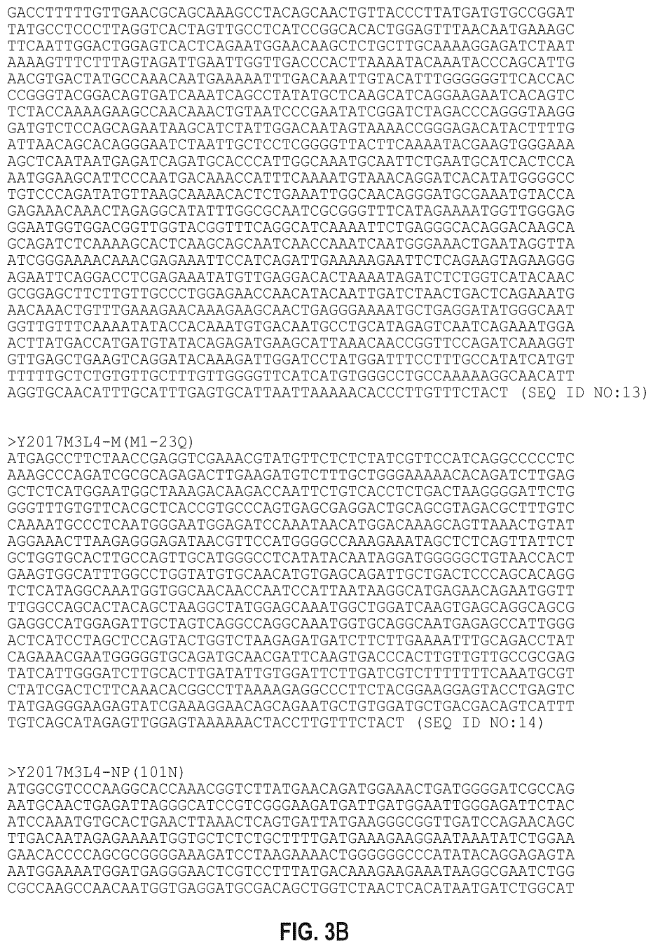

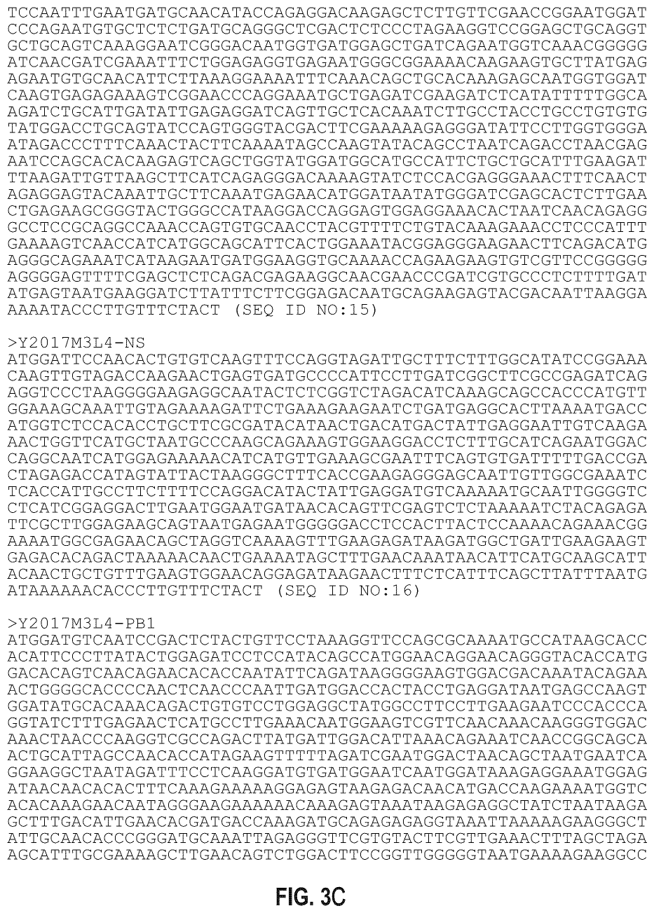

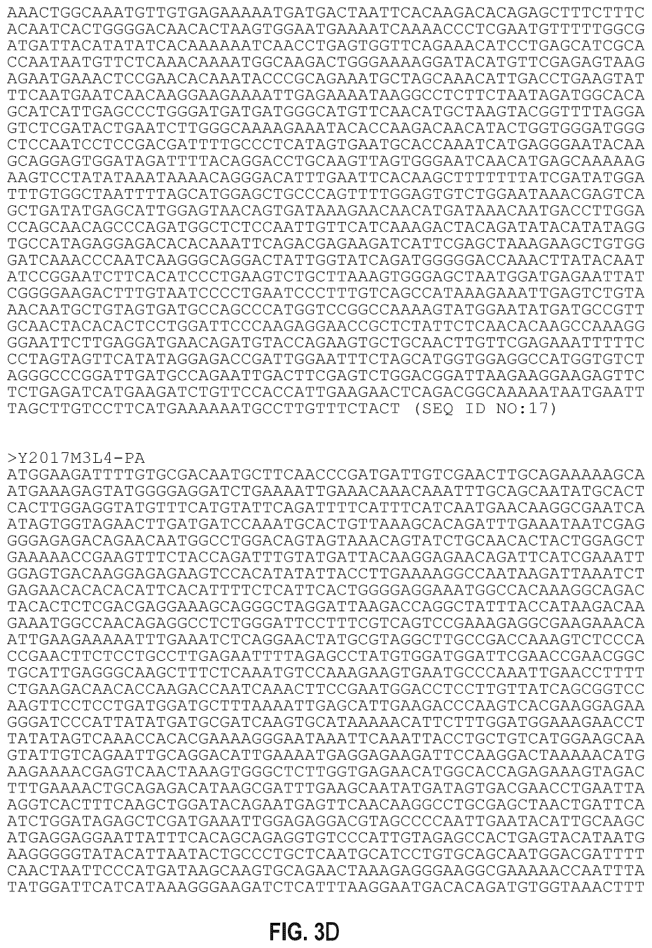

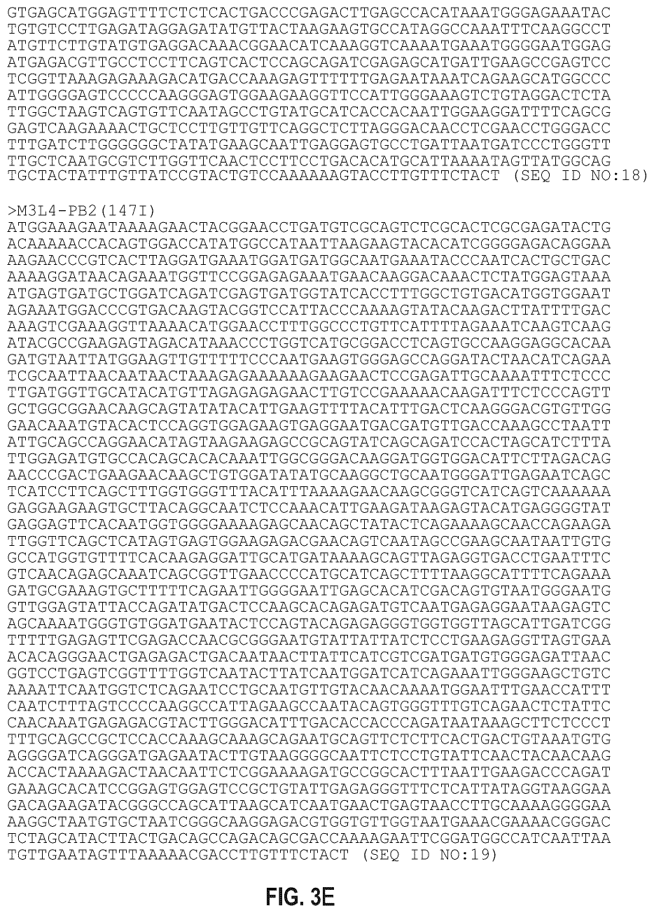

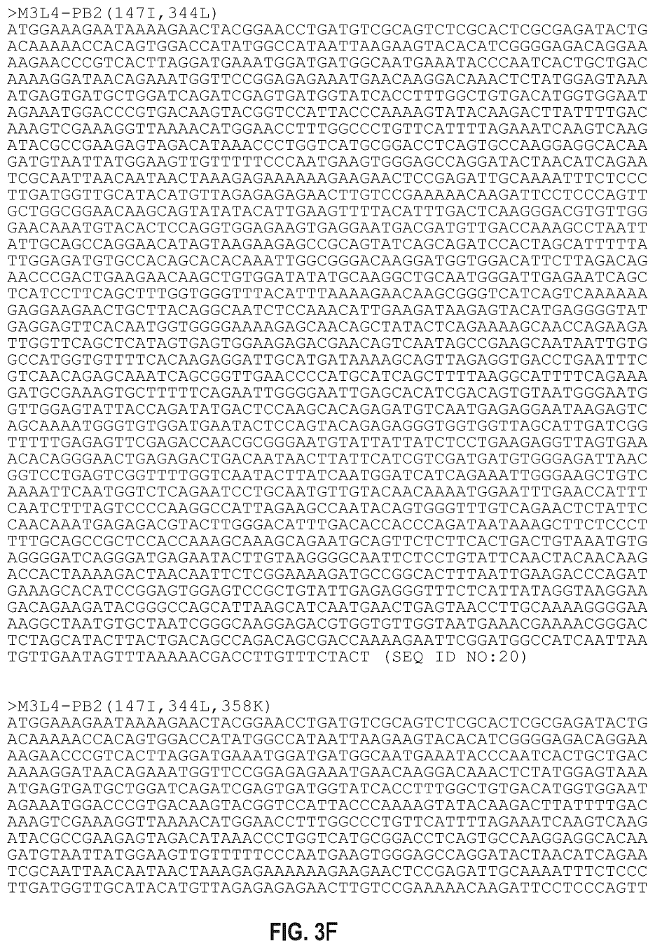

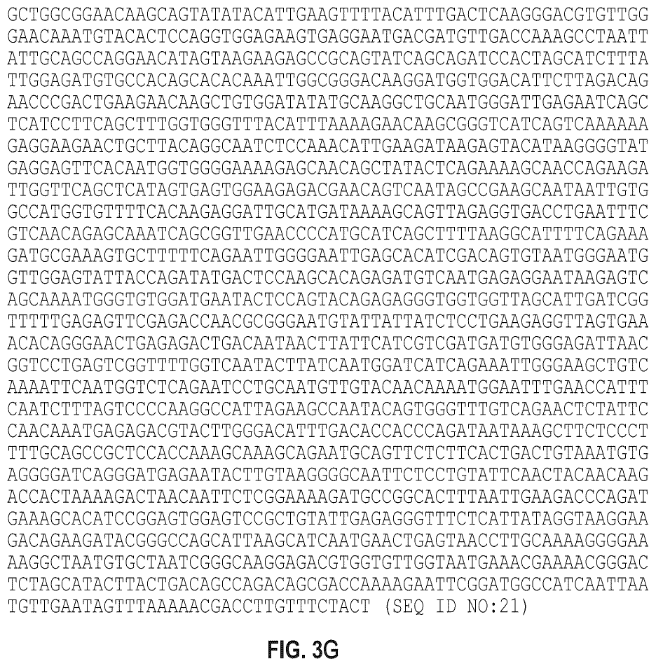

### DETAILED DESCRIPTION

Definitions

As used herein, the term “isolated” refers to in vitro preparation and/or isolation of a nucleic acid molecule, e.g., vector or plasmid, peptide or polypeptide (protein), or virus of the invention, so that it is not associated with in vivo substances, or is substantially purified from in vitro substances. An isolated virus preparation is generally obtained by in vitro culture and propagation, and/or via passage in eggs, and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the disclosure, by the methodology of genetic engineering.

As used herein, a “heterologous” influenza virus gene or viral segment is from an influenza virus source that is different than a majority of the other influenza viral genes or viral segments in a recombinant, e.g., reassortant, influenza virus.

The terms “isolated polypeptide”, “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See http://www.ncbi.n1m.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Influenza Virus Structure and Propagation

Influenza A viruses possess a genome of eight single-stranded negative-sense viral RNAs (vRNAs) that encode at least ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cRNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, influenza B virus does not have a M2 protein with ion channel activity but has BM2 and has a viral segment with both NA and NB sequences. Influenza C virus has only seven viral segments.

### Cells That Can Be Used to Produce Virus

Any cell, e.g., any avian or mammalian cell, such as avian eggs, a human, e.g., 293T or PER.C6® cells, or canine, bovine, equine, feline, swine, ovine, rodent, for instance mink, e.g., MvLu1 cells, or hamster, e.g., CHO cells, or non-human primate, e.g., Vero cells, including mutant cells, which supports efficient replication of influenza virus can be employed to isolate and/or propagate influenza viruses. Isolated viruses can be used to prepare a reassortant virus. In one embodiment, host cells for vaccine production are continuous mammalian or avian cell lines or cell strains. A complete characterization of the cells to be used, may be conducted so that appropriate tests for purity of the final product can be included. Data that can be used for the characterization of a cell includes (a) information on its origin, derivation, and passage history; (b) information on its growth and morphological characteristics; (c) results of tests of adventitious agents; (d) distinguishing features, such as biochemical, immunological, and cytogenetic patterns which allow the cells to be clearly recognized among other cell lines; and (e) results of tests for tumorigenicity. In one embodiment, the passage level, or population doubling, of the host cell used is as low as possible.

In one embodiment, the cells are WHO certified, or certifiable, continuous cell lines. The requirements for certifying such cell lines include characterization with respect to at least one of genealogy, growth characteristics, immunological markers, virus susceptibility tumorigenicity and storage conditions, as well as by testing in animals, eggs, and cell culture. Such characterization is used to confirm that the cells are free from detectable adventitious agents. In some countries, karyology may also be required. In addition, tumorigenicity may be tested in cells that are at the same passage level as those used for vaccine production. The virus may be purified by a process that has been shown to give consistent results, before vaccine production (see, e.g., World Health Organization, 1982).

Virus produced by the host cell may be highly purified prior to vaccine or gene therapy formulation. Generally, the purification procedures result in extensive removal of cellular DNA and other cellular components, and adventitious agents. Procedures that extensively degrade or denature DNA may also be used.

### Influenza Vaccines

A vaccine includes an isolated recombinant influenza virus of the invention, and optionally one or more other isolated viruses including other isolated influenza viruses, one or more immunogenic proteins or glycoproteins of one or more isolated influenza viruses or one or more other pathogens, e.g., an immunogenic protein from one or more bacteria, non-influenza viruses, yeast or fungi, or isolated nucleic acid encoding one or more viral proteins (e.g., DNA vaccines) including one or more immunogenic proteins of the isolated influenza virus of the invention. In one embodiment, the influenza viruses of the invention may be vaccine vectors for influenza virus or other pathogens.

A complete virion vaccine may be concentrated by ultrafiltration and then purified by zonal centrifugation or by chromatography. Viruses other than the virus of the invention, such as those included in a multivalent vaccine, may be inactivated before or after purification using formalin or beta-propiolactone, for instance.

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976); or a nonionic detergent such as that commercialized under the name TRITON X100. The hemagglutinin may also be isolated after treatment of the virions with a protease such as bromelin, and then purified. The subunit vaccine may be combined with an attenuated virus of the invention in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. The split vaccine may be combined with an attenuated virus of the invention in a multivalent vaccine.

Inactivated Vaccines. Inactivated influenza virus vaccines are provided by inactivating replicated virus using known methods, such as, but not limited to, formalin or β-propiolactone treatment. Inactivated vaccine types that can be used in the invention can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live Attenuated Virus Vaccines. Live, attenuated influenza virus vaccines, such as those including a recombinant virus of the invention can be used for preventing or treating influenza virus infection. Attenuation may be achieved in a single step by transfer of attenuated genes from an attenuated donor virus to a replicated isolate or reassorted virus according to known methods. Since resistance to influenza A virus is mediated primarily by the development of an immune response to the HA and/or NA glycoproteins, the genes coding for these surface antigens come from the reassorted viruses or clinical isolates. The attenuated genes are derived from an attenuated parent. In this approach, genes that confer attenuation generally do not code for the HA and NA glycoproteins.

Viruses (donor influenza viruses) are available that are capable of reproducibly attenuating influenza viruses, e.g., a cold adapted (ca) donor virus can be used for attenuated vaccine production. Live, attenuated reassortant virus vaccines can be generated by mating the ca donor virus with a virulent replicated virus. Reassortant progeny are then selected at 25° C. (restrictive for replication of virulent virus), in the presence of an appropriate antiserum, which inhibits replication of the viruses bearing the surface antigens of the attenuated ca donor virus. Useful reassortants are: (a) infectious, (b) attenuated for seronegative non-adult mammals and immunologically primed adult mammals, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible mammals both adults and non-adult.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene. Thus, new donor viruses can also be generated bearing attenuating mutations introduced by site-directed mutagenesis, and such new donor viruses can be used in the production of live attenuated reassortants vaccine candidates in a manner analogous to that described above for the ca donor virus. Similarly, other known and suitable attenuated donor strains can be reassorted with influenza virus to obtain attenuated vaccines suitable for use in the vaccination of mammals.

In one embodiment, such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The viruses in a multivalent vaccine can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g., amantadine or rimantidine); HA and NA activity and inhibition; and nucleic acid screening (such as probe hybridization or PCR) to confirm that donor genes encoding the antigenic determinants (e.g., HA or NA genes) are not present in the attenuated viruses.

### Pharmaceutical Compositions

Pharmaceutical compositions of the present invention, suitable for inoculation, e.g., nasal, parenteral or oral administration, comprise one or more influenza virus isolates, e.g., one or more attenuated or inactivated influenza viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. The composition of the invention is generally presented in the form of individual doses (unit doses).

Conventional vaccines generally contain about 0.1 to 200 μg, e.g., 30 to 100 μg, 0.1 to 2 μg, 0.5 to 5 μg, 1 to 10 μg, 10 μg to 20 μg , 15 μg to 30 μg, or 10 to 30 μg, of HA from each of the strains entering into their composition. The vaccine forming the main constituent of the vaccine composition of the invention may comprise a single influenza virus, or a combination of influenza viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art.

Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present invention is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

Heterogeneity in a vaccine may be provided by mixing replicated influenza viruses for at least two influenza virus strains, such as 2-20 strains or any range or value therein. Vaccines can be provided for variations in a single strain of an influenza virus, using techniques known in the art.

A pharmaceutical composition according to the present invention may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the invention which are vaccines are provided before any symptom or clinical sign of a pathogen infection becomes manifest. The prophylactic administration of the composition serves to prevent or attenuate any subsequent infection. When provided prophylactically, the gene therapy compositions of the invention, are provided before any symptom or clinical sign of a disease becomes manifest. The prophylactic administration of the composition serves to prevent or attenuate one or more symptoms or clinical signs associated with the disease.

When provided therapeutically, a viral vaccine is provided upon the detection of a symptom or clinical sign of actual infection. The therapeutic administration of the compound(s) serves to attenuate any actual infection. When provided therapeutically, a gene therapy composition is provided upon the detection of a symptom or clinical sign of the disease. The therapeutic administration of the compound(s) serves to attenuate a symptom or clinical sign of that disease.

Thus, a vaccine composition of the present invention may be provided either before the onset of infection (so as to prevent or attenuate an anticipated infection) or after the initiation of an actual infection. Similarly, for gene therapy, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one strain of an infectious influenza virus.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition of the present invention may confer resistance to one or more pathogens, e.g., one or more influenza virus strains, by either passive immunization or active immunization. In active immunization, an attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one influenza virus strain. A gene therapy composition of the present invention may yield prophylactic or therapeutic levels of the desired gene product by active immunization.

In one embodiment, the vaccine is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present invention thus includes methods for preventing or attenuating a disorder or disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease. As used herein, a gene therapy composition is said to prevent or attenuate a disease if its administration results either in the total or partial attenuation (i.e., suppression) of a clinical sign or condition of the disease, or in the total or partial immunity of the individual to the disease.

A composition having at least one influenza virus of the present invention, including one which is attenuated and one or more other isolated viruses, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present invention, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the invention and represent dose ranges.

The dosage of a live, attenuated or killed virus vaccine for an animal such as a mammalian adult organism may be from about 10²-10²⁰, e.g., 10³-10¹², 10²-10¹⁰, 10⁵-10¹¹, 10⁶-10¹⁵, 10²-10¹⁰, or 10¹⁵-10²⁰ plaque forming units (PFU)/kg, or any range or value therein. The dose of one viral isolate vaccine, e.g., in an inactivated vaccine, may range from about 0.1 to 1000, e.g., 0.1 to 10 μg, 1 to 20 μg, 30 to 100 μg, 10 to 50 μg, 50 to 200 μg, or 150 to 300 μg, of HA protein. However, the dosage should be a safe and effective amount as determined by conventional methods, using existing vaccines as a starting point.

The dosage of immunoreactive HA in each dose of replicated virus vaccine may be standardized to contain a suitable amount, e.g., 0.1 μg to 1 μg, 0.5 μg to 5 μg, 1 μg to 10 μg, 10 μg to 20 μg, 15 μg to 30 μg, or 30 μg to 100 μg or any range or value therein, or the amount recommended by government agencies or recognized professional organizations. The quantity of NA can also be standardized, however, this glycoprotein may be labile during purification and storage.

The dosage of immunoreactive HA in each dose of replicated virus vaccine can be standardized to contain a suitable amount, e.g., 1-50 μg or any range or value therein, or the amount recommended by the U.S. Public Health Service (PHS), which is usually 15 μg, per component for older children >3 years of age, and 7.5 μg per component for children <3 years of age. The quantity of NA can also be standardized, however, this glycoprotein can be labile during the processor purification and storage (Kendal et al., 1980; Kerr et al., 1975). Each 0.5-ml dose of vaccine may contain approximately 0.1 to 0.5 billion viral particles, 0.5 to 2 billion viral particles, 1 to 50 billion virus particles, 1 to 10 billion viral particles, 20 to 40 billion viral particles, 1 to 5 billion viral particles, or 40 to 80 billion viral particles.

### Exemplary Viruses

Useful modifications of influenza neuraminidase (NA) proteins are described herein that stabilize hemagglutinin (HA) protein during egg-passages of influenza viruses that express those modified neuraminidase proteins. Modified nucleic acids are also described that encode such modified neuraminidase proteins. The modifications can include deletions, substitutions and combinations thereof within the neuraminidase protein and nucleic acid sequences. Viruses that express such modified neuraminidase proteins exhibit significantly reduced acquisition of antigenicity-compromising mutations in hemagglutinin (HA) during growth of influenza in eggs.

For example, in some cases the modified neuraminidase can have at least one, or at least two, or at least three modifications. Amino acid positions within influenza neuraminidase proteins that can be modified include, for example, one or more amino acids within positions 29-35, one or more amino acids within positions 44-52, one or more amino acids within positions 144-154, one or more amino acid positions within 240-250, one or more amino acids within positions 326-333, one or more amino acid positions within 344-350, one or more amino acid positions within 365-375, and combinations thereof, based on N2 numbering. For example, the amino acid(s) can be any amino acid within these positions such as any of the amino acids listed in the table below.

| Original | Exemplary | Alternative |
|---|---|---|
| Residue | Substitutions | Substitutions |
| Ala (A) | val; leu; ile | Val |
| Arg (R) | lys; gln; asn | Lys |
| Asn (N) | gln; his; lys; arg | Gln |
| Asp (D) | Glu, Asn | Glu, Asn |
| Cys (C) | Ser | Ser |
| Gln (Q) | Asn | Asn |
| Glu (E) | Asp | Asp |
| Gly (G) | Pro | Pro |
| His (H) | asn; gln; lys; arg; gln; | Arg; Gln |
| Ile (I) | leu; val; met; ala; phe | Leu |
| | norleucine | |
| Leu (L) | norleucine; ile; val; met; ala; | Ile |
| | phe | |
| Lys (K) | arg; gln; asn | Arg |
| Met (M) | leu; phe; ile | Leu |
| Phe (F) | leu; val; ile; ala | Leu |
| Pro (P) | Gly | Gly |
| Ser (S) | Thr | Thr |
| Thr (T) | Ser, Ala | Ser, Als |
| Trp (W) | Tyr | Tyr |
| Tyr (Y) | trp; phe; thr; ser | Phe |
| Val (V) | ile; leu; met; phe; ala; | Leu |
| | norleucine | |

In some cases, a selected amino acid within positions 29-35, positions 44-52, positions 144-154, positions 326-333, positions within 344-350, positions within 365-375, can have a conservative substitution. However, in other cases, the selected amino acid within positions 29-35, positions 44-52, positions 144-150, positions 326-333, positions within 344-350, positions within 365-375, can have a non-conservative substitution.

For example, a modified neuraminidase can have a deletion of at least one proline, asparagine, glutamine, valine, or a combination of a proline, one or more asparagine(s), a glutamine, and a valine within positions 44-52 of the modified neuraminidase. A modified neuraminidase can have a substitution (replacement) of a threonine within positions 29-35, where the replacement is any amino acid. A modified neuraminidase can have a substitution (replacement) of a threonine or an aspartic acid within positions 145-154 or 365 to 375, where the replacement is any amino acid. A modified neuraminidase can have a substitution (replacement) of an asparagine within positions 326-333, where the replacement is any amino acid. A modified neuraminidase can have a substitution (replacement) of a histidine within positions 345-350, where the replacement is any amino acid. Exemplary substitutions (replacements) for various types of amino acids are provided in the table above.

One example of an influenza A virus (A/Yokohama/2013/2003(H3N2)) neuraminidase protein sequence is provided below

(SEQ ID NO: 75)

| | |
|---|---|
| 1 | MNPNQKIITI GSVSLTISTI CFFMQIAILI TTVTLHFKQY |
| 41 | EFNSPPNNQV MLCEPTIIER NITEIVYLTN TTIEKEICPK |
| 81 | LAEYRNWSKP QCNITGFAPF SKDNSIRLSA GGDIWVTREP |
| 121 | YVSCDPDKCY QFALGQGTTL NNVHSNDIVH DRTPYRTLLM |
| 161 | NELGVPFHLG TKQVCIAWSS SSCHDGKAWL HVCVTGDDEN |
| 201 | ATASFIYNGR LADSIVSWSK KILRTQESEC VCINGTCTVV |
| 241 | MTDGSASGKA DTKILFIEEG KIVHTSTLSG SAQHVEECSC |
| 281 | YPRYPGVRCV CRDNWKGSNR PIVDINIKDY SIVSSYVCSG |
| 321 | LVGDTPRKND SSSSSHCLDP NNEEGGHGVK GWAFDDGNDV |
| 361 | WMGRTISEKL RSGYETFKVI EGWSNPNSKL QINRQVIVDR |
| 401 | GNRSGYSGIF SVEGKSCINR CFYVELIRGR KQETEVLWTS |
| 441 | NSIVVFCGTS GTYGTGSWPD GADINLMPI |

Amino acids that can be modified to improve the stability of co-expressed HA are highlighted in bold and with underlining within the sequence shown above. A nucleic acid that encodes such an influenza A virus (A/Yokohama/2013/2003(H3N2)) neuraminidase protein sequence is shown below

(SEQ ID NO: 76)

| | |
|---|---|
| 1 | AGCAAAAGCA GGAGTAAAGA TGAATCCAAA TCAAAAGATA |
| 41 | ATAACGATTG GCTCTGTTTC CCTCACCATT TCCACAATAT |
| 81 | GCTTCTTCAT GCAAATTGCC ATCCTGATAA CTACTGTAAC |
| 121 | ATTGCATTTC AAGCAATATG AATTCAACTC CCCCCCAAAC |
| 161 | AACCAAGTGA TGCTGTGTGA ACCAACAATA ATAGAAAGAA |
| 201 | ACATAACAGA GATAGTGTAT CTGACCAACA CCACCATAGA |
| 241 | GAAGGAAATA TGCCCCAAAC TAGCAGAATA CAGAAATTGG |
| 281 | TCAAAGCCGC AATGTAACAT TACAGGATTT GCACCTTTTT |
| 321 | CTAAGGACAA TTCGATTCGG CTTTCCGCTG GTGGGGACAT |
| 361 | CTGGGTGACA AGAGAACCTT ATGTGTCATG CGATCCTGAC |
| 401 | AAGTGTTATC AATTTGCCCT TGGACAGGGA ACAACACTAA |
| 441 | ACAACGTGCA TTCAAATGAC ATAGTACATG ATAGGACCCC |
| 481 | TTATCGGACC CTATTGATGA ATGAGTTGGG TGTTCCATTT |
| 521 | CATCTGGGGA CCAAGCAAGT GTGCATAGCA TGGTCCAGCT |
| 561 | CAAGTTGTCA CGATGGAAAA GCATGGCTGC ATGTTTGTGT |
| 601 | AACGGGGGAT GATGAAAATG CAACTGCTAG CTTCATTTAC |
| 641 | AATGGGAGGC TTGCAGATAG TATTGTTTCA TGGTCCAAAA |
| 681 | AAATCCTCAG GACCCAGGAG TCAGAATGCG TTTGTATCAA |
| 721 | TGGAACTTGT ACAGTAGTAA TGACTGATGG GAGTGCTTCA |
| 761 | GGAAAAGCTG ATACTAAAAT ACTATTCATT GAGGAGGGGA |
| 801 | AAATTGTTCA TACTAGCACA TTATCAGGAA GTGCTCAGCA |
| 841 | TGTCGAGGAG TGCTCCTGTT ATCCTCGATA TCCTGGTGTC |
| 881 | AGATGTGTCT GCAGAGACAA CTGGAAAGGC TCCAATAGGC |
| 921 | CCATCGTAGA TATAAACATA AAGGATTATA GCATTGTTTC |
| 961 | CAGTTATGTG TGCTCAGGAC TTGTTGGAGA CACACCCAGA |
| 1001 | AAAAACGACA GCTCCAGCAG TAGCCATTGC TTGGATCCAA |
| 1041 | ACAATGAGGA AGGTGGTCAT GGAGTGAAAG GCTGGGCCTT |
| 1081 | TGATGATGGA AATGACGTGT GGATGGGAAG AACGATCAGC |
| 1121 | GAGAAGTTAC GCTCAGGATA TGAAACCTTC AAAGTCATTG |
| 1161 | AAGGCTGGTC CAACCCTAAC TCCAAATTGC AGATAAATAG |
| 1201 | GCAAGTCATA GTTGACAGAG GTAACAGGTC CGGTTATTCT |
| 1241 | GGTATTTTCT CTGTTGAAGG CAAAAGCTGC ATCAATCGGT |
| 1281 | GCTTTTATGT GGAGTTGATA AGGGGAAGAA AACAGGAAAC |
| 1321 | TGAAGTCTTG TGGACCTCAA ACAGTATTGT TGTGTTTTGT |
| 1361 | GGCACCTCAG GTACATATGG AACAGGCTCA TGGCCTGATG |
| 1401 | GGGCGGACAT CAATCTCATG CCTATATAAG CTTTCGCAAT |
| 1441 | TTTAGAAAAA AACTCCTTGT TTCTACT |

Modifications at the specified positions in neuraminidase can confer enhanced growth of the virus.

Another example of an influenza A virus (A/Yokohama/47/2002(H1N2))) neuraminidase sequence is shown below, with positions of modifications highlighted in bold and with underlining.

```
(SEQ ID NO: 77)
      10 20 30 40 50
MNPNQKIITI GSVSLTIATI CFLMQIAILV TTVTLHFKQY ECNSPPNNQV
      60 70 80 90 100
MLCEPTIIER NITEIVYLTN TTIEKEICPK LAEYRNWSKP QCNITGFAPF
      110 120 130 140 150
SKDNSIRLSA GGDIWVTREP YVSCDPDKCY QFALGQGTTL NNGHSNDTVH
      160 170 180 190 200
DRTPYRTLLM NELGVPFHLG TKQVCIAWSS SSCHDGKAWL HVCVTGDDGN
      210 220 230 240 250
ATASFIYNGR LVDSIGSWSK KILRTQESEC VCINGTCTVV MTDGSASGKA
      260 270 280 290 300
DTKILFIEEG KIVHTSLLSG SAQHVEECSC YPRYPGVRCV CRDNWKGSNR
      310 320 330 340 350
PIVDINVKDY SIVSSYVCSG LVGDTPRKND SSSSSHCLDP NNEEGGHGVK
    360 370 380 390 400
GWAFDDGNDV WMGRTISEKL RSGYETFKVI EGWSKPNSKL QINRQVIVDR
    410 420 430 440 450
GNRSGYSGIF SVEGKSCINR CFYVELIRGR NQETEVLWTS NSIVVFCGTS
      460
GTYGTGSWPD GADINLMPI
```

Amino acids that can be modified to improve the stability of co-expressed HA are highlighted in bold and with underlining within the sequence shown above.

In some cases, in one or more modifications can also be introduced into HA, PA, PB1, PB2, NP, M1, M2, NS2, PB1-F2, PA-X, and/or NS1 proteins (and nucleic acids encoding such proteins).

Enhanced growth of the virus when passaged through embryonated chicken eggs or cultured cells is observed when the modified NA proteins are expressed and such expression can result in significantly higher viral titers. Thus, the invention provides a method for making influenza viruses with enhanced replication in cell culture or embryonated eggs. The method includes providing cells suitable for influenza vaccine production; modifying nucleic acids encoding the neuraminidase; and isolating virus strains with enhanced growth relative to the one or more unmodified viral isolates. In some cases, a method for making influenza viruses with enhanced replication in cell culture can involve, serially culturing one or more influenza virus isolates in embryonated chicken eggs; and isolating serially cultured virus with enhanced growth relative to the one or more isolates prior to serial culture. In some cases, the viruses can be grown or passaged within cells in culture, e.g., MDCK or Vero cells.

The modified neuraminidases can be expressed in a variety of influenza strains. For example, A/Puerto Rico/8/34 (H1N1), “PR8,” virus often serves as the genetic backbone for generation of inactivated influenza vaccines. Some vaccine strains based on PR8 backbone can replicate to relatively low titers in eggs and cell culture, resulting in delayed vaccine production and vaccine shortage. However, expression of the modified neuraminidases described herein can improve replication of the PR8 (and other) influenza strains.

In one embodiment of the invention, vectors for vRNA production can include a vector comprising a promoter operably linked to a modified NA DNA linked to a transcription termination sequence, a vector comprising a promoter operably linked to an influenza virus PB1 DNA linked to a transcription termination sequence, a vector comprising a promoter operably linked to an influenza virus PB2 DNA linked to a transcription termination sequence, a vector comprising a promoter operably linked to an influenza virus HA DNA linked to a transcription termination sequence, a vector comprising a promoter operably linked to an influenza virus NP DNA linked to a transcription termination sequence, a vector comprising a promoter operably linked to an influenza virus NA DNA linked to a transcription termination sequence, a vector comprising a promoter operably linked to an influenza virus M DNA linked to a transcription termination sequence, and a vector comprising a operably linked to an influenza virus NS DNA linked to a transcription termination sequence. In one embodiment, the DNAs for vRNA production of PB1, PB2, PA, NP, M, and NS, have sequences from an influenza virus that replicates to high titers in cultured mammalian cells such as MDCK cells, Vero cells or PER.C6® cells or embryonated eggs, and/or from a vaccine virus, e.g., one that does not cause significant disease in humans. The DNA for vRNA production of NA may be from any NA, e.g., any of N1-N11, and the DNA for vRNA production of HA may be from any HA, e.g., H1-H18. In one embodiment, the DNAs for vRNA production may be for an influenza B or C virus. The DNAs for vRNA production of NA and HA may be from different strains or isolates (6:1:1 reassortants) or from the same strain or isolate (6:2 reassortants), or the NA may be from the same strain or isolate as that for the internal genes (7:1 reassortant). Vectors for mRNA production can include a vector encoding a modified NA, a vector encoding influenza virus PA, a vector encoding influenza virus PB1, a vector encoding influenza virus PB2, and a vector encoding influenza virus NP, and optionally one or more vectors encoding NP, NS, M, e.g., M1 and M2, HA or NA. The vectors encoding viral proteins may further include a transcription termination sequence.

Other reassortants with internal genes from other PR8 isolates or vaccine viruses may be employed in recombinant reassortant viruses of the invention. In particular, 5:1:2 reassortants having UW-PR8 PB1, PB2, PA, NP, and M (“5”) and PR8(Cam) NS (“1”); 6:1:1 reassortants having UW-PR8 (modified) NA, PB1, PB2, PA, NP, and M (“6”) and PR8(Cam) NS (“1”); and 7:1 reassortants having UW-PR8 PB1, PB2, PA, NP, M, (modified) NA, and NS (“7”) may be employed.

The neuraminidases that can be modified can have sequences that vary from those described herein. However, in some cases, the modified neuraminidases can have substantially the same activity as a corresponding polypeptide described by sequence herein. As used herein, “substantially the same activity” includes an activity that is about 0.1%, 1%, 10%, 30%, 50%, 90%, e.g., up to 100% or more activity, or a detectable protein level that is about 80%, 90% or more protein level, of the corresponding protein described herein. In one embodiment, the nucleic acid encodes a polypeptide which is substantially the same as, e.g., having at least 80%, e.g., 90%, 92%, 95%, 97%,98%, or 99%, including any integer between 80 and 99, contiguous amino acid sequence identity to a polypeptide encoded by one of sequences described herein. In one embodiment, the isolated and/or purified nucleic acid molecule comprises a nucleotide sequence which is substantially the same as, e.g., having at least 50%, e.g., 60%, 70%, 80% or 90%, including any integer between 50 and 100, or more contiguous nucleic acid sequence identity to one of the nucleic acid sequences described herein. In one embodiment, a nucleic acid also encodes a polypeptide having at least 80%, e.g., 90%, 92%, 95%, 97%,98%, or 99%, including any integer between 80 and 99, contiguous amino acid sequence identity to a polypeptide described herein.

In one embodiment, a modified influenza virus neuraminidase polypeptide has one or more, for instance, 2, 5, 10, 15, 20 or more, conservative amino acids substitutions, e.g., conservative substitutions of up to 10% or 20% of 2, 5, 10, 15, 20 or more, of a combination of conservative and non-conservative amino acids substitutions, e.g., conservative substitutions of up to 10% or 20% of the residues, or relative to a polypeptide with one of the sequences disclosed herein.

The invention thus includes the use of isolated and purified vectors or plasmids, which express or encode influenza virus proteins, or express or encode influenza vRNA, both native and recombinant vRNA. The vectors comprise influenza cDNA, e.g., influenza A (e.g., any influenza A gene including any of the 18 HA or 11 NA subtypes), B or C DNA (see Fields Virology (Fields et al. (eds.), Lippincott, Williams and Wickens (2006), which is specifically incorporated by reference herein). Any suitable promoter or transcription termination sequence may be employed to express a protein or peptide, e.g., a viral protein or peptide, a protein or peptide of a nonviral pathogen, or a therapeutic protein or peptide.

A composition or plurality of vectors of the invention may also comprise a heterologous gene or open reading frame of interest, e.g., a foreign gene encoding an immunogenic peptide or protein useful as a vaccine or in gene replacement, for instance, may encode an epitope useful in a cancer therapy or vaccine, or a peptide or polypeptide useful in gene therapy. When preparing virus, the vector or plasmid comprising the gene or cDNA of interest may substitute for a vector or plasmid for an influenza viral gene or may be in addition to vectors or plasmids for all influenza viral genes. Thus, another embodiment of the invention comprises a composition or plurality of vectors as described above in which one of the vectors is replaced with, or further comprises, 5′ influenza virus sequences optionally including 5′ influenza virus coding sequences or a portion thereof, linked to a desired nucleic acid sequence, e.g., a desired cDNA, linked to 3′ influenza virus sequences optionally including 3′ influenza virus coding sequences or a portion thereof. In one embodiment, the desired nucleic acid sequence such as a cDNA is in an antisense (antigenomic) orientation. The introduction of such a vector in conjunction with the other vectors described above to a host cell permissive for influenza virus replication results in recombinant virus comprising vRNA corresponding to the heterologous sequences of the vector.

The promoter in a vector for vRNA production may be a RNA polymerase I promoter, a RNA polymerase II promoter, a RNA polymerase III promoter, a T7 promoter, or a T3 promoter, and optionally the vector comprises a transcription termination sequence such as a RNA polymerase I transcription termination sequence, a RNA polymerase II transcription termination sequence, a RNA polymerase III transcription termination sequence, or a ribozyme. Ribozymes within the scope of the invention include, but are not limited to, tetrahymena ribozymes, RNase P, hammerhead ribozymes, hairpin ribozymes, hepatitis ribozyme, as well as synthetic ribozymes. In one embodiment, the RNA polymerase I promoter is a human RNA polymerase I promoter.

The promoter or transcription termination sequence in a vRNA or virus protein expression vector may be the same or different relative to the promoter or any other vector. In one embodiment, the vector or plasmid which expresses influenza vRNA comprises a promoter suitable for expression in at least one particular host cell, e.g., avian or mammalian host cells such as canine, feline, equine, bovine, ovine, or primate cells including human cells, or for expression in more than one host.

In one embodiment, at least one vector for vRNA comprises a RNA polymerase II promoter linked to a ribozyme sequence linked to viral coding sequences linked to another ribozyme sequences, optionally linked to a RNA polymerase II transcription termination sequence. In one embodiment, at least 2, e.g., 3, 4, 5, 6, 7 or 8, vectors for vRNA production comprise a RNA polymerase II promoter, a first ribozyme sequence, which is 5′ to a sequence corresponding to viral sequences including viral coding sequences, which is 5′ to a second ribozyme sequence, which is 5′ to a transcription termination sequence. Each RNA polymerase II promoter in each vRNA vector may be the same or different as the RNA polymerase II promoter in any other vRNA vector. Similarly, each ribozyme sequence in each vRNA vector may be the same or different as the ribozyme sequences in any other vRNA vector. In one embodiment, the ribozyme sequences in a single vector are not the same.

In one embodiment, the invention provides a plurality of influenza virus vectors for a reassortant, comprising a vector for vRNA production comprising a promoter operably linked to a modified influenza virus NA DNA linked to a transcription termination sequence, a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS cDNA linked to a transcription termination sequence, wherein the DNAs for the modified NA, PB1, PB2, PA, NP, NS, and M are from one or more influenza vaccine seed viruses and contain two or more of the characteristic residues at the specified position(s); and a vector for mRNA production comprising a promoter operably linked to a DNA segment encoding influenza virus NA, a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, a vector for mRNA production comprising a promoter operably linked to a DNA segment encoding influenza virus NS1, or a vector for mRNA production comprising a promoter operably linked to a DNA segment encoding influenza virus NS2. In one embodiment, at least one vector comprises sequences corresponding to those encoding PB1, PB2, PA, NP, M, or NS, or a portion thereof, having substantially the same activity as a corresponding polypeptide described herein or encoded by a nucleic acid described herein. Optionally, two vectors may be employed in place of the vector comprising a promoter operably linked to an influenza virus M cDNA linked to a transcription termination sequence, e.g., a vector comprising a promoter operably linked to an influenza virus M1 cDNA linked to a transcription termination sequence and a vector comprising a promoter operably linked to an influenza virus M2 cDNA linked to a transcription termination sequence.

A plurality of the vectors of the invention may be physically linked or each vector may be present on an individual plasmid or other, e.g., linear, nucleic acid delivery vehicle. In one embodiment, each vRNA production vector is on a separate plasmid. In one embodiment, each mRNA production vector is on a separate plasmid.

The invention also provides a method to prepare influenza virus. The method comprises contacting a cell with a plurality of the vectors of the invention, e.g., sequentially or simultaneously, in an amount effective to yield infectious influenza virus. The invention also includes isolating virus from a cell contacted with the plurality of vectors. Thus, the invention further provides isolated virus, as well as a host cell contacted with the plurality of vectors or virus of the invention. In another embodiment, the invention includes contacting the cell with one or more vectors, either vRNA or protein production vectors, prior to other vectors, either vRNA or protein production vectors. In one embodiment, the promoter for vRNA vectors employed in the method is a RNA polymerase I promoter, a RNA polymerase II promoter, a RNA polymerase III promoter, a T3 promoter or a T7 promoter. In one embodiment, the RNA polymerase I promoter is a human RNA polymerase I promoter. In one embodiment, each vRNA vector employed in the method is on a separate plasmid. In one embodiment, the vRNA vectors employed in the method are on one plasmid or on two or three different plasmids. In one embodiment, each mRNA vector employed in the method is on a separate plasmid. In one embodiment, the mRNA vectors for PA, PB1, PB2 and NP employed in the method are on one plasmid or on two or three different plasmids.

Exemplary viral sequences for a master vaccine strain (PR8UW)

HA
(SEQ ID NO: 22)
AGCAAAAGCAGGGGAAAATAAAAACAACCAAAATGAAGGCAAACCTACTGGTCCTGTTATGTGCACT
TGCAGCTGCAGATGCAGACACAATATGTATAGGCTACCATGCGAACAATTCAACCGACACTGTTGAC
ACAGTACTCGAGAAGAATGTGACAGTGACACACTCTGTTAACCTGCTCGAAGACAGCCACAACGGAA
AACTATGTAGATTAAAAGGAATAGCCCCACTACAATTGGGGAAATGTAACATCGCCGGATGGCTCTT
GGGAAACCCAGAATGCGACCCACTGCTTCCAGTGAGATCATGGTCCTACATTGTAGAAACACCAAAC
TCTGAGAATGGAATATGTTATCCAGGAGATTTCATCGACTATGAGGAGCTGAGGGAGCAATTGAGCT
CAGTGTCATCATTCGAAAGATTCGAAATATTTCCCAAAGAAAGCTCATGGCCCAACCACAACACAAAC
GGAGTAACGGCAGCATGCTCCCATGAGGGGAAAAGCAGTTTTTACAGAAATTTGCTATGGCTGACGG
AGAAGGAGGGCTCATACCCAAAGCTGAAAAATTCTTATGTGAACAAAAAAGGGAAAGAAGTCCTTGT
ACTGTGGGGTATTCATCACCCGCCTAACAGTAAGGAACAACAGAATCTCTATCAGAATGAAAATGCTT
ATGTCTCTGTAGTGACTTCAAATTATAACAGGAGATTTACCCCGGAAATAGCAGAAAGACCCAAAGTA
AGAGATCAAGCTGGGAGGATGAACTATTACTGGACCTTGCTAAAACCCGGAGACACAATAATATTTG
AGGCAAATGGAAATCTAATAGCACCAATGTATGCTTTCGCACTGAGTAGAGGCTTTGGGTCCGGCAT
CATCACCTCAAACGCATCAATGCATGAGTGTAACACGAAGTGTCAAACACCCCTGGGAGCTATAAAC
AGCAGTCTCCCTTACCAGAATATACACCCAGTCACAATAGGAGAGTGCCCAAAATACGTCAGGAGTG
CCAAATTGAGGATGGTTACAGGACTAAGGAACATTCCGTCCATTCAATCCAGAGGTCTATTTGGAGC
CATTGCCGGTTTTATTGAAGGGGGATGGACTGGAATGATAGATGGATGGTATGGTTATCATCATCAG
AATGAACAGGGATCAGGCTATGCAGCGGATCAAAAAAGCACACAAAATGCCATTAACGGGATTACAA
ACAAGGTGAACACTGTTATCGAGAAAATGAACATTCAATTCACAGCTGTGGGTAAAGAATTCAACAAA
TTAGAAAAAAGGATGGAAAATTTAAATAAAAAAGTTGATGATGGATTTCTGGACATTTGGACATATAAT
GCAGAATTGTTAGTTCTACTGGAAAATGAAAGGACTCTGGATTTCCATGACTCAAATGTGAAGAATCT
GTATGAGAAAGTAAAAAGCCAATTAAAGAATAATGCCAAAGAAATCGGAAATGGATGTTTTGAGTTCT
ACCACAAGTGTGACAATGAATGCATGGAAAGTGTAAGAAATGGGACTTATGATTATCCCAAATATTCA
GAAGAGTCAAAGTTGAACAGGGAAAAGGTAGATGGAGTGAAATTGGAATCAATGGGGATCTATCAGA
TTCTGGCGATCTACTCAACTGTCGCCAGTTCACTGGTGCTTTTGGTCTCCCTGGGGGCAATCAGTTT
CTGGATGTGTTCTAATGGATCTTTGCAGTGCAGAATATGCATCTGAGATTAGAATTTCAGAGATATGA
GGAAAAACACCCTTGTTTCTACT
NA
(SEQ ID NO: 23)
AGCAAAAGCAGGGGTTTAAAATGAATCCAAATCAGAAAATAATAACCATTGGATCAATCTGTCTGGTA
GTCGGACTAATTAGCCTAATATTGCAAATAGGGAATATAATCTCAATATGGATTAGCCATTCAATTCAA
ACTGGAAGTCAAAACCATACTGGAATATGCAACCAAAACATCATTACCTATAAAAATAGCACCTGGGT
AAAGGACACAACTTCAGTGATATTAACCGGCAATTCATCTCTTTGTCCCATCCGTGGGGGGCTATAT
ACAGCAAAGACAATAGCATAAGAATTGGTTCCAAAGGAGACGTTTTTGTCATAAGAGAGCCCTTTATT
TCATGTTCTCACTTGGAATGCAGGACCTTTTTTCTGACCCAAGGTGCCTTACTGAATGACAAGCATTC
AAGTGGGACTGTTAAGGACAGAAGCCCTTATAGGGCCTTAATGAGCTGCCCTGTCGGTGAAGCTCC
GTCCCCGTACAATTCAAGATTTGAATCGGTTGCTTGGTCAGCAAGTGCATGTCATGATGGCATGGGC
TGGCTAACAATCGGAATTTCAGGTCCAGATAATGGAGCAGTGGCTGTATTAAAATACAACGGCATAAT
AACTGAAACCATAAAAAGTTGGAGGAAGAAAATATTGAGGACACAAGAGTCTGAATGTGCCTGTGTAA
ATGGTTCATGTTTTACTATAATGACTGATGGCCCGAGTGATGGGCTGGCCTCGTACAAAATTTTCAAG
ATCGAAAAGGGGAAGGTTACTAAATCAATAGAGTTGAATGCACCTAATTCTCACTATGAGGAATGTTC
CTGTTACCCTGATACCGGCAAAGTGATGTGTGTGTGCAGAGACAATTGGCATGGTTCGAACCGGCCA
TGGGTGTCTTTCGATCAAAACCTGGATTATCAAATAGGATACATCTGCAGTGGGGTTTTCGGTGACAA
CCCGCGTCCCGAAGATGGAACAGGCAGCTGTGGTCCAGTGTATGTTGATGGAGCAAACGGAGTAAA
GGGATTTTCATATAGGTATGGTAATGGTGTTTGGATAGGAAGGACCAAAAGTCACAGTTCCAGACAT
GGGTTTGAGATGATTTGGGATCCTAATGGATGGACAGAGACTGATAGTAAGTTCTCTGTGAGGCAAG
ATGTTGTGGCAATGACTGATTGGTCAGGGTATAGCGGAAGTTTCGTTCAACATCCTGAGCTGACAGG
GCTAGACTGTATGAGGCCGTGCTTCTGGGTTGAATTAATCAGGGGACGACCTAAAGAAAAAACAATC
TGGACTAGTGCGAGCAGCATTTCTTTTTGTGGCGTGAATAGTGATACTGTAGATTGGTCTTGGCCAGA
CGGTGCTGAGTTGCCATTCAGCATTGACAAGTAGTCTGTTCAAAAAACTCCTTGTTTCTACT
PA
(SEQ ID NO: 24)
AGCGAAAGCA GGTACTGATC CAAAATGGAA GATTTTGTGC GACAATGCTT CAATCCGATG
ATTGTCGAGC TTGCGGAAAA AACAATGAAA GAGTATGGGG AGGACCTGAA AATCGAAACA
AACAAATTTG CAGCAATATG CACTCACTTG GAAGTATGCT TCATGTATTC AGATTTTCAC
TTCATCAATG AGCAAGGCGA GTCAATAATC GTAGAACTTG GTGATCCAAA TGCACTTTTG
AAGCACAGAT TTGAAATAAT CGAGGGAAGA GATCGCACAA TGGCCTGGAC AGTAGTAAAC
AGTATTTGCA ACACTACAGG GGCTGAGAAA CCAAAGTTTC TACCAGATTT GTATGATTAC
AAGGAGAATA GATTCATCGA AATTGGAGTA ACAAGGAGAG AAGTTCACAT ATACTATCTG
GAAAAGGCCA ATAAAATTAA ATCTGAGAAA ACACACATCC ACATTTTCTC GTTCACTGGG
GAAGAAATGG CCACAAAGGC AGACTACACT CTCGATGAAG AAAGCAGGGC TAGGATCAAA
ACCAGACTAT TCACCATAAG ACAAGAAATG GCCAGCAGAG GCCTCTGGGA TTCCTTTCGT
CAGTCCGAGA GAGGAGAAGA GACAATTGAA GAAAGGTTTG AAATCACAGG AACAATGCGC
AAGCTTGCCG ACCAAAGTCT CCCGCCGAAC TTCTCCAGCC TTGAAAATTT TAGAGCCTAT
GTGGATGGAT TOGAACCGAA CGGCTACATT GAGGGCAAGC TGTCTCAAAT GTCCAAAGAA
GTAAATGCTA GAATTGAACC TTTTTTGAAA ACAACACCAC GACCACTTAG ACTTCCGAAT
GGGCCTCCCT GTTCTCAGCG GTCCAAATTC CTGCTGATGG ATGCCTTAAA ATTAAGCATT
GAGGACCCAA GTCATGAAGG AGAGGGAATA CCGCTATATG ATGCAATCAA ATGCATGAGA
ACATTCTTTG GATGGAAGGA ACCCAATGTT GTTAAACCAC ACGAAAAGGG AATAAATCCA
AATTATCTTC TGTCATGGAA GCAAGTACTG GCAGAACTGC AGGACATTGA GAATGAGGAG
AAAATTCCAA AGACTAAAAA TATGAAGAAA ACAAGTCAGC TAAAGTGGGC ACTTGGTGAG
AACATGGCAC CAGAAAAGGT AGACTTTGAC GACTGTAAAG ATGTAGGTGA TTTGAAGCAA
TATGATAGTG ATGAACCAGA ATTGAGGTCG CTTGCAAGTT GGATTCAGAA TGAGTTTAAC
AAGGCATGCG AACTGACAGA TTCAAGCTGG ATAGAGCTCG ATGAGATTGG AGAAGATGTG
GCTCCAATTG AACACATTGC AAGCATGAGA AGGAATTATT TCACATCAGA GGTGTCTCAC
TGCAGAGCCA CAGAATACAT AATGAAGGGA GTGTACATCA ATACTGCCTT GOTTAATGCA
TCTTGTGCAG CAATGGATGA TTTCCAATTA ATTCCAATGA TAAGCAAGTG TAGAACTAAG
GAGGGAAGGC GAAAGACCAA CTTGTATGGT TTCATCATAA AAGGAAGATC CCACTTAAGG
AATGACACCG ACGTGGTAAA CTTTGTGAGC ATGGAGTTTT CTCTCACTGA CCCAAGACTT
GAACCACATA AATGGGAGAA GTACTGTGTT CTTGAGATAG GAGATATGCT TATAAGAAGT
GCCATAGGCC AGGTTTCAAG GCCCATGTTC TTGTATGTGA GAACAAATGG AACCTCAAAA
ATTAAAATGA AATGGGGAAT GGAGATGAGG CGTTGCCTCC TCCAGTCACT TCAACAAATT
GAGAGTATGA TTGAAGCTGA GTCCTCTGTC AAAGAGAAAG ACATGACCAA AGAGTTCTTT
GAGAACAAAT CAGAAACATG GCCCATTGGA GAGTCCCCCA AAGGAGTGGA GGAAAGTTCC
ATTGGGAAGG TCTGCAGGAC TTTATTAGCA AAGTCGGTAT TCAACAGCTT GTATGCATCT
CCACAACTAG AAGGATTTTC AGCTGAATCA AGAAAACTGC TTCTTATCGT TCAGGCTCTT
AGGGACAACC TGGAACCTGG GACCTTTGAT CTTGGGGGGC TATATGAAGC AATTGAGGAG
TGCCTGATTA ATGATCCCTG GGTTTTGCTT AATGCTTCTT GGTTCAACTC CTTCOTTACA
CATGCATTGA GTTAGTTGTG GCAGTGCTAC TATTTGCTAT CCATACTGTC CAAAAAAGTA
CCTTGTTTCT ACT
PB1
(SEQ ID NO: 25)
AGCGAAAGCAGGCAAACCATTTGAATGGATGTCAATCCGACCTTACTTTTCTTAAAAGTGCCAGCACA
AAATGCTATAAGCACAACTTTCCCTTATACTGGAGACCCTCCTTACAGCCATGGGACAGGAACAGGA
TACACCATGGATACTGTCAACAGGACACATCAGTACTCAGAAAAGGGAAGATGGACAACAAACACCG
AAACTGGAGCACCGCAACTCAACCCGATTGATGGGCCACTGCCAGAAGACAATGAACCAAGTGGTTA
TGCCCAAACAGATTGTGTATTGGAGGCGATGGCTTTCCTTGAGGAATCCCATCCTGGTATTTTTGAAA
ACTCGTGTATTGAAACGATGGAGGTTGTTCAGCAAACACGAGTAGACAAGCTGACACAAGGCCGACA
GACCTATGACTGGACTCTAAATAGAAACCAACCTGCTGCAACAGCATTGGCCAACACAATAGAAGTG
TTCAGATCAAATGGCCTCACGGCCAATGAGTCTGGAAGGCTCATAGACTTCCTTAAGGATGTAATGG
AGTCAATGAACAAAGAAGAAATGGGGATCACAACTCATTTTCAGAGAAAGAGACGGGTGAGAGACAA
TATGACTAAGAAAATGATAACACAGAGAACAATGGGTAAAAAGAAGCAGAGATTGAACAAAAGGAGTT
ATCTAATTAGAGCATTGACCCTGAACACAATGACCAAAGATGCTGAGAGAGGGAAGCTAAAACGGAG
AGCAATTGCAACCCCAGGGATGCAAATAAGGGGGTTTGTATACTTTGTTGAGACACTGGCAAGGAGT
ATATGTGAGAAACTTGAACAATCAGGGTTGCCAGTTGGAGGCAATGAGAAGAAAGCAAAGTTGGCAA
ATGTTGTAAGGAAGATGATGACCAATTCTCAGGACACCGAACTTTCTTTCACCATCACTGGAGATAAC
ACCAAATGGAACGAAAATCAGAATCCTCGGATGTTTTTGGCCATGATCACATATATGACCAGAAATCA
GCCCGAATGGTTCAGAAATGTTCTAAGTATTGCTCCAATAATGTTCTCAAACAAAATGGCGAGACTGG
GAAAAGGGTATATGTTTGAGAGCAAGAGTATGAAACTTAGAACTCAAATACCTGCAGAAATGCTAGCA
AGCATCGATTTGAAATATTTCAATGATTCAACAAGAAAGAAGATTGAAAAAATCCGACCGCTCTTAATA
GAGGGGACTGCATCATTGAGCCCTGGAATGATGATGGGCATGTTCAATATGTTAAGCACTGTATTAG
GCGTCTCCATCCTGAATCTTGGACAAAAGAGATACACCAAGACTACTTACTGGTGGGATGGTCTTCA
ATCCTCTGACGATTTTGCTCTGATTGTGAATGCACCCAATCATGAAGGGATTCAAGCCGGAGTCGAC
AGGTTTTATCGAACCTGTAAGCTACTTGGAATCAATATGAGCAAGAAAAAGTCTTACATAAACAGAAC
AGGTACATTTGAATTCACAAGTTTTTTCTATCGTTATGGGTTTGTTGCCAATTTCAGCATGGAGCTTCC
CAGTTTTGGGGTGTCTGGGATCAACGAGTCAGCGGACATGAGTATTGGAGTTACTGTCATCAAAAAC
AATATGATAAACAATGATCTTGGTCCAGCAACAGCTCAAATGGCCCTTCAGTTGTTCATCAAAGATTA
CAGGTACACGTACCGATGCCATATAGGTGACACACAAATACAAACCCGAAGATCATTTGAAATAAAGA
AACTGTGGGAGCAAACCCGTTCCAAAGCTGGACTGCTGGTCTCCGACGGAGGCCCAAATTTATACAA
CATTAGAAATCTCCACATTCCTGAAGTCTGCCTAAAATGGGAATTGATGGATGAGGATTACCAGGGG
CGTTTATGCAACCCACTGAACCCATTTGTCAGCCATAAAGAAATTGAATCAATGAACAATGCAGTGAT
GATGCCAGCACATGGTCCAGCCAAAAACATGGAGTATGATGCTGTTGCAACAACACACTCCTGGATC
CCCAAAAGAAATCGATCCATCTTGAATACAAGTCAAAGAGGAGTACTTGAGGATGAACAAATGTACCA
AAGGTGCTGCAATTTATTTGAAAAATTCTTCCCCAGCAGTTCATACAGAAGACCAGTCGGGATATCCA
GTATGGTGGAGGCTATGGTTTCCAGAGCCCGAATTGATGCACGGATTGATTTCGAATCTGGAAGGAT
AAAGAAAGAAGAGTTCACTGAGATCATGAAGATCTGTTCCACCATTGAAGAGCTCAGACGGCAAAAA
TAGTGAATTTAGCTTGTCCTTCATGAAAAAATGCCTTGTTTCTACT
PB2
(SEQ ID NO: 26)
AGCGAAAGCA GGTCAATTAT ATTCAATATG GAAAGAATAA AAGAACTACG AAATCTAATG
TCGCAGTCTC GCACCCGCGA GATACTCACA AAAACCACCG TGGACCATAT GGCCATAATC
AAGAAGTACA CATCAGGAAG ACAGGAGAAG AACCCAGCAC TTAGGATGAA ATGGATGATG
GCAATGAAAT ATCCAATTAC AGCAGACAAG AGGATAACGG AAATGATTCC TGAGAGAAAT
GAGCAAGGAC AAACTTTATG GAGTAAAATG AATGATGCCG GATCAGACCG AGTGATGGTA
TCACCTCTGG CTGTGACATG GTGGAATAGG AATGGACCAA TAACAAATAC AGTTCATTAT
CCAAAAATCT ACAAAACTTA TTTTGAAAGA GTCGAAAGGC TAAAGCATGG AACCTTTGGC
CCTGTCCATT TTAGAAACCA AGTCAAAATA CGTCGGAGAG TTGACATAAA TCCTGGTCAT
GCAGATCTCA GTGCCAAGGA GGCACAGGAT GTAATCATGG AAGTTGTTTT CCCTAACGAA
GTGGGAGCCA GGATACTAAC ATCGGAATCG CAACTAACGA TAACCAAAGA GAAGAAAGAA
GAACTCCAGG ATTGCAAAAT TTCTCCTTTG ATGGTTGCAT ACATGTTGGA GAGAGAACTG
GTCCGCAAAA CGAGATTCCT CCCAGTGGCT GGTGGAACAA GCAGTGTGTA CATTGAAGTG
TTGCATTTGA CTCAAGGAAC ATGCTGGGAA CAGATGTATA CTCCAGGAGG GGAAGTGAGG
AATGATGATG TTGATCAAAG CTTGATTATT GCTGCTAGGA ACATAGTGAG AAGAGCTGCA
GTATCAGCAG ATCCACTAGC ATCTTTATTG GAGATGTGCC ACAGCACACA GATTGGTGGA
ATTAGGATGG TAGACATCCT TAGGCAGAAC CCAACAGAAG AGCAAGCCGT GGATATATGC
AAGGCTGCAA TGGGACTGAG AATTAGCTCA TCCTTCAGTT TTGGTGGATT CACATTTAAG
AGAACAAGCG GATCATCAGT CAAGAGAGAG GAAGAGGTGC TTACGGGCAA TCTTCAAACA
TTGAAGATAA GAGTGCATGA GGGATATGAA GAGTTCACAA TGGTTGGGAG AAGAGCAACA
GCCATACTCA GAAAAGCAAC CAGGAGATTG ATTCAGCTGA TAGTGAGTGG GAGAGACGAA
CAGTCGATTG CCGAAGCAAT AATTGTGGCC ATGGTATTTT CACAAGAGGA TTGTATGATA
AAAGCAGTCA GAGGTGATCT GAATTTCGTC AATAGGGCGA ATCAACGATT GAATCCTATG
CATCAACTTT TAAGACATTT TCAGAAGGAT GCGAAAGTGC TTTTTCAAAA TTGGGGAGTT
GAACCTATCG ACAATGTGAT GGGAATGATT GGGATATTGC CCGACATGAC TCCAAGCATC
GAGATGTCAA TGAGAGGAGT GAGAATCAGC AAAATGGGTG TAGATGAGTA CTCCAGCACG
GAGAGGGTAG TGGTGAGCAT TGACCGTTTT TTGAGAATCC GGGACCAACG AGGAAATGTA
CTACTGTCTC CCGAGGAGGT CAGTGAAACA CAGGGAACAG AGAAACTGAC AATAACTTAC
TCATCGTCAA TGATGTGGGA GATTAATGGT CCTGAATCAG TGTTGGTCAA TACCTATCAA
TGGATCATCA GAAACTGGGA AACTGTTAAA ATTCAGTGGT CCCAGAACCC TACAATGCTA
TACAATAAAA TGGAATTTGA ACCATTTCAG TOTTTAGTAC CTAAGGCCAT TAGAGGCCAA
TACAGTGGGT TTGTAAGAAC TCTGTTCCAA CAAATGAGGG ATGTGCTTGG GACATTTGAT
ACCGCACAGA TAATAAAACT TOTTCCCTTC GCAGCCGCTC CACCAAAGCA AAGTAGAATG
CAGTTCTCCT CATTTACTGT GAATGTGAGG GGATCAGGAA TGAGAATACT TGTAAGGGGC
AATTCTCCTG TATTCAACTA TAACAAGGCC ACGAAGAGAC TCACAGTTCT CGGAAAGGAT
GCTGGCACTT TAACTGAAGA CCCAGATGAA GGCACAGCTG GAGTGGAGTC CGCTGTTCTG
AGGGGATTCC TCATTCTGGG CAAAGAAGAC AAGAGATATG GGCCAGCACT AAGCATCAAT
GAACTGAGCA ACCTTGCGAA AGGAGAGAAG GCTAATGTGC TAATTGGGCA AGGAGACGTG
GTGTTGGTAA TGAAACGGAA ACGGGACTCT AGCATACTTA CTGACAGCCA GACAGCGACC
AAAAGAATTC GGATGGCCAT CAATTAGTGT CGAATAGTTT AAAAACGACC TTGTTTCTAC T
NP
(SEQ ID NO: 27)
AGCAAAAGCA GGGTAGATAA TCACTCACTG AGTGACATCA AAATCATGGC GTCTCAAGGC
ACCAAACGAT CTTACGAACA GATGGAGACT GATGGAGAAC GCCAGAATGC CACTGAAATC
AGAGCATCCG TCGGAAAAAT GATTGGTGGA ATTGGACGAT TCTACATCCA AATGTGCACC
GAACTCAAAC TCAGTGATTA TGAGGGACGG TTGATCCAAA ACAGCTTAAC AATAGAGAGA
ATGGTGCTCT CTGCTTTTGA CGAAAGGAGA AATAAATACC TTGAAGAACA TOCCAGTGCG
GGGAAAGATC CTAAGAAAAC TGGAGGACCT ATATACAGGA GAGTAAACGG AAAGTGGATG
AGAGAACTCA TCCTTTATGA CAAAGAAGAA ATAAGGCGAA TCTGGCGCCA AGCTAATAAT
GGTGACGATG CAACGGCTGG TCTGACTCAC ATGATGATCT GGCATTCCAA TTTGAATGAT
GCAACTTATC AGAGGACAAG AGCTCTTGTT CGCACCGGAA TGGATCCCAG GATGTGCTCT
CTGATGCAAG GTTCAACTCT CCCTAGGAGG TCTGGAGCCG CAGGTGCTGC AGTCAAAGGA
GTTGGAACAA TGGTGATGGA ATTGGTCAGA ATGATCAAAC GTGGGATCAA TGATCGGAAC
TTCTGGAGGG GTGAGAATGG ACGAAAAACA AGAATTGOTT ATGAAAGAAT GTGCAACATT
CTCAAAGGGA AATTTCAAAC TGCTGCACAA AAAGCAATGA TGGATCAAGT GAGAGAGAGC
CGGAACCCAG GGAATGCTGA GTTCGAAGAT CTCACTTTTC TAGCACGGTC TGCACTCATA
TTGAGAGGGT CGGTTGCTCA CAAGTCCTGC CTGCCTGCCT GTGTGTATGG ACCTGCCGTA
GCCAGTGGGT ACGACTTTGA AAGGGAGGGA TACTCTCTAG TCGGAATAGA CCCTTTCAGA
CTGCTTCAAA ACAGCCAAGT GTACAGCCTA ATCAGACCAA ATGAGAATCC AGCACACAAG
AGTCAACTGG TGTGGATGGC ATGCCATTCT GCCGCATTTG AAGATCTAAG AGTATTAAGC
TTCATCAAAG GGACGAAGGT GCTCCCAAGA GGGAAGCTTT CCACTAGAGG AGTTCAAATT
GCTTCCAATG AAAATATGGA GACTATGGAA TCAAGTACAC TTGAACTGAG AAGCAGGTAC
TGGGCCATAA GGACCAGAAG TGGAGGAAAC ACCAATCAAC AGAGGGCATC TGCGGGCCAA
ATCAGCATAC AACCTACGTT CTCAGTACAG AGAAATCTCC CTTTTGACAG AACAACCATT
ATGGCAGCAT TCAATGGGAA TACAGAGGGG AGAACATCTG ACATGAGGAC CGAAATCATA
AGGATGATGG AAAGTGCAAG ACCAGAAGAT GTGTCTTTCC AGGGGGGGGG AGTCTTCGAG
CTCTOGGACG AAAAGGCAGC GAGCCCGATC GTGCCTTCCT TTGACATGAG TAATGAAGGA
TCTTATTTCT TCGGAGACAA TGCAGAGGAG TACGACAATT AAAGAAAAAT ACCCTTGTTT
CTACT
M
(SEQ ID NO: 28)
AGCAAAAGCA GGTAGATATT GAAAGATGAG TOTTCTAACC GAGGTCGAAA CGTACGTACT
CTCTATCATC CCGTCAGGCC CCCTCAAAGC CGAGATCGCA CAGAGACTTG AAGATGTCTT
TGCAGGGAAG AACACCGATC TTGAGGTTCT CATGGAATGG CTAAAGACAA GACCAATCCT
GTCACCTCTG ACTAAGGGGA TTTTAGGATT TGTGTTCACG CTCACCGTGC CCAGTGAGCG
AGGACTGCAG CGTAGACGCT TTGTCCAAAA TGCCCTTAAT GGGAACGGGG ATCCAAATAA
CATGGACAAA GCAGTTAAAC TGTATAGGAA GCTCAAGAGG GAGATAACAT TCCATGGGGC
CAAAGAAATC TCACTCAGTT ATTCTGCTGG TGCACTTGCC AGTTGTATGG GCCTCATATA
CAACAGGATG GGGGCTGTGA CCACTGAAGT GGCATTTGGC CTGGTATGTG CAACCTGTGA
ACAGATTGCT GACTCCCAGC ATCGGTCTCA TAGGCAAATG GTGACAACAA CCAATCCACT
AATCAGACAT GAGAACAGAA TGGTTTTAGC CAGCACTACA GCTAAGGCTA TGGAGCAAAT
GGCTGGATCG AGTGAGCAAG CAGCAGAGGC CATGGAGGTT GCTAGTCAGG CTAGACAAAT
GGTGCAAGCG ATGAGAACCA TTGGGACTCA TCCTAGCTCC AGTGCTGGTC TGAAAAATGA
TCTTCTTGAA AATTTGCAGG CCTATCAGAA ACGAATGGGG GTGCAGATGC AACGGTTCAA
GTGATCCTCT CACTATTGCC GCAAATATCA TTGGGATOTT GCACTTGACA TTGTGGATTC
TTGATCGTCT TTTTTTCAAA TGCATTTACC GTCGCTTTAA ATACGGACTG AAAGGAGGGC
CTTCTACGGA AGGAGTGCCA AAGTCTATGA GGGAAGAATA TCGAAAGGAA CAGCAGAGTG
CTGTGGATGC TGACGATGGT CATTTTGTCA GCATAGAGCT GGAGTAAAAA ACTACCTTGT
TTCTACT
NS
(SEQ ID NO: 29)
AGCAAAAGCA GGGTGACAAA AACATAATGG ATCCAAACAC TGTGTCAAGC TTTCAGGTAG
ATTGCTTTCT TTGGCATGTC CGCAAACGAG TTGCAGACCA AGAACTAGGC GATGCCCCAT
TCCTTGATCG GCTTCGCCGA GATCAGAAAT CCCTAAGAGG AAGGGGCAGT ACTCTCGGTC
TGGACATCAA GACAGCCACA CGTGCTGGAA AGCAGATAGT GGAGCGGATT CTGAAAGAAG
AATCCGATGA GGCACTTAAA ATGACCATGG CCTCTGTACC TGCGTCGCGT TACCTAACTG
ACATGACTCT TGAGGAAATG TCAAGGGACT GGTCCATGCT CATACCCAAG CAGAAAGTGG
CAGGCCCTCT TTGTATCAGA ATGGACCAGG CGATCATGGA TAAGAACATC ATACTGAAAG
CGAACTTCAG TGTGATTTTT GACCGGCTGG AGACTCTAAT ATTGCTAAGG GCTTTCACCG
AAGAGGGAGC AATTGTTGGC GAAATTTCAC CATTGCCTTC TOTTCCAGGA CATACTGCTG
AGGATGTCAA AAATGCAGTT GGAGTCCTCA TOGGAGGACT TGAATGGAAT GATAACACAG
TTCGAGTCTC TGAAACTCTA CAGAGATTCG CTTGGAGAAG CAGTAATGAG AATGGGAGAC
CTCCACTCAC TCCAAAACAG AAACGAGAAA TGGGGGGAAC AATTAGGTCA GAAGTTTGAA
GAAATAAGAT GGTTGATTGA AGAAGTGAGA CACAAACTGA AGATAACAGA GAATAGTTTT
GAGCAAATAA CATTTATGCA AGCCTTACAT CTATTGCTTG AAGTGGAGCA AGAGATAAGA
ACTTTCTCGT TTCAGCTTAT TTAGTACTAA AAAACACCCT TGTTTCTACT

### Exemplary Neuraminidase Modifications

### Materials

### Viruses: Y2017: A/Yokohama/2017/2003 (H3N2)

HK4801: A/Hong Kong/4801/2014(H3N2)

Y2017-M3L4: Y2017 passaged 7 times in eggs

HY-PR8: high yield PR8 (H1N1)

### Results

Y2017 virus was passaged 7 times in eggs (3 times in the amniotic cavity, followed by 4 times in the allantoic cavity). A progeny virus, Y2017-M3L4, grew efficiently in the allantoic cavity (10⁷ to about 10⁸ PFU/mL), whereas the original Y2017 virus did not grow at all (<10 PFU/mL).

Mutations observed in Y2017-M3L4 virus were as follows:

TABLE 1

| | PB2 | NA | NP | M1 |
|---|---|---|---|---|
| eggA | T147I, V344L and | del 46-50aa, T32A, | none | E23Q |
| | T147I, V344L, E358K | D147N, N329D, H347Q | | |
| eggB | T147I | del 46-50aa, T32A, | D101N | none |
| | | D147N, N329D, H347Q | | |
| eggC | T147I | del 46-50aa, T32A, | D101N | none |
| | | D147N, N329D, H347Q | | |

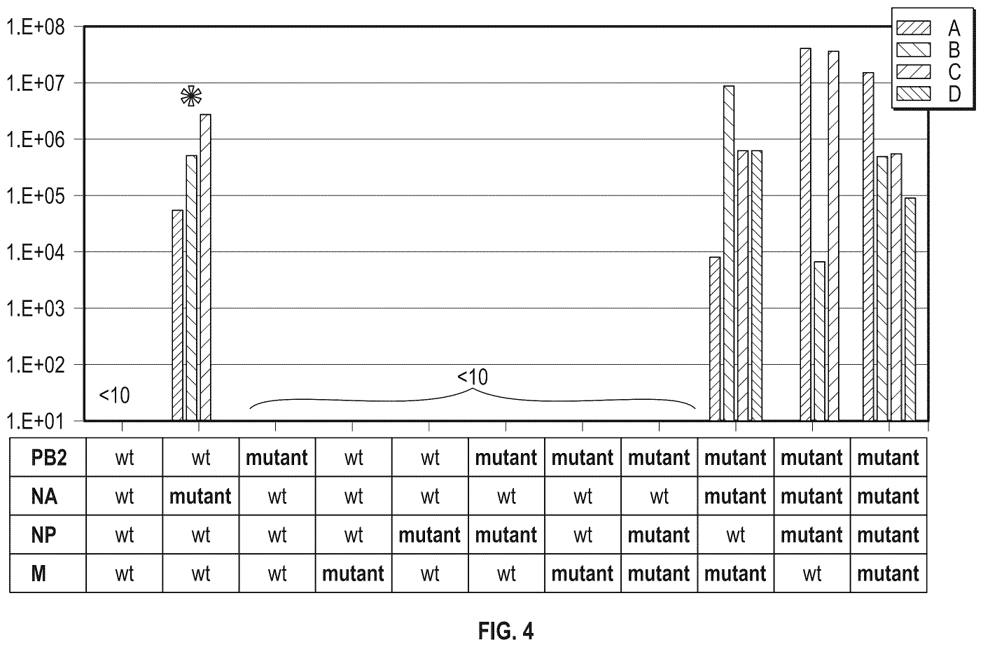

A comparison of the growth ability of mutant Y2017 viruses, generated by reverse genetics, in allantoic fluid revealed that NA mutations were responsible for the high growth of Y2017-M3L4 virus (FIG. 4). A plasmid with PB2-T147I was used for virus generation (PB2-T147I, V344L and PB2-T147I, V344L, E358K were not analyzed). Mutations were not observed in the HA gene of the virus possessing a mutated NA segment and its other genes from wild-type Y2017 after replication in allantoic fluid (FIG. 4).

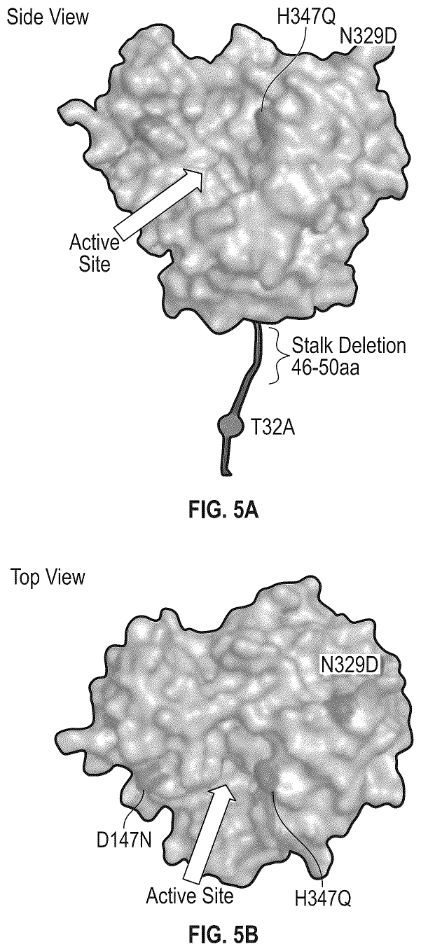

FIG. 5 shows the location of the NA mutations in Y2017-M3L4 in a 3D model.

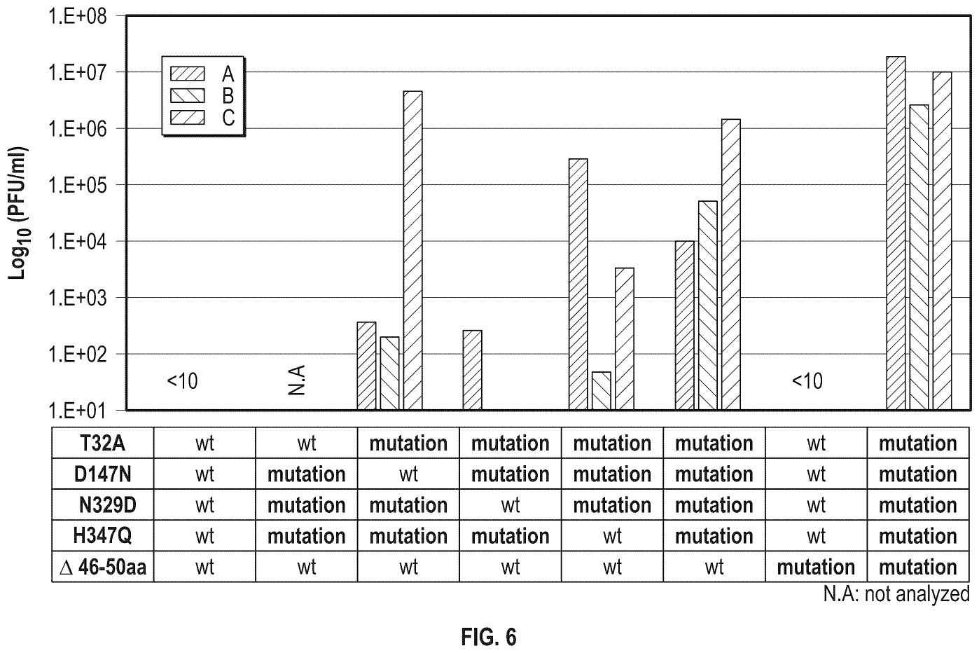

Comparison of the growth ability of Y2017 viruses with NA mutations revealed that NA-D147N, N329D, and H347Q generally contributed to the increased growth ability in allantoic fluid (FIG. 6).

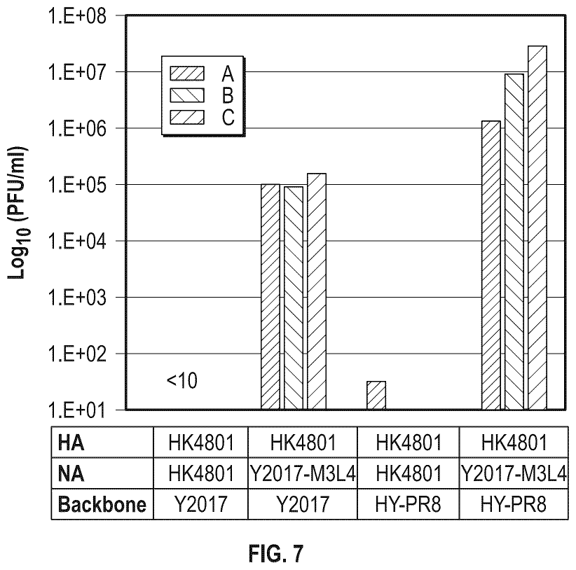

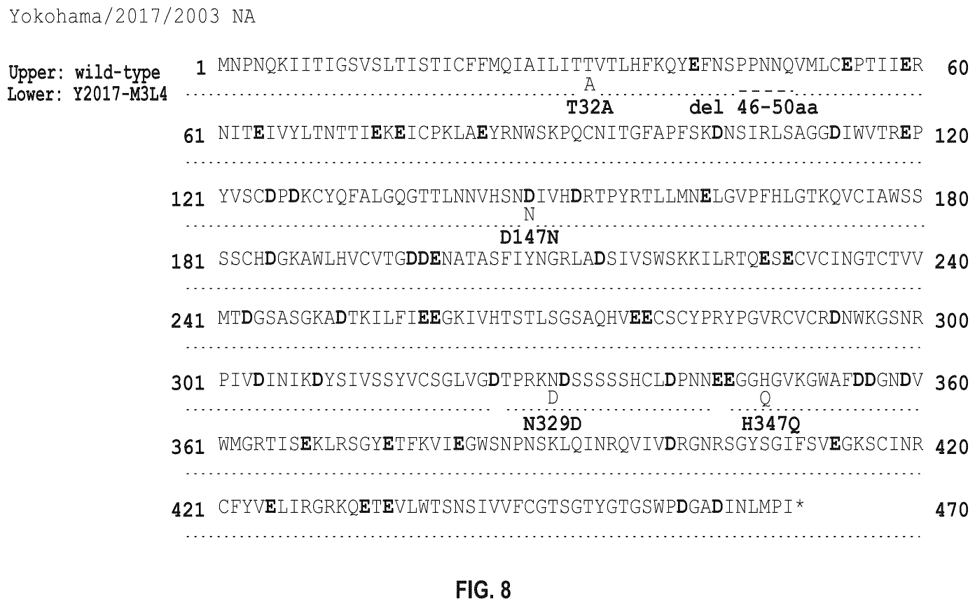

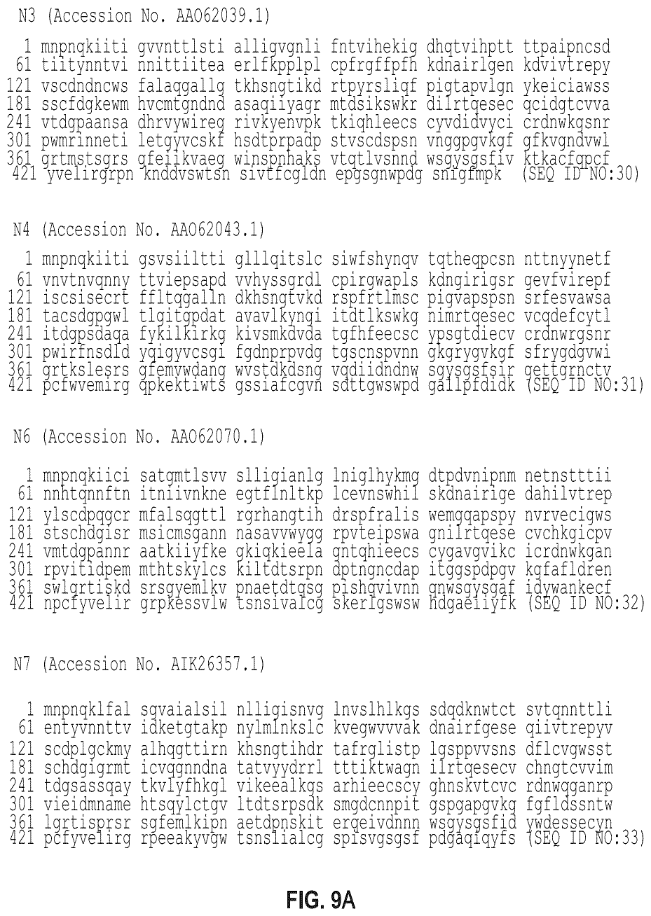

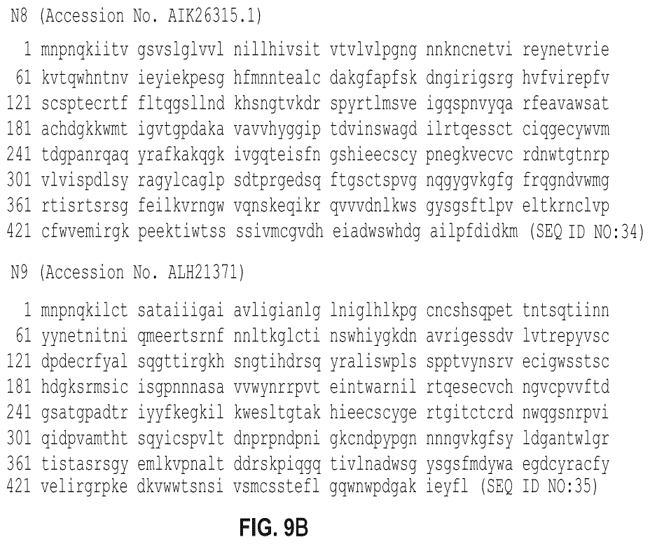

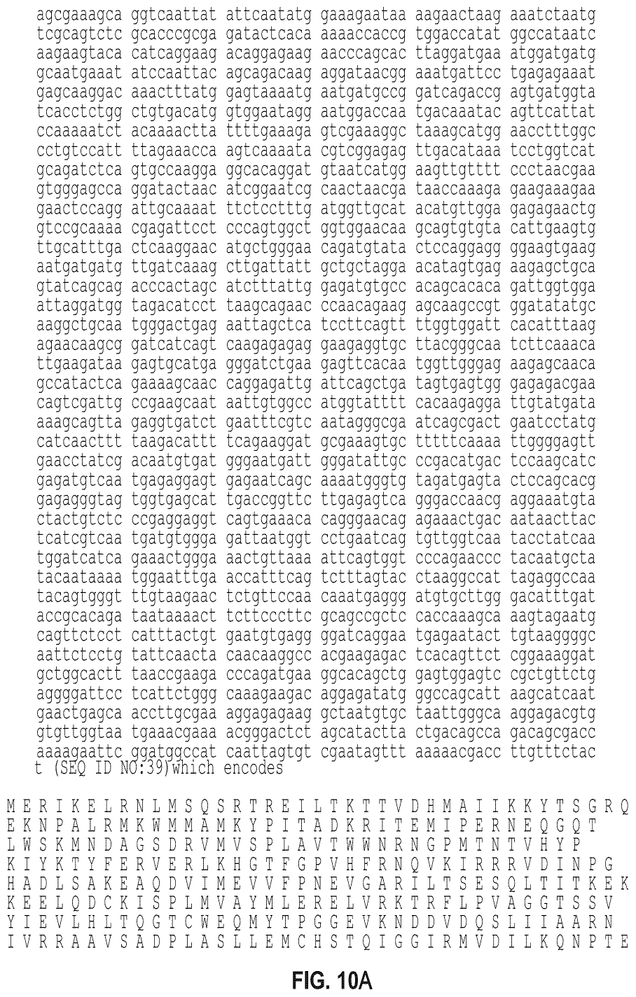

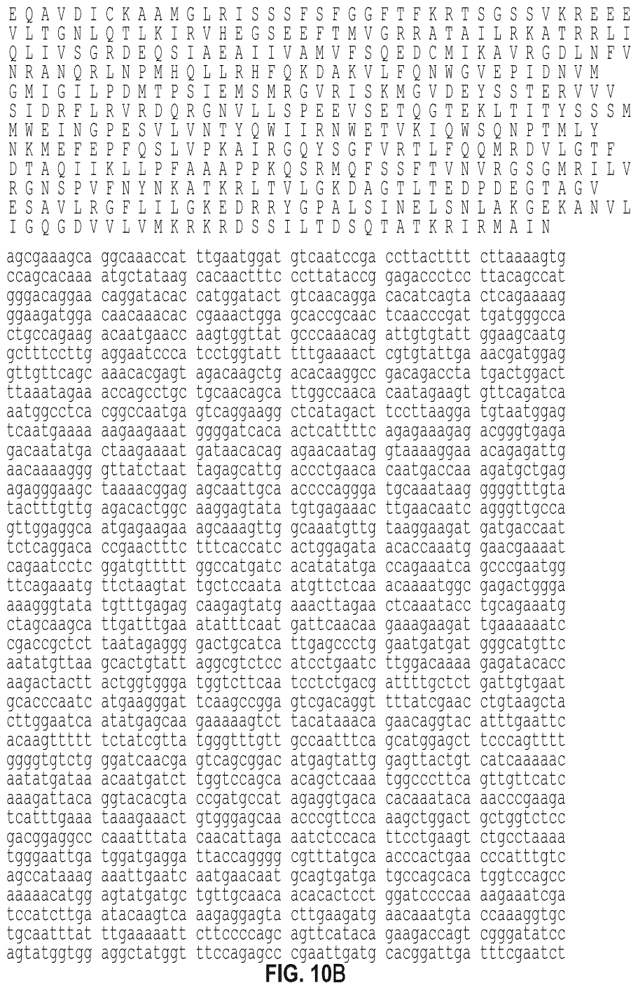

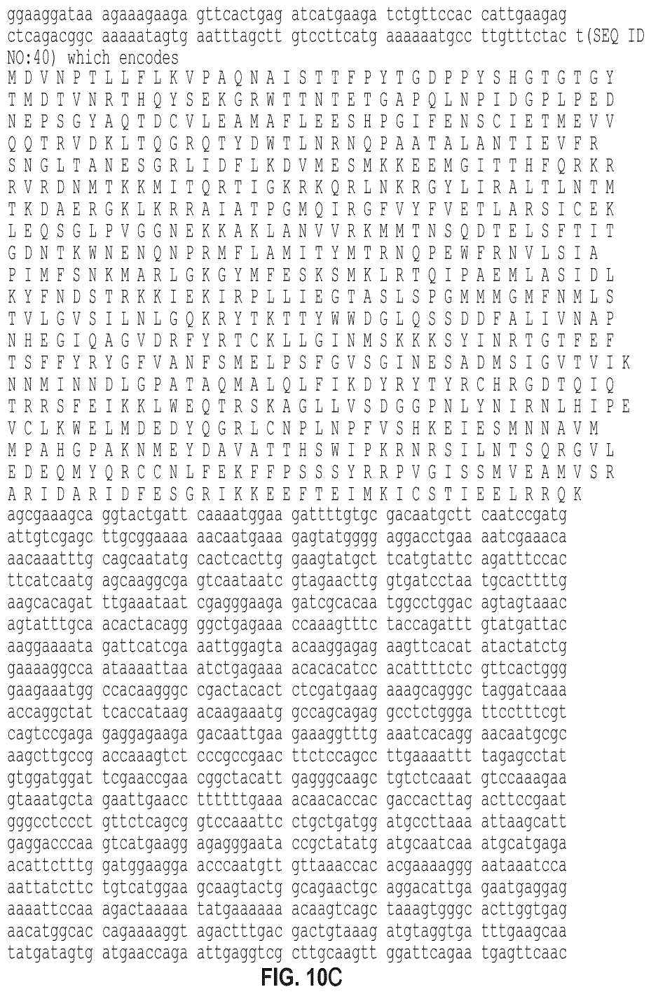

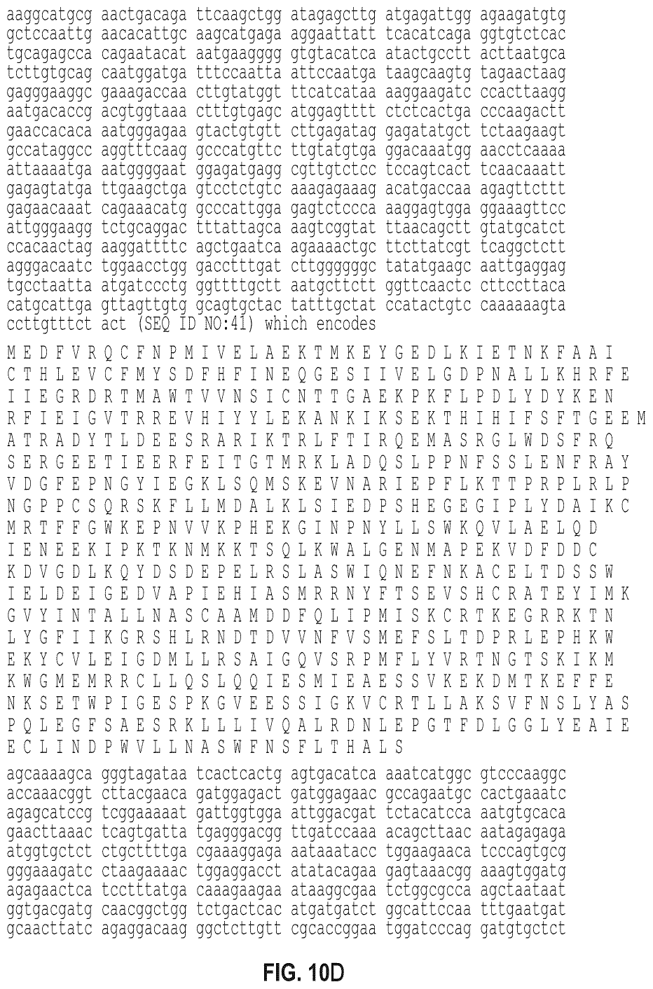

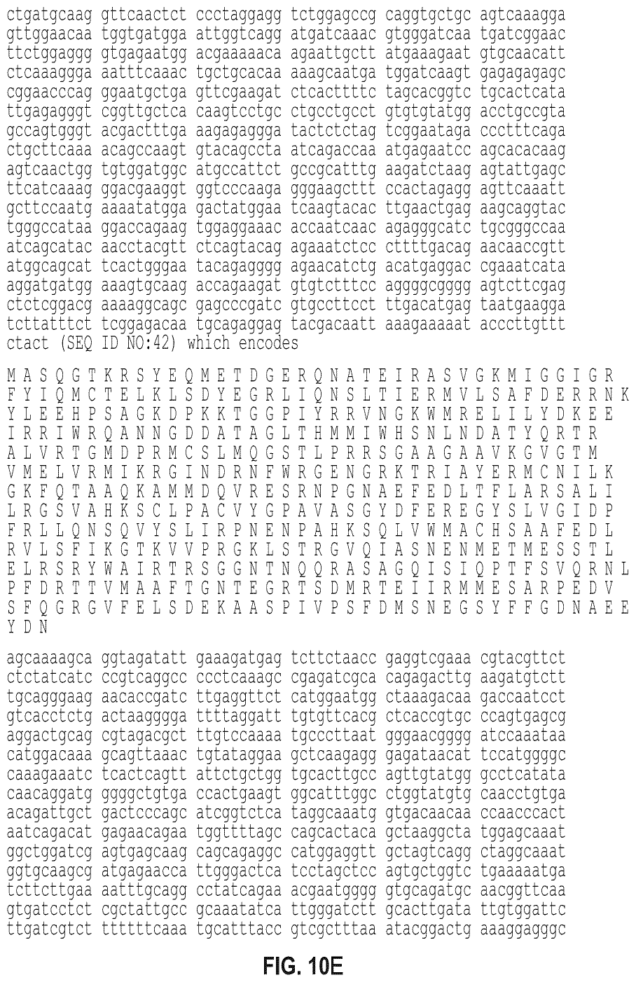

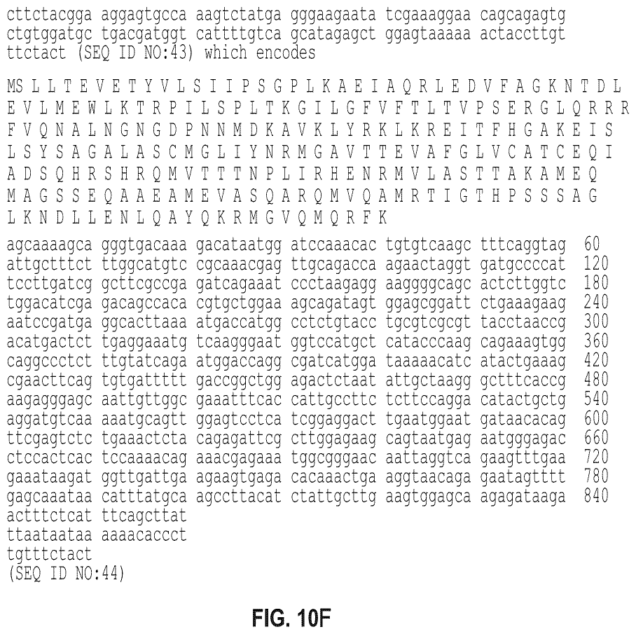

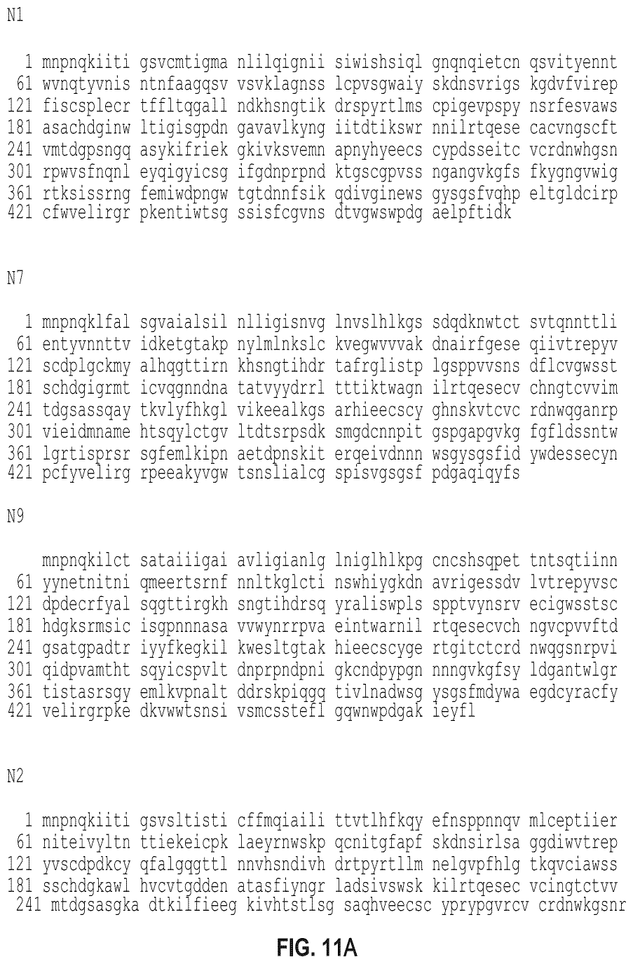

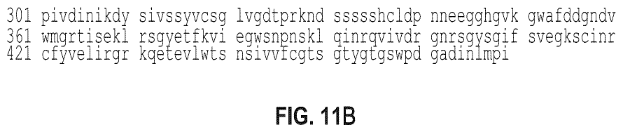

The NA of Y2017-M3L4 allowed virus possessing HK4801 HA to replicate efficiently in the allantoic cavity and the HY-PR8 backbone further enhanced the growth of this virus (FIG. 7).

In summary, described herein are influenza virus mutations that inhibit (e.g., prevent) the acquisition of antigenicity-compromising mutations in the hemagglutinin (HA) protein of influenza during growth in eggs and/or allow for enhanced replication. In one embodiment, the mutations are within the neuraminidase (NA) viral segment of human influenza viruses, and the mutant NA proteins stabilize the HA protein during egg-passages. Thus, in the presence of the mutant NA proteins, the HA protein does not acquire egg-adapting mutations. In some cases, the respective mutations in NA can also increase the yield of vaccine viruses.

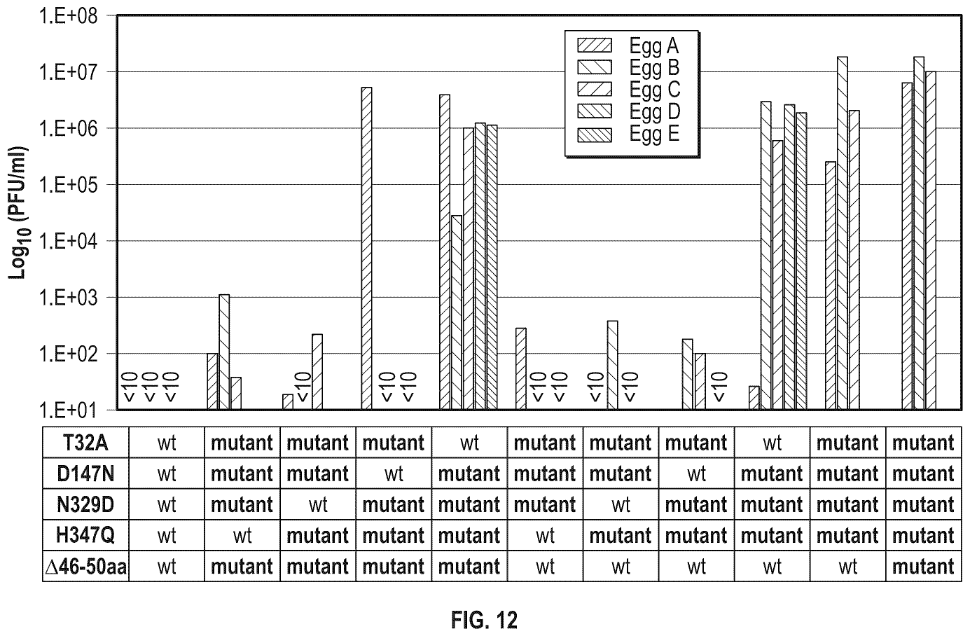

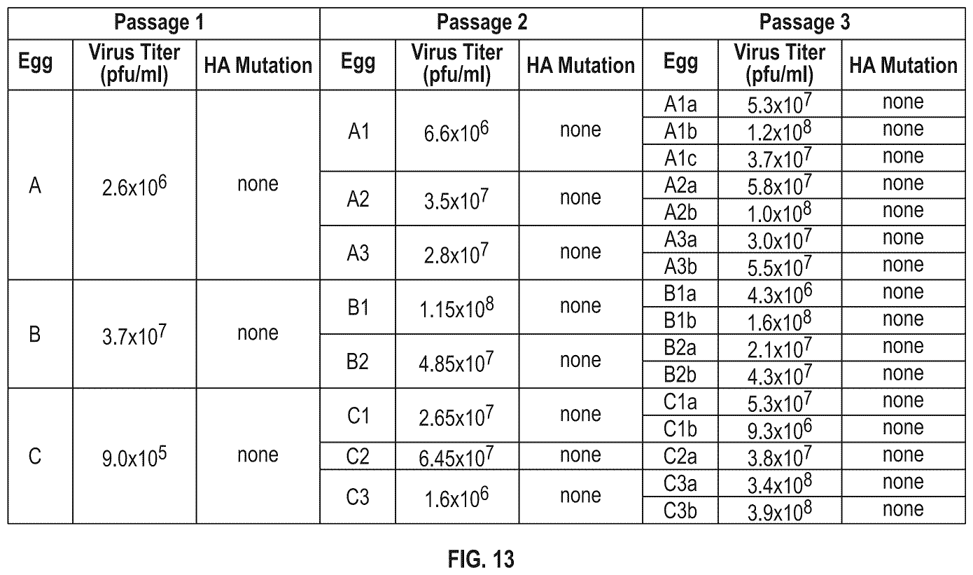

Analysis of the growth capability of NA mutant viruses revealed that NA-D147N, N329D, and H347Q contribute to the increased growth capability of the viruses in allantoic fluid (FIG. 12). HA mutations were not observed in the virus possessing HK4801HA, Y2017-M3L4NA, and the HY-PR8 backbone (FIG. 13) after 3 passages in the allantoic cavity.

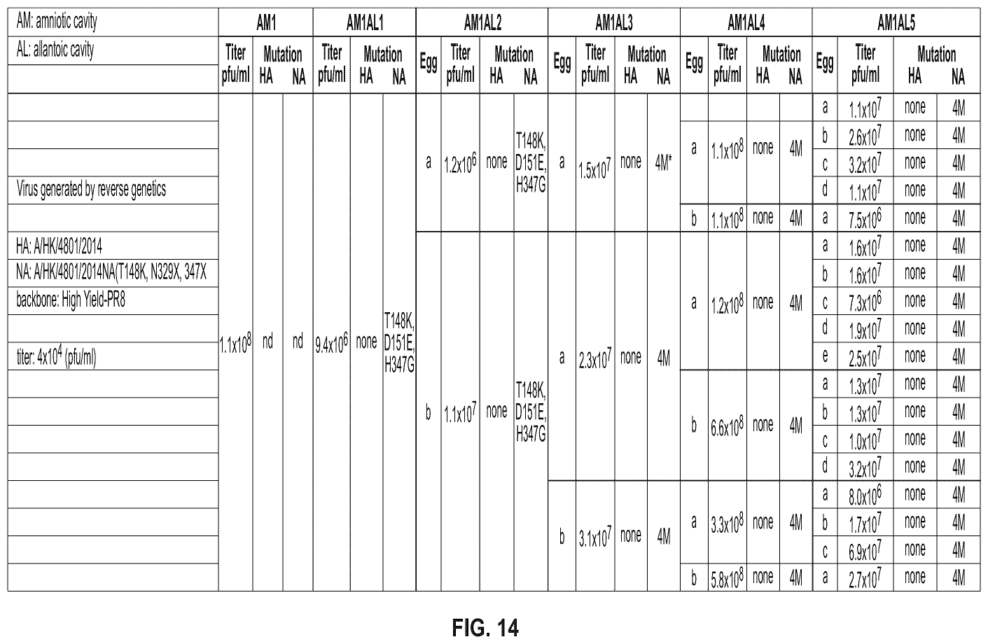

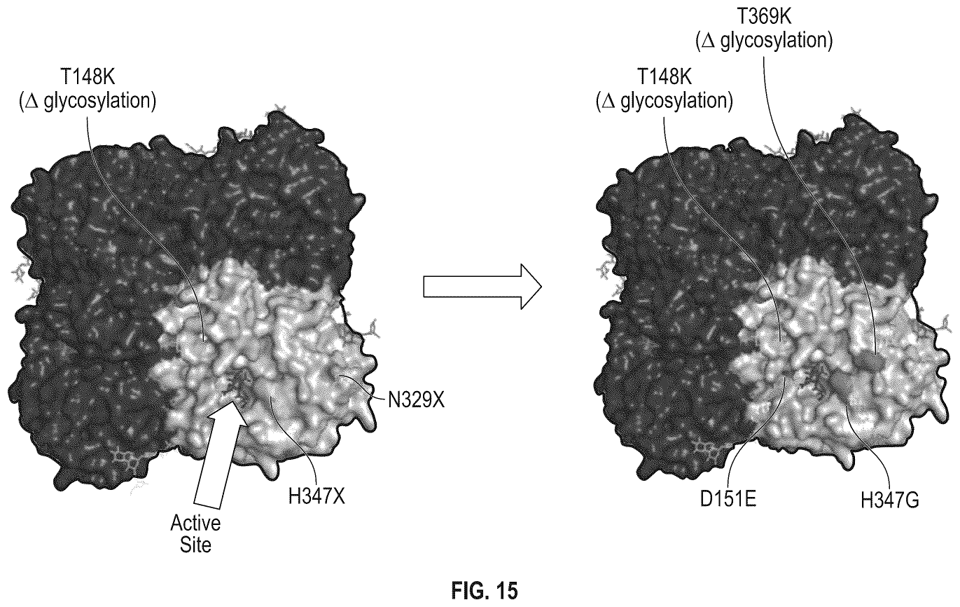

By passaging an HY-PR8 backbone virus possessing HK4801NA (T148K and the saturated mutations N329X and H347X) and HK4801HA in eggs, a virus possessing HK4801NA (T148K, D151E, H347G, and T369K) emerged that replicated efficiently in the allantoic cavity (FIG. 14; 4M=T148K, D151E, H347G, and T369K). HA mutations were not observed during passages in eggs (1× in the amniotic cavity then 5× in the allantoic cavity).

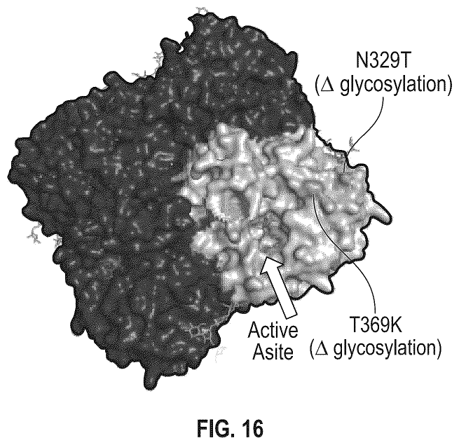

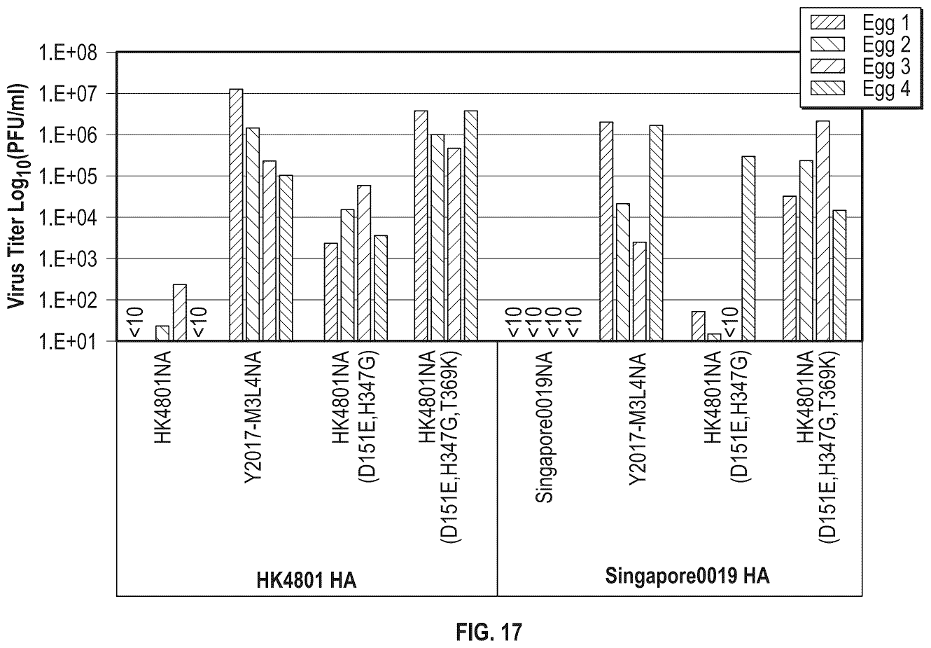

HK4801NA (T148K, D151E, H347G, and T369K) conferred efficient replication in the allantoic cavity to HY-PR8 backbone viruses possessing either HK4801HA or Singapore0019HA. Virus inoculation: 2×10³ pfu/egg into allantoic fluid, 72 h incubation at 37° C. (FIG. 16).

The HA coding nucleic acid sequence and NA coding nucleic acid and amino acid sequences for Singapore0019 are as follows:

A/Singapore/INFINH-16-0019/2016(H3N2) HA
(SEQ ID NO: 46)
atgaagactatcattgctttgagctacattctatgtctggttttcgctcaaaaaattcctggaaatgacaatagcacggcaacgctgtgccttgggcacca
tgcagtaccaaacggaacgatagtgaaaacaatcacaaatgaccgaattgaagttactaatgctactgagttggttcagaattcctcaataggtgaa
atatgcgacagtcctcatcagatccttgatggagagaactgcacactaatagatgctctattgggagaccctcagtgtgatggctttcaaaataagaa
atgggacctttttgttgaacgaagcaaagcctacagcaactgttacccttatgatgtgccggattatgcctcccttaggtcactagttgcctcatccggca
cactggagtttaaaaatgaaagcttcaattggactggagtcactcaaaacggaacaagttctgcttgcataaggggatctagtagtagtttctttagtag
attaaattggttgacccacttaaactacacatatccagcattgaacgtgactatgccaaacaaggaacaatttgacaaattgtacatttggggggttca
ccacccgggtacggacaaggaccaaatcttcctgtatgctcaatcatcaggaagaatcacagtatctaccaaaagaagccaacaagctgtaatcc
caaatatcggatctagacccagaataagggatatccctagcagaataagcatctattggacaatagtaaaaccgggagacatacttttgattaacag
cacagggaatctaattgctcctaggggttacttcaaaatacgaagtgggaaaagctcaataatgagatcagatgcacccattggcaaatgcaagtct
gaatgcatcactccaaatggaagcattcccaatgacaaaccattccaaaatgtaaacaggatcacatacggggcctgtcccagatatgttaagcat
agcactctgaaattggcaacaggaatgcgaaatgtaccagagaaacaaactagaggcatatttggcgcaatagcgggtttcatagaaaatggttg
ggagggaatggtggatggttggtacggtttcaggcatcaaaattctgagggaagaggacaagcagcagatctcaaaagcactcaagcagcaatc
gatcaaatcaatgggaagctgaataggttgatcggaaaaaccaacgagaaattccatcagattgaaaaagaattctcagaagtagaaggaagag
ttcaagaccttgagaaatatgttgaggacactaaaatagatctctggtcatacaacgcggagcttcttgttgccctggagaaccaacatacaattgatct
aactgactcagaaatgaacaaactgtttgaaaaaacaaagaagcaactgagggaaaatgctgaggatatgggaaatggttgtttcaaaatatacc
acaaatgtgacaatgcctgcatagaatcaataagaaatgaaacttatgaccacaatgtgtacagggatgaagcattgaacaaccggttccagatca
agggagttgagctgaagtcaggatacaaagattggatcctatggatttcctttgccatatcatgttttttgctttgtgttgctttgttggggttcatcatg
tgggcctgccaaaagggcaacattagatgcaacatttgcatttga
A/Singapore/INFINH-16-0019/2016(H3N2) NA
(SEQ ID NO: 47)
atgaatccaaatcaaaagataataacgattggctctgtttctctcaccatttccacaatatgcttcttcatgcaaattgccatcctgataactactgtaacat
tgcatttcaagcaatatgaattcaactcccccccaaacaaccaagtgatgctgtgtgaaccaacaataatagaaagaaacataacagagatagtgt
atttgaccaacaccaccatagagaaggaaatatgccccaaaccagcagaatacagaaattggtcaaaaccgcaatgtggcattacaggatttgca
cctttctctaaggacaattcgattaggctttccgctggtggggacatctgggtgacaagagaaccttatgtgtcatgcgatcctgacaagtgttatcaattt
gcccttggacagggaacaacactaaacaacgtgcattcaaataacacagtacgtgataggaccccttatcggactctattgatgaatgagttgggtgt
tcctttccatctggggaccaagcaagtgtgcatagcatggtccagctcaagttgtcacgatggaaaagcatggctgcatgtttgtataacgggggatg
ataaaaatgcaactgctagcttcatttacaatgggaggcttatagatagtgttgtttcatggtccaaagatattctcaggacccaggagtcagaatgcgtt
tgtatcaatggaacttgtacagtagtaatgactgatggaaatgctacaggaaaagctgatactaaaatactattcattgaggaggggaaaatcgttcat
actagcaaattgtcaggaagtgctcagcatgtcgaagagtgctcttgctatcctcgatatcctggtgtcagatgtgtctgcagagacaactggaaagg
atccaaccggcccatcgtagatataaacataaaggatcatagcattgtttccagttatgtgtgttcaggacttgttggagacacacccagaaaaaacg
acagctccagcagtagccattgtttgaatcctaacaatgaagaaggtggtcatggagtgaaaggctgggcctttgatgatggaaatgacgtgtggat
ggggagaacaatcaacgagacgtcacgcttagggtatgaaaccttcaaagtcgttgaaggctggtccaaccctaagtccaaattgcagataaata
ggcaagtcatagttgacagaggtgataggtccggttattctggtattttctctgttgaaggcaaaagctgcatcaatcggtgcttttatgtggagttgattag
gggaagaaaagaggaaactgaagtcttgtggacctcaaacagtattgttgtgttttgtggcacctcaggtacatatggaacaggctcatggcctgatg
gggcggacctcaatctcatgcatatataa
which encodes
(SEQ ID NO: 48)
M N P N Q K I I T I G S V S L T I S T I C F F M Q I A I L I T T V T L H F K Q Y E F N S P P N N Q V M L C
E P T I I E R N I T E I V Y L T N T T I E K E I C P K P A E Y R N W S K P Q C G I T G F A P F S K D N S I
R L S A G G D I W V T R E P Y V S C D P D K C Y Q F A L G Q G T T L N N V H S N N T V R D R T P Y
R T L L M N E L G V P F H L G T K Q V C I A W S S S S C H D G K A W L H V C I T G D D K N A T A S F
I Y N G R L I D S V V S W S K D I L R T Q E S E C V C I N G T C T V V M T D G N A T G K A D T K I L F I
E E G K I V H T S K L S G S A Q H V E E C S C Y P R Y P G V R C V C R D N W K G S N R P I V D I N I
K D H S I V S S Y V C S G L V G D T P R K N D S S S S S H C L N P N N E E G G H G V K G W A F D D
G N D V W M G R T I N E T S R L G Y E T F K V V E G W S N P K S K L Q I N R Q V I V D R G D R S G
Y S G I F S V E G K S C I N R C F Y V E L I R G R K E E T E V L W T S N S I V V F C G T S G T Y G T G
S W P D G A D L N L M H I.

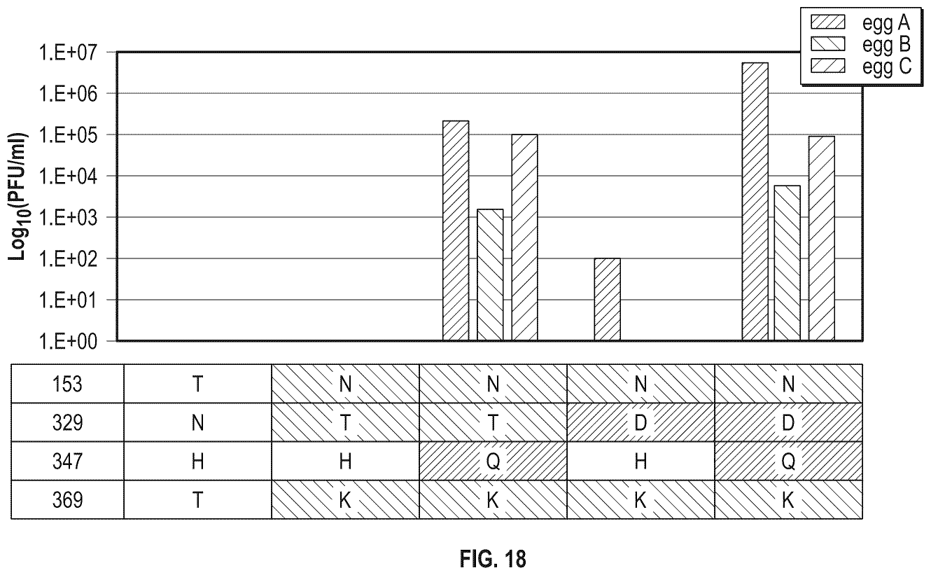

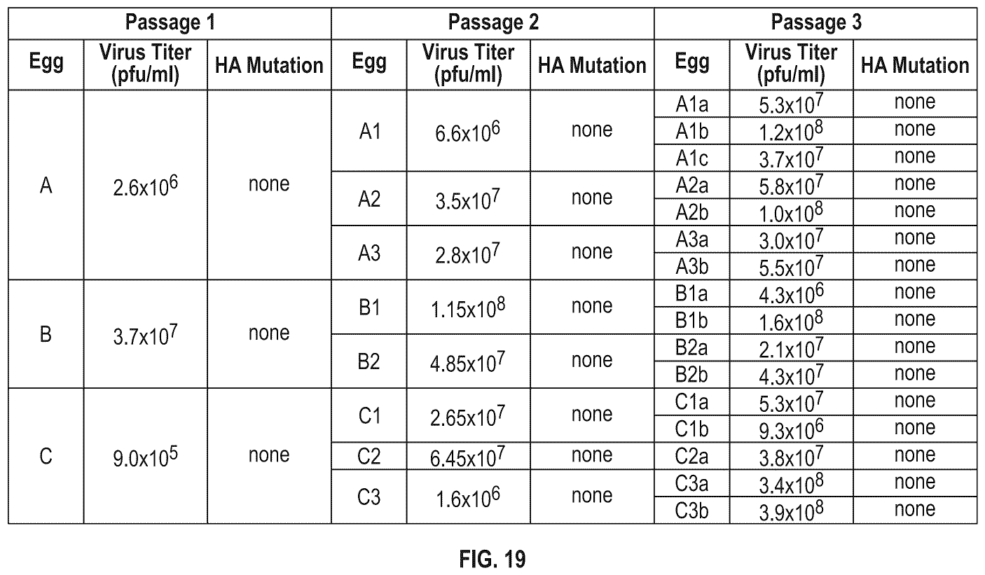

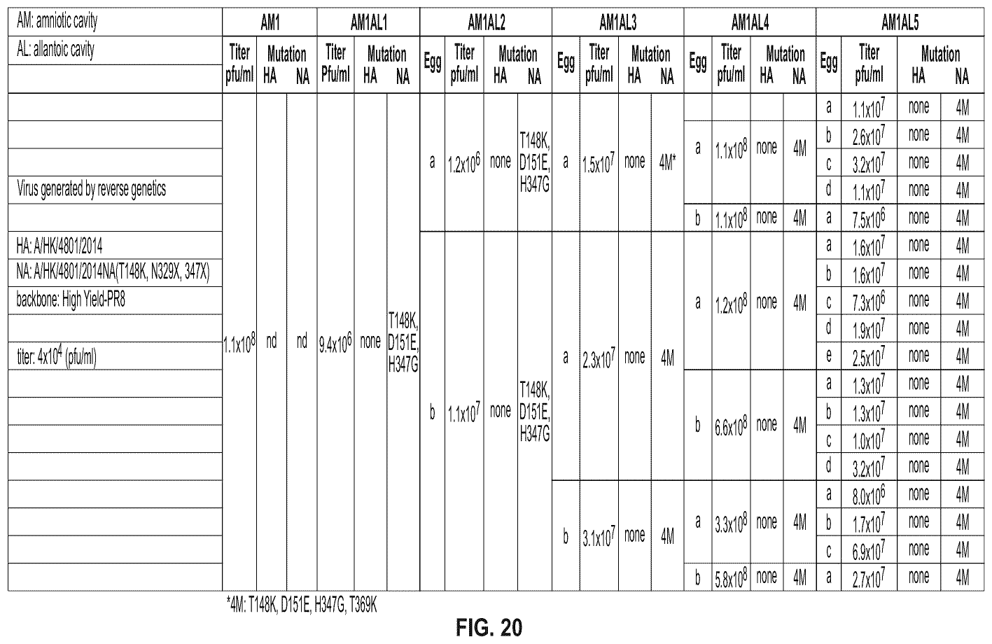

NA mutations T153N, N329T, and T369K allowed A/Saitama/102/2014 (H3N2) to replicate efficiently in the allantoic cavity (Kuwahara et al., 2018). Therefore, the effect of introducing NA-T153N, N329T (or D), T369K, and H347Q into HK4801NA (T148K) was examined. FIG. 18 reports on virus titers for different combinations of NA residues identified in screenings. FIGS. 19 and 20 report on virus titers for viruses with different combinations of selected NA residues.

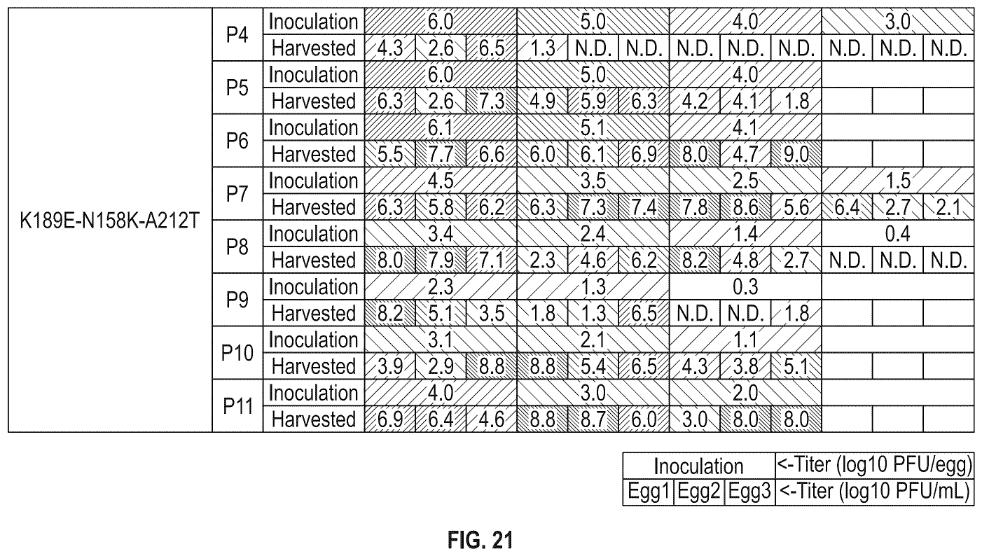

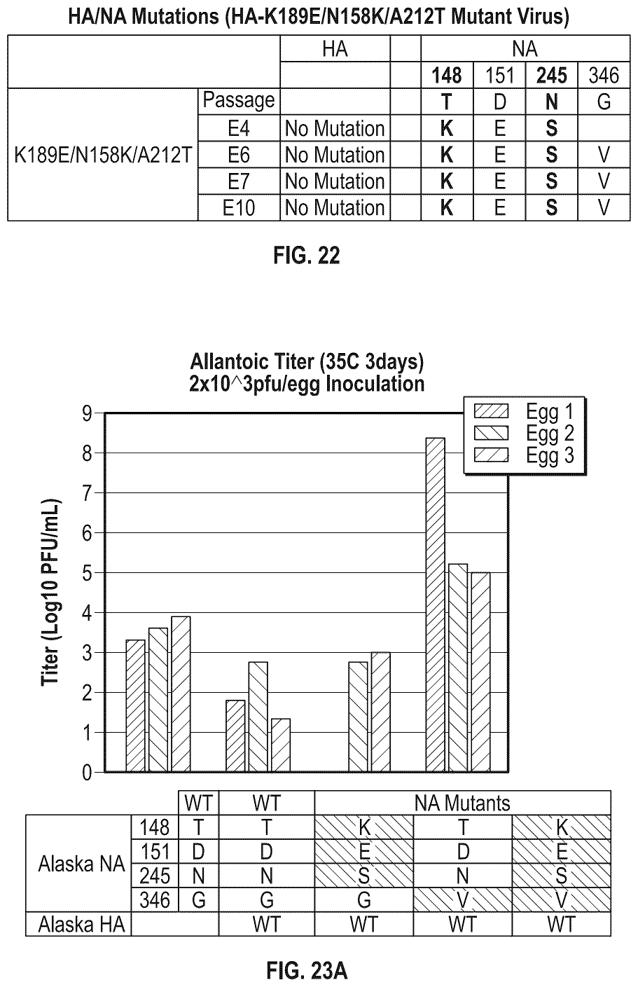

Alaska/232/2015_HY-PR8 (H3N2) WT/mutant virus were passaged in eggs and HA and NA segments sequenced. Alaska WT (a more recent H3N2 virus where WT has 245N, prior to 2015 H3N2 WT viruses had 245S), HA-R142S, -K189E viruses did not get mutations in HA, even after 3 amniotic and 10 allantoic passages. HA-K189E/N158K/A212T mutant did not get mutations in HA, but had some mutations in NA which exhibited improved growth in eggs since p6 (FIG. 21). The difference of NA mutations between p4 (normal growth) (NA-N245S mutation, virus grows more than 1000 fold better than with NA-245N) and p6 (better growth) was G346V (FIG. 22). Therefore, G346V may also contribute to adaptation to eggs.

The NA for A/Alaska/232/2015 has the following sequence:

(SEQ ID NO: 49)
mnpnqkiiti gsvsltisti cffmqiaili ttvtlhfkqy
efnsppnnqv mlceptiier niteivyltn ttiekeicpk
paeyrnwskp qcgitgfapf skdnsirlsa ggdiwvtrep
yvscdpdkcy qfalgqgttl nnvhsnntvr drtpyrtllm
nelgvpfhlg tkqvciawss sschdgkawl hvcitgddkn
atasfiyngr lvdsvvswsk dilrtqesec vcingtctvv
mtdgnatgka dtkilfieeg kivhtsklsg saqhveecsc
yprypgvrcv crdnwkgsnr pivdinikdh sivssyvcsg
lvgdtprknd ssssshclnp nneegghgvk gwafddgndv
wmgrtinets rlgyetfkvv egwsnpkskl qinrqvivdr
gdrsgysgif svegkscinr cfyvelirgr keetevlwts
nsivvfcgts gtygtgswpd gadlnlmhi.

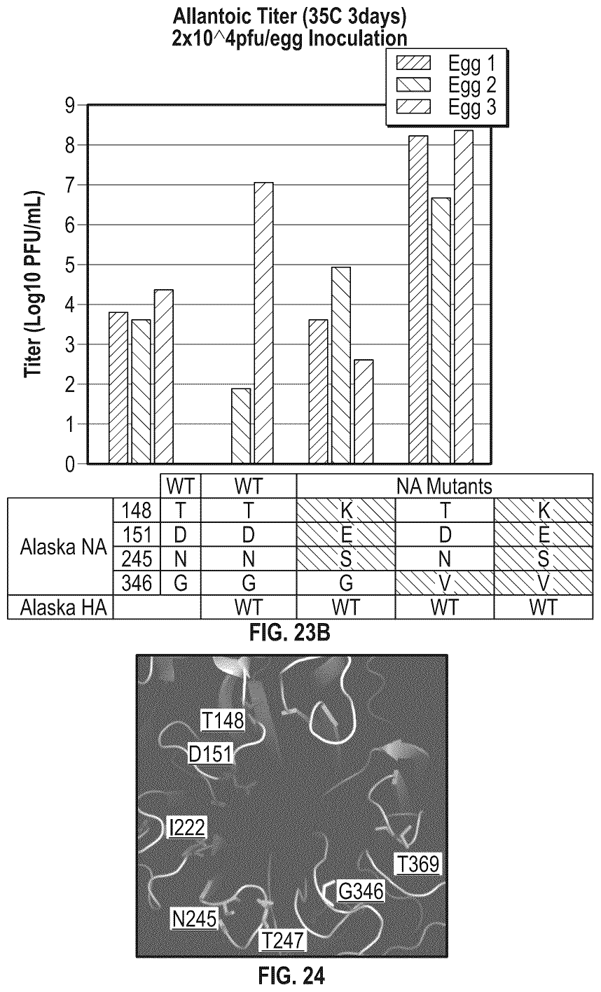

NA pHH21 plasmids were constructed: Alaska NA-T148K/D151E/N245S (found in E4); Alaska NA-G346V; and Alaska NA-T148K/D151E/N245S/G346V (found in E6). Mutant NAs were combined with WT Alaska HA or HY-PR8 backbone. Eggs were inoculated with the same dosage of WT/mutant Alaska viruses and harvested viruses titrated (FIG. 23). NA-T148K/D151E/N245S/G346V mutant virus grew to a higher titer than WT virus but the single mutation G346V did not increase virus growth compared to WT. These results suggested that a combination of G346V and one (or two to three) other mutations, e.g., 3 mutations such as T148K, D151E and N245S, may be important for virus Alaska virus to grow efficiently in eggs. Harvested virus samples with high titer (>5 Log10 PFU/mL) were sequenced however none had additional mutations in HA and NA.

The invention will be described by the following non-limiting examples.

### EXAMPLE I

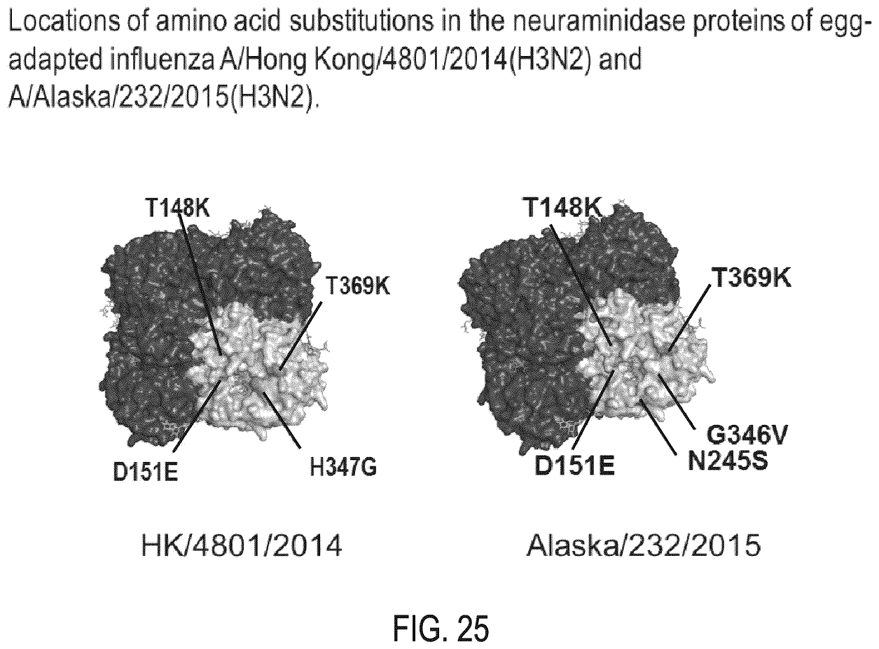

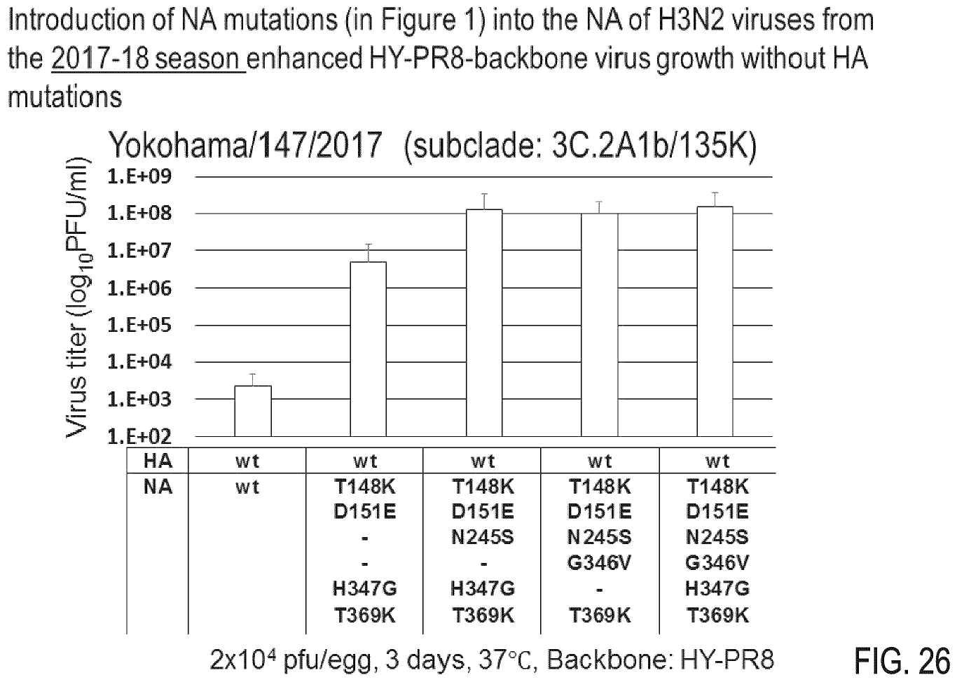

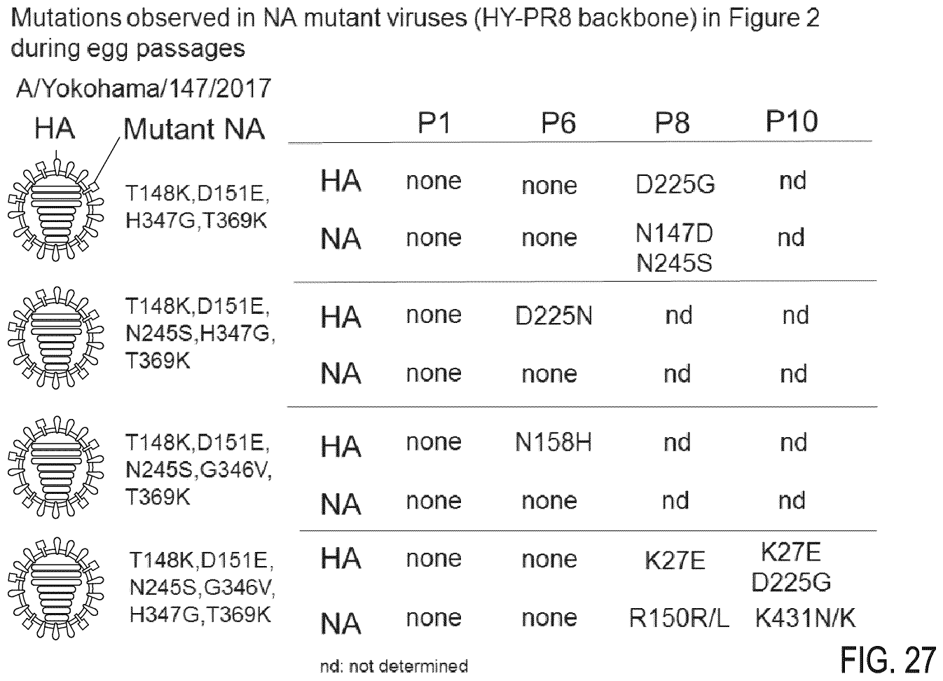

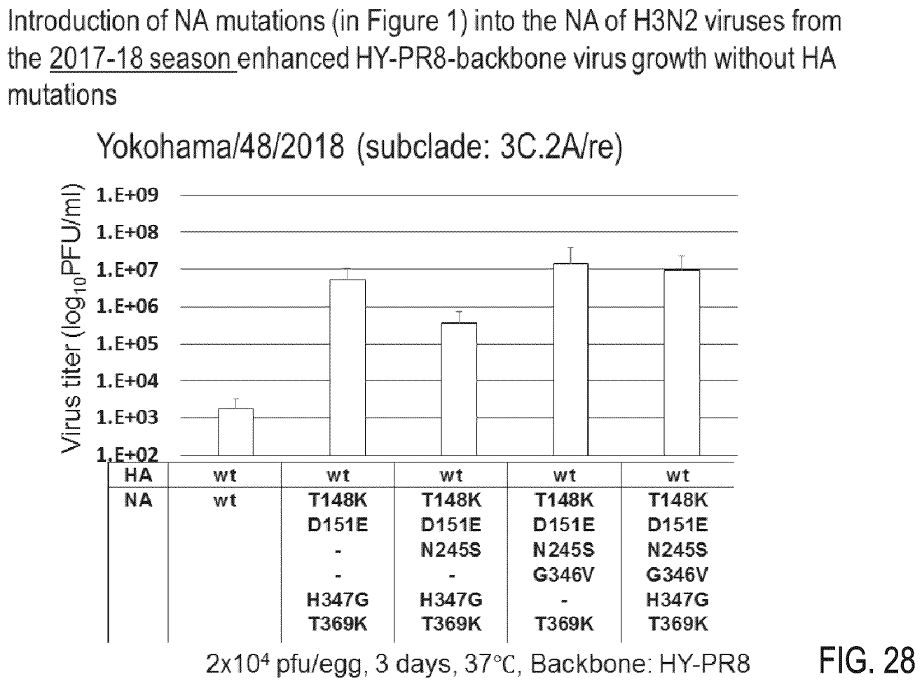

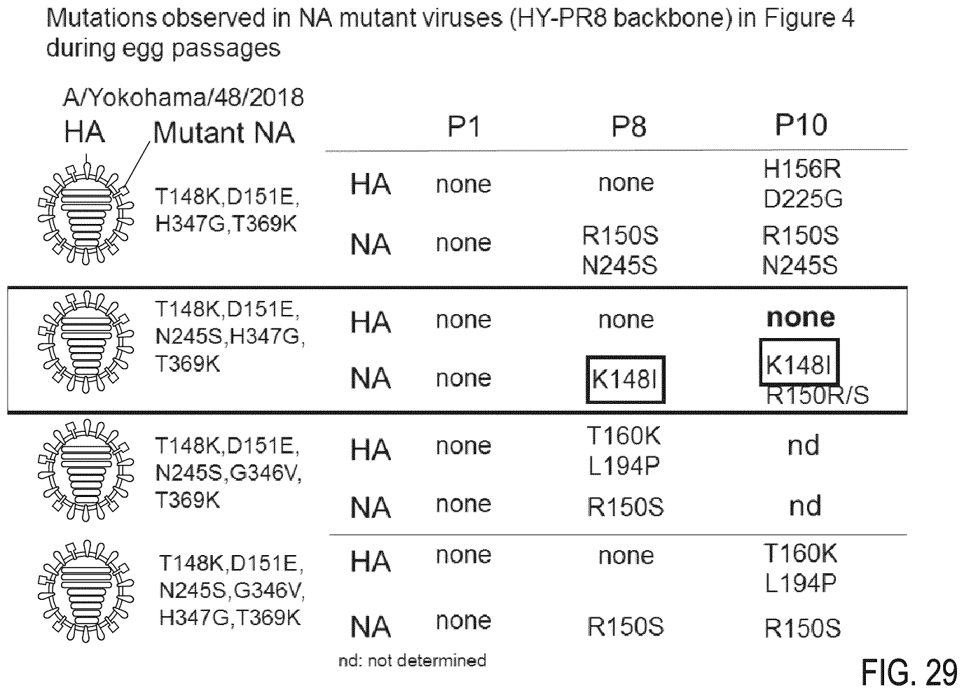

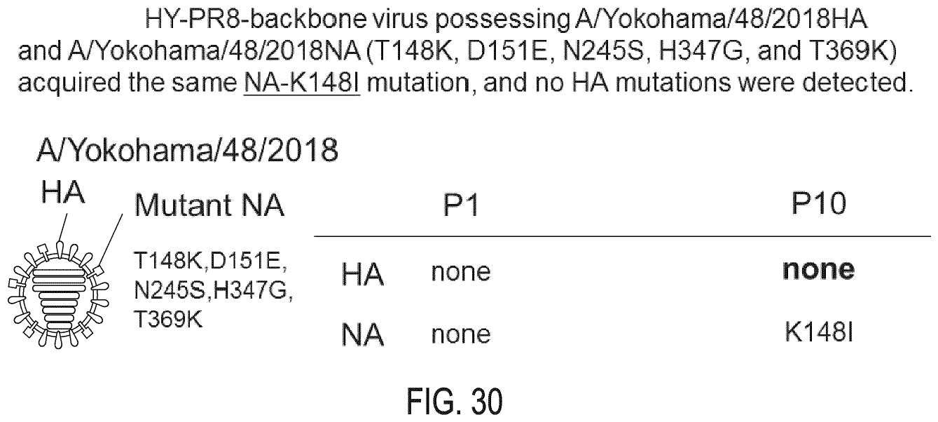

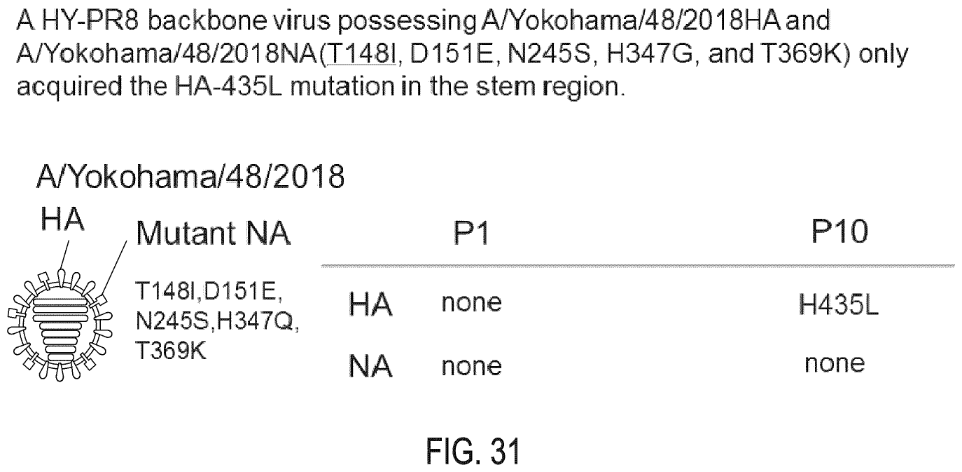

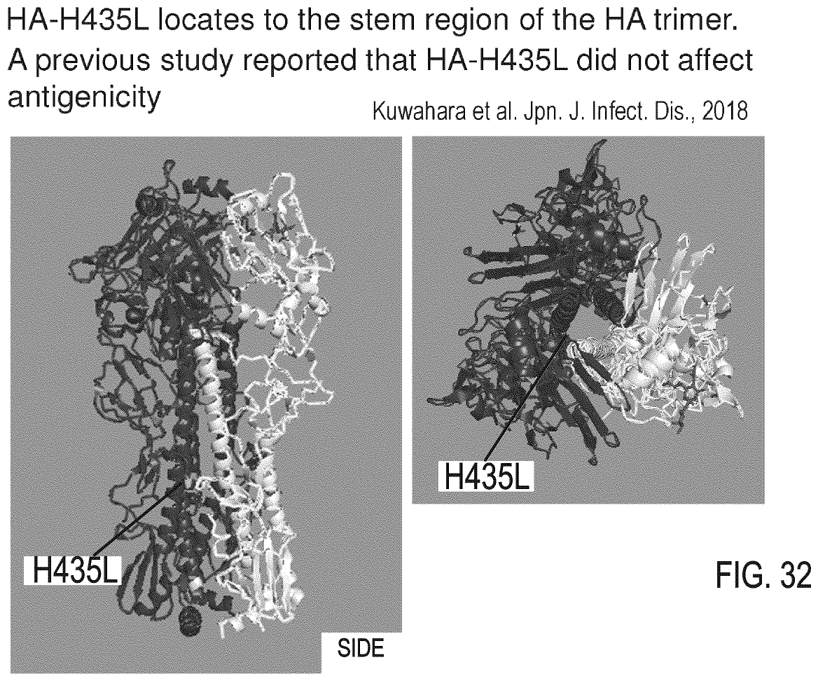

As shown in FIGS. 25-28, certain substitutions in N2 stabilized HA (e.g., did not allow for substitutions in HA) for up to about 8 passages in eggs in various H3N2 isolates from different influenza seasons. However, HA substitutions were found in some but not all isolates passaged in eggs for 10 passages (FIG. 29). Unexpectedly another change in NA (148I) was correlated with stabilizing HA even after more than 8 passages. The presence of that additional change in some cases resulted in a HA change but that change, in the stem region of HA (FIG. 32), is unlikely to alter antigenicity.

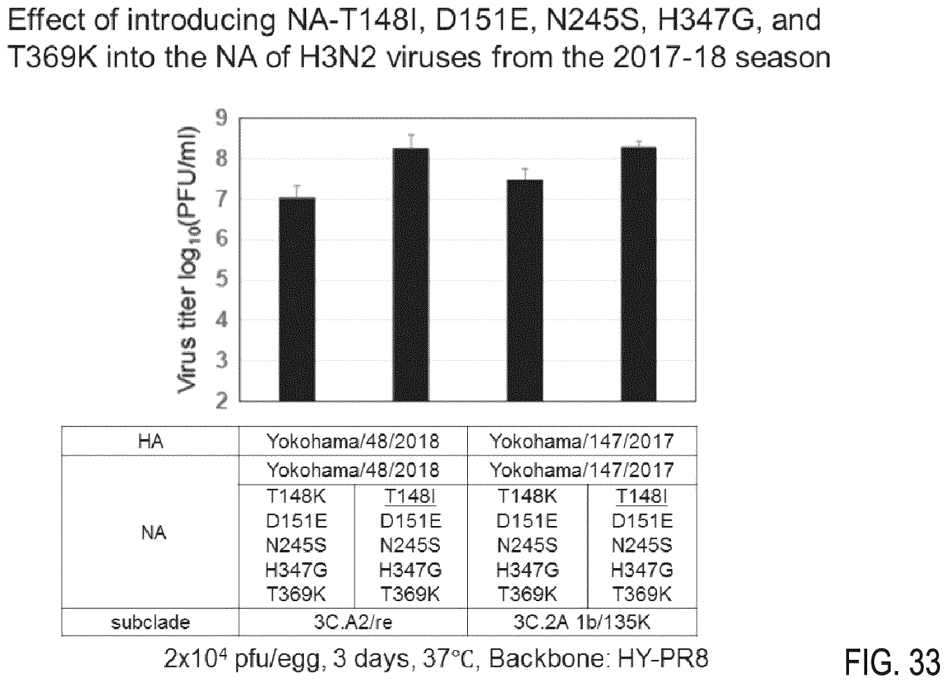

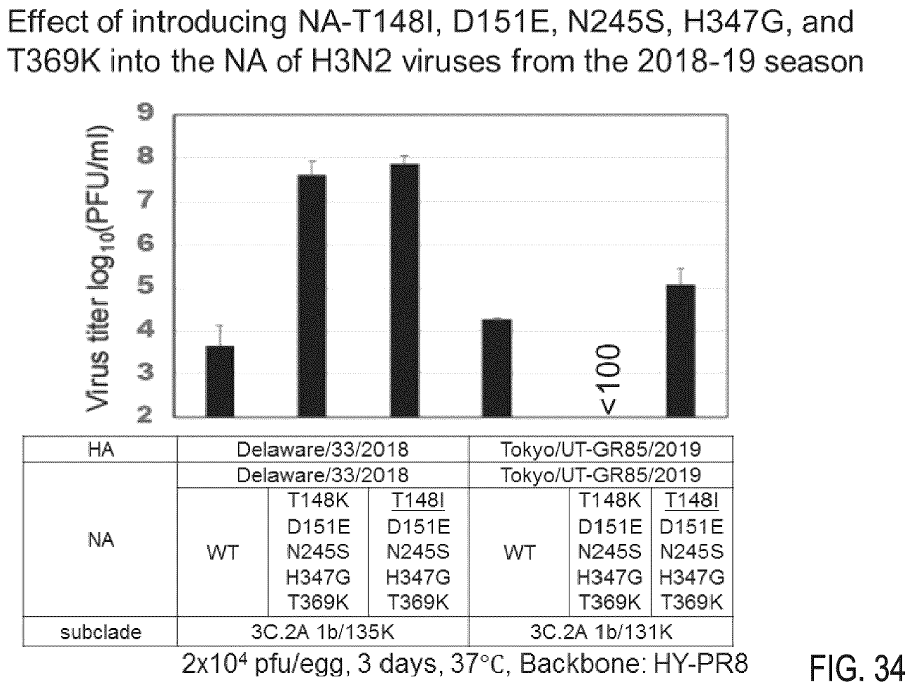

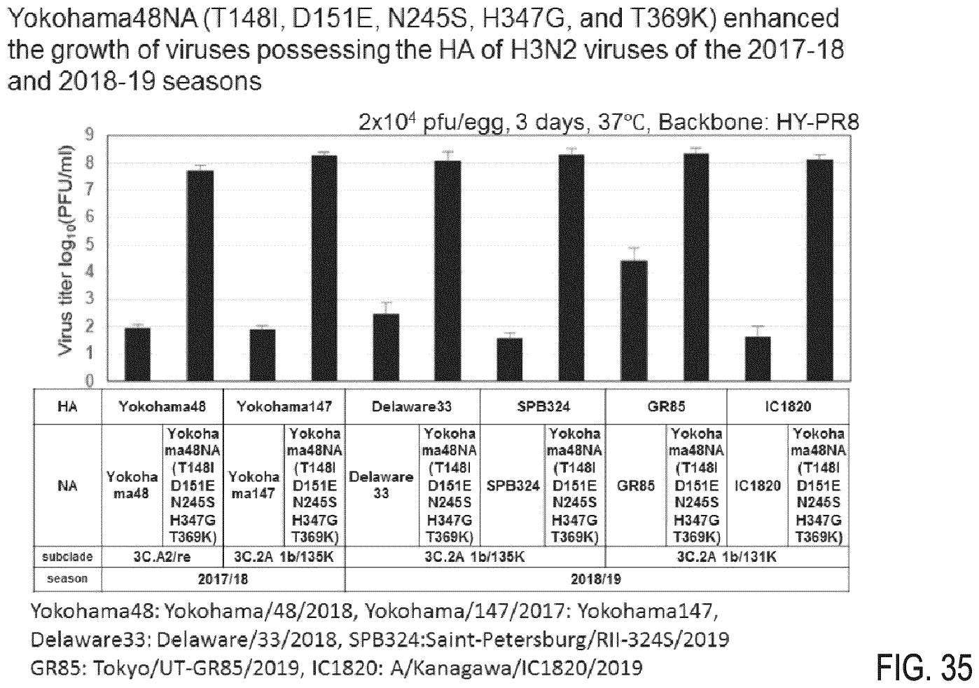

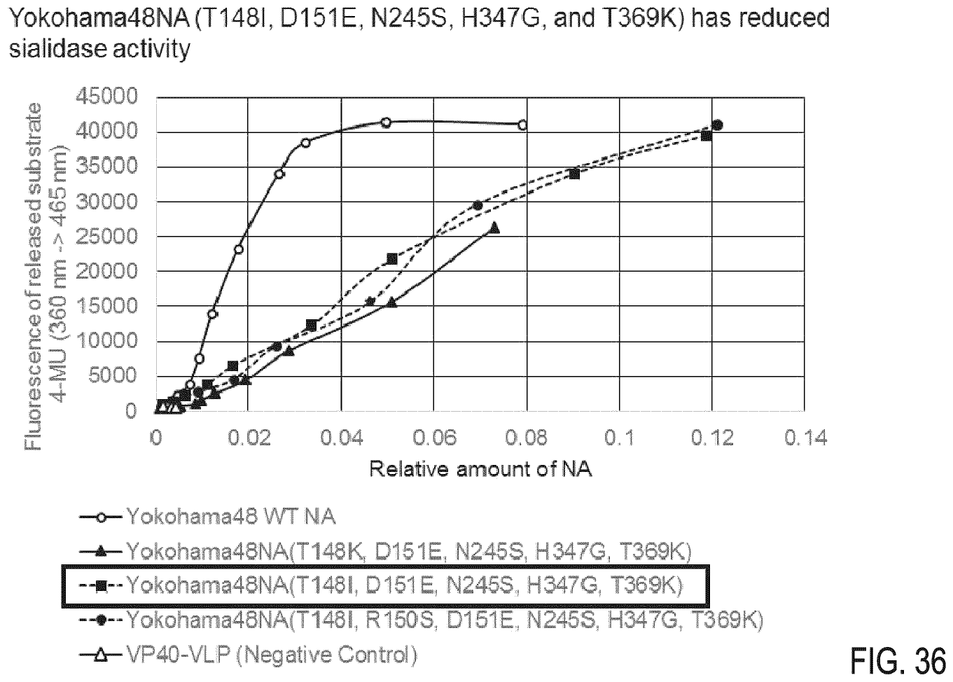

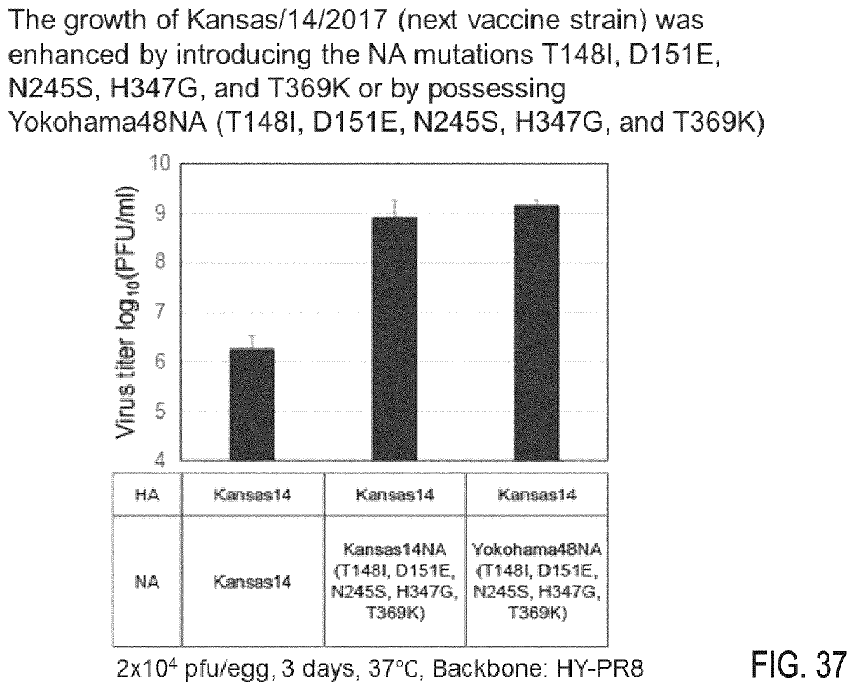

148I or 148K was introduced into the NA along with other substitutions that were identified (FIGS. 33-35). The addition of 148I did not substantially alter virus titer in eggs while 148K in some cases impacted titer. Interestingly, viruses having a NA with 148I in combination with other changes had reduced sialidase activity (FIG. 36). For example, viruses having a NA with 148I, 151E, 245S, 347G, and 369K and 148I, 150S, 151E, 245S, 347G and 369K, as well as viruses having a NA with 148K, 151E, 245S, 347G and 369K, had reduced sialidase activity.

### EXAMPLE II

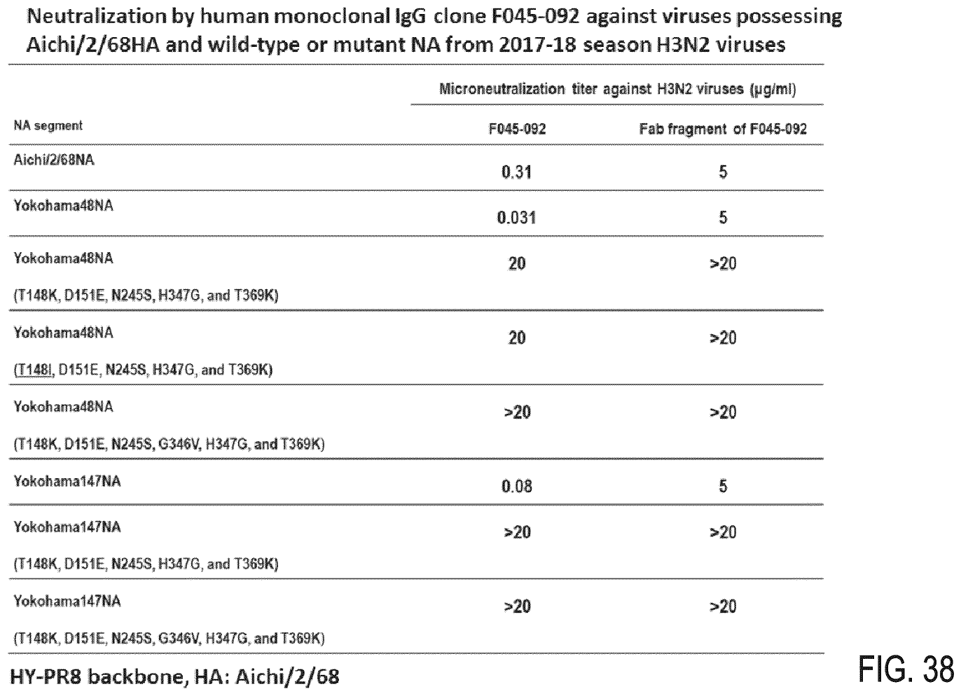

Mutations in the influenza surface glycoprotein neuraminidase (NA) confer efficient replication to recent human H3N2 viruses in eggs without the acquisition of mutations at the antigenic sites of the other surface glycoprotein, (hemagglutinin) HA. With NA mutations, the mutant NAs recognize sialic acid linked to galactose via alpha 2-3 linkages (Siaα2-3 Gal) prevalent on epithelial cells in the chorioallantoic membrane in chicken eggs. The NA mutations allow the viruses to attach and enter cells even under conditions where the interaction between HA and its receptor is inhibited (FIG. 38), suggesting that the mutant NA serves as a receptor-binding protein in place of HA.

By possessing an HA protein with disrupted or no receptor-binding activity, the disclosed mutant NA may confer to influenza viruses such as H3N2 viruses efficient growth in embryonated chicken eggs without the acquisition of any egg-adaptive HA mutations at antigenic sites. Because HA does not encounter selective pressure to recognize the Siaα2-3 Gal receptor in the presence of the mutant NA, the amino acid residues around the HA receptor-binding pocket remain unchanged during passages in embryonated chicken eggs.

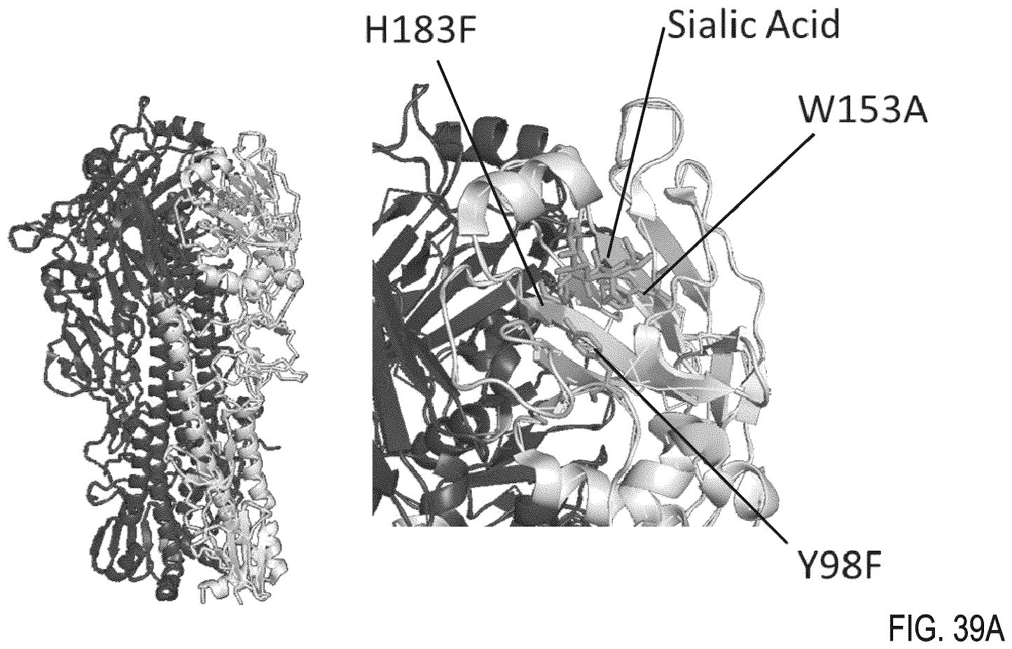

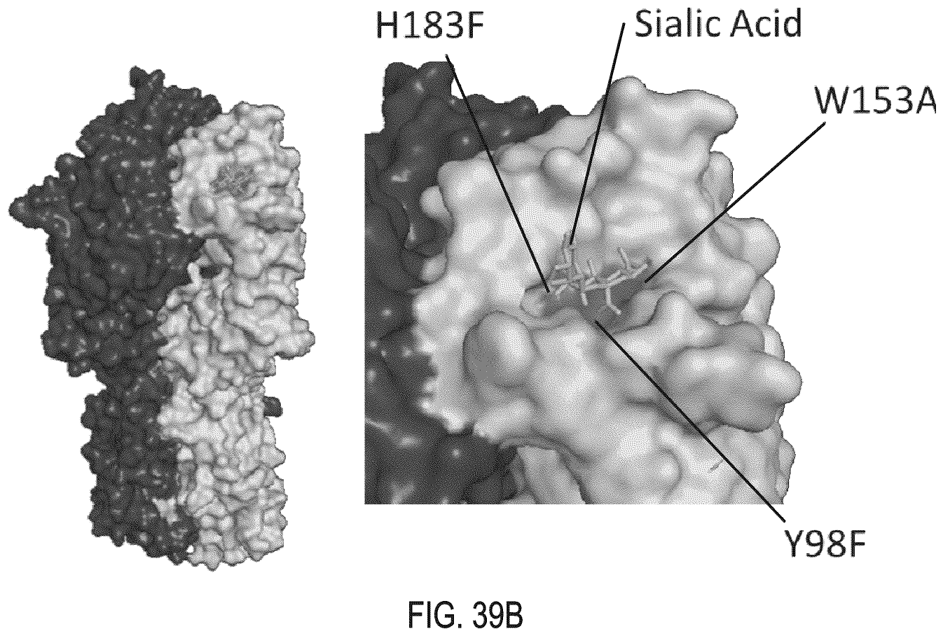

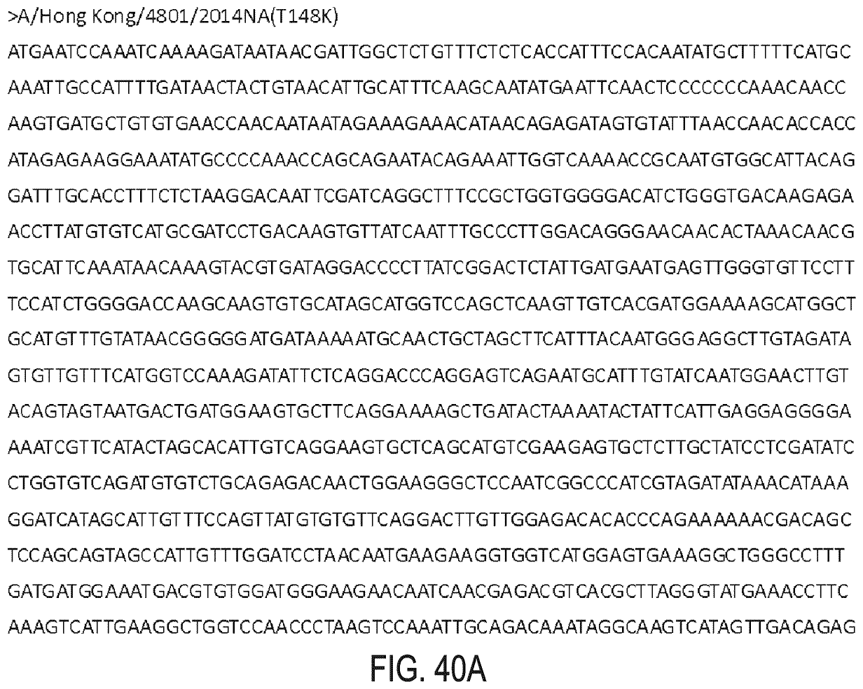

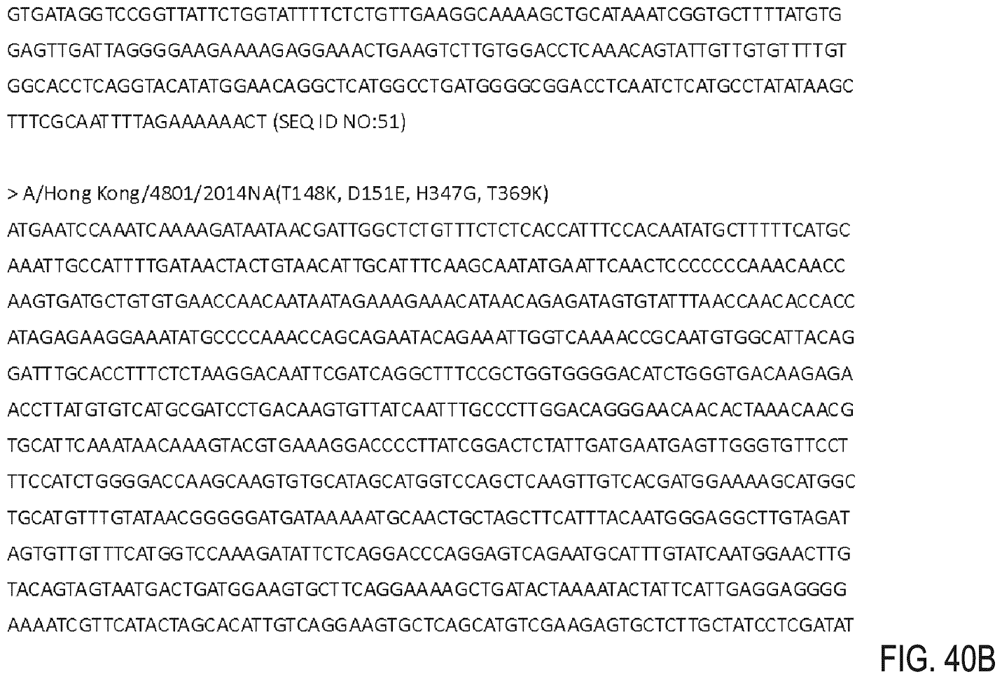

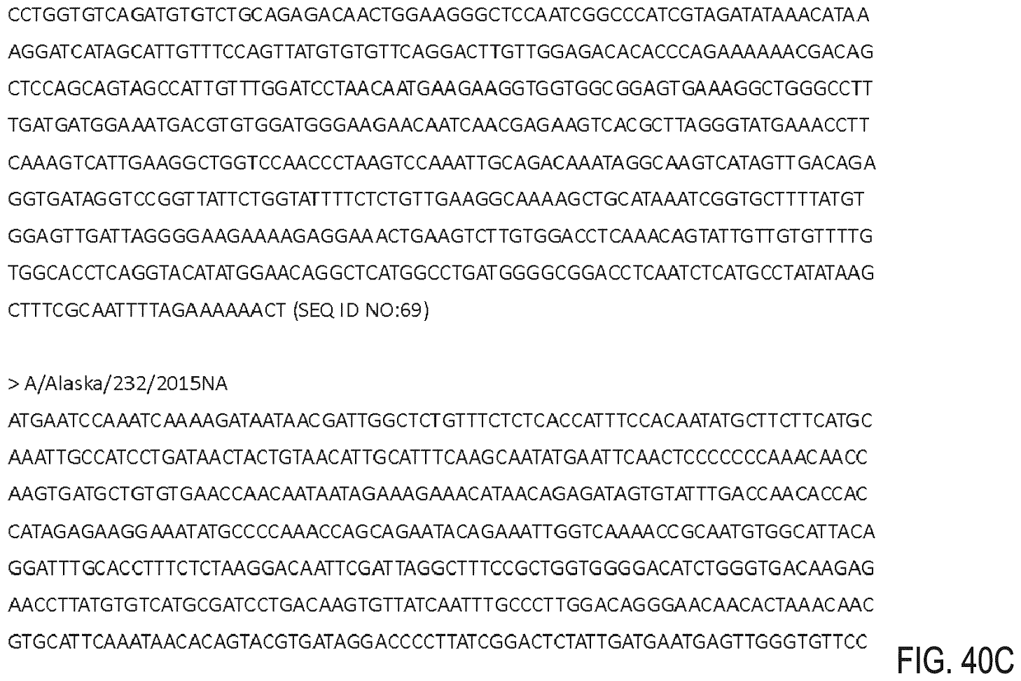

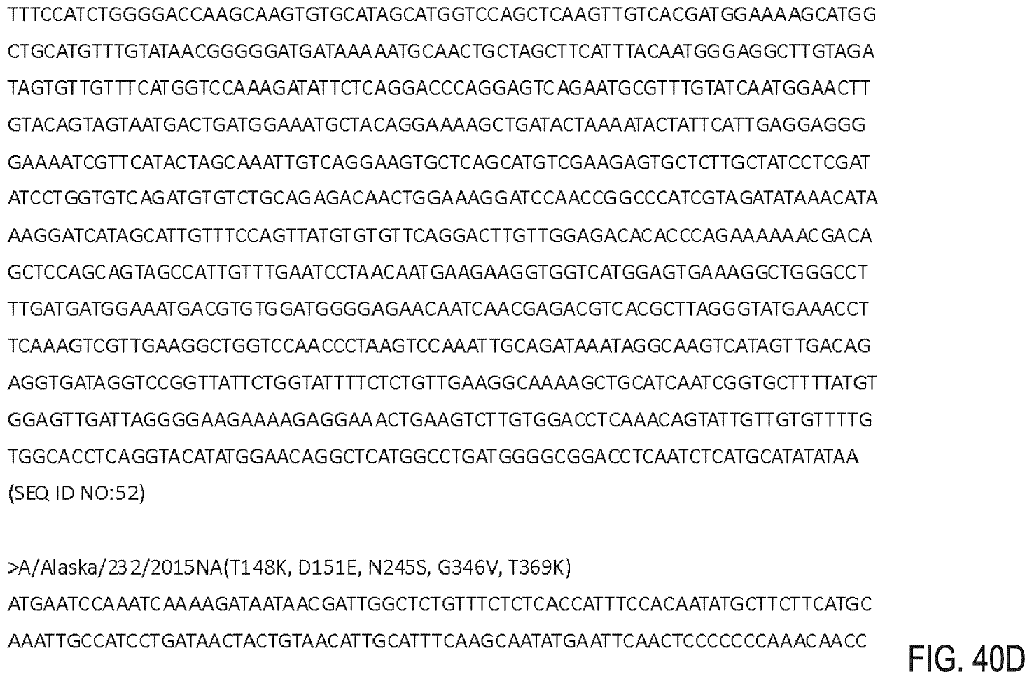

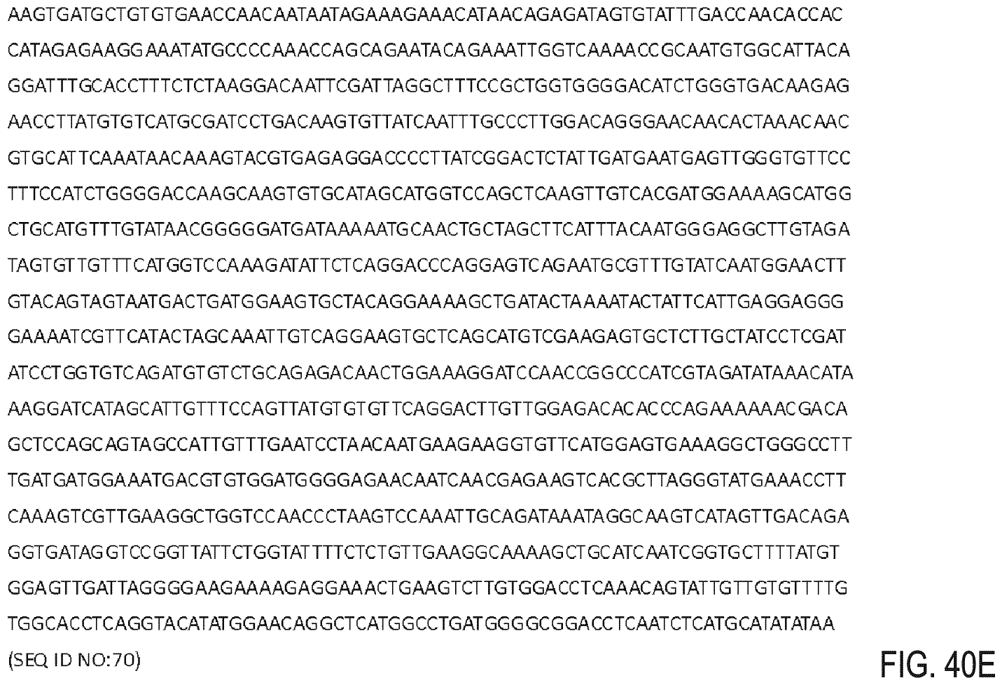

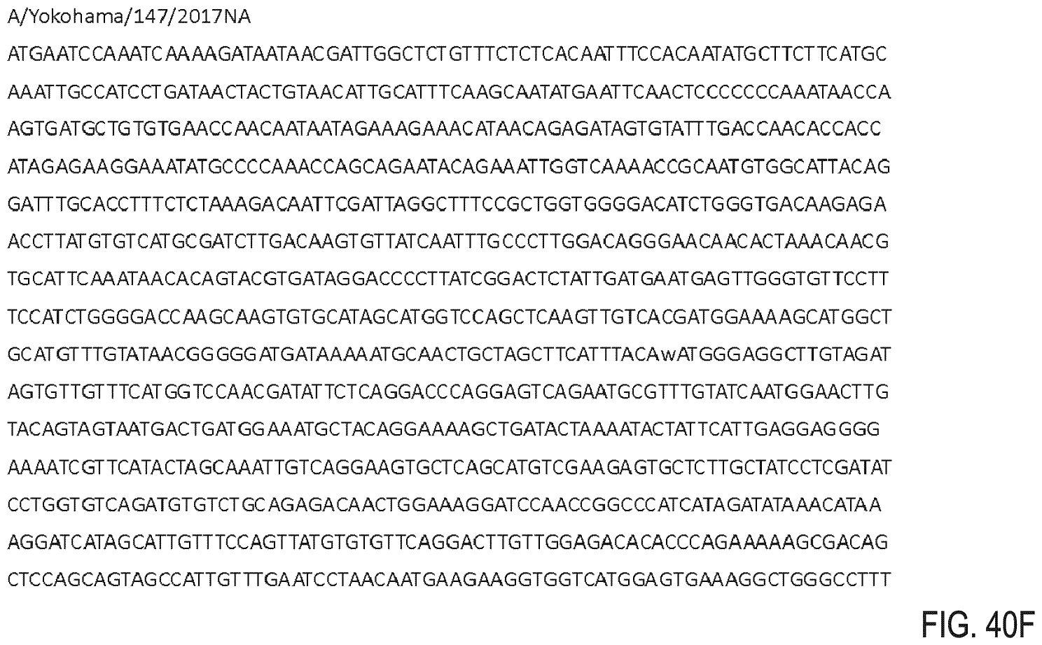

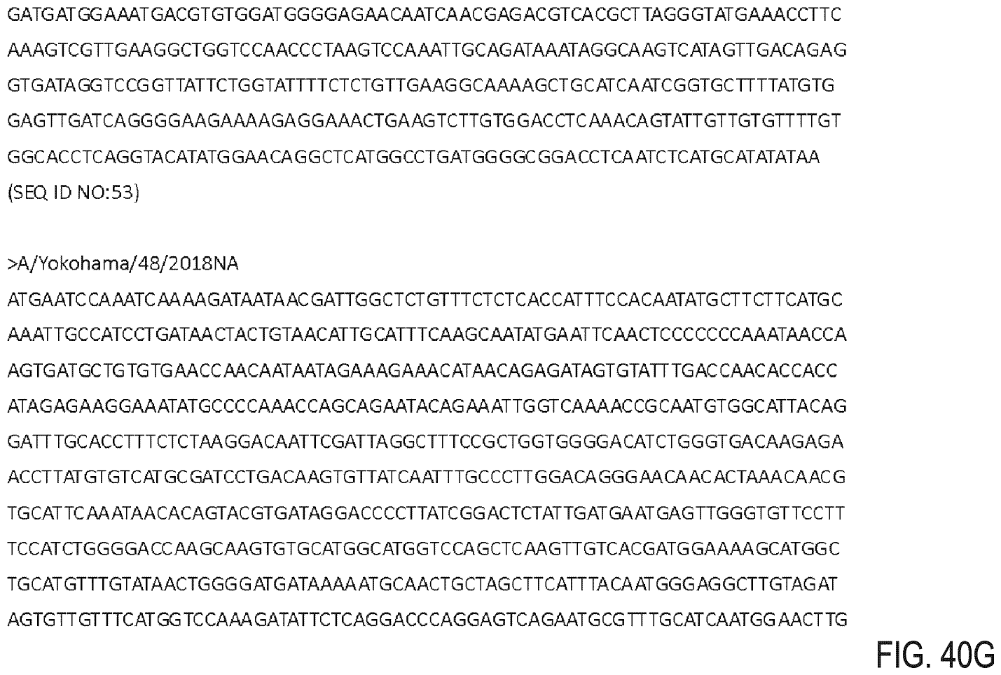

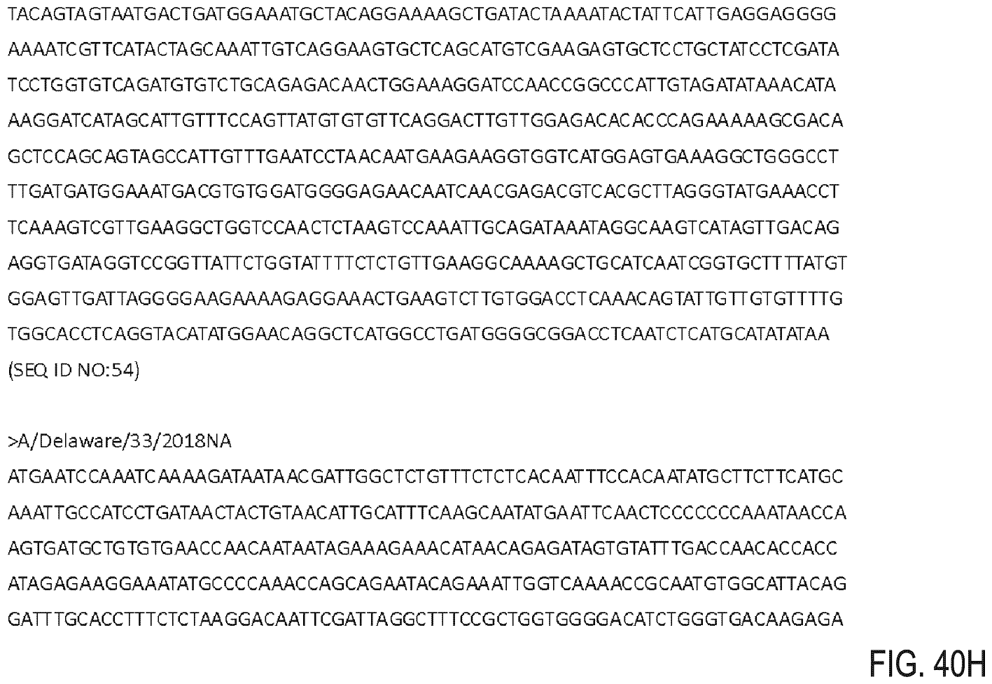

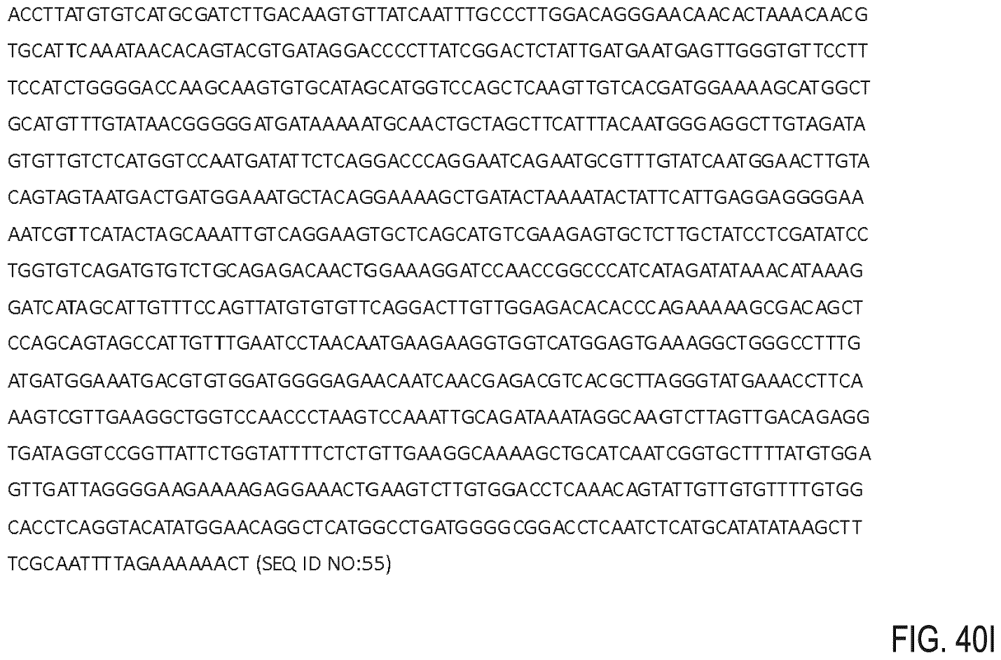

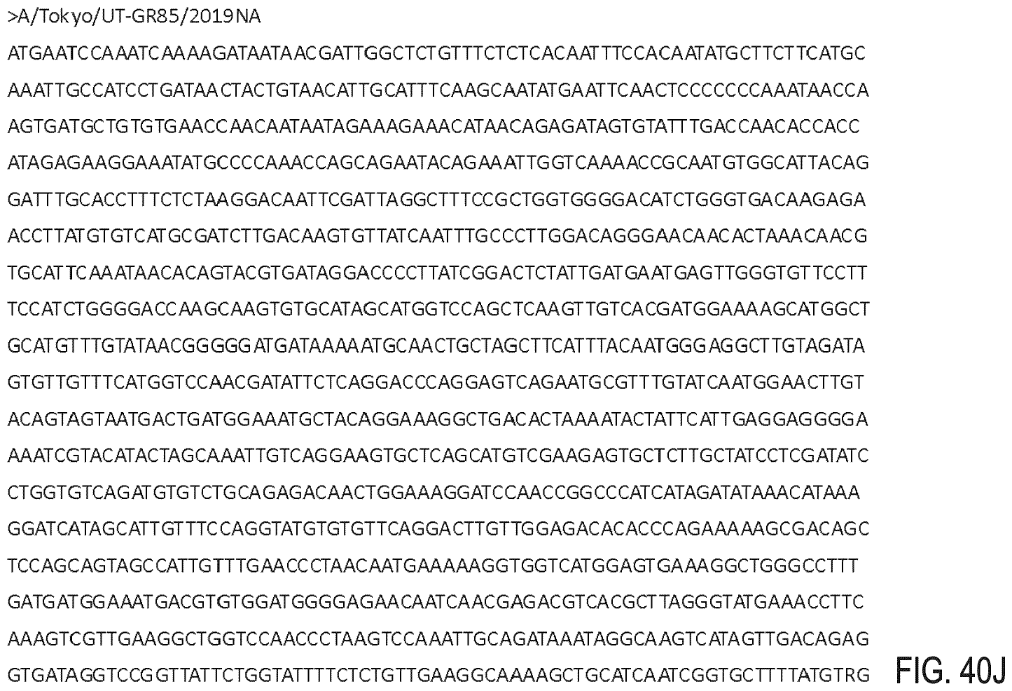

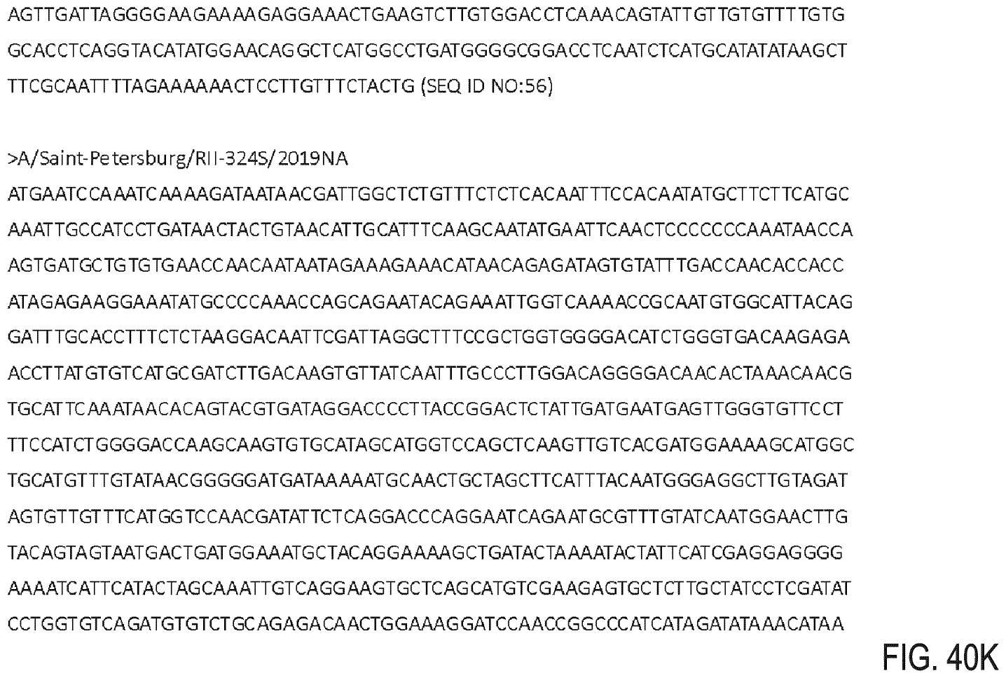

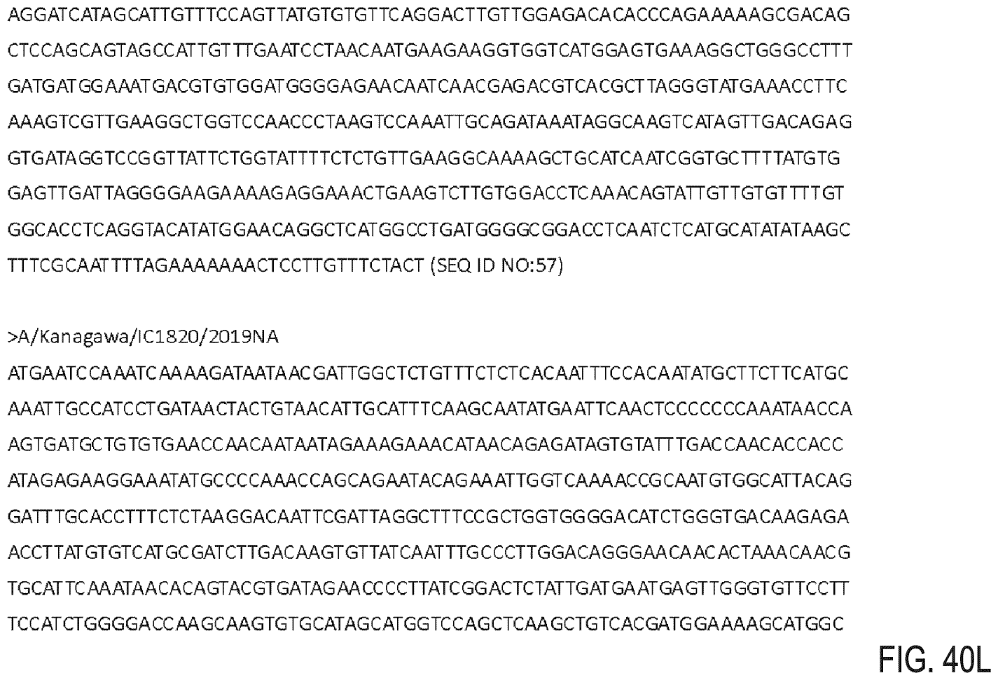

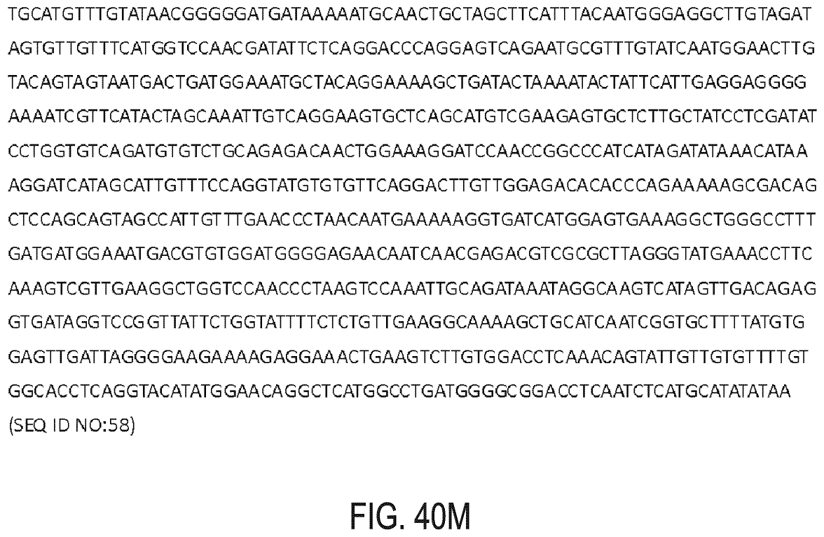

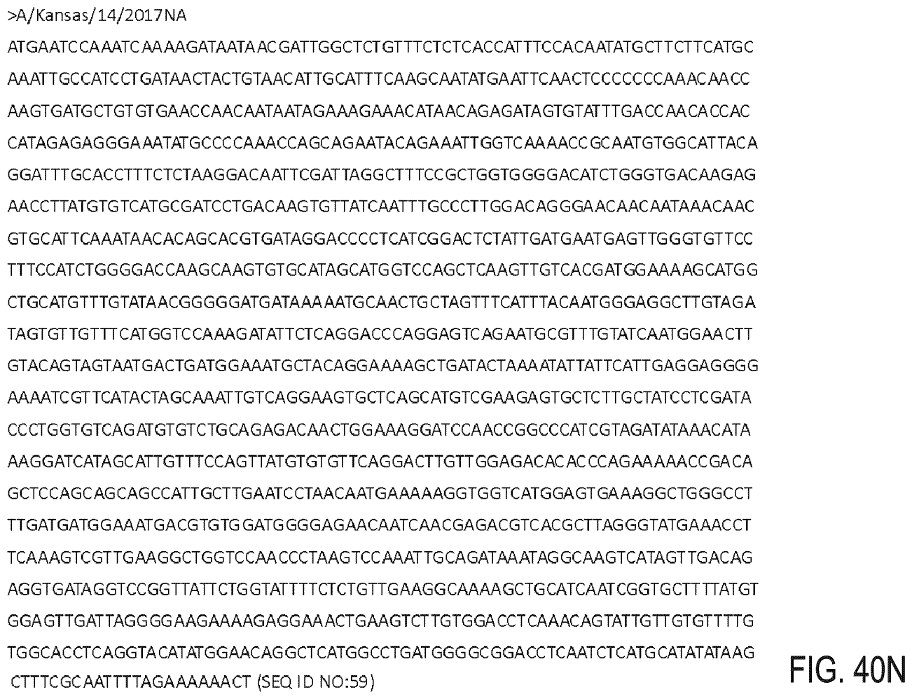

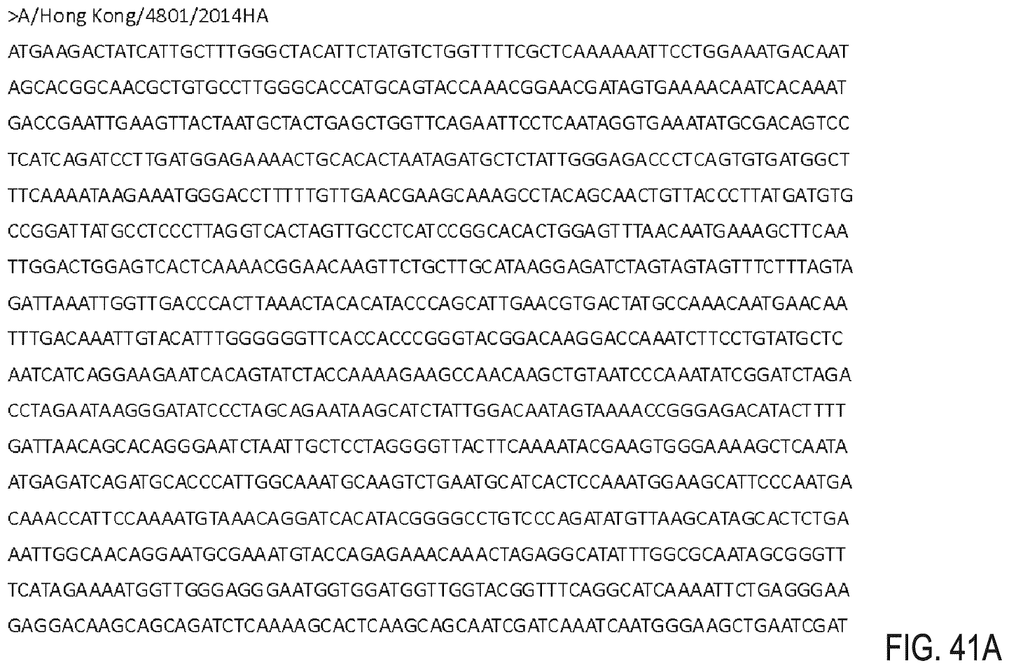

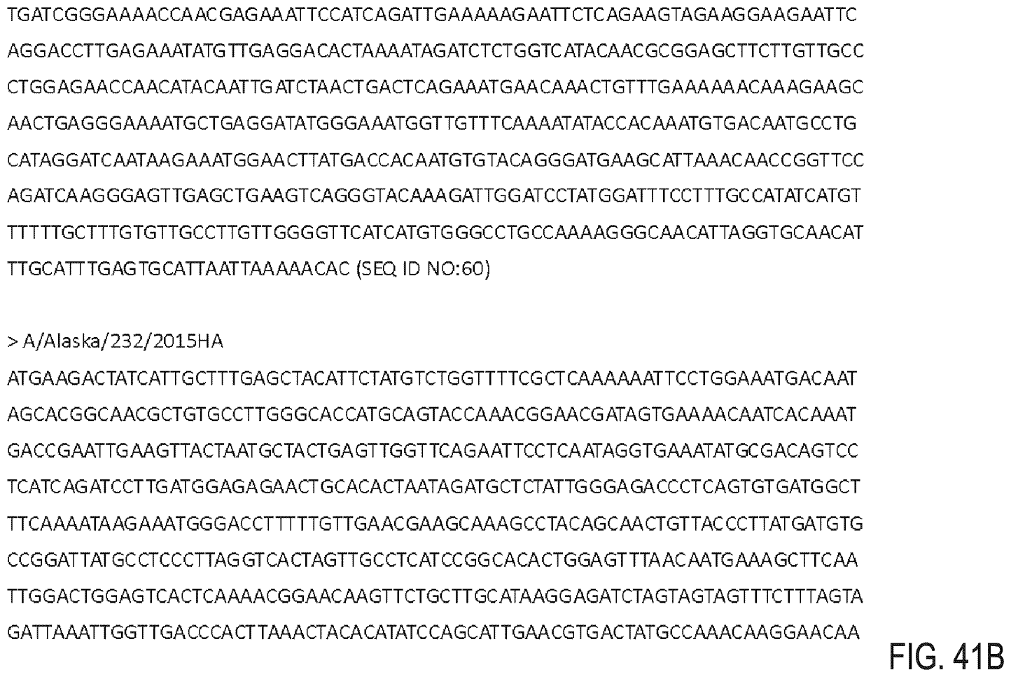

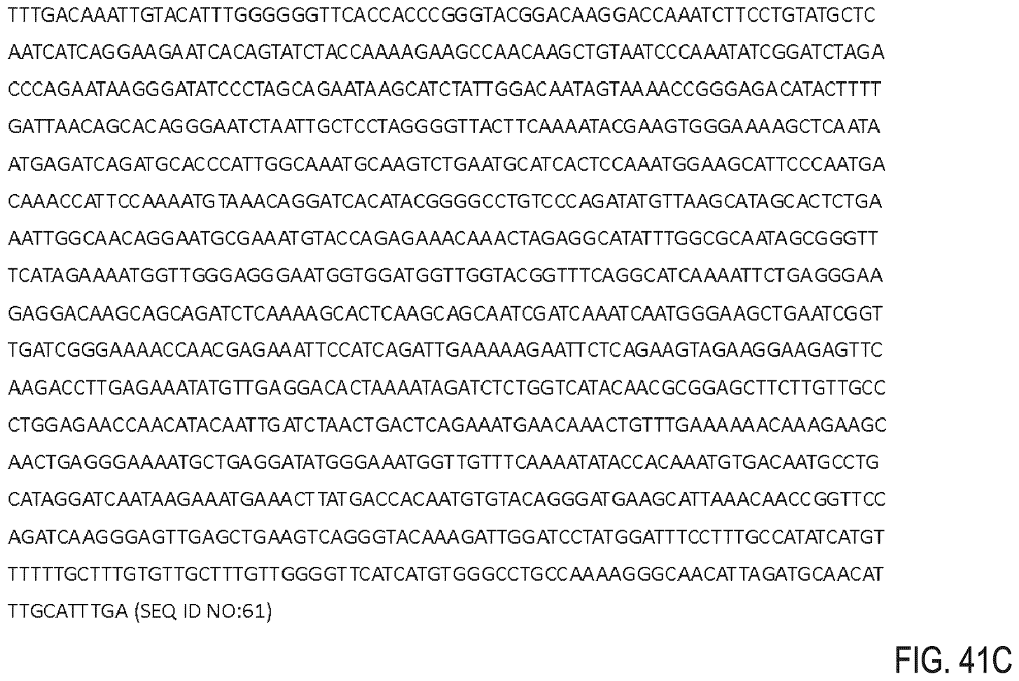

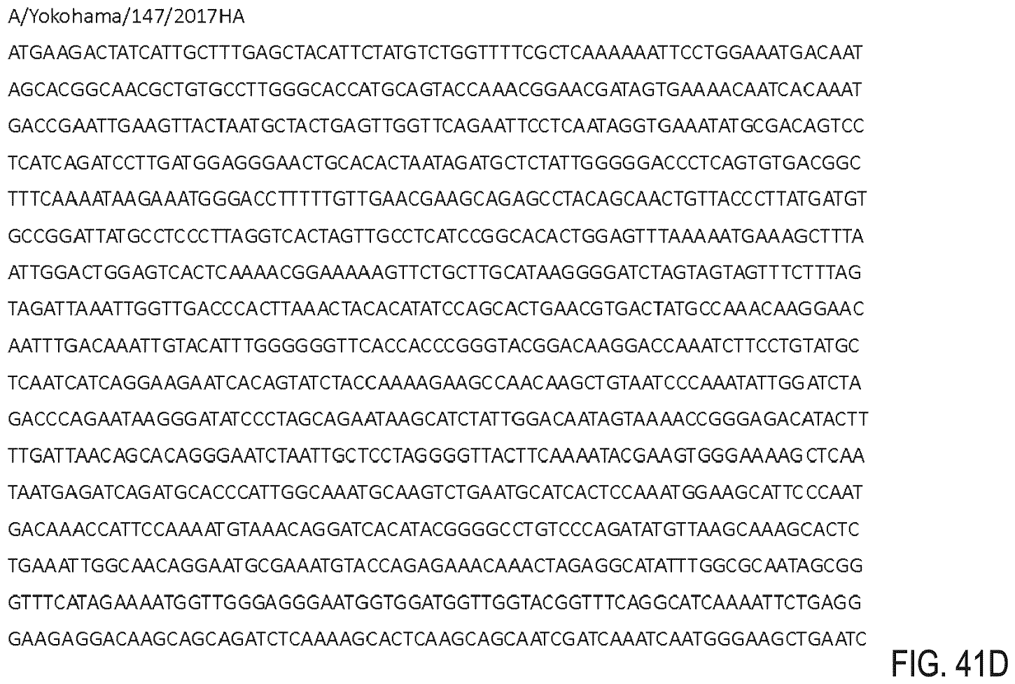

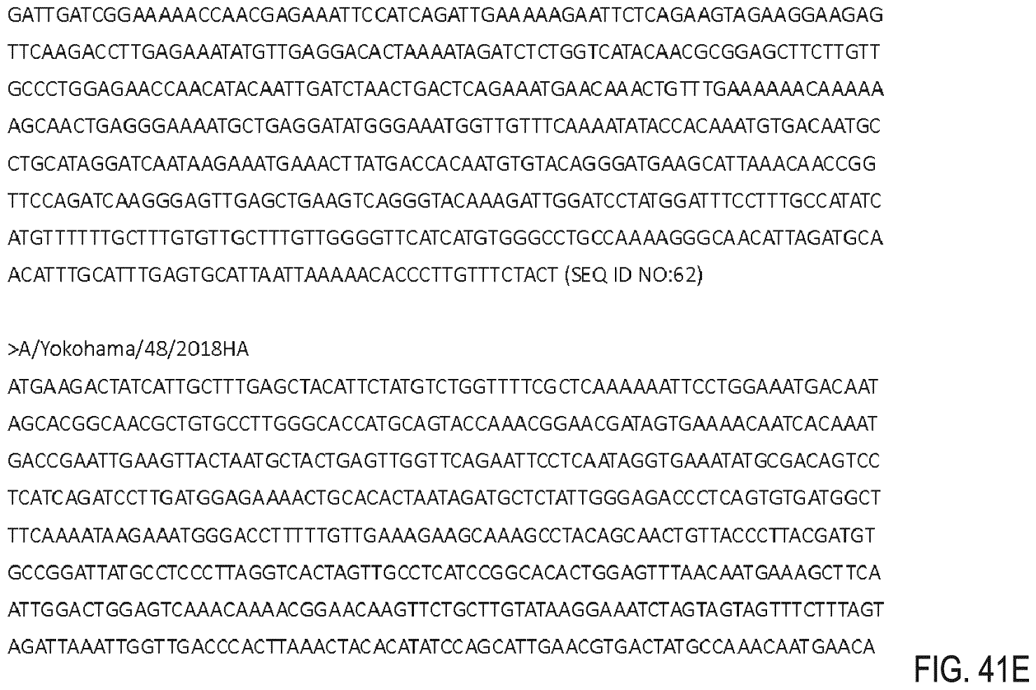

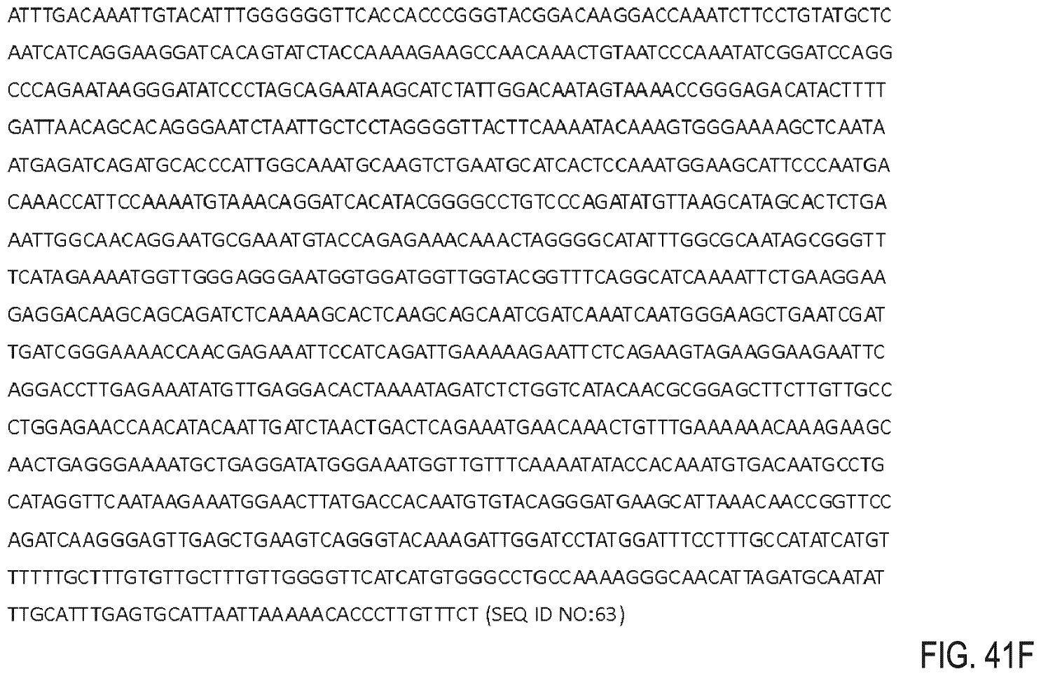

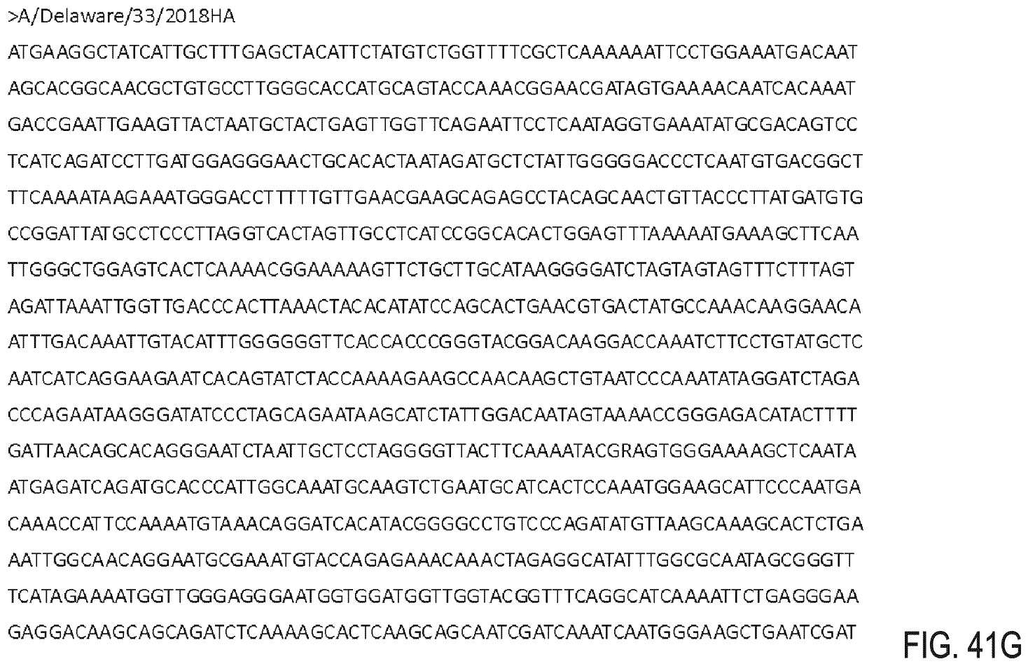

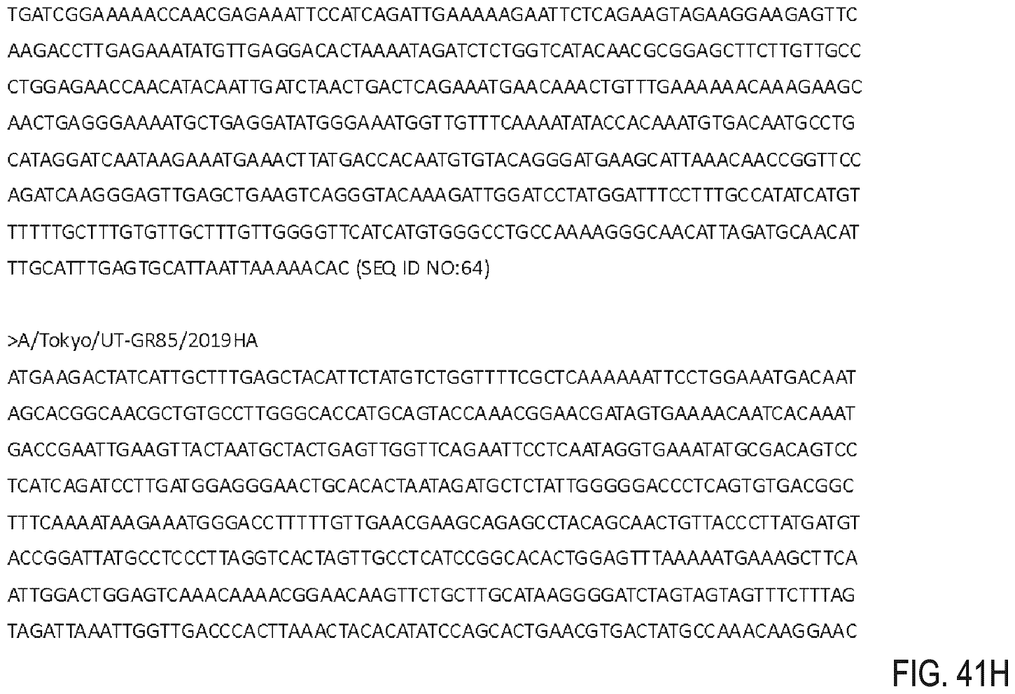

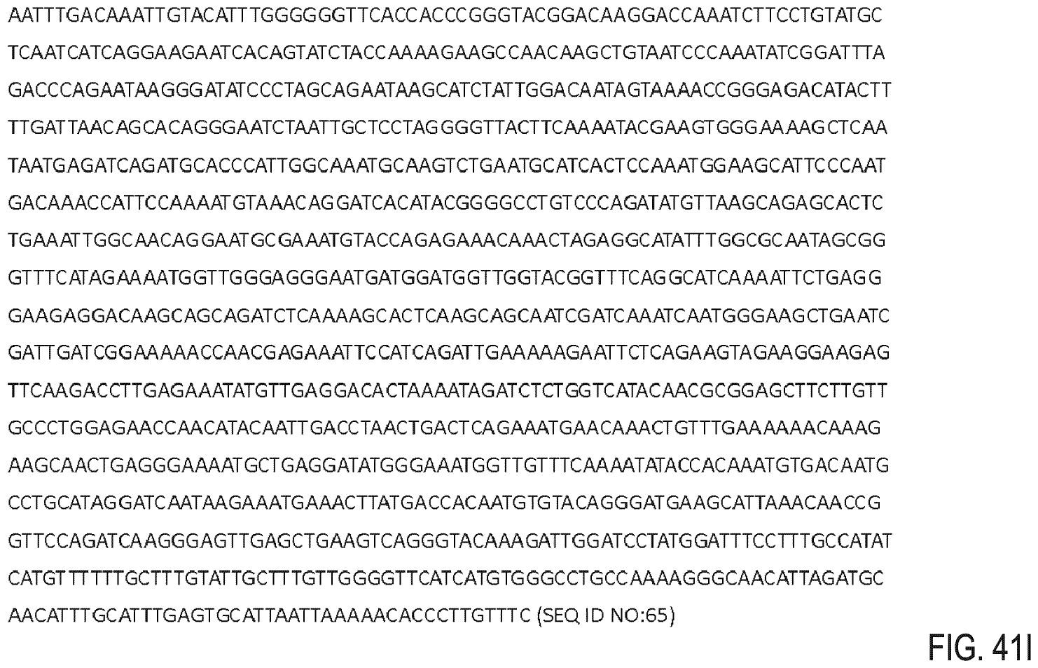

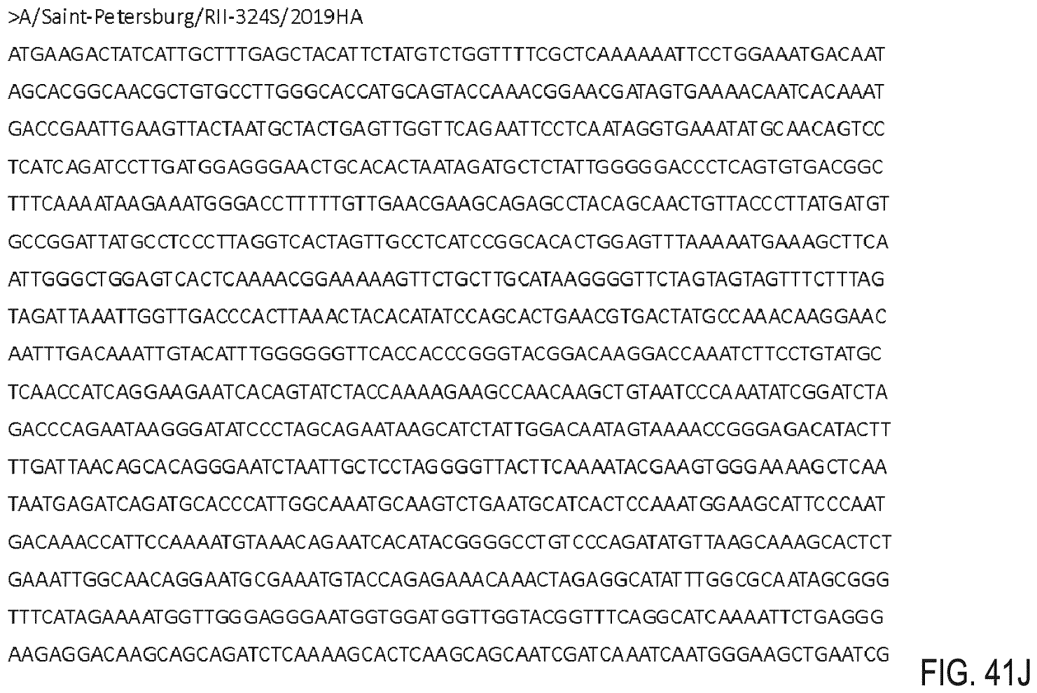

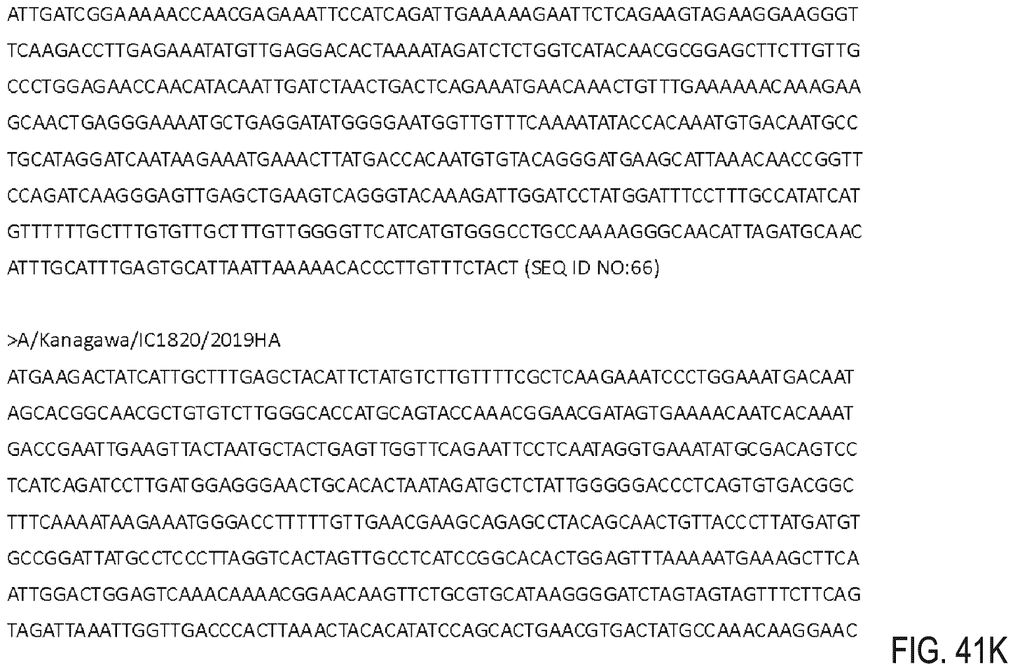

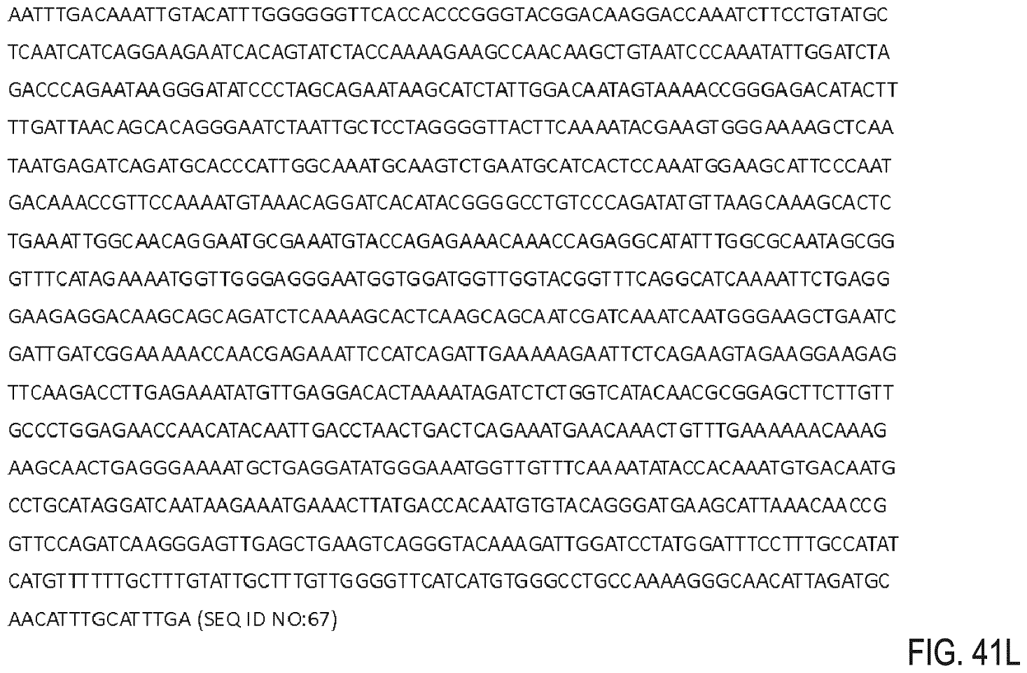

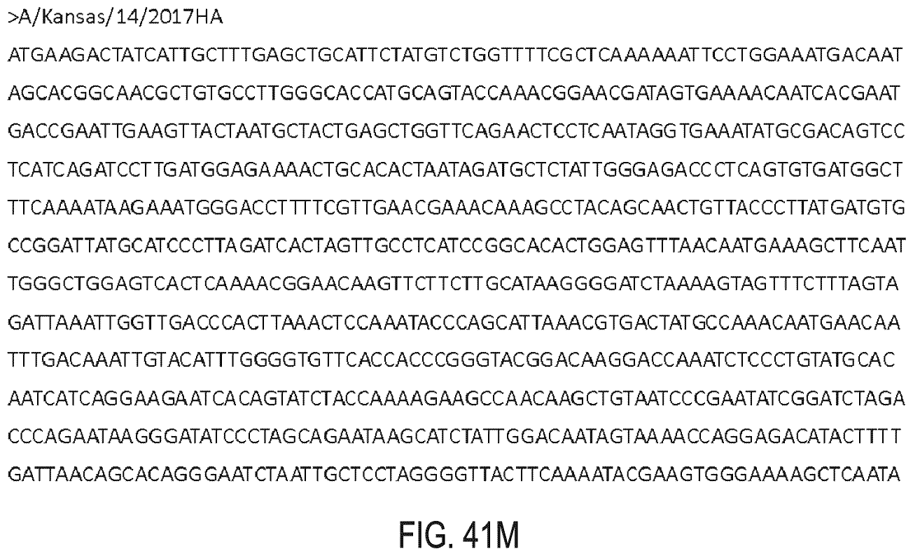

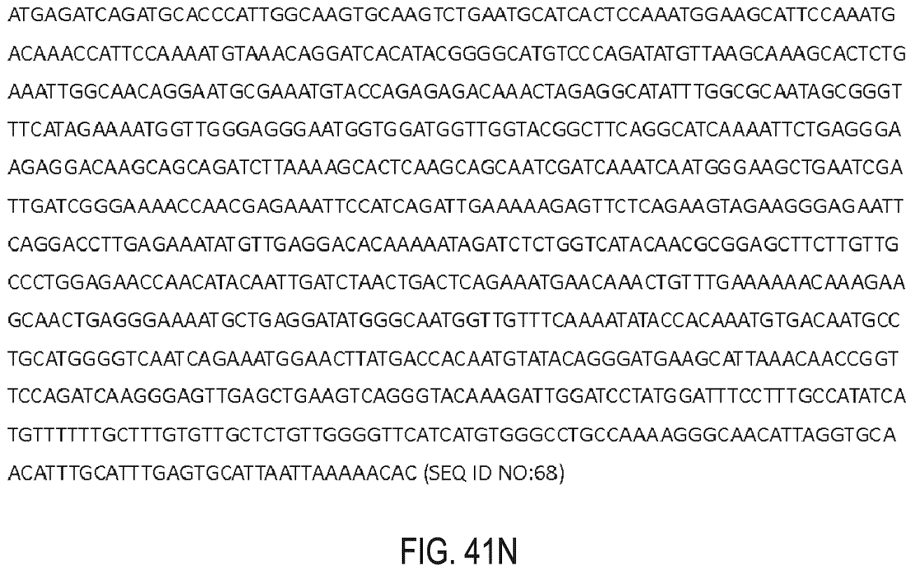

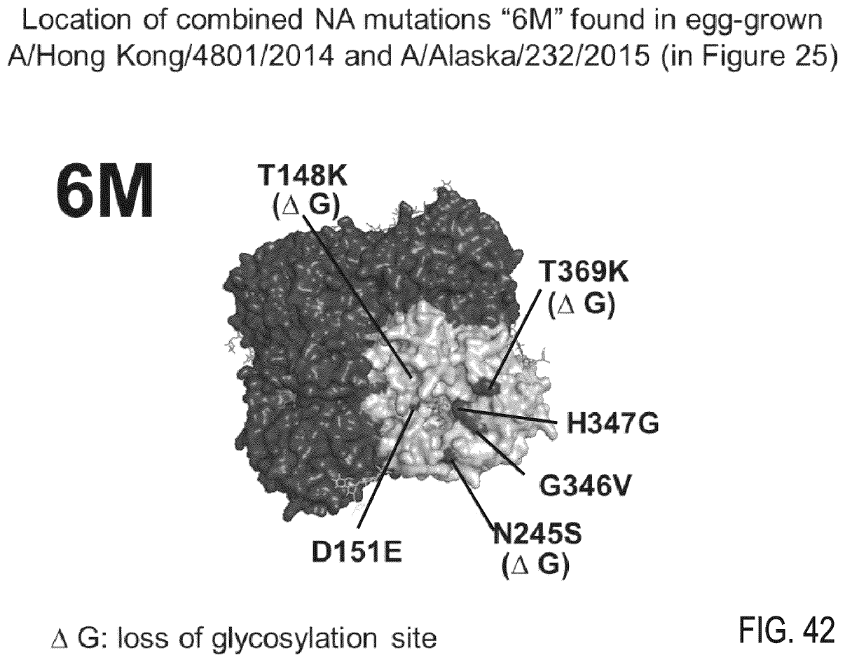

To this end, HA proteins are constructed that lacked or possessed limited receptor-binding activity but retained their antigenicity by introducing three mutations, e.g., Y98F, W153A, and H183F, at sites located inside the receptor-binding pocket that would not affect the antigenicity of the HA (FIGS. 39A-B).

### EXAMPLE III

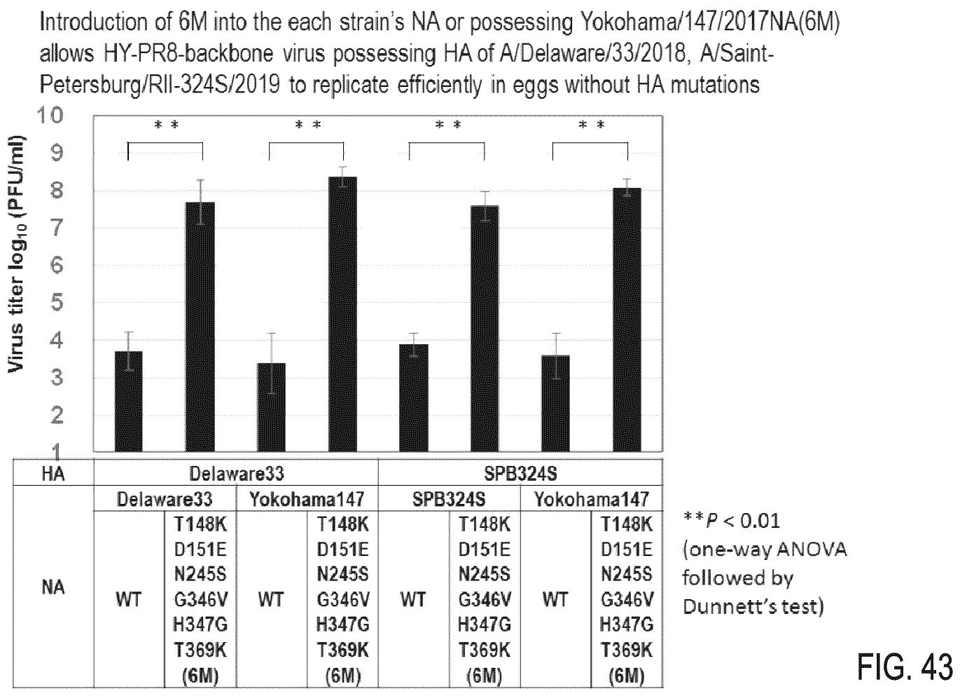

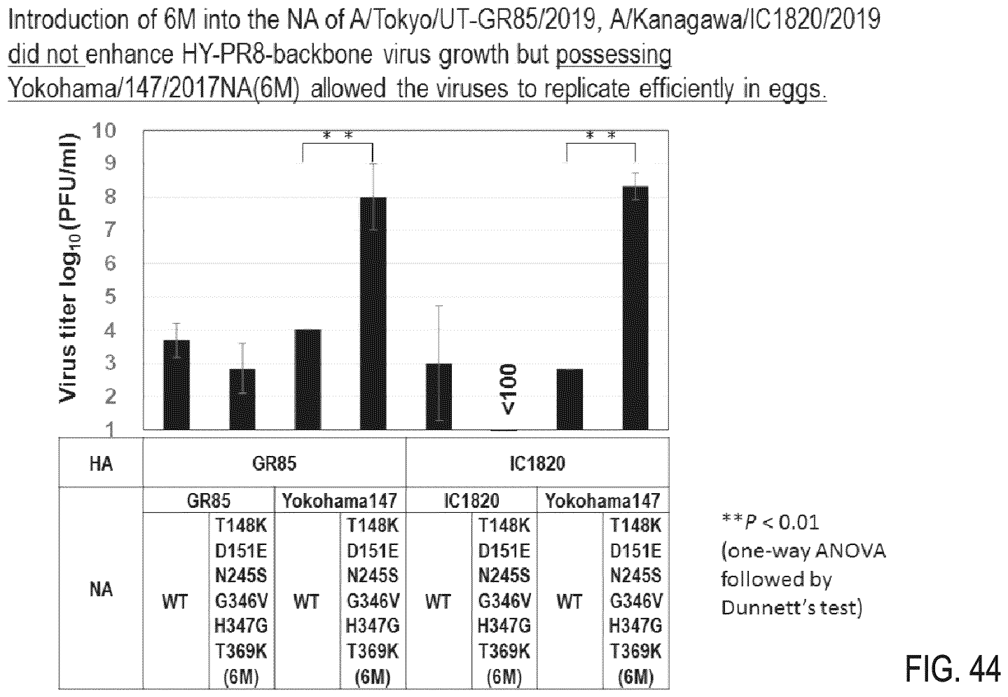

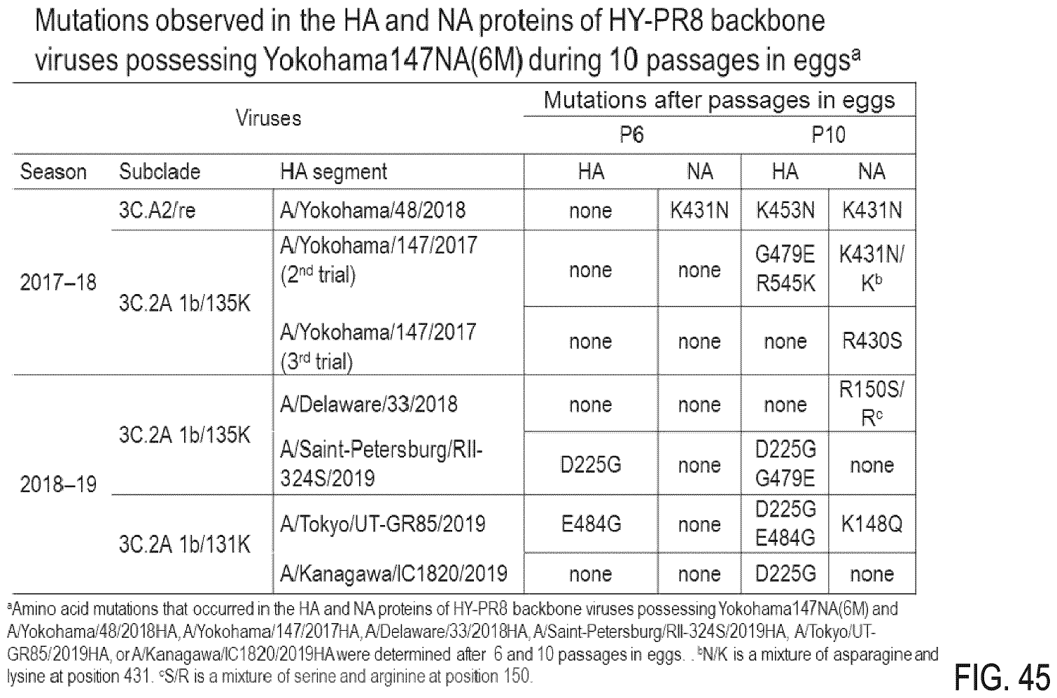

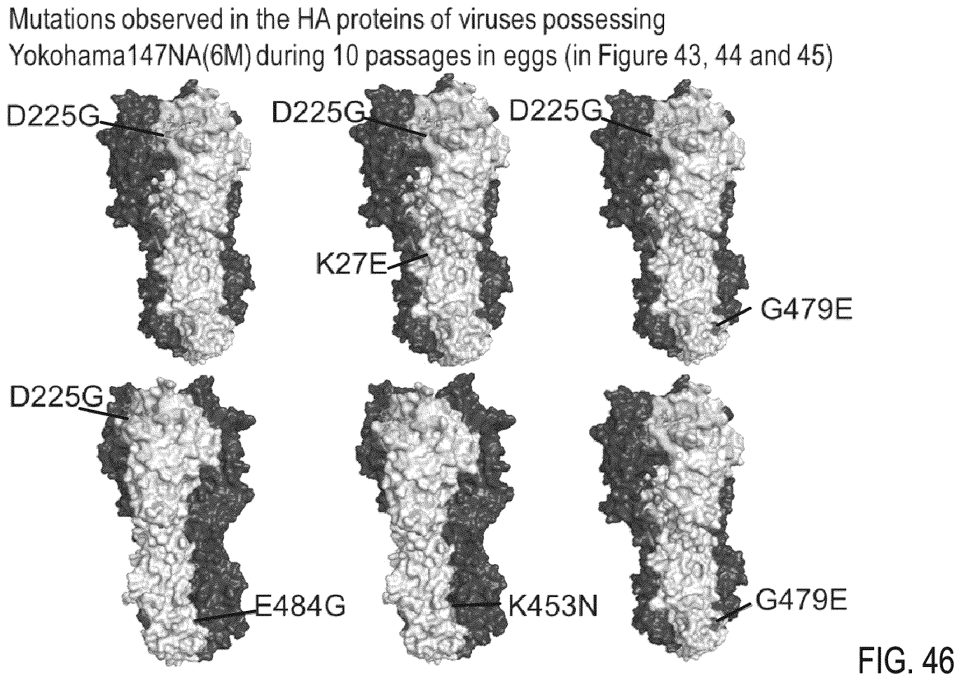

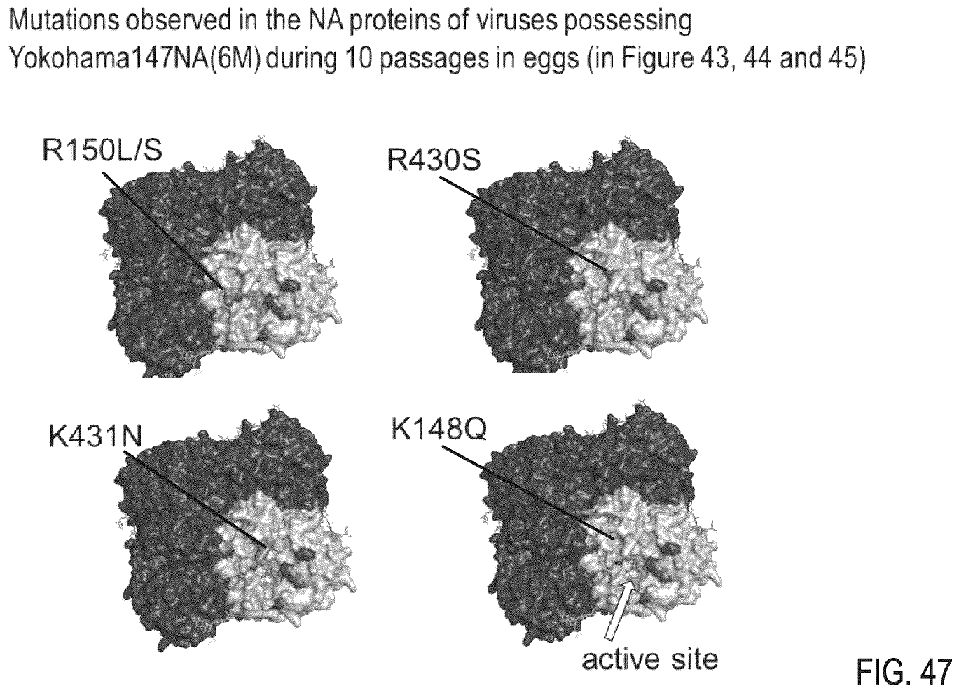

Exemplary NA residues were found in egg-grown A/Hong Kong/4801/2014 and A/Alaska/232/2015 (“6M”). Introducing 6M mutations into the NA of A/Yokohama/48/2018 and A/Yokohama/147/2017 enhanced HY-PR8-backbone virus growth. Therefore, the effect of introducing 6M mutations into other strains and the effect of possessing Yokohama147NA(6M) on the growth of viruses possessing HA from other strains was examined (FIGS. 43-44). Harvested viruses possessing each strain's NA(6M) or Yokohama/147/2017NA(6M) were sequenced. None had additional mutations in HA and NA were observed.

Viruses possessing A/Yokohama/147/2017 NA(6M) acquired HA-D225G and K27E after passage 10. HA-K27E is located in the stem region of HA protein, suggesting that K27E was unlikely to alter HA antigenicity. HA-D225G is located near receptor binding site. However, the reactivity of an H3N2 virus possessing HA-D225N with ferret antiserum differed from that of the wild-type virus by only two-fold, suggesting that HA-D225G alone was unlikely to alter HA antigenicity substantially (Chambers et al., Cell Rep. 2015).

It was analyzed whether the viruses possessing Yokohama147NA(6M) and HA from other strains can also replicate without acquiring HA mutations at major antigenic sites during egg passages. During egg passages, many of the tested viruses acquired HA-D225G but none of them acquired HA mutations at major antigenic sites. G479E, K453N, E484G (located in the stem region) and R545K (located in the cytoplasmic tail) were unlikely to alter HA antigenicity (FIG. 45).

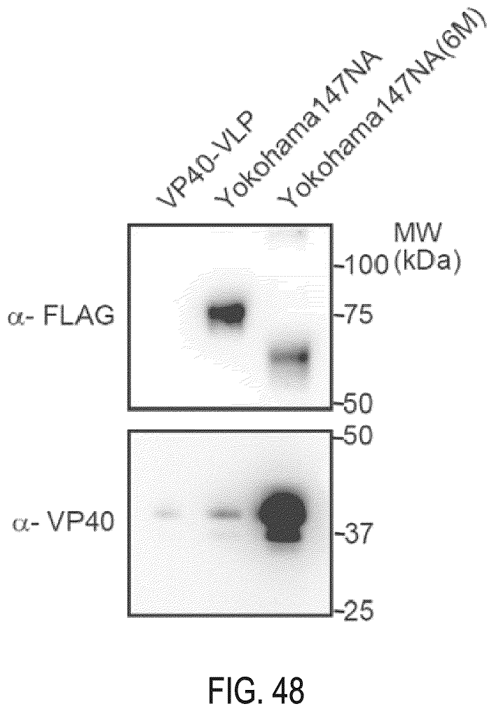

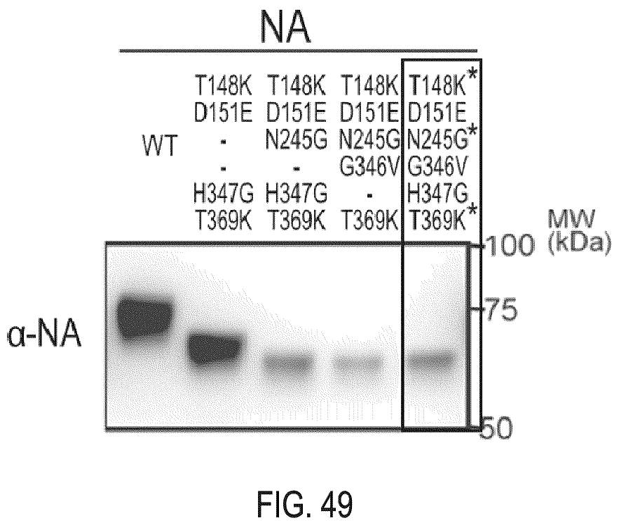

The mechanism of how the NA(6M) mutant viruses can replicate efficiently in eggs was investigated. VP40-induced VLPs bearing FLAG-tagged Yokohama147NA or Yokohama147NA(6M) were prepared. Immunoblotting analysis with anti-FLAG and anti-VP40 antibodies showed reduced molecular weight of Yokohama147NA(6M) protein compared to that of wild-type Yokohama147NA protein (FIG. 48). FIG. 49 shows another western blotting analysis suggesting the loss of glycosylation site of mutant NA protein due to the introduction of 6M mutations.

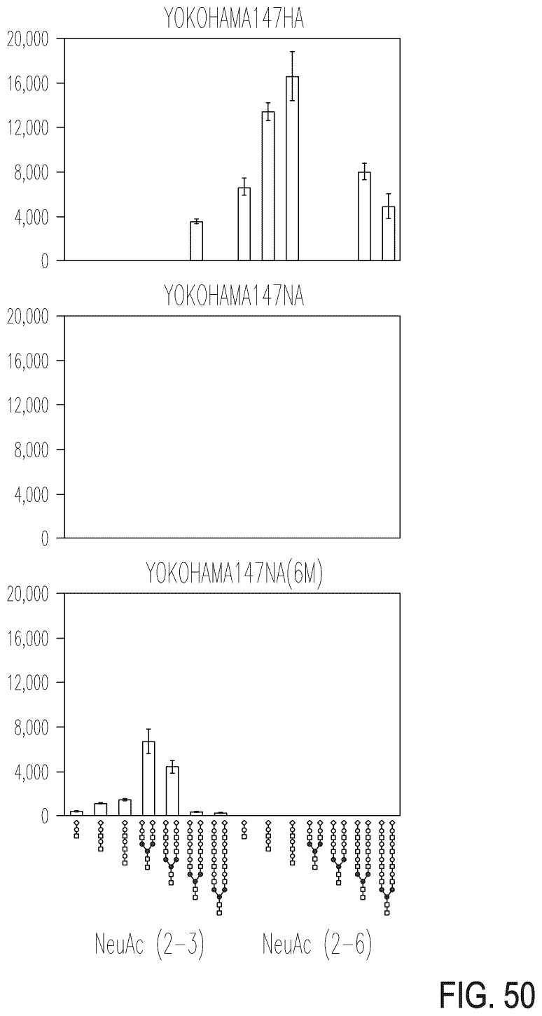

Next the receptor-binding specificities of Yokohama147HA, Yokohama147NA, and Yokohama147NA(6M) were analyzed using in a glycan microarray containing a library of α2-3 and α2-6 sialosides, including N-linked glycans representative of those found on chorioallantoic membranes of eggs. The analysis showed Yokohama147NA(6M) bound to α2-3 sialosides found on chorioallantoic membranes of eggs (FIG. 50).

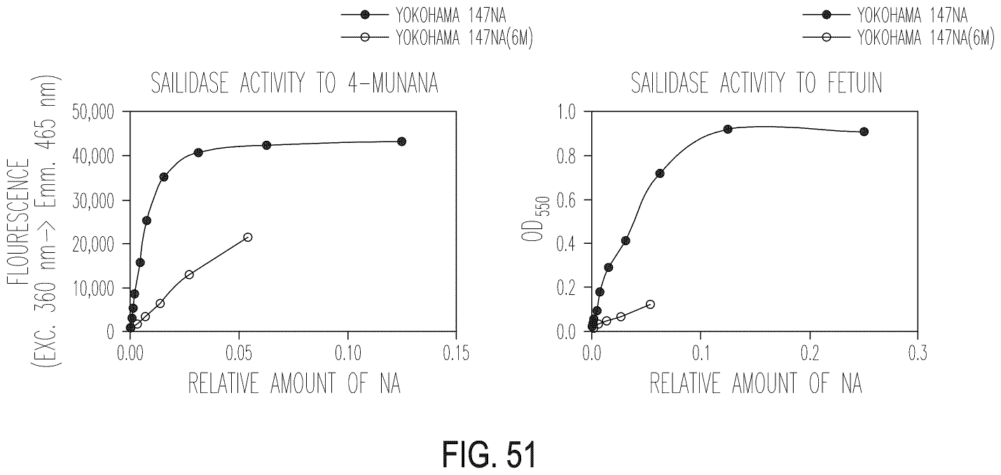

It was determined whether 6M mutations alter the NA sialidase activity (FIG. 51). Ebola VP40-based VLPs bearing Yokohama147NA or NA(6M) were serially diluted, incubated with the sialidase substrate 4-MUNANA, and the released 4-MU was quantified to assess sialidase activity. The analysis revealed that introduction of 6M into Yokohama147NA decreased its sialidase activity.

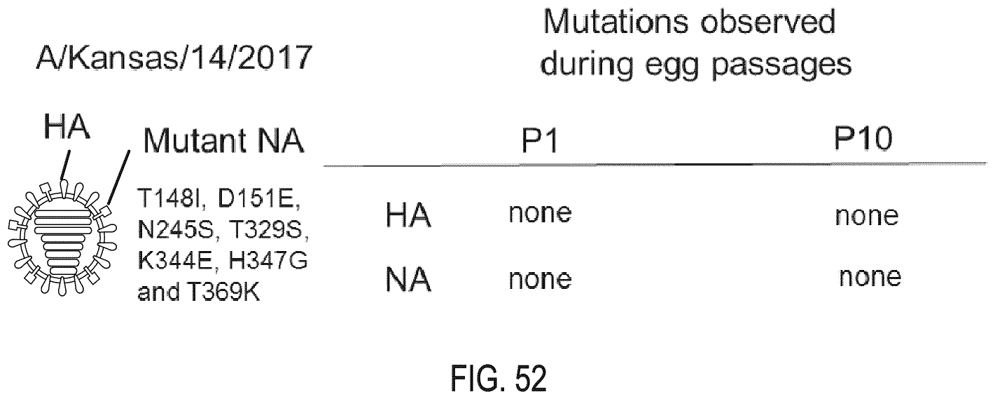

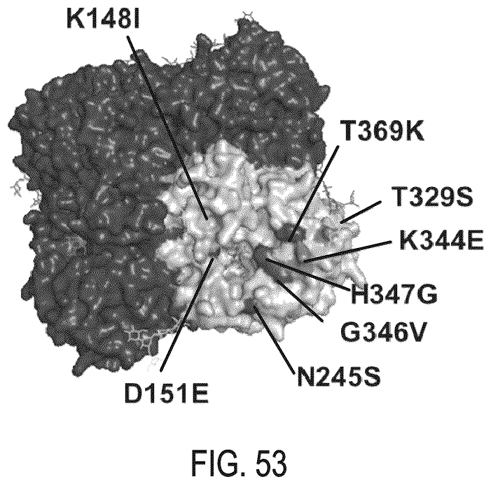

To identify further NA mutations that can allow viruses replicate efficiently in eggs without depending on HA receptor binding activity, HY-PR8 backbone viruses were generated that possess HA(del RBS) and A/Kansas/14/2017NA(6M) and then were passaged in eggs. During the passages, a mutant NA was obtained (T148I, D151E, N245S, T329S, K344E, G346V, H347G and T369K) (=6M+T148I+T329S+K344E). HY-PR8 backbone viruses possessing wild type HA and NA(6M+T148I+T329S+K344E) from A/Kansas/14/2017 were prepared and then analyzed to determine if the virus acquired the HA mutations during passages in eggs. The virus possessing NA(6M+T148I+T329S+K344E) did not acquire any of HA and NA mutations during 10 egg passages (FIG. 52).

### EXAMPLE IV

In one embodiment, an isolated recombinant influenza virus is provided comprising a selected NA viral segment encoding a plurality of selected residues or a deletion of residues in NA, wherein the selected NA viral segment does not encode a NA having a threonine (T) or lysine (K) at residue 148, and does not encode a threonine at residue 32, an aspartic acid (D) at position 151, an asparagine (N) at position 245, an asparagine at residue 329, a glycine (G) at position 346, a histidine at residue 347, or includes a NA having a deletion of one or more of residues 46 to 50, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine or lysine at residue 148 and has one or more of a threonine at residue 32, does not have a deletion of residues 46 to 50, an aspartic acid at position 147, an aspartic acid at residue 151, an asparagine at residue 245, an asparagine at residue 329, a glycine at residue 346, a histidine at residue 347, or any combination thereof. In one embodiment, the selected NA viral segment does not encode a NA having a threonine at residue 148, and does not encode a NA having an aspartic acid at position 151, an asparagine at position 245, a histidine at residue 347, or a threonine at residue 369, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine at residue 148, an aspartic acid at residue 151, an asparagine at residue 245, a histidine at residue 347, and a threonine at residue 369, or any combination thereof. In one embodiment, the selected NA viral segment does not encode a NA having a threonine or lysine at residue 148, and does not encode a NA having an aspartic acid at position 151, an asparagine at position 245, a histidine at residue 347, or a threonine at residue 369, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine or lysine at residue 148, an aspartic acid at residue 151, an asparagine at residue 245, a histidine at residue 347, and a threonine at residue 369, or any combination thereof. In one embodiment, the selected NA viral segment encodes a NA having an isoleucine (I), leucine (L), glycine or alanine (A) at residue 148. In one embodiment, the isolated recombinant influenza virus is a reassortant. In one embodiment, the NA viral segment encodes a NA that has at least 90% amino acid sequence identity to SEQ ID NO:1, SEQ ID NO:3, SEQ ID NO:48, SEQ ID NO:49, or SEQ ID NO:50. In one embodiment, the NA viral segment encodes a NA that has at least 90% amino acid sequence identity to SEQ ID NO:2. In one embodiment, the NA viral segment encodes a N2, N3, N7, or N9. In one embodiment, the NA viral segment encodes a N1, N4, N5, N6, N8, N10 or N11. In one embodiment, the residue at position 32 is A, I, G, or L, wherein the deletion is a deletion of residues 46 to 50, wherein the residue at position 147 is N or glutamine (Q), wherein the residue at position 329 is D or glutamic acid E, or wherein the residue at position 346 is serine (S), T, proline (P), tyrosine (Y), tryptophan (W), A, N, I, or L. In one embodiment, the residues at position 346 is V, S, I or L. In one embodiment, the residue at position 148 is I. In one embodiment, the residue at position 151 is E, N or Q. In one embodiment, the residue at position 245 is S, T, I, L, A, N, W, Y, P, V, or G. In one embodiment, the residue at position 347 is G, Q, S, T, Y, C or W. In one embodiment, the residue at position 369 is K, H, R, E, P, or D. In one embodiment, the residue at position 147 is N or Q, the residue at position 329 is D or E, the residue at position 347 is G, Q, S, T, Y, C or W, or any combination thereof. In one embodiment, the residue at position 147 is N or Q, the residue at position 329 is D or E, the residue at position 347 is G or Q, or any combination thereof. In one embodiment, the residue at position 148 is K, R or H, the residue at position 151 is E, N or Q, the residue at position 245 is S, T, I, L, A, W, Y, P, V, or G, or any combination thereof. In one embodiment, the residue at position 148 is K, R or H, the residue at position 151 is E, N or Q, the residue at position 245 is S, T, I, L, A, or V, and/or the residue at position 346 is S, T, P, Y, W, A, N, I, L, or V, or any combination thereof. In one embodiment, the selected NA viral segment does not encode a NA having a threonine at position 148, does not encode a NA having an aspartic acid at position 151, does not encode a NA having an asparagine at position 245, does not encode a NA having a histidine, arginine or an asparagine at residue 347, or any combination thereof. In one embodiment, the selected NA viral segment does not encode a NA having an aspartic acid at position 147, does not encode a NA having an asparagine at residue 329, does not encode a NA having a histidine, arginine or asparagine at residue 347, or any combination thereof. In one embodiment, the selected NA viral segment does not encode a NA having a threonine at position 148, does not encode a NA having an aspartic acid at position 151, does not encode a NA having an asparagine at position 245, does not encode a NA having a glycine at position 346, or any combination thereof. In one embodiment, the HA is H1, H3, H5, H7, or H9. In one embodiment, the virus is an influenza A virus. In one embodiment, the PA, PB1, PB2, NP, M, and NS viral segments have at least 85% nucleic acid sequence identity to SEQ ID Nos. 24 to 29 or 39 to 44 or encode a polypeptide having at least 80% amino acid sequence identity to a polypeptide encoded by SEQ ID Nos. 24 to 29 or 39 to 44. In one embodiment, the PB2 has I, A, L, or G at residue 147.

Also provided, in one embodiment, is an isolated recombinant nucleic acid comprising a nucleic acid sequence for an influenza virus NA viral segment that encodes a NA having a plurality of selected residues or a deletion of residues, wherein the selected NA viral segment does not encode a NA having a threonine or lysine at residue 148, and does not encode a threonine at residue 32, an aspartic acid at position 151, an asparagine at position 245, an asparagine at residue 329, a glycine at position 346, a histidine at residue 347, or include a NA having a deletion of one or more of residues 46 to 50, or any combination thereof, wherein the numbering is based on N2. In one embodiment, the selected NA viral segment does not encode a NA having a threonine at residue 148, and does not encode a NA having an aspartic acid at position 151, an asparagine at position 245, a histidine at residue 347, or a threonine at residue 369, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine at residue 148, an aspartic acid at residue 151, an asparagine at residue 245, a histidine at residue 347, and a threonine at residue 369, or any combination thereof. In one embodiment, the selected NA viral segment does not encode a NA having a threonine or lysine at residue 148, and does not encode a NA having an aspartic acid at position 151, an asparagine at position 245, a histidine at residue 347, or a threonine at residue 369, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine or lysine at residue 148, an aspartic acid at residue 151, an asparagine at residue 245, a histidine at residue 347, and a threonine at residue 369, or any combination thereof. In one embodiment, the selected NA viral segment encodes a NA having an isoleucine (I), leucine (L), glycine (G) or alanine (A) at residue 148. In one embodiment, the NA has at least 90% amino acid sequence identity to SEQ ID NO:1, SEQ ID NO:2 or SEQ ID NO:3. In one embodiment, the NA is a N2, N3, N7, or N9. In one embodiment, the NA is a N1, N4, N5, N6, N8, N10 or N11. In one embodiment, the HA is H1, H2, H3, H5, H7, or H9. In one embodiment, the residue at position 32 is A, I, G, or L, the residue at position 147 is N or Q, the residue at position 329 is D or E, the residue at position 151 is E, N or Q, the residue at position 148 is I, L, V, A, or G, the residue at position 245 is S, T, I, L, A, W, Y, P, V, or G, the residue at position 347 is G, Q, S, or T, the residue at position 346 is S, T, P, Y, W, A, N, I, L, or V, the residue at position 369 is K, H, R, E, P, or D, or any combination thereof. In one embodiment, the residue at position 151 is E, N or Q, the residue at position 148 is I, L, V, A, or G, the residue at position 245 is S, T, I, L, A, W, Y, P, V, or G, the residue at position 329 is S, I, L, A, W, Y, P, V, or G, the residue at position 347 is G, Q, S, or T, the residue at position 346 is S, T, P, Y, W, A, N, I, L, or V, the residue at position 369 is K, H, R, E, P, or D, or any combination thereof. In one embodiment, the NA has at least 90% amino acid sequence identity to SEQ ID NO:1, SEQ ID NO:2, SEQ ID NO:3, SEQ ID NO:48, or SEQ ID NO:49, or at least 90% amino acid sequence identity to a NA encoded by one of SEQ ID Nos. 51-59 or 69-70.

In one embodiment, a method to prepare influenza virus is provide comprising: contacting a cell with: a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS DNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production encodes a NA having a plurality of selected residues or a deletion of residues, wherein the selected NA viral segment does not encode one or more of: a threonine or lysine at residue 148, a threonine at residue 32, an aspartic acid at position 151, an asparagine at position 245, an asparagine or threonine at residue 329, a lysine at residue 344 a glycine at position 346, a histidine at residue 347, or include a NA having a deletion of one or more of residues 46 to 50, or any combination thereof, wherein the numbering is based on N2; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In one embodiment, the NA has at least 90% amino acid sequence identity to SEQ ID NO:1, SEQ ID NO:2, SEQ ID NO:3, SEQ ID NO:48 or SEQ ID NO:49. In one embodiment, the NA is N2, N3, N7, or N9. In one embodiment, the HA is H1, H3, H7, or H9. In one embodiment, the HA is H2, H4, H5, H6, H8, or any of H10-H18. In one embodiment, the residue at position 147 is N or Q, the residue at position 329 is D or E, the residue at position 347 is G, Q, N, S, T, Y, C or W, or the residue at position 346 is S, T, P, Y, W, A, N, I, L, or V. In one embodiment, the residue at position 151 is E, N or Q, the residue at position 148 is I, L, V, A, or G, the residue at position 245 is S, T, I, L, A, W, Y, P, V, or G, the residue at position 347 is G, Q, S, or T, the residue at position 346 is S, T, P, Y, W, A, N, I, L, or V, the residue at position 369 is K, H, D, E, or R, or any combination thereof. In one embodiment, the PA, PB1, PB2, NP, M, and NS viral segments have at least 85% nucleic acid sequence identity to SEQ ID Nos. 24 to 29 or 39 to 44 or encode a polypeptide having at least 80% amino acid sequence identity to a polypeptide encoded by SEQ ID Nos. 24 to 29 or 39 to 44.

Further provided is a method of immunizing an avian or a mammal, comprising: administering to the avian or the mammal a composition having an effective amount of the virus described herein. In one embodiment, the composition comprises at least one other different influenza virus. In one embodiment, the mammal is a human. In one embodiment, the composition is administered intranasally or via injection.

Viruses described herein may be passaged in eggs or other cells.

Exemplary backbone viral segments include but are not limited to: PB2, M202L, F323L; PB1, Q247H; PA, K142N; NP, R74K; M, V97A, Y100H; and NS, K55E, or PB2, I504V; PB1, M40L/G180W: PA, R401K; NP, I116L and NS1, A30P/R118K.

### EXAMPLE V

In one embodiment, a method to decrease influenza HA binding to cells is provided that includes altering one or more residues in the HA binding pocket of HA that binds to sialic acid on allantoic membranes. In one embodiment, nucleic acid encoding the HA is altered. In one embodiment, the HA is H1, H3, H7, or H9. In one embodiment, the HA is H2, H4, H5, H6, H8, or any of H10-H18. In one embodiment, the residue at position 98, 153 or 183 of HA is altered based on the numbering of H3 HA. In one embodiment, the residue at position 98 is not Y. In one embodiment, the residue at position 153 is not W. In one embodiment, the residue at position 183 is not H. In one embodiment, the residue at position 98 is F, G, I, V, T, H, W, or L. In one embodiment, the residue at position 153 is A, G, I, V, T, or L. In one embodiment, the residue at position 183 is F, A, G, I, L, V, Y, W, P, or T.

In one embodiment, a method to prepare an influenza virus that binds to cells via influenza neuraminidase is provided that includes providing a vector comprising a recombinant nucleic acid molecule comprising sequences for an influenza virus HA segment from a first influenza virus isolate, which segment encodes an HA with an amino acid other than tyrosine at position 98 in HA1, other than tryptophan at position 153 in HA1, other than histidine at position 183 in HA1, or any combination thereof, wherein the numbering for HA1 residues is that for H3; modifying the HA segment to encode F, G, I, V, T, H, W, or L at position 98, encode A, G, I, V, T, or L at position 153, encode F, A, G, I, L, V, Y, W, P, or T at position 183, or any combination thereof, thereby yielding a modified HA segment; and contacting a cell with a vector comprising promoter that yields full length, genomic influenza virus RNA or its complement operably linked to an influenza virus PA segment DNA linked to a transcription termination sequence, a vector comprising a promoter that yields full length, genomic influenza virus RNA or its complement operably linked to an influenza virus PB1 segment DNA linked to a transcription termination sequence, a vector comprising a promoter that yields full length, genomic influenza virus RNA or its complement operably linked to an influenza virus PB2 segment DNA linked to a transcription termination sequence, a vector comprising a promoter that yields full length, genomic influenza virus RNA or its complement operably linked to the modified HA segment linked to a transcription termination sequence, a vector comprising a promoter that yields full length, genomic influenza virus RNA or its complement operably linked to an influenza virus NP segment DNA linked to a transcription termination sequence, a vector comprising a promoter that yields full length, genomic influenza virus RNA or its complement operably linked to an influenza virus NA segment DNA linked to a transcription termination sequence, a vector comprising a promoter that yields full length, genomic influenza virus RNA or its complement operably linked to an influenza virus M segment DNA linked to a transcription termination sequence, and a vector comprising a promoter that yields full length, genomic influenza virus RNA or its complement operably linked to an influenza virus NS segment DNA linked to a transcription termination sequence; and a vector comprising a promoter that yields mRNA operably linked to a DNA segment encoding influenza virus PA, a vector comprising a promoter that yields mRNA operably linked to a DNA segment encoding influenza virus PB1, a vector comprising a promoter that yields mRNA operably linked to a DNA segment encoding influenza virus PB2, and a vector comprising a promoter that yields mRNA operably linked to a DNA segment encoding influenza virus NP, and optionally a vector comprising a promoter that yields mRNA operably linked to a DNA segment encoding influenza virus HA, a vector comprising a promoter that yields mRNA operably linked to a DNA segment encoding influenza virus NA, a vector comprising a promoter that yields mRNA operably linked to a DNA segment encoding influenza virus M1, a vector comprising a promoter that yields mRNA operably linked to a DNA segment encoding influenza virus M2, or a vector comprising a promoter that yields mRNA operably linked to a DNA segment encoding influenza virus NS1 or a vector comprising a promoter that yields mRNA operably linked to a DNA segment encoding influenza virus NS2; in an amount effective to yield infectious influenza virus that binds to cells via the NA.

### EXAMPLE VI

In one embodiment, an isolated recombinant influenza virus comprising a selected NA viral segment encoding a plurality of selected residues, a HA viral segment, and one or more of a PB1 viral segment, a PB2 viral segment, a PA viral segment, a NP viral segment, a M viral segment and a NS viral segment. In one embodiment, the selected NA viral segment does not encode a NA having a threonine or lysine at residue 148, does not encode an aspartic acid (D) at position 151, does not encode an asparagine at position 245, does not encode a threonine at position 329, does not encode a lysine at position 344, does not encode a glycine at position 346, does not encode a histidine at residue 347, and/or does not encode a threonine at position 369, wherein the numbering is based on N2, wherein the recombinant influenza virus has enhanced replication in avian eggs, has reduced sialidase activity, has increased binding to certain sialic acid residues and/or has a reduction in HA mutations when grown in avian eggs relative to a corresponding influenza virus that has a NA that encodes a threonine or lysine at residue 148, encodes an aspartic acid at residue 151, encodes an asparagine at residue 245, encodes a threonine at residue 329, encodes a lysine at residue 344, encodes a glycine at residue 346, encodes a histidine at residue 347, or encodes a threonine at position 369, or any combination thereof. In one embodiment, the NA segment of the recombinant virus has at position 329 a serine (S), valine (V), alanine (A), G, cysteine (C), methionine (M), isoleucine (I) or leucine (L) or at position 346 a V, S, T, proline (P), tyrosine (Y), tryptophan (W), A, N, I, or L. In one embodiment, the NA segment of the recombinant virus has at position 148 an I. In one embodiment, the NA segment of the recombinant virus has at position 151 an E, N or Q. In one embodiment, the NA segment of the recombinant virus has at position 245 a S, T, I, L, A, W, Y, P, V, or G. In one embodiment, the NA segment of the recombinant virus has at position 329 a S, I, L, A, W, Y, P, V, or G. In one embodiment, the NA segment of the recombinant virus has at position 344 an E, H, D, N or Q. In one embodiment, the NA segment of the recombinant virus has at position 346 a V, S, T, I, L, A, W, Y, or P. In one embodiment, the NA segment of the recombinant virus has at position 347 a G, Q, S, T, Y, C or W. In one embodiment, the NA segment of the recombinant virus has at position 369 a K, H, R, E, P, or D. In one embodiment, the recombinant virus is a reassortant. In one embodiment, the NA viral segment encodes a NA that has at least 90% amino acid sequence identity to SEQ ID NO:1, SEQ ID NO:3, SEQ ID NO:48, or SEQ ID NO:49, or has at least 90% amino acid sequence identity to a NA encoded by any one of SEQ ID Nos. 51-59. In one embodiment, the NA viral segment encodes a NA that has at least 90% amino acid sequence identity to SEQ ID NO:2. In one embodiment, the NA viral segment encodes a N2, N3, N7, or N9. In one embodiment, the NA viral segment encodes a N1, N4, N5, N6, N8, N10 or N11. In one embodiment, the HA is H2 or H3. In one embodiment, the virus is an influenza A virus. In one embodiment, the PA, PB1, PB2, NP, M, and NS viral segments have at least 85% nucleic acid sequence identity to SEQ ID Nos. 24 to 29 or 39 to 44 or encode a polypeptide having at least 80% amino acid sequence identity to a polypeptide encoded by SEQ ID Nos. 24 to 29 or 39 to 44. In one embodiment, PB2 has I, A, L, or G at residue 147.

Further provided is an isolated recombinant nucleic acid comprising a nucleic acid sequence for an influenza virus NA viral segment that encodes a NA having a plurality of selected residues, wherein the selected NA viral segment, does not encode a NA having a threonine or lysine at residue 148, does not encode an aspartic acid at position 151, does not encode an asparagine at position 245, does not encode a threonine at position 329, does not encode a lysine at position 344, does not encode a glycine at position 346, does not encode a histidine at residue 347, and/or does not encode a threonine at position 369, wherein the numbering is based on N2. In one embodiment, the NA has at least 90% amino acid sequence identity to SEQ ID NO:1, SEQ ID NO:2, SEQ ID NO:3, SEQ ID NO:48, or SEQ ID NO:49, or at least 90% amino acid sequence identity to a NA encoded by one of SEQ ID Nos. 51-59. In one embodiment, the NA is a N2, N3, N7, or N9. In one embodiment, the NA is a N1, N4, N5, N6, N8, N10 or N11. In one embodiment, the residue at position 148 is I, the residue at position 329 is S, the residue at position 151 is E, N or Q, the residue at position 245 is S, T, I, L, A, V, or G, the residue at position 347 is G, Q, S, or T, the residue at position 346 is S, T, P, Y, W, A, N, I, L, or V, the residue at position 369 is K,

H, R, E, P, or D, or any combination thereof. In one embodiment, the residue at position 151 is E, N or Q, the residue at position 148 is I or K, the residue at position 245 is S, T, I, L, A, W, Y, P, V, or G, the residue at position 347 is G, Q, S, or T, the residue at position 346 is S, T, P, Y, W, A, N, I, L, or V, the residue at position 369 is K, H, R, E, P, or D, or any combination thereof.

In one embodiment, a method to prepare influenza virus is provided. The method includes contacting a cell with a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS DNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production encodes a NA having a plurality of selected residues, wherein the selected NA viral segment does not encode a NA having a threonine or lysine at residue 148, does not encode an aspartic acid at position 151, does not encode an asparagine at position 245, does not encode a threonine at position 329, does not encode a lysine at position 344, does not encode a glycine at position 346, does not encode a histidine at residue 347, and/or does not encode a threonine at position 369, wherein the numbering is based on N2; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In one embodiment, the NA has at least 90% amino acid sequence identity to SEQ ID NO:1, SEQ ID NO:2, SEQ ID NO:3, SEQ ID NO:48 or SEQ ID NO:49 or at least 90% amino acid sequence identity to a NA encoded by one of SEQ D Nos. 51-59. In one embodiment, the NA is N2, N3, N7, or N9. In one embodiment, the HA is H1, H2, H3, H7, or H9. In one embodiment, HA is H2, H4, H5, H6, H8, or any of H10-H18. In one embodiment, the residue at position 329 is S, A, I, L or G, the residue at position 347 is G, Q, N, S, T, Y, C or W, or the residue at position 346 is S, T, P, Y, W, A, N, I, L, or V. In one embodiment, the residue at position 151 is E, N or Q, the residue at position 148 is K, H, D or E, the residue at position 245 is S, T, I, L, A, W, Y, P, V, or G, the residue at position 347 is G, Q, S, or T, the residue at position 346 is V, S, T, P, Y, W, A, N, I, or L, the residue at position 369 is K, H, D, E, or R, or any combination thereof. In one embodiment, PA, PB1, PB2, NP, M, and NS viral segments have at least 85% nucleic acid sequence identity to SEQ ID Nos. 24 to 29 or 39 to 44 or encode a polypeptide having at least 80% amino acid sequence identity to a polypeptide encoded by SEQ ID Nos. 24 to 29 or 39 to 44. Also provided is isolated virus prepared by the method.

The recombinant virus may be employed in a method of immunizing an avian or a mammal, which includes administering to the avian or the mammal a composition having an effective amount of the virus. In one embodiment, the composition comprises at least one other different influenza virus. In one embodiment, the mammal is a human. In one embodiment, the composition is administered intranasally or via injection.

### EXAMPLE VII

In one embodiment, an isolated recombinant influenza virus comprising a selected NA viral segment encoding a plurality of selected residues or a deletion of residues in NA is provided. The virus includes the selected NA viral segment encoding the plurality of selected residues, a HA viral segment, and one or more of a PB1 viral segment, a PB2 viral segment, a PA viral segment, a NP viral segment, a M viral segment and a NS viral segment. In one embodiment, the selected NA viral segment does not encode a NA having a threonine or lysine at residue 148, does not encode an aspartic acid at position 151, does not encode an asparagine at position 245, does not encode a threonine at position 329, does not encode a lysine at position 344, does not encode a glycine at position 346, does not encode a histidine at residue 347, and does not encode a threonine at position 369, wherein the numbering is based on N2, wherein the recombinant influenza virus has enhanced replication in avian eggs, has reduced sialidase activity, enhanced binding to a2-3 sialosides, or has a reduction in HA mutations when grown in avian eggs relative to a corresponding influenza virus that has a NA that encodes a threonine or lysine at residue 148, encodes an aspartic acid at residue 151, encodes an asparagine at residue 245, encodes a threonine at residue 329, encodes a lysine at residue 344, encodes a glycine at residue 346, encodes a histidine at residue 347, and encodes a threonine at position 369. In one embodiment, the selected NA viral segment does not encode a NA having a threonine or lysine at residue 148, does not encode a threonine at residue 32, does not encode an aspartic acid at position 151, does not encode an asparagine at position 245, does not encode an asparagine or a threonine at residue 329, does not encode a lysine at position 344, does not encode a glycine at position 346, does not encode a histidine at residue 347, and/or does not encode a threonine at residue 369, or includes a NA having a deletion of one or more of residues 46 to 50, or any combination thereof, wherein the numbering is based on N2, wherein the recombinant influenza virus has enhanced replication in avian eggs, has reduced sialidase activity, enhanced binding to α2-3 sialosides, or has a reduction in HA mutations when grown in avian eggs relative to a corresponding influenza virus that has a NA that encodes a threonine or lysine at residue 148 and a threonine at residue 32, does not have a deletion of residues 46 to 50, has an aspartic acid at position 147, has an aspartic acid at residue 151, has an asparagine at residue 245, has an asparagine or threonine at residue 329, has a glycine at residue 346, has a histidine at residue 347, has a threonine at residue 369, or any combination thereof. In one embodiment, the selected NA segment encodes two or more of positions 148, 151, 245, 329, 344, 347, or 369 having lysine or isoleucine at residue 148, glutamic acid at residue 151, serine, threonine, glycine, alanine, leucine or isoleucine at residue 245 or serine, glycine, alanine, leucine or isoleucine residue 329, glutamic acid, aspartic acid, glutamine, asparagine or histidine at residue 344, valine, leucine, isoleucine, threonine or serine at reside 346, glycine, alanine, valine, leucine, isoleucine or threonine at residue 347, or lysine, histidine, aspartic acid or glutamic acid at residue 369. In one embodiment, wherein the selected NA segment encodes two or more of positions 148, 151, 245, 329, 344, 347, or 369 having isoleucine (I) at residue 148, glutamic acid at residue 151, serine, threonine, leucine or isoleucine at residue 245 or serine, leucine or isoleucine at residue 329, glutamic acid, aspartic acid or histidine at residue 344, valine, leucine, or isoleucine at reside 346, glycine, alanine, valine, leucine, or isoleucine at residue 347, or lysine, aspartic acid or glutamic acid at residue 369. In one embodiment, the selected NA segment does not encode threonine at residue 148, does not encode asparagine at residue 245, does not encode threonine at residue 369, does not encode aspartic acid at residue 151, does not encode a lysine at residue 344, does not encode glycine at reside 346, does not encode histidine at residue 347, and does not encode threonine at residue 369. In one embodiment, the selected NA segment encodes lysine or isoleucine (I) at residue 148, encodes glutamic acid (E) at residue 151, encodes serine (S), threonine, glycine, alanine (A), leucine (L) or isoleucine at residue 245, encodes serine, glycine, alanine, leucine or isoleucine at residue 329, encodes glutamic acid, arginine (R), aspartic acid (D) or histidine at residue 344, encodes valine, leucine, isoleucine, threonine or serine at reside 346, encodes glycine, alanine, valine, leucine, isoleucine or threonine at residue 347, or encodes lysine, histidine, aspartic acid or glutamic acid at residue 369. In one embodiment, the selected NA viral segment does not encode a NA having a threonine at residue 148, and does not encode a NA having an aspartic acid at position 151, an asparagine at position 245, a valine, serine, isoleucine or leucine at residue 346, a histidine at residue 347, or a threonine at residue 369, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine at residue 148, an aspartic acid at residue 151, an asparagine at residue 245, a histidine at residue 347, and a threonine at residue 369, or any combination thereof; or wherein the selected NA viral segment does not encode a NA having a threonine or lysine at residue 148, and does not encode a NA having an aspartic acid at position 151, an asparagine at position 245, a valine, serine, isoleucine or leucine at residue 346, a histidine at residue 347, or a threonine at residue 369, or any combination thereof, wherein the numbering is based on N2, wherein the recombinant influenza virus has enhanced replication in avian eggs or has a reduction in HA mutations when grown in avian eggs relative to a corresponding influenza virus that has a NA that encodes a threonine or lysine at residue 148, an aspartic acid at residue 151, an asparagine at residue 245, a glycine at residue 346, a histidine at residue 347, and a threonine at residue 369, or any combination thereof. In one embodiment,_the selected NA viral segment encodes a NA having an isoleucine, leucine, glycine or alanine at residue 148. In one embodiment, the residue at position 32 is A, I, G, or L, the deletion is a deletion of residues 46 to 50, wherein the residue at position 147 is N or glutamine (Q), wherein the residue at position 329 is D or glutamic acid, or wherein the residue at position 346 is serine, T, proline (P), tyrosine (Y), tryptophan (W), A, N, I, or L. In one embodiment, the residue at position 148 is I, the residue at position 151 is E, N or Q, the residue at position 245 is S, T, I, L, A, N, W, Y, P, V, or G, the residue at position 347 is G, Q, S, T, Y, C or W, the residue at position 369 is K, H, R, E, P, or D, or any combination thereof. In one embodiment, the residue at position 329 is serine, valine, alanine, G, cysteine (C), methionine (M), isoleucine or leucine or wherein the residue at position 346 is V, S, T, proline (P), tyrosine (Y), tryptophan (W), A, N, I, or L. In one embodiment, the residue at position 148 is I, the residue at position 151 is E, N or Q, the residue at position 245 is S, T, I, L, A, W, Y, P, V, or G, the residue at position 329 is S, I, L, A, W, Y, P, V, or G, the residue at position 344 is E, H, D, N or Q, the residue at position 346 is V, S, T, I, L, A, W, Y, or P, the residue at position 347 is G, Q, S, T, Y, C or W, or the residue at position 369 is K, H, R, E, P, or D. In one embodiment, the isolated recombinant influenza virus is a reassortant. In one embodiment, the NA viral segment encodes a NA that has at least 90% amino acid sequence identity to SEQ ID NO:1, SEQ ID NO:2, SEQ ID NO:3, SEQ ID NO:48, or SEQ ID NO:49, or has at least 90% amino acid sequence identity to a NA encoded by any one of SEQ ID Nos. 51-59. In one embodiment, the NA viral segment encodes a N2, N3, N7, or N9 NA. In one embodiment, the NA viral segment encodes a N1, N4, N5, N6, N8, N10 or N11 NA. In one embodiment, the recombinant virus has a H1, H2, H3, H5, H7, or H9 HA. In one embodiment, the isolated recombinant influenza virus is an influenza A virus. In one embodiment, the PA, PB1, PB2, NP, M, and NS viral segments have at least 85% nucleic acid sequence identity to SEQ ID Nos. 24 to 29 or 39 to 44 or encode a polypeptide having at least 80% amino acid sequence identity to a polypeptide encoded by SEQ ID Nos. 24 to 29 or 39 to 44. In one embodiment, the PB2 has I, A, L, or G. In one embodiment, the virus has one or more of PB2-I504V, PB1-M40L/G180W, PA-R401K, NP-I116L, or NS1-A30P/R118K. In one embodiment, the virus has PB2-I504V, PB1-M40L/G180W, PA-R401K, NP-I116L, and NS1-A30P/R118K.

In one embodiment, an isolated recombinant nucleic acid is provided comprising a nucleic acid sequence for an influenza virus NA viral segment that encodes a NA having a plurality of selected residues or a deletion of residues, wherein the selected NA viral segment does not encode a NA having a threonine or lysine at residue 148, and does not encode a threonine at residue 32, an aspartic acid at position 151, an asparagine at position 245, an asparagine or threonine at residue 329, a glycine at position 346, a histidine at residue 347, or include a NA having a deletion of one or more of residues 46 to 50, or any combination thereof, or wherein the selected NA viral segment does not encode a NA having a threonine or lysine at residue 148, does not encode an aspartic acid at position 151, does not encode an asparagine at position 245, does not encode a threonine at position 329, does not encode a lysine at position 344, does not encode a glycine at position 346, does not encode a histidine at residue 347, and does not encode a threonine at position 369, wherein the numbering is based on N2. In one embodiment, the selected NA viral segment does not encode a NA having a threonine or lysine at residue 148, does not encode a NA having an aspartic acid at position 151, does not encode a NA having an asparagine at position 245, does not encode a NA having a glycine at residue 346, does not encode a NA having a histidine at residue 347, or does not encode a NA having a threonine at residue 369, or any combination thereof. In one embodiment, wherein the residue at position 151 is E, N or Q. In one embodiment, the residue at position 148 is I, L, V, A, or G. In one embodiment, the residue at position 245 is S, T, I, L, A, W, Y, P, V, or G or at position 329 is S, I, L, A, W, Y, P, V, or G. In one embodiment, the residue at position 347 is G, Q, S, or T. In one embodiment, the residue at position 346 is S, T, P, Y, W, A, N, I, L, or V. In one embodiment, the residue at position 369 is K, H, R, E, P, or D. In one embodiment, the residue at position 32 is A, I, G, or L, the residue at position 147 is N or Q, the residue at position 329 is D or E, the residue at position 151 is E, N or Q, the residue at position 148 is I, L, V, A, or G, the residue at position 245 is S, T, I, L, A, W, Y, P, V, or G, the residue at position 347 is G, Q, S, or T, the residue at position 346 is S, T, P, Y, W, A, N, I, L, or V, the residue at position 369 is K, H, R, E, P, or D, or any combination thereof. In one embodiment, the residue at position 151 is E, N or Q, the residue at position 148 is I, L, V, A, or G, the residue at position 245 is S, T, I, L, A, W, Y, P, V, or G, the residue at position 347 is G, Q, S, or T, the residue at position 346 is S, T, P, Y, W, A, N, I, L, or V, the residue at position 369 is K, H, R, E, P, or D, or any combination thereof. In one embodiment, the NA has at least 90% amino acid sequence identity to SEQ ID NO:1, SEQ ID NO:2, SEQ ID NO:3, SEQ ID NO:48, or SEQ ID NO:49, or at least 90% amino acid sequence identity to a NA encoded by one of SEQ ID Nos. 51-59. In one embodiment, the NA is a N2, N3, N7, or N9. In one embodiment, the NA is a N1, N4, N5, N6, N8, N10 or N11.

In one embodiment, a method to prepare influenza virus is provided. The method includes contacting a cell with a vector for vRNA production comprising a promoter operably linked to an influenza virus PA DNA linked to a transcription termination sequence, a vector for vRNA production comprising a promoter operably linked to an influenza virus PB1 DNA linked to a transcription termination sequence, a vector for vRNA production comprising a promoter operably linked to an influenza virus PB2 DNA linked to a transcription termination sequence, a vector for vRNA production comprising a promoter operably linked to an influenza virus HA DNA linked to a transcription termination sequence, a vector for vRNA production comprising a promoter operably linked to an influenza virus NP DNA linked to a transcription termination sequence, a vector for vRNA production comprising a promoter operably linked to an influenza virus NA DNA linked to a transcription termination sequence, a vector for vRNA production comprising a promoter operably linked to an influenza virus M DNA linked to a transcription termination sequence, and a vector for vRNA production comprising a promoter operably linked to an influenza virus NS DNA linked to a transcription termination sequence, wherein the PB1, PB2, PA, NP, NS, and M DNAs in the vectors for vRNA production are from one or more influenza vaccine virus isolates, wherein the NA DNA in the vector for vRNA production encodes a NA having a plurality of selected residues or a deletion of residues, wherein the selected NA viral segment does not encode a NA having a threonine or lysine at residue 148, does not encode an aspartic acid at position 151, does not encode an asparagine at position 245, does not encode an asparagine or threonine at residue 329, does not encode a lysine at position 344, does not encode a glycine at position 346, does not encode a histidine at residue 347, and does not encode a threonine at residue 369, or any combination thereof, wherein the numbering is based on N2; and a vector for mRNA production comprising a promoter operably linked to a DNA segment encoding influenza virus PA, a vector for mRNA production comprising a promoter operably linked to a DNA segment encoding influenza virus PB1, a vector for mRNA production comprising a promoter operably linked to a DNA segment encoding influenza virus PB2, and a vector for mRNA production comprising a promoter operably linked to a DNA segment encoding influenza virus NP, and optionally a vector for mRNA production comprising a promoter operably linked to a DNA segment encoding influenza virus HA, a vector for mRNA production comprising a promoter operably linked to a DNA segment encoding influenza virus NA, a vector for mRNA production comprising a promoter operably linked to a DNA segment encoding influenza virus M1, a vector for mRNA production comprising a promoter operably linked to a DNA segment encoding influenza virus M2, or a vector for mRNA production comprising a promoter operably linked to a DNA segment encoding influenza virus NS2; in an amount effective to yield infectious influenza virus. In one embodiment, the NA has at least 90% amino acid sequence identity to SEQ ID NO:1, SEQ ID NO:2, SEQ ID NO:3, SEQ ID NO:48 or SEQ ID NO:49 or at least 90% amino acid sequence identity to a NA encoded by one of SEQ D Nos. 51-59. In one embodiment, the NA is N2, N3, N7, or N9. In one embodiment, the HA is H2 or H3. In one embodiment, the residue at position 329 is S, the residue at position 347 is G, and the residue at position 346 is V. In one embodiment, the residue at position 151 is E, N or Q, the residue at position 148 is I, L, V, A, or G, the residue at position 245 is S, T, I, L, A, V or G, the residue at position 344 is E, D, N, H or Q, the residue at position 347 is G, L, I, V, A, S, or T, the residue at position 346 is V, S, T, A, N, I, L, or V, the residue at position 369 is K, H, D, E, or R, or any combination thereof. In one embodiment, the PA, PB1, PB2, NP, M, and NS viral segments have at least 85% nucleic acid sequence identity to SEQ ID Nos. 24 to 29 or 39 to 44 or encode a polypeptide having at least 80% amino acid sequence identity to a polypeptide encoded by SEQ ID Nos. 24 to 29 or 39 to 44. Also provided is isolated virus prepared by the method.

In one embodiment, a method of immunizing an avian or a mammal is provided, comprising: administering to the avian or the mammal a composition having an effective amount of the virus. In one embodiment, the composition comprises at least one other different influenza virus. In one embodiment, the mammal is a human. In one embodiment, the composition is administered intranasally or via injection.

Further provided is a method comprising passaging the virus in eggs.

### REFERENCES

Avery's Drug Treatment: Principles and Practice of Clinical Pharmacology and Therapeutics, 3rd edition, ADIS Press, Ltd., Williams and Wilkins, Baltimore, MD (1987).

Aymard-Henry et al., Virology: A Practical Approach, Oxford IRL Press, Oxford, 119-150 (1985).

Bachmeyer, Intervirology, 5:260 (1975).

Berkow et al., eds., The Merck Manual, 16th edition, Merck & Co., Rahway, NJ (1992).

Hatta et al., Science, 293:1840 (2001).

Horimoto et al., J. Virol., 68:3120 (1994).

Horimoto et al., Vaccine, 24:3669 (2006).

Keitel et al., in Textbook of Influenza, eds. Nickolson, K. G., Webster, R. G., and Hay, A. (Blackwell, Oxford), pp. 373-390 (1998).

Kuwahara et al., Jpn. J. Infect. Dis., 71:234 (2018).

Laver & Webster, Virology, 69:511 (1976).

Neumann et al., Adv. Virus Res., 53:265 (1999).

Neumann et al., J. Gen. Virol., 83:2635 (2002).

Neumann et al., J. Virol., 71:9690 (1997).

Neumann et al., Proc. Natl. Acad. Sci. USA, 96:9345 (1999).

Neumann et al., Virology, 287:243 (2001).

Osol (ed.), Remington's Pharmaceutical Sciences, Mack Publishing Co., Easton, PA 1324-1341 (1980).

Sugawara et al., Biologicals, 30:303 (2002).

Webby & Webster et al., Science, 302:1519 (2003).

Wood & Robertson, Nat. Rev. Microbiol., 2:842 (2004).

World Health Organization TSR No. 673 (1982).

World Health Organization. Confirmed human cases of avian influenza A (H5N1). http://www.who.inticsr/disease/avian_influenza/country/en/index.html

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method to decrease influenza HA binding to cells, comprising: altering one or more residues in the HA binding pocket of HA that binds to sialic acid on allantoic membranes.

2. The method of claim 1 wherein the residue at position 98, 153 or 183 of the HA is altered based on the numbering of H3 HA.

3. The method of claim 1 wherein the residue at position 98 is F, G, I, V, T, H, W, or L; the residue at position 153 is A, G, I, V, T, or L; and/or the residue at position 183 is F, A, G, I, L, V, Y, W, P, or T; or the residue at position 98 is F, H, P, or W; the residue at position 153 is A, G, I, V, or L; and/or the residue at position 183 is F, Y, W, or P.
